(19) United States
(12) Patent Application Publication
(10) Pub. No.: US 2007/0149466 A1
(43) Pub. Date: Jun. 28, 2007

(54) Methods and related compositions for treating or preventing obesity, insulin resistance disorders, and mitochondrial-associated disorders

(76) Inventors: Michael Milburn, Cary, NC (US); Jill Milne, Brookline, MA (US); Johan Auwerx, Hindisheim (FR); Carmen Argmann, Strasbourg (FR); Marie Lagouge, Strasbourg (FR); Michelle Dipp, Cambridge, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 11/374,295

(22) Filed: Mar. 16, 2006

Related U.S. Application Data
(60) Provisional application No. 60/753,606, filed on Dec. 23, 2005.
(60) Provisional application No. 60/736,528, filed on Nov. 14, 2005.
(60) Provisional application No. 60/697,443, filed on Jul. 7, 2005.

(51) Int. Cl. A61K 31/7056; A61K 31/05

(52) U.S. Cl. 514/43; 514/733

(57) ABSTRACT

Provided herein are methods and compositions for treating or preventing metabolic disorders, such as obesity and diabetes. Methods may comprise modulating the activity or level of a sirtuin, such as SIRT1 or Sir2. Exemplary methods comprise contacting a cell with a sirtuin activating compound, such as a flavone, stilbene, flavanone, isoflavone, catechin, chalcone, tannin or anthocyanidin, or an inhibitory compound, such as nicotinamide.

### BACKGROUND

Obesity is a chronic condition that is characterized by a body mass index (BMI) over 25. Both congenital and environmental factors, such as exercise and eating habits, contribute to the disease. For instance, the hormone leptin has been shown to be involved in fat accumulation and regulating eating behavior. Several animal models of obesity result from mutations in the leptin and/or leptin receptor gene. In addition to affecting the lifestyle of an individual, obesity can lead to a number of complications and diseases, including insulin resistance, Type II diabetes, gallbladder disease, hypertension, cardiovascular disease, hyperlipidemia, sleep apnea, coronary artery disease, knee osteoarthritis, gout, infertility, breast cancer, endometrial cancer, colon cancer and lower back pain.

Diabetes is a disease that shows an acute symptom due to a remarkably high blood sugar or ketoacidosis, or as well as chronic, general metabolic abnormalities arising from a prolonged high blood sugar status or a decrease in glucose tolerance. Both congenital and environmental factors, such as exercise and eating habits, contribute to the disease. The pathogenic causes of diabetes are insulin productive disorders, secretion disorders or reductions in activities and sensitivities of the secreted insulin. Diabetes is largely grouped into the following two types: insulin-dependent diabetes mellitus (also known as Type I diabetes) and non-insulin-dependent diabetes mellitus (also known as Type II diabetes). The incidence of Type II diabetes is remarkably increased in obese patients.

Treatments for obesity are generally directed to suppressing the appetite of the subject. Whereas a number of appetite suppressants are available (diethylpropion tenuate, mazindol, orlistat, phendimetrazine, phentermine, sibutramine), these compounds may not be effective in all subjects or may be of limited efficacy. Accordingly, new treatments for obesity are needed.

A number of treatments for diabetes are well known and include oral hypoglycemic agents such as sulfonylureas that increase insulin secretion (for example, tolbutamide, chlorpropamide and glibenclamide), biguanides (for example, metformin and buformin) that increase glucose uptake and utilization and α-glucosidase inhibitors (for example, acarbose and voglibose). In addition, thiazolidinediones, such as troglitazone, rosiglitazone and pioglitazone, are used to ameliorate insulin-resistance. However, thiazolidinedione intake is usually associated with a weight gain. Thus, there is a still a need for more effective therapies for diabetes. Currently 8% and 15% of adults in the United States are diabetic or obese, respectively. With the number of individuals affected with diabetes, particularly with type II diabetes, and obesity on the increase, there is a dire need for medications that prevent and treat these conditions.

### SUMMARY

In one aspect, the invention provides methods for treating and/or preventing metabolic disorders, such as diabetes and obesity, by administering to a subject a high dose a sirtuin activator. The sirtuin activator may be administered alone or in combination with another lipid-lowering, anti-obesity and/or anti-diabetes agent. When administering a sirtuin activator as a combination with another therapeutic agent, it may be possible to administer a lower dose of the therapeutic agent than is typically required. By using a lower dose of the therapeutic agent, it is possible to reduce or eliminate undesirable side effects, such as, hypertension, elevated heart rate, etc. that may be associated with such agents. In certain embodiments, co-administration of a sirtuin activating agent with an anti-diabetic or anti-obesity drug may reduce or eliminate side effects because the activity of the sirtuin activator counteracts or prevents the side effects associated with the therapeutic agent.

In other aspects, the invention provides pharmaceutical compositions comprising a high dose of a sirtuin activator in a single dosage form. Such pharmaceutical compositions may be formulated for sustained release over at least about 6 to 48 hours or more. Also provided are neutraceuticals, such as food or beverages, that are supplemented with a sirtuin activator.

In another aspect, the invention provides methods for treating or preventing a variety diseases or disorders by administering to a subject a high dose of a sirtuin activating compound. Exemplary diseases and disorders that may be treated with a high dose of a sirtuin activating compound include, for example, diseases or disorders related to aging or stress, diabetes, obesity, neurodegenerative diseases, diseases or disorders associated with mitochondrial dysfunction, chemotherapeutic induced neuropathy, neuropathy associated with an ischemic event, ocular diseases and/or disorders, cardiovascular disease, blood clotting disorders, inflammation, and/or flushing, etc. As described further below, the methods comprise administering to a subject in need thereof a high dose of a sirtuin activating compound.

In certain aspects, a high dose of a sirtuin activating compound may be administered alone or in combination with other compounds, including other sirtuin-modulating compounds, or other therapeutic agents.

### BRIEF DESCRIPTION OF THE DRAWINGS

FIG. 1 shows examples of plant polyphenol sirtuin 1 (SIRT1) activators.

FIG. 2 shows examples of stilbene and chalcone SIRT1 activators.

FIG. 3 shows examples of flavone SIRT1 activators.

FIG. 4 shows examples of flavone SIRT1 modulators

FIG. 5 shows examples of isoflavone, flavanone and anthocyanidin SIRT1 modulators.

FIG. 6 shows examples of catechin (Flavan-3-ol) SIRT1 modulators.

FIG. 7 shows examples of free radical protective SIRT1 modulators.

FIG. 8 shows examples of SIRT1 modulators.

FIG. 9 shows examples of SIRT1 modulators.

FIG. 10 shows examples of resveratrol analog SIRT1 activators.

FIG. 11 shows further examples of resveratrol analog SIRT1 activators.

FIG. 12 shows further examples of resveratrol analog SIRT1 activators.

FIG. 13 shows examples of resveratrol analog SIRT1 modulators.

FIG. 14 shows further examples of resveratrol analog SIRT1 modulators.

FIGS. 15A-G shows examples of sirtuin activators.

FIG. 16 shows examples of sirtuin inhibitors.

FIGS. 17A-C are graphs showing that the Sirt-1 activator resveratrol (400 mg/kg/day), co-administered with a high fat diet, prevents diet-induced obesity in male C57BL/6J mice. (A) Food intake of mice expressed as kcal per 24 h. (B) Body weight evolution over time. (C) Comparison of body fat content, as analyzed by dexa scanning, at week 1 and week 12 of treatment. Values are represented as the mean±SEM (n=10). Significant differences are indicated (p value).

FIGS. 18A-C are graphs showing that the Sirt-1 activator resveratrol (400 mg/kg/day) increases energy expenditure in male C57BL/6J mice when co-administrated with a high fat diet. (A) Average oxygen consumption (V02) in 8 male mice over a period of 13 h where time 0 is 7:00 pm. The mean area under the curve is represented in the adjacent histogram. (B) Respiratory quotient (R.Q.) i.e. VCO2/VO2 (n=8). (C) Body temperature as measured at room temperature (n=10). Values are represented as the mean±SEM. Significant differences are indicated (p value).

FIGS. 19A-C are graphs showing that the Sirt-1 activator resveratrol (400 mg/kg/day) significantly diminishes the circadian locomotor activity in male C57BL/6J mice. Resting heart rate (A) and blood pressure (B) in control and resveratrol treated mice on a high fat diet. (C) Circadian activity including total ambulatory locomotor activity (top graph) and number of rears (bottom graph). The adjacent histograms represent the circadian activity measurement as area under the curve. Values are represented as the mean±SEM (n=8). Significant differences are indicated (p value).

FIGS. 20A-B are graphs showing that the Sirt-1 activator resveratrol (400 mg/kg/day) increases glucose tolerance in high fat diet fed C57BL/6J mice. (A) Blood glucose levels during an intraperitoneal glucose tolerance test (2 g glucose/kg) and (B) during an oral glucose tolerance test (2 g glucose/kg). The adjacent histograms represent the mean area under the curve and body weight of the experimental groups. Values are represented as the mean±SEM (n=5). Significant differences are indicated (p value).

FIG. 21 shows the results of an intraperitoneal glucose tolerance test in mice.

FIG. 22 shows the Sirt1 activator resveratrol (400 mg/kg/day, R400) coadministered with a high fat diet (HF) in male C57BL/6 mice enhances adaptive thermogenesis. Curves represent the body temperature of mice as measured hourly during a 6 h cold test and are presented as the mean+/−SEM, with p<0.05.

FIG. 23 shows the results of an oral glucose tolerance test in Zucker diabetic fatty rats treated with resveratrol for 42 days.

FIG. 24 shows that the Sirt-1 activator resveratrol (400 mg/kg/day), co-administered with a high fat diet, prevents diet-induced obesity in male C57BL/6J mice. The top left panel shows a graph of body weight evolution for mice in the four dietary groups over a nine week period. The top right panel shows a graph of food intake of mice in the four dietary groups expressed as kcal per 24 h. The bottom panels show comparisons of body fat content, as analyzed by dexa scanning, at week 9 of treatment for mice in the four dietary groups. Values are represented as the mean±SEM (n=10). BAT is brown adipose tissue (bottom right panel); Inguinal WAT is inguinal white adipose tissue (bottom left panel); and Retroperitoneal WAT is retroperitoneal white adipose tissue (bottom middle panel). Significant differences are indicated (p value). Animals were maintained on a control diet (C), control diet plus 400 mg/kg/day resveratrol (C+R400), high fat diet (HF) or high fat diet plus 400 mg/kg/day resveratrol (HF+R400) diets for the indicated period.

FIG. 25 shows the results of serum biochemical analysis of animals following 16 weeks on control (C), high fat (HF) or high fat plus 400 mg/kg/day resveratrol (HF+R400) diets (values are average of 10 animals from each group).

FIG. 26 shows hematoxylin and eosin staining of liver and epididymal adipose tissue sections of animals following 16 weeks on control (C), high fat (HF) or high fat plus 400 mg/kg/day resveratrol (HF+R400) diets.

FIG. 27 shows hematoxylin and eosin staining of brown adipose tissue and gastrocnemius muscle sections of animals following 16 weeks on control (C), high fat (HF) or high fat plus 400 mg/kg/day resveratrol (HF+R400) diets.

FIG. 28 shows succinate dehydrogenase staining of brown adipose tissue and gastrocnemius and soleus muscle of animals following 16 weeks on high fat (HF) or high fat plus 400 mg/kg/day resveratrol (HF+R400) diets.

FIG. 29 shows transmission electron microscopy of gastrocnemius muscle (non-oxidative fibers) of animals following 16 weeks on control (C), high fat (HF) or high fat supplemented with 400 mg/kg/day resveratrol (HF+R400) diets at 10,000 and 20,000 magnification. Inset shows schematic of muscle fiber anatomy.

FIG. 30 shows transmission electron microscopy of brown adipose tissue of animals following 16 weeks on control (C), high fat (HF) or high fat plus 400 mg/kg/day resveratrol (HF+R400) diets at 4,000 and 20,000 magnifications.

FIG. 31 shows Sirt1 mRNA level measured in the brown adipose tissue, liver and muscle of animals treated with control (C), high fat (HF) or high fat plus 400 mg/kg/day resveratrol (HF+R400) diets (values are average of 6 animals from each group). Values are expressed relative to the housekeeping gene 18s and then expressed relative to chow diet (arbitrarily equal to 1).

FIG. 32 shows relative gene expression of PEPCK, glucose-6-phosphatase, Foxol, PGC1-alpha and Sirt1 in liver, brown adipose tissue and muscle on either control (unshaded), high fat (light shading) or high fat plus 400 mg/kg resveratrol (dark shading) diets (n=pool of 6 animals for each condition).

FIG. 33 shows the results of an immunoblot indicating that resveratrol increases PGC1 alpha deacetylation. IP is immunoprecipitation; IB is immunoblot; HF is high fat diet; and HF+R400 is high fat diet plus 400 mg/kg resveratrol.

FIG. 34 shows an analysis of the fecal lipid content for mice fed diets of chow (C), high fat (HF), or high fat plus 400 mg/kg resveratrol (HF+R400). The left panel shows the total fecal weight per mouse. The right panel shows the amount of cholesterol and triglycerides excreted by the animals in the different diet groups.

FIG. 35 are graphs showing that the Sirt-1 activator resveratrol (400 mg/kg/day), co-administered with a high fat diet, prevents diet-induced obesity in male C57BL/6J mice. Left Panel: Body weight evolution over time (graphs from top to bottom are: HF, HF+R400, C, and C+R400). Right Panel: Area under the curve for the graphs shown in the panel to the left.

FIG. 36 is a diagram illustrating the treadmill endurance protocol for mice fed chow (top line) and high fat (bottom line) diets.

FIG. 37 is a graph showing the results of the endurance test for mice fed chow or high fat diets. Each line on the graph shows an individual animal tested using the endurance protocol illustrated in FIG. 33.

FIG. 38 shows the effect of resveratrol on insulin sensitivity as measured by hyperinsulinemic (18 mU/kg/min) euglycemic (5.5 mmol/l) clamp. The left hand panel shows glucose infusion rates (GIR) for groups of animals following 14 weeks on either a control diet (C), control diet plus 400 mg/kg resveratrol (C+R400), high fat diet (HF) or high fat diet plus 400 mg/kg resvertrol (HF+R400). The right hand panel shows average GIR at steady state clamp.

### DETAILED DESCRIPTION

Definitions

As used herein, the following terms and phrases shall have the meanings set forth below. Unless defined otherwise, all technical and scientific terms used herein have the same meaning as commonly understood to one of ordinary skill in the art.

The singular forms “a,” “an,” and “the” include plural reference unless the context clearly dictates otherwise.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

A “form that is naturally occurring”, when referring to a compound, means a compound that is in a form, e.g., a composition, in which it can be found naturally. For example, since resveratrol can be found in red wine, it is present in red wine in a form that is naturally occurring. A compound is not in a form that is naturally occurring if, e.g., the compound has been purified and separated from at least some of the other molecules that are found with the compound in nature. A “naturally occurring compound” refers to a compound that can be found in nature, i.e., a compound that has not been designed by man. A naturally occurring compound may have been made by man or by nature.

“Sirtuin modulator” refers to a compound that up regulates (e.g., activate or stimulate), down regulates (e.g., inhibit or suppress) or otherwise changes a functional property or biological activity of a sirtuin protein. Sirtuin modulators may act to modulate a sirtuin protein either directly or indirectly. In certain embodiments, a sirtuin modulator may be a sirtuin activator or a sirtuin inhibitor.

The terms “sirtuin activator” or “sirtuin activating compound” refer to a compound that increases the level of a sirtuin protein and/or increases at least one activity of a sirtuin protein. In an exemplary embodiment, a sirtuin activator may increase at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells. Exemplary sirtuin activating compounds include, for example, compounds having a formula selected from the group of formulas 1-25, 30, 32-65, and 69-88.

A “high dose of a sirtuin activating compound” refers to a quantity of a sirtuin activator having a sirtuin activating effect equal to or greater than the sirtuin activating effect of 18 mg/kg resveratrol (e.g., in humans). In certain embodiments, a high dose of a sirtuin activating compound refers to a quantity of a sirtuin activator having a sirtuin activating effect equal to or greater than the sirtuin activating effect of 18 mg/kg of resveratrol which is administered (i) orally, (ii) released from a sustained release form over 6 to 48 hours, and/or (iii) for an equivalent amount of time. In certain embodiments, a high dose of a sirtuin activating compound refers to a quantity of a sirtuin activator having a sirtuin activating effect equal to or greater than the sirtuin activating effect of at least about 20, 25, 30, 35,40, 50, 60, 75, 100, 150 mg/kg, or more, or resveratrol.

“Sirtuin activating effect” refers to the level or extent of one or more therapeutic effects obtained upon administration of a high dose of a sirtuin activating compound. Therapeutic effects include, for example, (i) preventing or inhibiting weight gain upon consuming a diet having an increased fat and/or calorie content without an increase in activity, heart rate, and/or blood pressure; and/or (ii) improved blood glucose levels. Such therapeutic effects include, for example, the therapeutic effects illustrated in the Examples.

“Sirtuin inhibitor” refers to a compound that decreases the level of a sirtuin protein and/or decreases at least one activity of a sirtuin protein. In an exemplary embodiment, a sirtuin inhibitor may decrease at least one biological activity of a sirtuin protein by at least about 10%, 25%, 50%, 75%, 100%, or more. Exemplary biological activities of sirtuin proteins include deacetylation, e.g., of histones and p53; extending lifespan; increasing genomic stability; silencing transcription; and controlling the segregation of oxidized proteins between mother and daughter cells.

“Sirtuin protein” refers to a member of the sirtuin deacetylase protein family or preferably to the Sir2 family, which include yeast Sir2 (GenBank Accession No. P53685), C. elegans Sir-2.1 (GenBank Accession No. NP_{—}501912), and human SIRT1 (GenBank Accession No. NM_{—}012238 and NP_{—}036370 (or AF083106)) and SIRT2 (GenBank Accession No. NM_{—}030593 and AF083107) proteins. Other family members include the four additional yeast Sir2-like genes termed “HST genes” (homologues of Sir two) HST1, HST2, HST3 and HST4, and the five other human homologues hSIRT3, hSIRT4, hSIRT5, hSIRT6 and hSIRT7 (Brachmann et al. (1995) Genes Dev. 9:2888 and Frye et al. (1999) BBRC 260:273). Preferred sirtuins are those that share more similarities with SIRT1, i.e., hSIRT1, and/or Sir2 than with SIRT2, such as those members having at least part of the N-terminal sequence present in SIRT1 and absent in SIRT2 such as SIRT3 has.

“SIRT1 protein” refers to a member of the sir2 family of sirtuin deacetylases. In one embodiment, a SIRT1 protein includes yeast Sir2 (GenBank Accession No. P53685), C. elegans Sir-2.1 (GenBank Accession No. NP_{—}501912), human SIRT1 (GenBank Accession No. NM_{—}012238 and NP_{—}036370 (or AF083106)), human SIRT2 (GenBank Accession No. NM_{—}012237, NM_{—}030593, NP_{—}036369, NP_{—}085096, and AF083107) proteins, and equivalents and fragments thereof. In another embodiment, a SIRT1 protein includes a polypeptide comprising a sequence consisting of, or consisting essentially of, the amino acid sequence set forth in GenBank Accession Nos. NP_{—}036370, NP_{—}501912, NP_{—}085096, NP_{—}036369, and P53685. SIRT1 proteins include polypeptides comprising all or a portion of the amino acid sequence set forth in GenBank Accession Nos. NP_{—}036370, NP_{—}501912, NP_{—}085096, NP_{—}036369, and P53685; the amino acid sequence set forth in GenBank Accession Nos. NP_{—}036370, NP_{—}501912, NP_{—}085096, NP_{—}036369, and P53685 with 1 to about 2, 3, 5, 7, 10, 15, 20, 30, 50, 75 or more conservative amino acid substitutions; an amino acid sequence that is at least 60%, 70%, 80%, 90%, 95%, 96%, 97%, 98%, or 99% identical to GenBank Accession Nos. NP_{—}036370, NP_{—}501912, NP_{—}085096, NP_{—}036369, and P53685 and functional fragments thereof. Polypeptides of the invention also include homologs (e.g., orthologs and paralogs), variants, or fragments, of GenBank Accession Nos. NP_{—}036370, NP_{—}501912, NP_{—}085096, NP_{—}036369, and P53685.

“Biologically active portion of a sirtuin” refers to a portion of a sirtuin protein having a biological activity, such as the ability to deacetylate. Biologically active portions of sirtuins may comprise the core domain of sirtuins. For example, amino acids 62-293 of the SIRT1 protein sequence, which are encoded by nucleotides 237 to 932 of the SIRT1 nucleic acid sequence, encompass the NAD⁺ binding domain and the substrate binding domain. Therefore, this region is sometimes referred to as the core domain. Other biologically active portions of SIRT1, also sometimes referred to as core domains, include about amino acids 261 to 447 of the SIRT1 protein sequence, which are encoded by nucleotides 834 to 1394 of the SIRT1 nucleic acid sequence; about amino acids 242 to 493 of the SIRT1 protein sequence, which are encoded by nucleotides 777 to 1532 of the SIRT1 nucleic acid sequence; or about amino acids 254 to 495 of the SIRT1 protein sequence, which are encoded by nucleotides 813 to 1538 of the SIRT1 nucleic acid sequence.

A “direct activator” of a sirtuin is a molecule that activates a sirtuin by binding to it. A “direct inhibitor” of a sirtuin is a molecule that inhibits a sirtuin by binding to it.

The terms “comprise” and “comprising” are used in the inclusive, open sense, meaning that additional elements may be included.

The term “including” is used to mean “including but not limited to”. “Including” and “including but not limited to” are used interchangeably.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a nonnatural arrangement.

A “patient”, “subject” or “host” refers to either a human or a non-human animal. Non-human animals include farm animals (e.g., cows, horses, pigs, sheep, goats) and companion animals (e.g., dogs, cats).

The term “substantially homologous” when used in connection with amino acid sequences, refers to sequences which are substantially identical to or similar in sequence with each other, giving rise to a homology of conformation and thus to retention, to a useful degree, of one or more biological (including immunological) activities. The term is not intended to imply a common evolution of the sequences.

The term “modulation” is art-recognized and refers to up regulation (i.e., activation or stimulation), down regulation (i.e., inhibition or suppression) of a response, or the two in combination or apart.

The term “prophylactic” or “therapeutic” treatment is art-recognized and refers to administration of a drug to a host. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

The term “mammal” is known in the art, and exemplary mammals include humans, primates, bovines, porcines, canines, felines, and rodents (e.g., mice and rats).

The term “bioavailable” when referring to a compound is art-recognized and refers to a form of a compound that allows for it, or a portion of the amount of compound administered, to be absorbed by, incorporated to, or otherwise physiologically available to a subject or patient to whom it is administered.

The term “pharmaceutical” refers to any compound having a pharmacological effect. For example, the term pharmaceutical encompasses natural compounds as well as nonnatural compounds that have a pharmacological effect.

The term “pharmaceutically-acceptable salts” is art-recognized and refers to the relatively non-toxic, inorganic and organic acid addition salts of compounds, as well as solvates, co-crystals, polymorphs and the like of the salts, including, for example, those contained in compositions described herein.

The term “pharmaceutically acceptable carrier” is art-recognized and refers to a pharmaceutically-acceptable material, composition or vehicle, such as a liquid or solid filler, diluent, excipient, solvent or encapsulating material. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “systemic administration,” “administered systemically,” “peripheral administration” and “administered peripherally” are art-recognized and refer to the administration of a subject composition, therapeutic or other material other than directly into the central nervous system, such that it enters the patient's system and, thus, is subject to metabolism and other like processes.

The terms “parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articulare, subcapsular, subarachnoid, intraspinal, and intrasternal injection and infusion.

“Transcriptional regulatory sequence” is a generic term used throughout the specification to refer to DNA sequences, such as initiation signals, enhancers, and promoters, which induce or control transcription of protein coding sequences with which they are operable linked. In preferred embodiments, transcription of one of the recombinant genes is under the control of a promoter sequence (or other transcriptional regulatory sequence) which controls the expression of the recombinant gene in a cell-type which expression is intended. It will also be understood that the recombinant gene can be under the control of transcriptional regulatory sequences which are the same or which are different from those sequences which control transcription of the naturally-occurring forms of genes as described herein.

A “vector” is a self-replicating nucleic acid molecule that transfers an inserted nucleic acid molecule into and/or between host cells. The term includes vectors that function primarily for insertion of a nucleic acid molecule into a cell, replication of vectors that function primarily for the replication of nucleic acid, and expression vectors that function for transcription and/or translation of the DNA or RNA. Also included are vectors that provide more than one of the above functions. As used herein, “expression vectors” are defined as polynucleotides which, when introduced into an appropriate host cell, can be transcribed and translated into a polypeptide(s). An “expression system” usually connotes a suitable host cell comprised of an expression vector that can function to yield a desired expression product.

An “indicator of mitochondrial function” is any parameter that is indicative of mitochondrial function that can be measured by one skilled in the art. In certain embodiments, the indicator of mitochondrial function is a mitochondrial electron transport chain enzyme, a Krebs cycle enzyme, a mitochondrial matrix component, a mitochondrial membrane component or an ATP biosynthesis factor. In other embodiments, the indicator of mitochondrial function is mitochondrial number per cell or mitochondrial mass per cell. In other embodiments, the indicator of mitochondrial function is an ATP biosynthesis factor. In other embodiments, the indicator of mitochondrial function is the amount of ATP per mitochondrion, the amount of ATP per unit mitochondrial mass, the amount of ATP per unit protein or the amount of ATP per unit mitochondrial protein. In other embodiments, the indicator of mitochondrial function comprises free radical production. In other embodiments, the indicator of mitochondrial function comprises a cellular response to elevated intracellular calcium. In other embodiments, the indicator of mitochondrial function is the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the realtive or absolute amount of mitochondrial DNA per cell in the patient.

“Improving mitochondrial function” or “altering mitochondrial function” may refer to (a) substantially (e.g., in a statistically significant manner, and preferably in a manner that promotes a statistically significant improvement of a clinical parameter such as prognosis, clinical score or outcome) restoring to a normal level at least one indicator of glucose responsiveness in cells having reduced glucose responsiveness and reduced mitochondrial mass and/or impaired mitochondrial function; or (b) substantially (e.g., in a statistically significant manner, and preferably in a manner that promotes a statistically significant improvement of a clinical parameter such as prognosis, clinical score or outcome) restoring to a normal level, or increasing to a level above and beyond normal levels, at least one indicator of mitochondrial function in cells having impaired mitochondrial function, or in cells having normal mitochondrial function, respectively. Improved or altered mitochondrial function may result from changes in extramitochondrial structures or events, as well as from mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

“Impaired mitochondrial function” may include a full or partial decrease, inhibition, diminution, loss or other impairment in the level and/or rate of any respiratory, metabolic or other biochemical or biophysical activity in some or all cells of a biological source. As non-limiting examples, markedly impaired electron transport chain (ETC) activity may be related to impaired mitochondrial function, as may be generation of increased reactive oxygen species (ROS) or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of mitochondrial function. These and other non-limiting examples of impaired mitochondrial function are described in greater detail below.

“Treating” a condition or disease refers to curing as well as ameliorating at least one symptom of the condition or disease.

The term “cis” is art-recognized and refers to the arrangement of two atoms or groups around a double bond such that the atoms or groups are on the same side of the double bond. Cis configurations are often labeled as (Z) configurations.

The term “trans” is art-recognized and refers to the arrangement of two atoms or groups around a double bond such that the atoms or groups are on the opposite sides of a double bond. Trans configurations are often labeled as (E) configurations.

The term “covalent bond” is art-recognized and refers to a bond between two atoms where electrons are attracted electrostatically to both nuclei of the two atoms, and the net effect of increased electron density between the nuclei counterbalances the internuclear repulsion. The term covalent bond includes coordinate bonds when the bond is with a metal ion.

The term “therapeutic agent” is art-recognized and refers to any compound that is a biologically, physiologically, or pharmacologically active substance that acts locally or systemically in a subject. The term also means any substance intended for use in the diagnosis, cure, mitigation, treatment or prevention of disease or in the enhancement of desirable physical or mental development and/or conditions in an animal or human.

The term “therapeutic effect” is art-recognized and refers to a local or systemic effect in animals, particularly mammals, and more particularly humans caused by a pharmacologically active substance. The phrase “therapeutically-effective amount” means that amount of such a substance that produces some desired local or systemic effect at a reasonable benefit/risk ratio applicable to any treatment. The therapeutically effective amount of such substance will vary depending upon the subject and disease condition being treated, the weight and age of the subject, the severity of the disease condition, the manner of administration and the like, which can readily be determined by one of ordinary skill in the art. For example, certain compositions described herein may be administered in a sufficient amount to produce a desired effect on metabolic disorders or diabetes or complications thereof, at a reasonable benefit/risk ratio applicable to such treatment.

The term “synthetic” is art-recognized and refers to production by in vitro chemical or enzymatic synthesis.

The term “meso compound” is art-recognized and refers to a chemical compound which has at least two chiral centers but is achiral due to a plane or point of symmetry.

The term “chiral” is art-recognized and refers to molecules which have the property of non-superimposability of the mirror image partner, while the term “achiral” refers to molecules which are superimposable on their mirror image partner. A “prochiral molecule” is a molecule which has the potential to be converted to a chiral molecule in a particular process.

The term “stereoisomers” is art-recognized and refers to compounds which have identical chemical constitution, but differ with regard to the arrangement of the atoms or groups in space. In particular, “enantiomers” refer to two stereoisomers of a compound which are non-superimposable mirror images of one another. “Diastereomers”, on the other hand, refers to stereoisomers with two or more centers of dissymmetry and whose molecules are not mirror images of one another.

Furthermore, a “stereoselective process” is one which produces a particular stereoisomer of a reaction product in preference to other possible stereoisomers of that product. An “enantioselective process” is one which favors production of one of the two possible enantiomers of a reaction product.

The term “regioisomers” is art-recognized and refers to compounds which have the same molecular formula but differ in the connectivity of the atoms. Accordingly, a “regioselective process” is one which favors the production of a particular regioisomer over others, e.g., the reaction produces a statistically significant increase in the yield of a certain regioisomer.

The term “epimers” is art-recognized and refers to molecules with identical chemical constitution and containing more than one stereocenter, but which differ in configuration at only one of these stereocenters.

The term “ED₅₀” is art-recognized. In certain embodiments, ED₅₀ means the dose of a drug which produces 50% of its maximum response or effect, or alternatively, the dose which produces a pre-determined response in 50% of test subjects or preparations. The term “LD₅₀” is art-recognized. In certain embodiments, LD₅₀ means the dose of a drug which is lethal in 50% of test subjects. The term “therapeutic index” is an art-recognized term which refers to the therapeutic index of a drug, defined as LD₅₀/ED₅₀.

The term “structure-activity relationship” or “SAR” is art-recognized and refers to the way in which altering the molecular structure of a drug or other compound alters its biological activity, e.g., its interaction with a receptor, enzyme, nucleic acid or other target and the like.

The term “aliphatic” is art-recognized and refers to a linear, branched, cyclic alkane, alkene, or alkyne. In certain embodiments, aliphatic groups in the present compounds are linear or branched and have from 1 to about 20 carbon atoms.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

The term “aralkyl” is art-recognized and refers to an alkyl group substituted with an aryl group (e.g., an aromatic or heteroaromatic group).

The terms “alkenyl” and “alkynyl” are art-recognized and refer to unsaturated aliphatic groups analogous in length and possible substitution to the alkyls described above, but that contain at least one double or triple bond respectively.

Unless the number of carbons is otherwise specified, “lower alkyl” refers to an alkyl group, as defined above, but having from one to about ten carbons, alternatively from one to about six carbon atoms in its backbone structure. Likewise, “lower alkenyl” and “lower alkynyl” have similar chain lengths.

The term “heteroatom” is art-recognized and refers to an atom of any element other than carbon or hydrogen. Illustrative heteroatoms include boron, nitrogen, oxygen, phosphorus, sulfur and selenium.

The term “aryl” is art-recognized and refers to 5-, 6- and 7-membered single-ring aromatic groups that may include from zero to four heteroatoms, for example, benzene, naphtalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls. In compounds 77-88, “aryl” is intended to refer to both carbocyclic and heterocyclic aromatic groups.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” are art-recognized and refer to 3- to about 10-membered ring structures, alternatively 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthene, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” are art-recognized and refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle” is art-recognized and refers to an aromatic or non-aromatic ring in which each atom of the ring is carbon.

The term “nitro” is art-recognized and refers to —NO₂; the term “halogen” is art-recognized and refers to —F, —Cl, —Br or —I; the term “sulfhydryl” is art-recognized and refers to —SH; the term “hydroxyl” means —OH; and the term “sulfonyl” is art-recognized and refers to —SO₂—. “Halide” designates the corresponding anion of the halogens, and “pseudohalide” has the definition set forth on 560 of “Advanced Inorganic Chemistry” by Cotton and Wilkinson.

The terms “amine” and “amino” are art-recognized and refer to both unsubstituted and substituted amines, e.g., a moiety that may be represented by the general formulas: 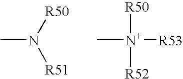
wherein R50, R51 and R52 each independently represent a hydrogen, an alkyl, an alkenyl, —(CH₂)ₘ—R61, or R50 and R51, taken together with the N atom to which they are attached complete a heterocycle having from 4 to 8 atoms in the ring structure; R61 represents an aryl, a cycloalkyl, a cycloalkenyl, a heterocycle or a polycycle; and m is zero or an integer in the range of 1 to 8. In certain embodiments, only one of R50 or R51 may be a carbonyl, e.g., R50, R51 and the nitrogen together do not form an imide. In other embodiments, R50 and R51 (and optionally R52) each independently represent a hydrogen, an alkyl, an alkenyl, or —(CH₂)ₘ—R61. Thus, the term “alkylamine” includes an amine group, as defined above, having a substituted or unsubstituted alkyl attached thereto, i.e., at least one of R50 and R51 is an alkyl group.

The term “acylamino” is art-recognized and refers to a moiety that may be represented by the general formula: 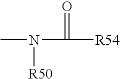
wherein R50 is as defined above, and R54 represents a hydrogen, an alkyl, an alkenyl or —(CH₂)ₘ—R61, where m and R61 are as defined above.

The term “amido” is art recognized as an amino-substituted carbonyl and includes a moiety that may be represented by the general formula: 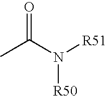
wherein R50 and R51 are as defined above. Certain embodiments of amides may not include imides which may be unstable.

The term “alkylthio” refers to an alkyl group, as defined above, having a sulfur radical attached thereto. In certain embodiments, the “alkylthio” moiety is represented by one of —S-alkyl, —S-alkenyl, —S-alkynyl, and —S—(CH₂)ₘ—R61, wherein m and R61 are defined above. Representative alkylthio groups include methylthio, ethyl thio, and the like.

The term “carbonyl” is art recognized and includes such moieties as may be represented by the general formulas: 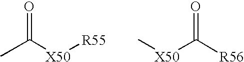
wherein X50 is a bond or represents an oxygen or a sulfur, and R55 and R56 represents a hydrogen, an alkyl, an alkenyl, —(CH₂)ₘ—R61 or a pharmaceutically acceptable salt, R56 represents a hydrogen, an alkyl, an alkenyl or —(CH₂)ₘ—R61, where m and R61 are defined above. Where X50 is an oxygen and R55 or R56 is not hydrogen, the formula represents an “ester”. Where X50 is an oxygen, and R55 is as defined above, the moiety is referred to herein as a carboxyl group, and particularly when R55 is a hydrogen, the formula represents a “carboxylic acid”. Where X50 is an oxygen, and R56 is hydrogen, the formula represents a “formate”. In general, where the oxygen atom of the above formula is replaced by sulfur, the formula represents a “thiolcarbonyl” group. Where X50 is a sulfur and R55 or R56 is not hydrogen, the formula represents a “thiolester.” Where X50 is a sulfur and R55 is hydrogen, the formula represents a “thiolcarboxylic acid.” Where X50 is a sulfur and R56 is hydrogen, the formula represents a “thiolformate.” On the other hand, where X50 is a bond, and R55 is not hydrogen, the above formula represents a “ketone” group. Where X50 is a bond, and R55 is hydrogen, the above formula represents an “aldehyde” group.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R61, where m and R61 are described above.

The term “sulfonate” is art recognized and refers to a moiety that may be represented by the general formula: 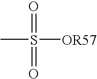
in which R57 is an electron pair, hydrogen, alkyl, cycloalkyl, or aryl.

The term “sulfate” is art recognized and includes a moiety that may be represented by the general formula: 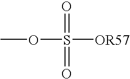
in which R57 is as defined above.

The term “sulfonamido” is art recognized and includes a moiety that may be represented by the general formula: 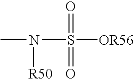
in which R50 and R56 are as defined above.

The term “sulfamoyl” is art-recognized and refers to a moiety that may be represented by the general formula: 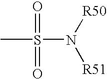
in which R50 and R51 are as defined above.

The term “sulfonyl” is art-recognized and refers to a moiety that may be represented by the general formula: 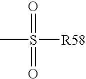
in which R58 is one of the following: hydrogen, alkyl, alkenyl, alkynyl, cycloalkyl, heterocyclyl, aryl or heteroaryl.

The term “sulfoxido” is art-recognized and refers to a moiety that may be represented by the general formula: 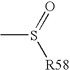
in which R58 is defined above.

The term “phosphoryl” is art-recognized and may in general be represented by the formula: 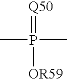
wherein Q50 represents S or O, and R59 represents hydrogen, a lower alkyl or an aryl. When used to substitute, e.g., an alkyl, the phosphoryl group of the phosphorylalkyl may be represented by the general formulas: 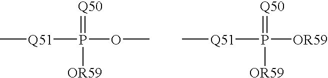
wherein Q50 and R59, each independently, are defined above, and Q51 represents O, S or N. When Q50 is S, the phosphoryl moiety is a “phosphorothioate”.

The term “phosphoramidite” is art-recognized and may be represented in the general formulas: 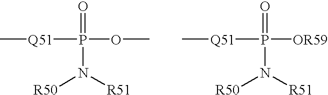
wherein Q51, R50, R51 and R59 are as defined above.

The term “phosphonamidite” is art-recognized and may be represented in the general formulas: 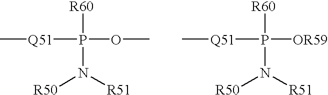
wherein Q51, R50, R51 and R59 are as defined above, and R60 represents a lower alkyl or an aryl.

Analogous substitutions may be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls. The definition of each expression, e.g. alkyl, m, n, and the like, when it occurs more than once in any structure, is intended to be independent of its definition elsewhere in the same structure.

The term “selenoalkyl” is art-recognized and refers to an alkyl group having a substituted seleno group attached thereto. Exemplary “selenoethers” which may be substituted on the alkyl are selected from one of —Se-alkyl, —Se-alkenyl, —Se-alkynyl, and —Se—(CH₂)ₘ—R61, m and R61 being defined above.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

Certain compounds contained in compositions described herein may exist in particular geometric or stereoisomeric forms. In addition, compounds may also be optically active. Contemplated herein are all such compounds, including cis- and trans-isomers, R- and S-enantiomers, diastereomers, (D)-isomers, (L)-isomers, the racemic mixtures thereof, and other mixtures thereof. Additional asymmetric carbon atoms may be present in a substituent such as an alkyl group. All such isomers, as well as mixtures thereof, are encompassed herein.

If, for instance, a particular enantiomer of a compound is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

It is understood that compounds disclosed herein are intended to represent the compound itself, along with solvates, co-crystals, polymorphs and the like of the compound.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. Heteroatoms such as nitrogen may have hydrogen substituents and/or any permissible substituents of organic compounds described herein which satisfy the valences of the heteroatoms. Compounds are not intended to be limited in any manner by the permissible substituents of organic compounds.

The chemical elements are identified in accordance with the Periodic Table of the Elements, CAS version, Handbook of Chemistry and Physics, 67th Ed., 1986-87, inside cover.

The term “protecting group” is art-recognized and refers to temporary substituents that protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed by Greene and Wuts in Protective Groups in Organic Synthesis (2^{nd} ed., Wiley: New York, 1991).

The term “hydroxyl-protecting group” is art-recognized and refers to those groups intended to protect a hydrozyl group against undesirable reactions during synthetic procedures and includes, for example, benzyl or other suitable esters or ethers groups known in the art.

The term “carboxyl-protecting group” is art-recognized and refers to those groups intended to protect a carboxylic acid group, such as the C-terminus of an amino acid or peptide or an acidic or hydroxyl azepine ring substituent, against undesirable reactions during synthetic procedures and includes. Examples for protecting groups for carboxyl groups involve, for example, benzyl ester, cyclohexyl ester, 4-nitrobenzyl ester, t-butyl ester, 4-pyridylmethyl ester, and the like.

The term “amino-blocking group” is art-recognized and refers to a group which will prevent an amino group from participating in a reaction carried out on some other functional group, but which can be removed from the amine when desired. Such groups are discussed by in Ch. 7 of Greene and Wuts, cited above, and by Barton, Protective Groups in Organic Chemistry ch. 2 (McOmie, ed., Plenum Press, New York, 1973). Examples of suitable groups include acyl protecting groups such as, to illustrate, formyl, dansyl, acetyl, benzoyl, trifluoroacetyl, succinyl, methoxysuccinyl, benzyl and substituted benzyl such as 3,4-dimethoxybenzyl, o-nitrobenzyl, and triphenylmethyl; those of the formula —COOR where R includes such groups as methyl, ethyl, propyl, isopropyl, 2,2,2-trichloroethyl, 1-methyl-1-phenylethyl, isobutyl, t-butyl, t-amyl, vinyl, allyl, phenyl, benzyl, p-nitrobenzyl, o-nitrobenzyl, and 2,4-dichlorobenzyl; acyl groups and substituted acyl such as formyl, acetyl, chloroacetyl, dichloroacetyl, trichloroacetyl, trifluoroacetyl, benzoyl, and p-methoxybenzoyl; and other groups such as methanesulfonyl, p-toluenesulfonyl, p-bromobenzenesulfonyl, p-nitrophenylethyl, and p-toluenesulfonyl-aminocarbonyl. Preferred amino-blocking groups are benzyl (—CH₂C₆H₅), acyl [C(O)R1] or SiR1₃ where R1 is C₁-C₄ alkyl, halomethyl, or 2-halo-substituted-(C₂-C₄ alkoxy), aromatic urethane protecting groups as, for example, carbonylbenzyloxy (Cbz); and aliphatic urethane protecting groups such as t-butyloxycarbonyl (Boc) or 9-fluorenylmethoxycarbonyl (FMOC).

The definition of each expression, e.g. lower alkyl, m, n, p and the like, when it occurs more than once in any structure, is intended to be independent of its definition elsewhere in the same structure.

The term “electron-withdrawing group” is art-recognized, and refers to the tendency of a substituent to attract valence electrons from neighboring atoms, i.e., the substituent is electronegative with respect to neighboring atoms. A quantification of the level of electron-withdrawing capability is given by the Hammett sigma (σ) constant. This well known constant is described in many references, for instance, March, Advanced Organic Chemistry 251-59 (McGraw Hill Book Company: New York, 1977). The Hammett constant values are generally negative for electron donating groups (σ(P)=−0.66 for NH₂) and positive for electron withdrawing groups (σ(P)=0.78 for a nitro group), σ(P) indicating para substitution. Exemplary electron-withdrawing groups include nitro, acyl, formyl, sulfonyl, trifluoromethyl, cyano, chloride, and the like. Exemplary electron-donating groups include amino, methoxy, and the like.

I. Exemplary Methods and Compositions for Increasing the Activity or Protein Level of Sirtuin Proteins

In one embodiment, exemplary sirtuin-activating compounds are those described in Howitz et al. (2003) Nature 425: 191 and include, for example, resveratrol (3,5,4′-Trihydroxy-trans-stilbene), butein (3,4,2′,4′-Tetrahydroxychalcone), piceatannol (3,5,3′,4′-Tetrahydroxy-trans-stilbene), isoliquiritigenin (4,2′,4′-Trihydroxychalcone), fisetin (3,7,3′,4′-Tetrahyddroxyflavone), quercetin (3,5,7,3′,4′-Pentahydroxyflavone), Deoxyrhapontin (3,5-Dihydroxy-4′-methoxystilbene 3-O-β-D-glucoside); trans-Stilbene; Rhapontin (3,3′,5-Trihydroxy-4′-methoxystilbene 3-O-β-D-glucoside); cis-Stilbene; Butein (3,4,2′,4′-Tetrahydroxychalcone); 3,4,2′4′6′-Pentahydroxychalcone; Chalcone; 7,8,3′,4′-Tetrahydroxyflavone; 3,6,2′,3′-Tetrahydroxyflavone; 4′-Hydroxyflavone; 5,4′-Dihydroxyflavone; 5,7-Dihydroxyflavone; Morin (3,5,7,2′,4′-Pentahydroxyflavone); Flavone; 5-Hydroxyflavone; (−)-Epicatechin (Hydroxy Sites: 3,5,7,3′,4′); (−)-Catechin (Hydroxy Sites: 3,5,7,3′,4′); (−)-Gallocatechin (Hydroxy Sites: 3,5,7,3′,4′,5′) (+)-Catechin (Hydroxy Sites: 3,5,7,3′,4′); 5,7,3′,4′,5′-pentahydroxyflavone; Luteolin (5,7,3′,4′-Tetrahydroxyflavone); 3,6,3′,4′-Tetrahydroxyflavone; 7,3′,4′,5′-Tetrahydroxyflavone; Kaempferol (3,5,7,4′-Tetrahydroxyflavone); 6-Hydroxyapigenin (5,6,7,4′-Tetrahydoxyflavone); Scutellarein); Apigenin (5,7,4′-Trihydroxyflavone); 3,6,2′,4′-Tetrahydroxyflavone; 7,4′-Dihydroxyflavone; Daidzein (7,4′-Dihydroxyisoflavone); Genistein (5,7,4′-Trihydroxyflavanone); Naringenin (5,7,4′-Trihydroxyflavanone); 3,5,7,3′,4′-Pentahydroxyflavanone; Flavanone; Pelargonidin chloride (3,5,7,4′-Tetrahydroxyflavylium chloride); Hinokitiol (b-Thujaplicin; 2-hydroxy-4-isopropyl-2,4,6-cycloheptatrien-1-one); L-(+)-Ergothioneine ((S)-a-Carboxy-2,3-dihydro-N,N,N-trimethyl-2-thioxo-1H-imidazole-4-ethanaminium inner salt); Caffeic Acid Phenyl Ester; MCI-186 (3-Methyl-1-phenyl-2-pyrazolin-5-one); HBED (N,N′-Di-(2-hydroxybenzyl)ethylenediamine-N,N′-diacetic acid.H2O); Ambroxol (trans-4-(2-Amino-3,5-dibromobenzylamino)cyclohexane.HCl; and U-83836E ((−)-2-((4-(2,6-di-1-Pyrrolidinyl-4-pyrimidinyl)-1-piperzainyl)methyl)-3,4-dihydro-2,5,7,8-tetramethyl-2H-1-benzopyran-6-ol.2HCl). Analogs and derivatives thereof can also be used.

Other sirtuin-activating compounds may have any of formulas 1-25, 30, 32-65, and 69-88 below. In one embodiment, a sirtuin-activating compound is a stilbene or chalcone compound of formula 1: 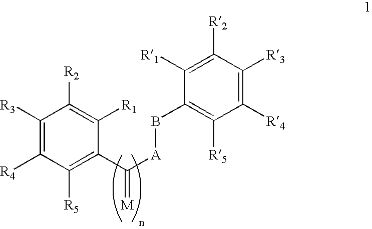
wherein, independently for each occurrence,

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents O, NR, or S;

A-B represents a bivalent alkyl, alkenyl, alkynyl, amido, sulfonamido, diazo, ether, alkylamino, alkylsulfide, hydroxylamine, or hydrazine group; and

n is 0 or 1.

In a further embodiment, a sirtuin-activating compound is a compound of formula 1 and the attendant definitions, wherein n is 0. In a further embodiment, a sirtuin-activating compound is a compound of formula 1 and the attendant definitions, wherein n is 1. In a further embodiment, a sirtuin-activating compound is a compound of formula 1 and the attendant definitions, wherein A-B is ethenyl. In a further embodiment, a sirtuin-activating compound is a compound of formula 1 and the attendant definitions, wherein A-B is —CH₂CH(Me)CH(Me)CH₂—. In a further embodiment, a sirtuin-activating compound is a compound of formula 1 and the attendant definitions, wherein M is O. In a further embodiment, the methods comprises a compound of formula 1 and the attendant definitions, wherein R₁, R₂, R₃, R₄, R₅, R′₁, R′₂, R′₃, R′₄, and R′₅ are H. In a further embodiment, a sirtuin-activating compound is a compound of formula 1 and the attendant definitions, wherein R₂, R₄, and R′₃ are OH. In a further embodiment, a sirtuin-activating compound is a compound of formula 1 and the attendant definitions, wherein R₂, R₄, R′₂ and R′₃ are OH. In a further embodiment, a sirtuin-activating compound is a compound of formnula 1 and the attendant definitions, wherein R₃, R₅, R′₂ and R′₃ are OH. In a further embodiment, a sirtuin-activating compound is a compound of formula 1 and the attendant definitions, wherein R₁, R₃, R₅, R′₂ and R′₃ are OH. In a further embodiment, a sirtuin-activating compound is a compound of formula 1 and the attendant definitions, wherein R₂ and R′₂ are OH; R₄ is O-β-D-glucoside; and R′₃ is OCH₃. In a further embodiment, a sirtuin-activating compound is a compound of formula 1 and the attendant definitions, wherein R₂ is OH; R₄ is O-β-D-glucoside; and R′₃ is OCH₃.

In a further embodiment, a sirtuin-activating compound is a compound of formula 1 and the attendant definitions, wherein n is 0; A-B is ethenyl; and R₁, R₂, R₃, R₄, R₅, R′₁, R′₂, R′₃, R′₄, and R′₅ are H (trans stilbene). In a further embodiment, a sirtuin-activating compound is a compound of formula 1 and the attendant definitions, wherein n is 1; A-B is ethenyl; M is O; and R₁, R₂, R₃, R₄, R₅, R′₁, R′₂, R′₃, R′₄, and R′₅ are H (chalcone). In a further embodiment, a sirtuin-activating compound is a compound of formula 1 and the attendant definitions, wherein n is 0; A-B is ethenyl; R₂, R₄, and R′₃ are OH; and R₁, R₃, R₅, R′₁, R′₂, R′₄, and R′₅ are H (resveratrol). In a further embodiment, a sirtuin-activating compound is a compound of formula 1 and the attendant definitions, wherein n is 0; A-B is ethenyl; R₂, R₄, R′₂ and R′₃ are OH; and R₁, R₃, R₅, R′₁, R′₄ and R′₅ are H (piceatannol). In a further embodiment, a sirtuin-activating compound is a compound of formula 1 and the attendant definitions, wherein n is 1; A-B is ethenyl; M is O; R₃, R₅, R′₂ and R′₃ are OH; and R₁, R₂, R₄, R′₁, R′₄, and R′₅ are H (butein). In a further embodiment, a sirtuin-activating compound is a compound of formula 1 and the attendant definitions, wherein n is 1; A-B is ethenyl; M is O; R₁, R₃, R₅, R′₂ and R′₃ are OH; and R₂, R₄, R′₁, R′₄, and R′₅ are H (3,4,2′,4′,6′-pentahydroxychalcone). In a further embodiment, a sirtuin-activating compound is a compound of formula 1 and the attendant definitions, wherein n is 0; A-B is ethenyl; R₂ and R′₂ are OH, R₄ is O-β-D-glucoside, R′₃ is OCH₃; and R₁, R₃, R₅, R′₁, R′₄, and R′₅ are H (rhapontin). In a further embodiment, a sirtuin-activating compound is a compound of formula 1 and the attendant definitions, wherein n is 0; A-B is ethenyl; R₂ is OH, R₄ is O-β-D-glucoside, R′₃ is OCH₃; and R₁, R₃, R₅, R′₁, R′₂, R′₄, and R′₅ are H (deoxyrhapontin). In a further embodiment, a sirtuin-activating compound is a compound of formula 1 and the attendant definitions, wherein n is 0; A-B is —CH₂CH(Me)CH(Me)CH₂—; R₂, R₃, R′₂, and R′₃ are OH; and R₁, R₄, R₅, R′₁, R′₄, and R′₅ are H (NDGA).

In another embodiment, a sirtuin-activating compound is a flavanone compound of formula 2:

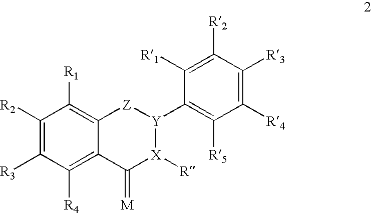

wherein, independently for each occurrence,

R₁, R₂, R₃, R₄, R′₁, R′₂, R′₃, R′₄, R′₅, and R″ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S;

X represents CR or N; and

Y represents CR or N.

In a further embodiment, a sirtuin-activating compound is a compound of formula 2 and the attendant definitions, wherein X and Y are both CH. In a further embodiment, a sirtuin-activating compound is a compound of formula 2 and the attendant definitions, wherein M is O. In a further embodiment, a sirtuin-activating compound is a compound of formula 2 and the attendant definitions, wherein M is H₂. In a further embodiment, a sirtuin-activating compound is a compound of formula 2 and the attendant definitions, wherein Z is O. In a further embodiment, a sirtuin-activating compound is a compound of formula 2 and the attendant definitions, wherein R″ is H. In a further embodiment, a sirtuin-activating compound is a compound of formula 2 and the attendant definitions, wherein R″ is OH. In a further embodiment, a sirtuin-activating compound is a compound of formula 2 and the attendant definitions, wherein R″ is an alkoxycarbonyl. In a further embodiment, a sirtuin-activating compound is a compound of formula 2 and the attendant definitions, wherein R₁ is 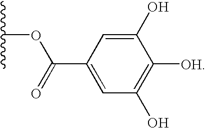
In a further embodiment, a sirtuin-activating compound is a compound of formula 2 and the attendant definitions, wherein R₁, R₂, R₃, R₄, R′₁, R′₂, R′₃, R′₄, R′₅ and R″ are H. In a further embodiment, a sirtuin-activating compound is a compound of formula 2 and the attendant definitions, wherein R₂, R₄, and R′₃ are OH. In a further embodiment, a sirtuin-activating compound is a compound of formula 2 and the attendant definitions, wherein R₄, R′₂, R′₃, and R″ are OH. In a further embodiment, a sirtuin-activating compound is a compound of formula 2 and the attendant definitions, wherein R₂, R₄, R′₂, R′₃, and R″ are OH. In a further embodiment, a sirtuin-activating compound is a compound of formula 2 and the attendant definitions, wherein R₂, R₄, R′₂, R′₃, R′₄, and R″ are OH.

In a further embodiment, a sirtuin-activating compound is a compound of formula 2 and the attendant definitions, wherein X and Y are CH; M is O; Z and O; R″ is H; and R₁, R₂, R₃, R₄, R′₁, R′₂, R′₃, R′₄, R′₅ and R″ are H (flavanone). In a further embodiment, a sirtuin-activating compound is a compound of formula 2 and the attendant definitions, wherein X and Y are CH; M is O; Z and O; R″ is H; R₂, R₄, and R′₃ are OH; and R₁, R₃, R′₁, R′₂, R′₄, and R′₅ are H (naringenin). In a further embodiment, a sirtuin-activating compound is a compound of formula 2 and the attendant definitions, wherein X and Y are CH; M is O; Z and O; R″ is OH; R₂, R₄, R′₂, and R′₃ are OH; and R₁, R₃, R′₁, R′₄, and R′₅ are H (3,5,7,3′,4′-pentahydroxyflavanone). In a further embodiment, a sirtuin-activating compound is a compound of formula 2 and the attendant definitions, wherein X and Y are CH; M is H₂; Z and O; R″ is OH; R₂, R₄, R′₂, and R′₃, are OH; and R₁, R₃, R′₁, R′₄ and R′₅ are H (epicatechin). In a further embodiment, a sirtuin-activating compound is a compound of formula 2 and the attendant definitions, wherein X and Y are CH; M is H₂; Z and O; R″ is OH; R₂, R₄, R′₂, R′₃, and R′₄ are OH; and R₁, R₃, R′₁, and R′₅ are H (gallocatechin). In a further embodiment, a sirtuin-activating compound is a compound of formula 2 and the attendant definitions, wherein X and Y are CH; M is H₂; Z and O; R″ is 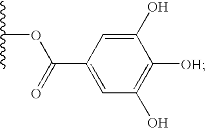
R₂, R₄, R′₂, R′₃, R′₄, and R″ are OH; and R₁, R₃, R′₁, and R′₅ are H (epigallocatechin gallate).

In another embodiment, a sirtuin-activating compound is an isoflavanone compound of formula 3:

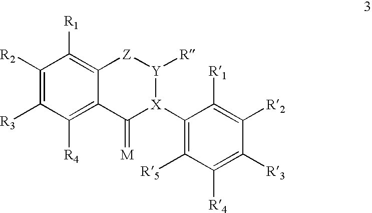

wherein, independently for each occurrence,

R₁, R₂, R₃, R₄, R′₁, R′₂, R′₃, R′₄, R′₅, and R″₁ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S;

X represents CR or N; and

Y represents CR or N.

In another embodiment, a sirtuin-activating compound is a flavone compound of formula 4:

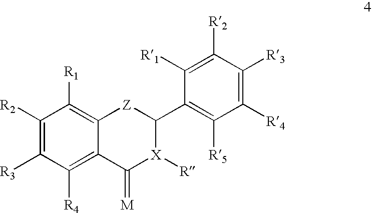

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents CR, O, NR, or S; and

X represents CR″ or N, wherein

R″ is H, alkyl, aryl, heteroaryl, alkaryl, heteroaralkyl, halide, NO₂, SR, OR, N(R)₂, or carboxyl.

In a further embodiment, a sirtuin-activating compound is a compound of formula 4 and the attendant definitions, wherein X is C. In a further embodiment, a sirtuin-activating compound is a compound of formula 4 and the attendant definitions, wherein X is CR. In a further embodiment, a sirtuin-activating compound is a compound of formula 4 and the attendant definitions, wherein Z is O. In a further embodiment, a sirtuin-activating compound is a compound of formula 4 and the attendant definitions, wherein M is O. In a further embodiment, a sirtuin-activating compound is a compound of formula 4 and the attendant definitions, wherein R″ is H. In a further embodiment, a sirtuin-activating compound is a compound of formula 4 and the attendant definitions, wherein R″ is OH. In a further embodiment, a sirtuin-activating compound is a compound of formula 4 and the attendant definitions, wherein R₁, R₂, R₃, R₄, R′₁, R′₂, R′₃, R′₄, and R′₅ are H. In a further embodiment, a sirtuin-activating compound is a compound of formula 4 and the attendant definitions, wherein R₂, R′₂, and R′₃ are OH. In a further embodiment, a sirtuin-activating compound is a compound of formula 4 and the attendant definitions, wherein R₂, R₄, R′₂, R′₃, and R′₄ are OH. In a further embodiment, a sirtuin-activating compound is a compound of formula 4 and the attendant definitions, wherein R₂, R₄, R′₂, and R′₃ are OH. In a further embodiment, a sirtuin-activating compound is a compound of formula 4 and the attendant definitions, wherein R₃, R′₂, and R′₃ are OH. In a further embodiment, a sirtuin-activating compound is a compound of formula 4 and the attendant definitions, wherein R₂, R₄, R′₂, and R′₃ are OH. In a further embodiment, a sirtuin-activating compound is a compound of formula 4 and the attendant definitions, wherein R₂, R′₂, R′₃, and R′₄ are OH. In a further embodiment, a sirtuin-activating compound is a compound of formula 4 and the attendant definitions, wherein R₂, R₄, and R′₃ are OH. In a further embodiment, a sirtuin-activating compound is a compound of formula 4 and the attendant definitions, wherein R₂, R₃, R₄, and R′₃ are OH. In a further embodiment, a sirtuin-activating compound is a compound of formula 4 and the attendant definitions, wherein R₂, R₄, and R′₃ are OH. In a further embodiment, a sirtuin-activating compound is a compound of formula 4 and the attendant definitions, wherein R₃, R′₁, and R′₃ are OH. In a further embodiment, a sirtuin-activating compound is a compound of formula 4 and the attendant definitions, wherein R₂ and R′₃ are OH. In a further embodiment, a sirtuin-activating compound is a compound of formula 4 and the attendant definitions, wherein R₁, R₂, R′₂, and R′₃ are OH. In a further embodiment, a sirtuin-activating compound is a compound of formula 4 and the attendant definitions, wherein R₃, R′₁, and R′₂ are OH. In a further embodiment, a sirtuin-activating compound is a compound of formula 4 and the attendant definitions, wherein R′₃ is OH. In a further embodiment, a sirtuin-activating compound is a compound of formula 4 and the attendant definitions, wherein R₄ and R′₃ are OH. In a further embodiment, a sirtuin-activating compound is a compound of formula 4 and the attendant definitions, wherein R₂ and R₄ are OH. In a further embodiment, a sirtuin-activating compound is a compound of formula 4 and the attendant definitions, wherein R₂, R₄, R′₁, and R′₃ are OH. In a further embodiment, a sirtuin-activating compound is a compound of formula 4 and the attendant definitions, wherein R₄ is OH. In a further embodiment, a sirtuin-activating compound is a compound of formula 4 and the attendant definitions, wherein R₂, R₄, R′₂, R′₃, and R′₄ are OH. In a further embodiment, a sirtuin-activating compound is a compound of formula 4 and the attendant definitions, wherein R₂, R′₂, R′₃, and R′₄ are OH. In a further embodiment, a sirtuin-activating compound is a compound of formula 4 and the attendant definitions, wherein R₁, R₂, R₄, R′₂, and R′₃ are OH.

In a further embodiment, a sirtuin-activating compound is a compound of formula 4 and the attendant definitions, wherein X is CH; Z is O; M is O; and R₁, R₂, R₃, R₄, R′₁, R′₂, R′₃, R′₄, and R′₅ are H (flavone). In a further embodiment, a sirtuin-activating compound is a compound of formula 4 and the attendant definitions, wherein X is COH; Z is O; M is O; R₂, R′₂, and R′₃ are OH; and R₁, R₃, R₄, R′₁, R′₄, and R′₅ are H (fisetin). In a further embodiment, a sirtuin-activating compound is a compound of formula 4 and the attendant definitions, wherein X is CH; Z is O; M is O; R₂, R₄, R′₂, R′₃, and R′₄ are OH; and R₁, R₃, R′₁, and R′₅ are H (5,7,3′,4′,5′-pentahydroxyflavone). In a further embodiment, a sirtuin-activating compound is a compound of formula 4 and the attendant definitions, wherein X is CH; Z is O; M is O; R₂, R₄, R′₂, and R′₃ are OH; and R₁, R₃, R′₁, R′₄, and R′₅ are H (luteolin). In a further embodiment, a sirtuin-activating compound is a compound of formula 4 and the attendant definitions, wherein X is COH; Z is O; M is O; R₃, R′₂, and R′₃ are OH; and R₁, R₂, R₄, R′₁, R′₄, and R′₅ are H (3,6,3′,4′-tetrahydroxyflavone). In a further embodiment, a sirtuin-activating compound is a compound of formula 4 and the attendant definitions, wherein X is COH; Z is O; M is O; R₂, R₄, R′₂, and R′₃ are OH; and R₁, R₃, R′₁, R′₄, and R′₅ are H (quercetin). In a further embodiment, a sirtuin-activating compound is a compound of formula 4 and the attendant definitions, wherein X is CH; Z is O; M is O; R₂, R′₂, R′₃, and R′₄ are OH; and R₁, R₃, R₄, R′₁, and R′₅ are H. In a further embodiment, a sirtuin-activating compound is a compound of formula 4 and the attendant definitions, wherein X is COH; Z is O; M is O; R₂, R₄, and R′₃ are OH; and R₁, R₃, R′₁, R′₂, R′₄, and R′₅ are H. In a further embodiment, a sirtuin-activating compound is a compound of formula 4 and the attendant definitions, wherein X is CH; Z is O; M is O; R₂, R₃, R₄, and R′₃ are OH; and R₁, R′₁, R′₂, R′₄, and R′₅ are H. In a further embodiment, a sirtuin-activating compound is a compound of formula 4 and the attendant definitions, wherein X is CH; Z is O; M is O; R₂, R₄, and R′₃ are OH; and R₁, R₃, R′₁, R′₂, R′₄, and R′₅ are H. In a further embodiment, a sirtuin-activating compound is a compound of formula 4 and the attendant definitions, wherein X is COH; Z is O; M is O; R₃, R′₁, and R′₃ are OH; and R₁, R₂, R₄, R′₂, R′₄, and R′₅ are H. In a further embodiment, a sirtuin-activating compound is a compound of formula 4 and the attendant definitions, wherein X is CH; Z is O; M is O; R₂ and R′₃ are OH; and R₁, R₃, R₄, R′₁, R′₂, R′₄, and R′₅ are H. In a further embodiment, a sirtuin-activating compound is a compound of formula 4 and the attendant definitions, wherein X is COH; Z is O; M is O; R₁, R₂, R′₂, and R′₃ are OH; and R₁, R₂, R₄, R′₃, R′₄, and R′₅ are H. In a further embodiment, a sirtuin-activating compound is a compound of formula 4 and the attendant definitions, wherein X is COH; Z is O; M is O; R₃, R′₁, and R′₂ are OH; and R₁, R₂, R₄; R′₃, R′₄, and R′₅ are H. In a further embodiment, a sirtuin-activating compound is a compound of formula 4 and the attendant definitions, wherein X is CH; Z is O; M is O; R′₃ is OH; and R₁, R₂, R₃, R₄, R′₁, R′₂, R′₄, and R′₅ are H. In a further embodiment, a sirtuin-activating compound is a compound of formula 4 and the attendant definitions, wherein X is CH; Z is O; M is O; R₄ and R′₃ are OH; and R₁, R₂, R₃, R′₁, R′₂, R′₄, and R′₅ are H. In a further embodiment, a sirtuin-activating compound is a compound of formula 4 and the attendant definitions, wherein X is CH; Z is O; M is O; R₂ and R₄ are OH; and R₁, R₃, R′₁, R′₂, R′₃, R′₄, and R′₅ are H. In a further embodiment, a sirtuin-activating compound is a compound of formula 4 and the attendant definitions, wherein X is COH; Z is O; M is O; R₂, R₄, R′₁, and R′₃ are OH; and R₁, R₃, R′₂, R′₄, and R′₅ are H. In a further embodiment, a sirtuin-activating compound is a compound of formula 4 and the attendant definitions, wherein X is CH; Z is O; M is O; R₄ is OH; and R₁, R₂, R₃, R′₁, R′₂, R′₃, R′₄, and R′₅ are H. In a further embodiment, a sirtuin-activating compound is a compound of formula 4 and the attendant definitions, wherein X is COH; Z is O; M is O; R₂, R₄, R′₂, R′₃, and R′₄ are OH; and R₁, R₃, R′₁, and R′₅ are H. In a further embodiment, a sirtuin-activating compound is a compound of formula 4 and the attendant definitions, wherein X is COH; Z is O; M is O; R₂, R′₂, R′₃, and R′₄ are OH; and R₁, R₃, R₄, R′₁, and R′₅ are H. In a further embodiment, a sirtuin-activating compound is a compound of formula 4 and the attendant definitions, wherein X is COH; Z is O; M is O; R₁, R₂, R₄, R′₂, and R′₃ are OH; and R₃, R′₁, R′₄, and R′₅ are H.

In another embodiment, a sirtuin-activating compound is an isoflavone compound of formula 5:

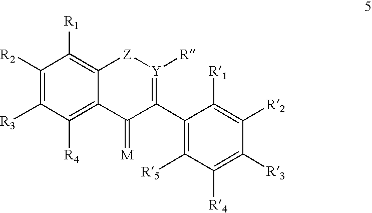

wherein, independently for each occurrence,

R₁, R₂, R₃, R₄, R′₁, R′₂, R′₃, R′₄, and R′₅, represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl;

M represents H₂, O, NR, or S;

Z represents C(R)₂, O, NR, or S; and

Y represents CR″ or N, wherein

R″ represents H, alkyl, aryl, heteroaryl, alkaryl, heteroaralkyl, halide, NO₂, SR, OR, N(R)₂, or carboxyl.

In a further embodiment, a sirtuin-activating compound is a compound of formula 5 and the attendant definitions, wherein Y is CR″. In a further embodiment, a sirtuin-activating compound is a compound of formula 5 and the attendant definitions, wherein Y is CH. In a further embodiment, a sirtuin-activating compound is a compound of formula 5 and the attendant definitions, wherein Z is O. In a further embodiment, a sirtuin-activating compound is a compound of formula 5 and the attendant definitions, wherein M is O. In a further embodiment, a sirtuin-activating compound is a compound of formula 5 and the attendant definitions, wherein R₂ and R′₃ are OH. In a further embodiment, a sirtuin-activating compound is a compound of formula 5 and the attendant definitions, wherein R₂, R₄, and R′₃ are OH.

In a further embodiment, a sirtuin-activating compound is a compound of formula 5 and the attendant definitions, wherein Y is CH; Z is O; M is O; R₂ and R′₃ are OH; and R₁, R₃, R₄, R′₁, R′₂, R′₄, and R′₅ are H. In a further embodiment, a sirtuin-activating compound is a compound of formula 5 and the attendant definitions, wherein Y is CH; Z is O; M is O; R₂, R₄, and R′₃ are OH; and R₁, R₃, R′₁, R′₂, R′₄, and R′₅ are H.

In another embodiment, a sirtuin-activating compound is an anthocyanidin compound of formula 6:

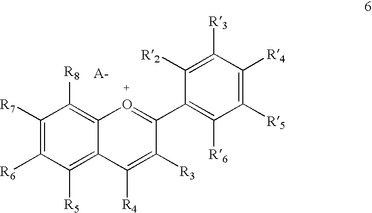

wherein, independently for each occurrence,

R₃, R₄, R₅, R₆, R₇, R₈, R′₂, R′₃, R′₄, R′₅, and R′₆ represent H, alkyl, aryl, heteroaryl, aralkyl, alkaryl, heteroaralkyl, halide, NO₂, SR, OR, N(R)₂, or carboxyl;

R represents H, alkyl, aryl, heteroaryl, or aralkyl; and

A⁻ represents an anion selected from the following: Cl⁻, Br⁻, or I⁻.

In a further embodiment, a sirtuin-activating compound is a compound of formula 6 and the attendant definitions, wherein A⁻ is Cl⁻. In a further embodiment, a sirtuin-activating compound is a compound of formula 6 and the attendant definitions, wherein R₃, R₅, R₇, and R′₄ are OH. In a further embodiment, a sirtuin-activating compound is a compound of formula 6 and the attendant definitions, wherein R₃, R₅, R₇, R′₃, and R′₄ are OH. In a further embodiment, a sirtuin-activating compound is a compound of formula 6 and the attendant definitions, wherein R₃, R₅, R₇, R′₃, R′₄, and R′₅ are OH.

In a further embodiment, a sirtuin-activating compound is a compound of formula 6 and the attendant definitions, wherein A⁻ is Cl⁻; R₃, R₅, R₇, and R′₄ are OH; and R₄, R₆, R₈, R′₂, R′₃, R′₅, and R′₆ are H. In a further embodiment, a sirtuin-activating compound is a compound of formula 6 and the attendant definitions, wherein A⁻ is Cl⁻; R₃, R₅, R₇, R′₃, and R′₄ are OH; and R₄, R₆, R₈, R′₂, R′₅, and R′₆ are H. In a further embodiment, a sirtuin-activating compound is a compound of formula 6 and the attendant definitions, wherein A⁻ is Cl⁻; R₃, R₅, R₇, R′₃, R′₄, and R′₅ are OH; and R₄, R₆, R₈, R′₂, and R′₆ are H.

In a further embodiment, a sirtuin-activating compound is a stilbene, chalcone, or flavone compound represented by formula 7:

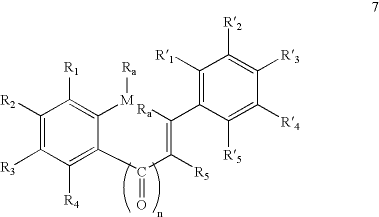

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, aryl, heteroaryl, aralkyl; and

n is 0 or 1.

In a further embodiment, a sirtuin-activating compound is an activating compound represented by formula 7 and the attendant definitions, wherein n is 0. In a further embodiment, a sirtuin-activating compound is an activating compound represented by formula 7 and the attendant definitions, wherein n is 1. In a further embodiment, a sirtuin-activating compound is an activating compound represented by formula 7 and the attendant definitions, wherein M is absent. In a further embodiment, a sirtuin-activating compound is an activating compound represented by formula 7 and the attendant definitions, wherein M is O. In a further embodiment, a sirtuin-activating compound is an activating compound represented by formula 7 and the attendant definitions, wherein Rₐ is H. In a further embodiment, a sirtuin-activating compound is an activating compound represented by formula 7 and the attendant definitions, wherein M is O and the two Rₐ form a bond.

In a further embodiment, a sirtuin-activating compound is an activating compound represented by formula 7 and the attendant definitions, wherein R₅ is H. In a further embodiment, a sirtuin-activating compound is an activating compound represented by formula 7 and the attendant definitions, wherein R₅ is OH. In a further embodiment, a sirtuin-activating compound is an activating compound represented by formula 7 and the attendant definitions, wherein R₁, R₃, and R′₃ are OH. In a further embodiment, a sirtuin-activating compound is an activating compound represented by formula 7 and the attendant definitions, wherein R₂, R₄, R′₂, and R′₃ are OH. In a further embodiment, a sirtuin-activating compound is an activating compound represented by formula 7 and the attendant definitions, wherein R₂, R′₂, and R′₃ are OH. In a further embodiment, a sirtuin-activating compound is an activating compound represented by formula 7 and the attendant definitions, wherein R₂ and R₄ are OH.

In a further embodiment, a sirtuin-activating compound is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin-activating compound is an activating compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin-activating compound is an activating compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

Other sirtuin-activating compounds include compounds having a formula selected from the group consisting of formulas 8-25 and 30 set forth below.

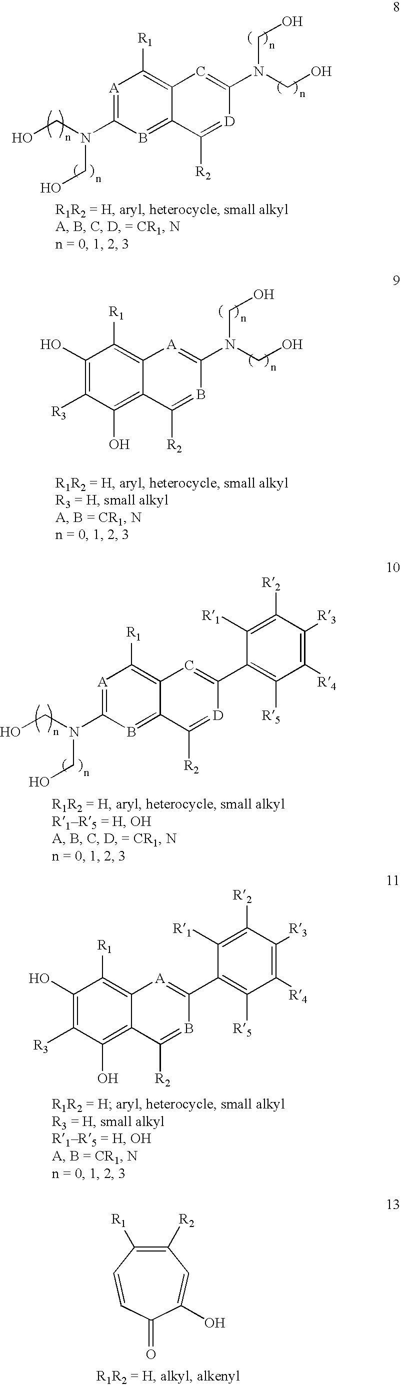

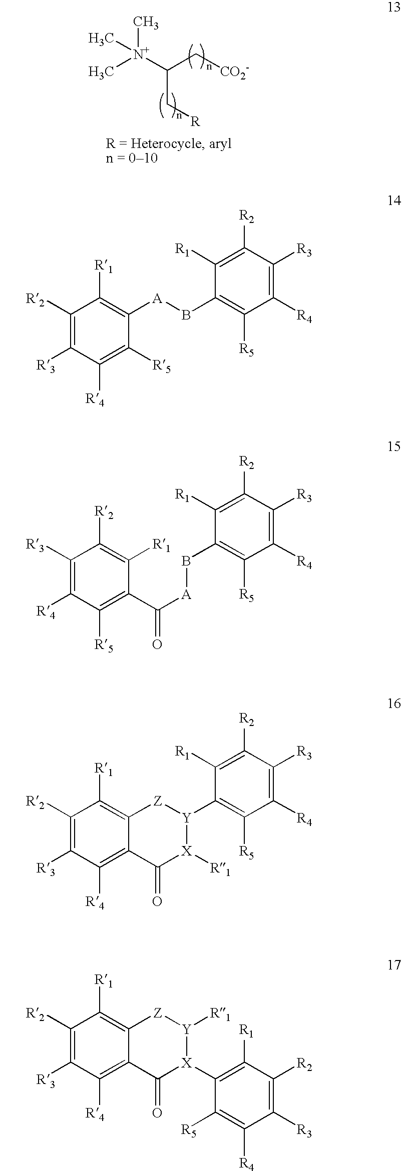

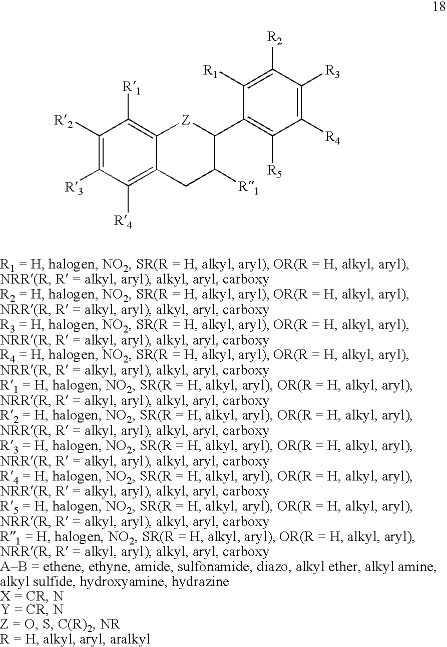

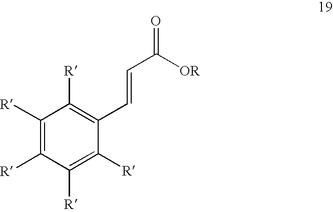

wherein, independently for each occurrence,

R=H, alkyl, aryl, heterocyclyl, heteroaryl, or aralkyl; and

R′=H, halogen, NO₂, SR, OR, NR₂, alkyl, aryl, or carboxy.

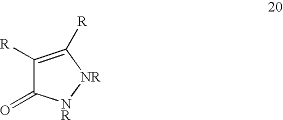

wherein, independently for each occurrence,

R=H, alkyl, aryl, heterocyclyl, heteroaryl, or aralkyl.

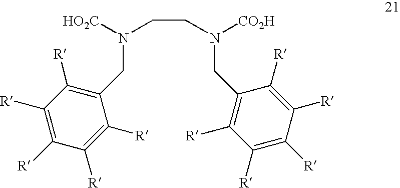

wherein, independently for each occurrence,

R′=H, halogen, NO₂, SR, OR, NR₂, alkyl, aryl, aralkyl, or carboxy; and

R=H, alkyl, aryl, heterocyclyl, heteroaryl, or aralkyl.

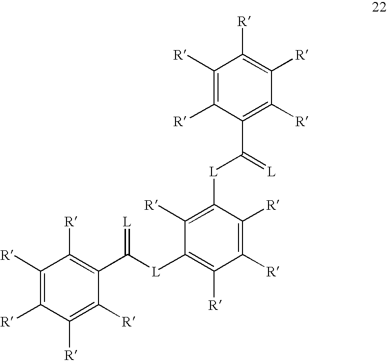

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

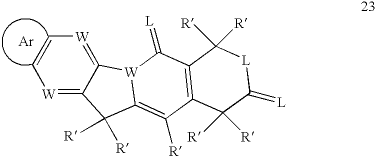

wherein, independently for each occurrence,

L represents CR₂, O, NR, or S;

W represents CR or N;

R represents H, alkyl, aryl, aralkyl, or heteroaralkyl;

Ar represents a fused aryl or heteroaryl ring; and

R′ represents H, halogen, NO₂, SR, OR, NR₂, alkyl, aryl, aralkyl, or carboxy.

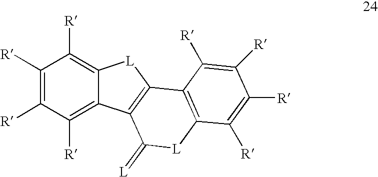

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

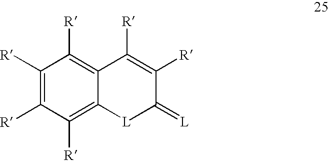

wherein, independently for each occurrence,

L represents CR₂, O, NR, or S;

R represents H, alkyl, aryl, aralkyl, or heteroaralkyl; and

R′ represents H, halogen, NO₂, SR, OR, NR₂, alkyl, aryl, aralkyl, or carboxy.

In a further embodiment, a sirtuin-activating compound is a stilbene, chalcone, or flavone compound represented by formula 30:

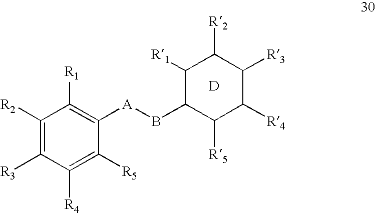

wherein, independently for each occurrence,

D is a phenyl or cyclohexyl group;

R₁, R₂, R₃, R₄, R₅, R′₁, R′₂, R′₃, R′₄, and R′₅ represent H, alkyl, aryl, heteroaryl, alkaryl, heteroaralkyl, halide, NO₂, SR, OR, N(R)₂, carboxyl, azide, ether; or any two adjacent R or R′ groups taken together form a fused benzene or cyclohexyl group;

R represents H, alkyl, aryl, or aralkyl; and

A-B represents an ethylene, ethenylene, or imine group;

provided that when A-B is ethenylene, D is phenyl, and R′₃ is H: R₃ is not OH when R₁, R₂, R₄, and R₅ are H; and R₂ and R₄ are not OMe when R₁, R₃, and R₅ are H; and R₃ is not OMe when R₁, R₂, R₄, and R₅ are H.

In a further embodiment, a sirtuin-activating compound is a compound represented by formula 30 and the attendant definitions, wherein D is a phenyl group.

In a further embodiment, a sirtuin-activating compound is a compound represented by formula 30 and the attendant definitions, wherein A-B is an ethenylene or imine group.

In a further embodiment, a sirtuin-activating compound is a compound represented by formula 30 and the attendant definitions, wherein A-B is an ethenylene group.

In a further embodiment, a sirtuin-activating compound is a compound represented by formula 30 and the attendant definitions, wherein R₂ is OH.

In a further embodiment, a sirtuin-activating compound is a compound represented by formula 30 and the attendant definitions, wherein R₄ is OH In a further embodiment, a sirtuin-activating compound is a compound represented by formula 30 and the attendant definitions, wherein R₂ and R₄ are OH.

In a further embodiment, a sirtuin-activating compound is a compound represented by formula 30 and the attendant definitions, wherein D is a phenyl group; and A-B is an ethenylene group.

In a further embodiment, a sirtuin-activating compound is a compound represented by formula 30 and the attendant definitions, wherein D is a phenyl group; A-B is an ethenylene group; and R₂ and R₄ are OH.

In a further embodiment, a sirtuin-activating compound is a compound represented by formula 30 and the attendant definitions, wherein A-B is ethenylene; D is a phenyl ring; R₂ and R₄ are OH; and R′₃ is Cl.

In a further embodiment, a sirtuin-activating compound is a compound represented by formula 30 and the attendant definitions, wherein A-B is ethenylene; D is a phenyl ring; R₂ and R₄ are OH; and R′₃ is OH.

In a further embodiment, a sirtuin-activating compound is a compound represented by formula 30 and the attendant definitions, wherein A-B is ethenylene; D is a phenyl ring; R₂ and R₄ are OH; and R′₃ is H.

In a further embodiment, a sirtuin-activating compound is a compound represented by formula 30 and the attendant definitions, wherein A-B is ethenylene; D is a phenyl ring; R₂ and R₄ are OH; and R′₃ is CH₂CH₃.

In a further embodiment, a sirtuin-activating compound is a compound represented by formula 30 and the attendant definitions, wherein A-B is ethenylene; D is a phenyl ring; R₂ and R₄ are OH; and R′₃ is F.

In a further embodiment, a sirtuin-activating compound is a compound represented by formula 30 and the attendant definitions, wherein A-B is ethenylene; D is a phenyl ring; R₂ and R₄ are OH; and R′₃ is Me.

In a further embodiment, a sirtuin-activating compound is a compound represented by formula 30 and the attendant definitions, wherein A-B is ethenylene; D is a phenyl ring; R₂ and R₄ are OH; and R′₃ is an azide.

In a further embodiment, a sirtuin-activating compound is a compound represented by formula 30 and the attendant definitions, wherein A-B is ethenylene; D is a phenyl ring; R₂ and R₄ are OH; and R′₃ is SMe.

In a further embodiment, a sirtuin-activating compound is a compound represented by formula 30 and the attendant definitions, wherein A-B is ethenylene; D is a phenyl ring; R₂ and R₄ are OH; and R′₃ is NO₂.

In a further embodiment, a sirtuin-activating compound is a compound represented by formula 30 and the attendant definitions, wherein A-B is ethenylene; D is a phenyl ring; R₂ and R₄ are OH; and R′₃ is CH(CH₃)₂.

In a further embodiment, a sirtuin-activating compound is a compound represented by formula 30 and the attendant definitions, wherein A-B is ethenylene; D is a phenyl ring; R₂ and R₄ are OH; and R′₃ is OMe.

In a further embodiment, a sirtuin-activating compound is a compound represented by formula 30 and the attendant definitions, wherein A-B is ethenylene; D is a phenyl ring; R₂ and R₄ are OH; R′₂ is OH; and R′₃ is OMe.

In a further embodiment, a sirtuin-activating compound is a compound represented by formula 30 and the attendant definitions, wherein A-B is ethenylene; D is a phenyl ring; R₂ is OH; R₄ is carboxyl; and R′₃ is OH.

In a further embodiment, a sirtuin-activating compound is a compound represented by formula 30 and the attendant definitions, wherein A-B is ethenylene; D is a phenyl ring; R₂ and R₄ are OH; and R′₃ is carboxyl.

In a further embodiment, a sirtuin-activating compound is a compound represented by formula 30 and the attendant definitions, wherein A-B is ethenylene; D is a phenyl ring; R₂ and R₄ are OH; and R′₃ and R′₄ taken together form a fused benzene ring.

In a further embodiment, a sirtuin-activating compound is a compound represented by formula 30 and the attendant definitions, wherein A-B is ethenylene; D is a phenyl ring; and R₄ is OH.

In a further embodiment, a sirtuin-activating compound is a compound represented by formula 30 and the attendant definitions, wherein A-B is ethenylene; D is a phenyl ring; R₂ and R₄ are OCH₂OCH₃; and R′₃ is SMe.

In a further embodiment, a sirtuin-activating compound is a compound represented by formula 30 and the attendant definitions, wherein A-B is ethenylene; D is a phenyl ring; R₂ and R₄ are OH; and R′₃ is carboxyl.

In a further embodiment, a sirtuin-activating compound is a compound represented by formula 30 and the attendant definitions, wherein A-B is ethenylene; D is a cyclohexyl ring; and R₂ and R₄ are OH.

In a further embodiment, a sirtuin-activating compound is a compound represented by formula 30 and the attendant definitions, wherein A-B is ethenylene; D is a phenyl ring; and R₃ and R₄ are OMe.

In a further embodiment, a sirtuin-activating compound is a compound represented by formula 30 and the attendant definitions, wherein A-B is ethenylene; D is a phenyl ring; R₂ and R₄ are OH; and R′₃ is OH.

In another embodiment, a sirtuin-activating compound is a compound of formula 32: 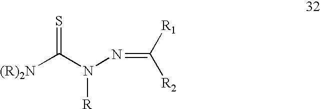
wherein, independently for each occurrence:

R is H, or a substituted or unsubstituted alkyl, aryl, aralkyl, heterocyclyl, heterocyclylalkyl, heteroaryl, or heteroaralkyl; and

R₁ and R₂ are a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 32 and the attendant definitions wherein R is H.

In a further embodiment, a sirtuin-activating compound is a compound of formula 32 and the attendant definitions wherein R₁ is 3-hydroxyphenyl.

In a further embodiment, a sirtuin-activating compound is a compound of formula 32 and the attendant definitions wherein R₂ is methyl.

In a further embodiment, a sirtuin-activating compound is a compound of formula 32 and the attendant definitions wherein R is H and R₁ is 3-hydroxyphenyl.

In a further embodiment, a sirtuin-activating compound is a compound of formula 32 and the attendant definitions wherein R is H, R₁ is 3-hydroxyphenyl, and R₂ is methyl.

In another embodiment, a sirtuin-activating compound is a compound of formula 33: 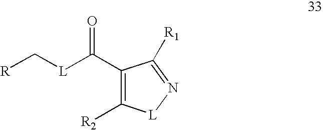
wherein, independently for each occurrence:

R is H, or a substituted or unsubstituted alkyl, alkenyl, or alkynyl;

R₁ and R₂ are a substituted or unsubstituted alkyl, aryl, aralkyl, heterocyclyl, heterocyclylalkyl, heteroaryl, or heteroaralkyl; and

L is O, S, or NR.

In a further embodiment, a sirtuin-activating compound is a compound of formula 33 and the attendant definitions wherein R is alkynyl.

In a further embodiment, a sirtuin-activating compound is a compound of formula 33 and the attendant definitions wherein R₁ is 2,6-dichlorophenyl.

In a further embodiment, a sirtuin-activating compound is a compound of formula 33 and the attendant definitions wherein R₂ is methyl.

In a further embodiment, a sirtuin-activating compound is a compound of formula 33 and the attendant definitions wherein L is O.

In a further embodiment, a sirtuin-activating compound is a compound of formula 33 and the attendant definitions wherein R is alkynyl and R₁ is 2,6-dichlorophenyl.

In a further embodiment, a sirtuin-activating compound is a compound of formula 33 and the attendant definitions wherein R is alkynyl, R₁ is 2,6-dichlorophenyl, and R₂ is methyl.

In a further embodiment, a sirtuin-activating compound is a compound of formula 33 and the attendant definitions wherein R is alkynyl, R₁ is 2,6-dichlorophenyl, R₂ is methyl, and L is O.

In another embodiment, a sirtuin-activating compound is a compound of formula 34: 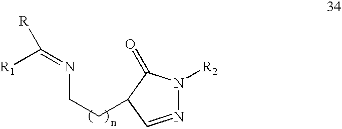
wherein, independently for each occurrence:

R, R₁, and R₂ are H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin-activating compound is a compound of formula 34 and the attendant definitions wherein R is 3,5-dichloro-2-hydroxyphenyl.

In a further embodiment, a sirtuin-activating compound is a compound of formula 34 and the attendant definitions wherein R₁ is H.

In a further embodiment, a sirtuin-activating compound is a compound of formula 34 and the attendant definitions wherein R₂ is H.

In a further embodiment, a sirtuin-activating compound is a compound of formula 34 and the attendant definitions wherein n is 1.

In a further embodiment, a sirtuin-activating compound is a compound of formula 34 and the attendant definitions wherein R is 3,5-dichloro-2-hydroxyphenyl and R₁ is H.

In a further embodiment, a sirtuin-activating compound is a compound of formula 34 and the attendant definitions wherein R is 3,5-dichloro-2-hydroxyphenyl, R₁ is H, and R₂ is H.

In a further embodiment, a sirtuin-activating compound is a compound of formula 34 and the attendant definitions wherein R is 3,5-dichloro-2-hydroxyphenyl, R₁ is H, R₂ is H, and n is 1.

In another embodiment, a sirtuin-activating compound is a compound of formula 35: 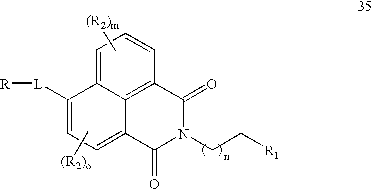
wherein, independently for each occurrence:

R is H or a substituted or unsubstituted alkyl, aryl, aralkyl, heterocyclyl, heterocyclylalkyl, heteroaryl, or heteroaralkyl;

R₁ is a substituted or unsubstituted alkyl, aryl, aralkyl, heterocyclyl, heterocyclylalkyl, heteroaryl, or heteroaralkyl;

R₂ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

L is O, NR, or S;

m is an integer from 0 to 3 inclusive;

n is an integer from 0 to 5 inclusive; and

o is an integer from 0 to 2 inclusive.

In a further embodiment, a sirtuin-activating compound is a compound of formula 35 and the attendant definitions wherein R is phenyl.

In a further embodiment, a sirtuin-activating compound is a compound of formula 35 and the attendant definitions wherein R₁ is pyridine.

In a further embodiment, a sirtuin-activating compound is a compound of formula 35 and the attendant definitions wherein L is S.

In a further embodiment, a sirtuin-activating compound is a compound of formula 35 and the attendant definitions wherein m is 0.

In a further embodiment, a sirtuin-activating compound is a compound of formula 35 and the attendant definitions wherein n is 1.

In a further embodiment, a sirtuin-activating compound is a compound of formula 35 and the attendant definitions wherein o is 0.

In a further embodiment, a sirtuin-activating compound is a compound of formula 35 and the attendant definitions wherein R is phenyl and R₁ is pyridine.

In a further embodiment, a sirtuin-activating compound is a compound of formula 35 and the attendant definitions wherein R is phenyl, R₁ is pyridine, and L is S.

In a further embodiment, a sirtuin-activating compound is a compound of formula 35 and the attendant definitions wherein R is phenyl, R₁ is pyridine, L is S, and m is 0.

In a further embodiment, a sirtuin-activating compound is a compound of formula 35 and the attendant definitions wherein R is phenyl, R₁ is pyridine, L is S, m is 0, and n is 1.

In a further embodiment, a sirtuin-activating compound is a compound of formula 35 and the attendant definitions wherein R is phenyl, R₁ is pyridine, L is S, m is 0, n is 1, and o is 0.

In another embodiment, a sirtuin-activating compound is a compound of formula 36: 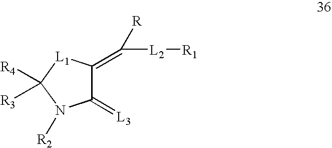
wherein, independently for each occurrence:

R, R₃, and R₄ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ and R₂ are H or a substituted or unsubstituted alkyl, aryl, aralkyl, heterocyclyl, heterocyclylalkyl, heteroaryl, heteroaralkyl;

L₁ is O, NR₁, S, C(R)₂, or SO₂; and

L₂ and L₃ are O, NR₁, S, or C(R)₂.

In a further embodiment, a sirtuin-activating compound is a compound of formula 36 and the attendant definitions wherein R is H.

In a further embodiment, a sirtuin-activating compound is a compound of formula 36 and the attendant definitions wherein R₁ is 4-chlorophenyl.

In a further embodiment, a sirtuin-activating compound is a compound of formula 36 and the attendant definitions wherein R₂ is 4-chlorophenyl.

In a further embodiment, a sirtuin-activating compound is a compound of formula 36 and the attendant definitions wherein R₃ is H.

In a further embodiment, a sirtuin-activating compound is a compound of formula 36 and the attendant definitions wherein R₄ is H.

In a further embodiment, a sirtuin-activating compound is a compound of formula 36 and the attendant definitions wherein L₁ is SO₂.

In a further embodiment, a sirtuin-activating compound is a compound of formula 36 and the attendant definitions wherein L₂ is NH.

In a further embodiment, a sirtuin-activating compound is a compound of formula 36 and the attendant definitions wherein L₃ is O.

In a further embodiment, a sirtuin-activating compound is a compound of formula 36 and the attendant definitions wherein R is H and R₁ is 4-chlorophenyl.

In a further embodiment, a sirtuin-activating compound is a compound of formula 36 and the attendant definitions wherein R is H, R₁ is 4-chlorophenyl, and R₂ is 4-chlorophenyl.

In a further embodiment, a sirtuin-activating compound is a compound of formula 36 and the attendant definitions wherein R is H, R₁ is 4-chlorophenyl, R₂ is 4-chlorophenyl, and R₃ is H.

In a further embodiment, a sirtuin-activating compound is a compound of formula 36 and the attendant definitions wherein R is H, R₁ is 4-chlorophenyl, R₂ is 4-chlorophenyl, R₃ is H, and R₄ is H.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, and L₁ is SO₂.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, L₁ is SO₂, and L₂ is NH.

In a further embodiment, a sirtuin-activating compound is a compound of formula 36 and the attendant definitions wherein R is H, R₁ is 4-chlorophenyl, R₂ is 4-chlorophenyl, R₃ is H, R₄ is H, L₁ is SO₂, L₂ is NH, and L₃ is O.

In another embodiment, a sirtuin-activating compound is a compound of formula 37: 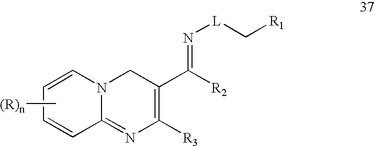
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, heteroaralkyl;

R₁ is H or a substituted or unsubstituted alkyl, aryl, aralkyl, heterocyclyl, heterocyclylalkyl, heteroaryl, heteroaralkyl;

R₂ and R₃ are H or a substituted or unsubstituted alkyl, aryl, aralkyl, heterocyclyl, heterocyclylalkyl, heteroaryl, heteroaralkyl;

L is O, NR₁, or S; and

n is an integer from 0 to 4 inclusive.

In a further embodiment, a sirtuin-activating compound is a compound of formula 37 and the attendant definitions wherein R is methyl.

In a further embodiment, a sirtuin-activating compound is a compound of formula 37 and the attendant definitions wherein n is 1.

In a further embodiment, a sirtuin-activating compound is a compound of formula 37 and the attendant definitions wherein R₁ is 3-fluorophenyl.

In a further embodiment, a sirtuin-activating compound is a compound of formula 37 and the attendant definitions wherein R₂ is H.

In a further embodiment, a sirtuin-activating compound is a compound of formula 37 and the attendant definitions wherein R₃ is 4-chlorophenyl.

In a further embodiment, a sirtuin-activating compound is a compound of formula 37 and the attendant definitions wherein L is O.

In a further embodiment, a sirtuin-activating compound is a compound of formula 37 and the attendant definitions wherein R is methyl and n is 1.

In a further embodiment, a sirtuin-activating compound is a compound of formula 37 and the attendant definitions wherein R is methyl, n is 1, and R₁ is 3-fluorophenyl.

In a further embodiment, a sirtuin-activating compound is a compound of formula 37 and the attendant definitions wherein R is methyl, n is 1, R₁ is 3-fluorophenyl, and R₂ is H.

In a further embodiment, a sirtuin-activating compound is a compound of formula 37 and the attendant definitions wherein R is methyl, n is 1, R₁ is 3-fluorophenyl, R₂ is H, and R₃ is 4-chlorophenyl.

In another embodiment, a sirtuin-activating compound is a compound of formula 38: 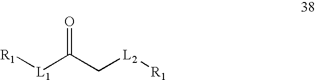
wherein, independently for each occurrence:

R and R₁ are H or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin-activating compound is a compound of formula 38 and the attendant definitions wherein R is 3-methoxyphenyl.

In a further embodiment, a sirtuin-activating compound is a compound of formula 38 and the attendant definitions wherein R₁ is 4-t-butylphenyl.

In a further embodiment, a sirtuin-activating compound is a compound of formula 38 and the attendant definitions wherein L₁ is NH.

In a further embodiment, a sirtuin-activating compound is a compound of formula 38 and the attendant definitions wherein L₂ is O.

In a further embodiment, a sirtuin-activating compound is a compound of formula 38 and the attendant definitions wherein R is 3-methoxyphenyl and R₁ is 4-t-butylphenyl.

In a further embodiment, a sirtuin-activating compound is a compound of formula 38 and the attendant definitions wherein R is 3-methoxyphenyl, R₁ is 4-t-butylphenyl, and L₁ is NH.

In a further embodiment, a sirtuin-activating compound is a compound of formula 38 and the attendant definitions wherein R is 3-methoxyphenyl, R₁ is 4-t-butylphenyl, L₁ is NH, and L₂ is O.

In another embodiment, a sirtuin-activating compound is a compound of formula 39: 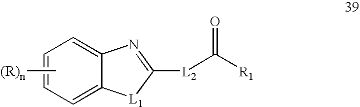
wherein, independently for each occurrence:

R is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ is H or a substituted or unsubstituted alkyl, aryl, alkaryl, heterocyclyl, heterocyclylalkyl, heteroaryl, or heteroaralkyl;

L₁ and L₂ are O, NR, or S; and

n is an integer from 0 to 4 inclusive.

In a further embodiment, a sirtuin-activating compound is a compound of formula 39 and the attendant definitions wherein R is methyl.

In a further embodiment, a sirtuin-activating compound is a compound of formula 39 and the attendant definitions wherein n is 1.

In a further embodiment, a sirtuin-activating compound is a compound of formula 39 and the attendant definitions wherein R₁ is 3,4,5-trimethoxyphenyl.

In a further embodiment, a sirtuin-activating compound is a compound of formula 39 and the attendant definitions wherein L₁ is S.

In a further embodiment, a sirtuin-activating compound is a compound of formula 39 and the attendant definitions wherein L₂ is NH.

In a further embodiment, a sirtuin-activating compound is a compound of formula 39 and the attendant definitions wherein R is methyl and n is 1.

In a further embodiment, a sirtuin-activating compound is a compound of formula 39 and the attendant definitions wherein R is methyl, n is 1, and R₁ is 3,4,5-trimethoxyphenyl.

In a further embodiment, a sirtuin-activating compound is a compound of formula 39 and the attendant definitions wherein R is methyl, n is 1, R₁ is 3,4,5-trimethoxyphenyl, and L₁ is S.

In a further embodiment, a sirtuin-activating compound is a compound of formula 39 and the attendant definitions wherein R is methyl, n is 1, R₁ is 3,4,5-trimethoxyphenyl, L₁ is S, and L₂ is NH.

In another embodiment, a sirtuin-activating compound is a compound of formula 40: 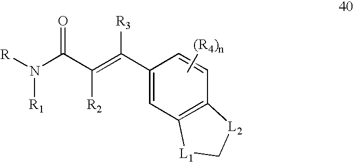
wherein, independently for each occurrence:

R, R₁, R₂, R₃ are H or a substituted or unsubstituted alkyl, aryl, alkaryl, heterocyclyl, heterocyclylalkyl, heteroaryl, or heteroaralkyl;

R₄ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR, or S; and

n is an integer from 0 to 3 inclusive.

In a further embodiment, a sirtuin-activating compound is a compound of formula 40 and the attendant definitions wherein R is H.

In a further embodiment, a sirtuin-activating compound is a compound of formula 40 and the attendant definitions wherein R₁ is perfluorophenyl.

In a further embodiment, a sirtuin-activating compound is a compound of formula 40 and the attendant definitions wherein R₂ is H.

In a further embodiment, a sirtuin-activating compound is a compound of formula 40 and the attendant definitions wherein R₃ is H.

In a further embodiment, a sirtuin-activating compound is a compound of formula 40 and the attendant definitions wherein L₁ is O.

In a further embodiment, a sirtuin-activating compound is a compound of formula 40 and the attendant definitions wherein L₂ is O.

In a further embodiment, a sirtuin-activating compound is a compound of formula 40 and the attendant definitions wherein n is 0.

In a further embodiment, a sirtuin-activating compound is a compound of formula 40 and the attendant definitions wherein R is H and R₁ is perfluorophenyl.

In a further embodiment, a sirtuin-activating compound is a compound of formula 40 and the attendant definitions wherein R is H, R₁ is perfluorophenyl, and R₂ is H.

In a further embodiment, a sirtuin-activating compound is a compound of formula 40 and the attendant definitions R is H, R₁ is perfluorophenyl, R₂ is H, and R₃ is H.

In a further embodiment, a sirtuin-activating compound is a compound of formula 40 and the attendant definitions wherein R is H, R₁ is perfluorophenyl, R₂ is H, R₃ is H, and L₁ is O.

In a further embodiment, a sirtuin-activating compound is a compound of formula 40 and the attendant definitions wherein R is H, R₁ is perfluorophenyl, R₂ is H, R₃ is H, L₁ is O, and L₂ is O.

In a further embodiment, a sirtuin-activating compound is a compound of formula 40 and the attendant definitions wherein R is H, R₁ is perfluorophenyl, R₂ is H, R₃ is H, L₁ is O, L₂ is O, and n is 0.

In another embodiment, a sirtuin-activating compound is a compound of formula 41: 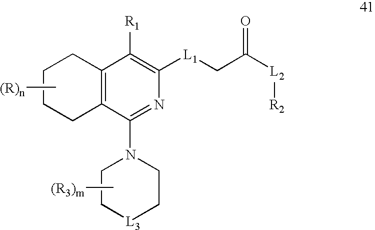
wherein, independently for each occurrence:

R, R₁, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H or a substituted or unsubstituted alkyl, aryl, aralkyl, heterocyclyl, heterocyclylalkyl, heteroaryl, or heteroaralkyl;

L₁, L₂, and L₃ are O, NR₂, or S; and

m and n are integers from 0 to 8 inclusive.

In a further embodiment, a sirtuin-activating compound is a compound of formula 41 and the attendant definitions wherein n is 0.

In a further embodiment, a sirtuin-activating compound is a compound of formula 41 and the attendant definitions wherein R₁ is cyano.

In a further embodiment, a sirtuin-activating compound is a compound of formula 41 and the attendant definitions wherein R₂ is ethyl.

In a further embodiment, a sirtuin-activating compound is a compound of formula 41 and the attendant definitions wherein m is 0.

In a further embodiment, a sirtuin-activating compound is a compound of formula 41 and the attendant definitions wherein L₁ is S.

In a further embodiment, a sirtuin-activating compound is a compound of formula 41 and the attendant definitions wherein L₂ is O.

In a further embodiment, a sirtuin-activating compound is a compound of formula 41 and the attendant definitions wherein L₃ is O.

In a further embodiment, a sirtuin-activating compound is a compound of formula 41 and the attendant definitions wherein n is 0 and R₁ is cyano.

In a further embodiment, a sirtuin-activating compound is a compound of formula 41 and the attendant definitions wherein n is 0, R₁ is cyano, and R₂ is ethyl.

In a further embodiment, a sirtuin-activating compound is a compound of formula 41 and the attendant definitions wherein n is 0, R₁ is cyano, R₂ is ethyl, and m is 0.

In a further embodiment, a sirtuin-activating compound is a compound of formula 41 and the attendant definitions wherein n is 0, R₁ is cyano, R₂ is ethyl, m is 0, and L₁ is S.

In a further embodiment, a sirtuin-activating compound is a compound of formula 41 and the attendant definitions wherein n is 0, R₁ is cyano, R₂ is ethyl, m is 0, L₁ is S, and L₂ is O.

In a further embodiment, a sirtuin-activating compound is a compound of formula 41 and the attendant definitions wherein n is 0, R₁ is cyano, R₂ is ethyl, m is 0, L₁ is S, L₂ is O, and L₃ is O.

In another embodiment, a sirtuin-activating compound is a compound of formula 42: 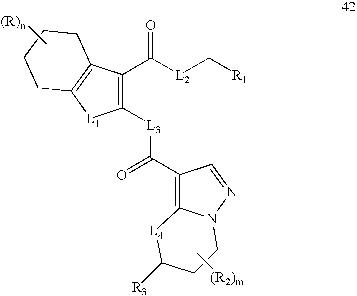
wherein, independently for each occurrence:

R and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₃ are H or a substituted or unsubstituted alkyl, aryl, aralkyl, heterocyclyl, heterocyclylalkyl, heteroaryl, or heteroaralkyl;

L₁, L₂, L₃, and L₄ are O, NR₁, or S;

m is an integer from 0 to 6 inclusive; and

n is an integer from 0 to 8 inclusive.

In a further embodiment, a sirtuin-activating compound is a compound of formula 42 and the attendant definitions wherein n is 0.

In a further embodiment, a sirtuin-activating compound is a compound of formula 42 and the attendant definitions wherein R₁ is methyl.

In a further embodiment, a sirtuin-activating compound is a compound of formula 42 and the attendant definitions wherein R₂ is CF₃ and m is 1.

In a further embodiment, a sirtuin-activating compound is a compound of formula 42 and the attendant definitions wherein R₃ is 4-methylphenyl.

In a further embodiment, a sirtuin-activating compound is a compound of formula 42 and the attendant definitions wherein L₁ is S.

In a further embodiment, a sirtuin-activating compound is a compound of formula 42 and the attendant definitions wherein L₂ is O.

In a further embodiment, a sirtuin-activating compound is a compound of formula 42 and the attendant definitions wherein L₃ is NR₁.

In a further embodiment, a sirtuin-activating compound is a compound of formula 42 and the attendant definitions wherein L₄ is NR₁.

In a further embodiment, a sirtuin-activating compound is a compound of formula 42 and the attendant definitions wherein n is 0 and R₁ is methyl.

In a further embodiment, a sirtuin-activating compound is a compound of formula 42 and the attendant definitions wherein n is 0, R₁ is methyl, R₂ is CF₃, and m is 1.

In a further embodiment, a sirtuin-activating compound is a compound of formula 42 and the attendant definitions wherein n is 0, R₁ is methyl, R₂ is CF₃, m is 1; and R₃ is 4-methylphenyl.

In a further embodiment, a sirtuin-activating compound is a compound of formula 42 and the attendant definitions wherein n is 0, R₁ is methyl, R₂ is CF₃, m is 1; R₃ is 4-methylphenyl; and L₁ is S.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, and L₂ is O.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, L₂ is O; and L₃ is NR₁.

In a further embodiment, a sirtuin-activating compound is a compound of formula 42 and the attendant definitions wherein n is 0, R₁ is methyl, R₂ is CF₃, m is 1; R₃ is 4-methylphenyl; L₁ is S, L₂ is O; L₃ is NR₁, and L₄ is NR₁.

In another embodiment, a sirtuin-activating compound is a compound of formula 43: 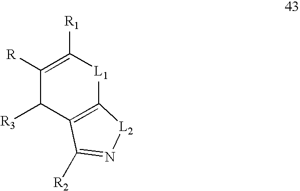
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ and R₃ are H or a substituted or unsubstituted alkyl, aryl, aralkyl, heterocyclyl, heterocyclylalkyl, heteroaryl, or heteroaralkyl; and

L₁ and L₂ are O, NR₂, or S.

In a further embodiment, a sirtuin-activating compound is a compound of formula 43 and the attendant definitions wherein R is cyano.

In a further embodiment, a sirtuin-activating compound is a compound of formula 43 and the attendant definitions wherein R₁ is NH₂.

In a further embodiment, a sirtuin-activating compound is a compound of formula 43 and the attendant definitions wherein R₂ is 4-bromophenyl.

In a further embodiment, a sirtuin-activating compound is a compound of formula 43 and the attendant definitions wherein R₃ is 3-hydroxy-4-methoxyphenyl.

In a further embodiment, a sirtuin-activating compound is a compound of formula 43 and the attendant definitions wherein L₁ is O.

In a further embodiment, a sirtuin-activating compound is a compound of formula 43 and the attendant definitions wherein L₂ is NR₂.

In a further embodiment, a sirtuin-activating compound is a compound of formula 43 and the attendant definitions wherein R is cyano and R₁ is NH₂.

In a further embodiment, a sirtuin-activating compound is a compound of formula 43 and the attendant definitions wherein R is cyano, R₁ is NH₂, and R₂ is 4-bromophenyl.

In a further embodiment, a sirtuin-activating compound is a compound of formula 43 and the attendant definitions wherein R is cyano, R₁ is NH₂, R₂ is 4-bromophenyl, and R₃ is 3-hydroxy-4-methoxyphenyl.

In a further embodiment, a sirtuin-activating compound is a compound of formula 43 and the attendant definitions wherein R is cyano, R₁ is NH₂, R₂ is 4-bromophenyl, R₃ is 3-hydroxy-4-methoxyphenyl, and L₁ is O.

In a further embodiment, a sirtuin-activating compound is a compound of formula 43 and the attendant definitions wherein R is cyano, R₁ is N12, R₂ is 4-bromophenyl, R₃ is 3-hydroxy-4-methoxyphenyl, L₁ is O, and L₂ is NR₂.

In another embodiment, a sirtuin-activating compound is a compound of formula 44: 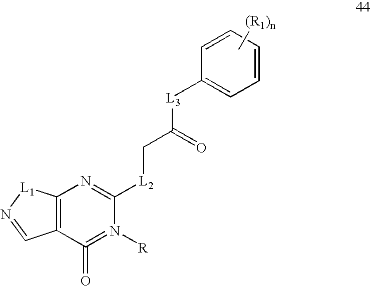
wherein, independently for each occurrence:

R is H or a substituted or unsubstituted alkyl, aryl, aralkyl, heterocyclyl, heterocyclylalkyl, heteroaryl, or heteroaralkyl;

R₁ is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S; and

n is an integer from 0 to 5 inclusive.

In a further embodiment, a sirtuin-activating compound is a compound of formula 44 and the attendant definitions wherein R is 3-trifluoromethylphenyl.

In a further embodiment, a sirtuin-activating compound is a compound of formula 44 and the attendant definitions wherein R₁ is C(O)OCH₃.

In a further embodiment, a sirtuin-activating compound is a compound of formula 44 and the attendant definitions wherein L₁ is NR.

In a further embodiment, a sirtuin-activating compound is a compound of formula 44 and the attendant definitions wherein L₂ is S.

In a further embodiment, a sirtuin-activating compound is a compound of formula 44 and the attendant definitions wherein L₃ is NR.

In a further embodiment, a sirtuin-activating compound is a compound of formula 44 and the attendant definitions wherein n is 2.

In a further embodiment, a sirtuin-activating compound is a compound of formula 44 and the attendant definitions wherein R is 3-trifluoromethylphenyl and R₁ is C(O)OCH₃.

In a further embodiment, a sirtuin-activating compound is a compound of formula 44 and the attendant definitions wherein R is 3-trifluoromethylphenyl, R₁ is C(O)OCH₃, and L₁ is NR.

In a further embodiment, a sirtuin-activating compound is a compound of formula 44 and the attendant definitions wherein R is 3-trifluoromethylphenyl, R₁ is C(O)OCH₃, L₁ is NR, and L₂ is S.

In a further embodiment, a sirtuin-activating compound is a compound of formula 44 and the attendant definitions wherein R is 3-trifluoromethylphenyl, R₁ is C(O)OCH₃, L₁ is NR, L₂ is S, and L₃ is NR.

In a further embodiment, a sirtuin-activating compound is a compound of formula 44 and the attendant definitions wherein R is 3-trifluoromethylphenyl, R₁ is C(O)OCH₃, L₁ is NR, L₂ is S, L₃ is NR, and n is 2.

In another embodiment, a sirtuin-activating compound is a compound of formula 45: 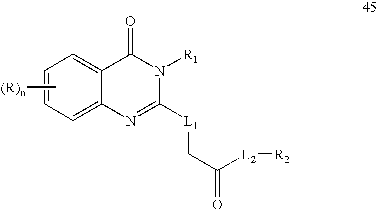
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H or a substituted or unsubstituted alkyl, aryl, aralkyl, heterocyclyl, heterocyclylalkyl, heteroaryl, or heteroaralkyl;

L₁ and L₂ are O, NR₁, or S; and

n is an integer from 0 to 4 inclusive.

In a further embodiment, a sirtuin-activating compound is a compound of formula 45 and the attendant definitions wherein n is 0.

In a further embodiment, a sirtuin-activating compound is a compound of formula 45 and the attendant definitions wherein R₁ is 2-tetrahydrofuranylmethyl.

In a further embodiment, a sirtuin-activating compound is a compound of formula 45 and the attendant definitions wherein R₂ is —CH₂CH₂C₆H₄SO₂NH₂.

In a further embodiment, a sirtuin-activating compound is a compound of formula 45 and the attendant definitions wherein L₁ is S.

In a further embodiment, a sirtuin-activating compound is a compound of formula 45 and the attendant definitions wherein L₂ is NR₁.

In a further embodiment, a sirtuin-activating compound is a compound of formula 45 and the attendant definitions wherein n is 0 and R₁ is 2-tetrahydrofuranylmethyl.

In a further embodiment, a sirtuin-activating compound is a compound of formula 45 and the attendant definitions wherein n is 0, R₁ is 2-tetrahydrofuranylmethyl, and R₂ is —CH₂CH₂C₆H₄SO₂NH₂.

In a further embodiment, a sirtuin-activating compound is a compound of formula 45 and the attendant definitions wherein n is 0, R₁ is 2-tetrahydrofuranylmethyl, R₂ is —CH₂CH₂C₆H₄SO₂NH₂, and L₁ is S.

In a further embodiment, a sirtuin-activating compound is a compound of formula 45 and the attendant definitions wherein n is 0, R₁ is 2-tetrahydrofuranylmethyl, R₂ is —CH₂CH₂C₆H₄SO₂NH₂, L₁ is S, and L₂ is NR₁.

In another embodiment, a sirtuin-activating compound is a compound of formula 46: 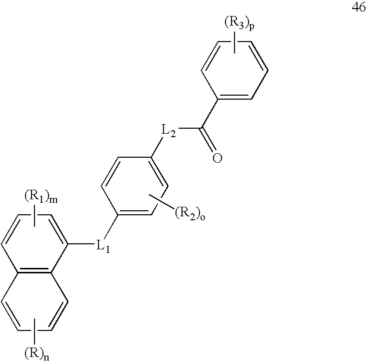
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl;

n is an integer from 0 to 4 inclusive;

m is an integer from 0 to 3 inclusive;

o is an integer from 0 to 4 inclusive; and

p is an integer from 0 to 5 inclusive.

In a further embodiment, a sirtuin-activating compound is a compound of formula 46 and the attendant definitions wherein n is 0.

In a further embodiment, a sirtuin-activating compound is a compound of formula 46 and the attendant definitions wherein m is 1.

In a further embodiment, a sirtuin-activating compound is a compound of formula 46 and the attendant definitions wherein R₁ is Cl.

In a further embodiment, a sirtuin-activating compound is a compound of formula 46 and the attendant definitions wherein o is 1.

In a further embodiment, a sirtuin-activating compound is a compound of formula 46 and the attendant definitions wherein R₂ is Cl.

In a further embodiment, a sirtuin-activating compound is a compound of formula 46 and the attendant definitions wherein p is 3.

In a further embodiment, a sirtuin-activating compound is a compound of formula 46 and the attendant definitions wherein R₃ is OH or I.

In a further embodiment, a sirtuin-activating compound is a compound of formula 46 and the attendant definitions wherein n is 0 and m is 1.

In a further embodiment, a sirtuin-activating compound is a compound of formula 46 and the attendant definitions wherein n is 0, m is 1, and o is 1.

In a further embodiment, a sirtuin-activating compound is a compound of formula 46 and the attendant definitions wherein n is 0, m is 1, o is 1, and R₁ is Cl.

In a further embodiment, a sirtuin-activating compound is a compound of formula 46 and the attendant definitions wherein n is 0, m is 1, o is 1, R₁ is Cl, and p is 3.

In a further embodiment, a sirtuin-activating compound is a compound of formula 46 and the attendant definitions wherein n is 0, m is 1, o is 1, R₁ is Cl, p is 3, and R₂ is OH or I.

In another embodiment, a sirtuin-activating compound is a compound of formula 47: 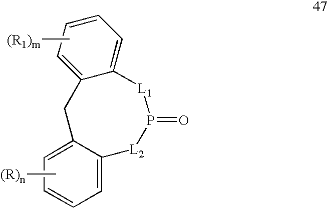
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₄, or S;

R₄ is H or a substituted or unsubstituted alkyl, aryl, aralkyl, heterocyclyl, heterocyclylalkyl, heteroaryl, or heteroaralkyl; and

m and n are integers from 0 to 4 inclusive.

In a further embodiment, a sirtuin-activating compound is a compound of formula 47 and the attendant definitions wherein n is 2.

In a further embodiment, a sirtuin-activating compound is a compound of formula 47 and the attendant definitions wherein R is methyl or t-butyl.

In a further embodiment, a sirtuin-activating compound is a compound of formula 47 and the attendant definitions wherein m is 2.

In a further embodiment, a sirtuin-activating compound is a compound of formula 47 and the attendant definitions wherein R₁ is methyl or t-butyl.

In a further embodiment, a sirtuin-activating compound is a compound of formula 47 and the attendant definitions wherein L₁ is O.

In a further embodiment, a sirtuin-activating compound is a compound of formula 47 and the attendant definitions wherein L₂ is O.

In a further embodiment, a sirtuin-activating compound is a compound of formula 47 and the attendant definitions wherein n is 2 and R is methyl or t-butyl.

In a further embodiment, a sirtuin-activating compound is a compound of formula 47 and the attendant definitions wherein n is 2, R is methyl or t-butyl, and m is 2.

In a further embodiment, a sirtuin-activating compound is a compound of formula 47 and the attendant definitions wherein n is 2, R is methyl or t-butyl, m is 2, and R₁ is methyl or t-butyl.

In a further embodiment, a sirtuin-activating compound is a compound of formula 47 and the attendant definitions wherein n is 2, R is methyl or t-butyl, m is 2, R₁ is methyl or t-butyl, and L₁ is O.

In a further embodiment, a sirtuin-activating compound is a compound of formula 47 and the attendant definitions wherein n is 2, R is methyl or t-butyl, m is 2, R₁ is methyl or t-butyl, L₁ is O, and L₂ is O.

In another embodiment, a sirtuin-activating compound is a compound of formula 48: 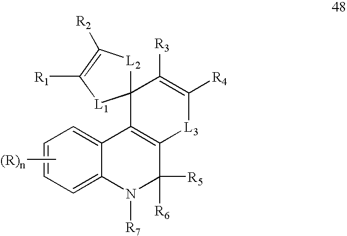
wherein, independently for each occurrence:

R, R₁, R₂, R₃, R₄, R₅, and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₇ is H or a substituted or unsubstituted alkyl, acyl, aryl, aralkyl, heterocyclyl, heterocyclylalkyl, heteroaryl, or heteroaralkyl;

L₁, L₂, and L₃ are O, NR₇, or S and

n is an integer from 0 to 4 inclusive.

In a further embodiment, a sirtuin-activating compound is a compound of formula 48 and the attendant definitions wherein n is 1.

In a further embodiment, a sirtuin-activating compound is a compound of formula 48 and the attendant definitions wherein R is methyl.

In a further embodiment, a sirtuin-activating compound is a compound of formula 48 and the attendant definitions wherein R₁ is C(O)OCH₃.

In a further embodiment, a sirtuin-activating compound is a compound of formula 48 and the attendant definitions wherein R₂ is C(O)OCH₃.

In a further embodiment, a sirtuin-activating compound is a compound of formula 48 and the attendant definitions wherein R₃ is C(O)OCH₃.

In a further embodiment, a sirtuin-activating compound is a compound of formula 48 and the attendant definitions wherein R₄ is C(O)OCH₃.

In a further embodiment, a sirtuin-activating compound is a compound of formula 48 and the attendant definitions wherein R₅ is methyl.

In a further embodiment, a sirtuin-activating compound is a compound of formula 48 and the attendant definitions wherein R₆ is methyl.

In a further embodiment, a sirtuin-activating compound is a compound of formula 48 and the attendant definitions wherein R₇ is C(O)CF₃.

In a further embodiment, a sirtuin-activating compound is a compound of formula 48 and the attendant definitions wherein L₁ is S.

In a further embodiment, a sirtuin-activating compound is a compound of formula 48 and the attendant definitions wherein L₂ is S.

In a further embodiment, a sirtuin-activating compound is a compound of formula 48 and the attendant definitions wherein L₃ is S.

In a further embodiment, a sirtuin-activating compound is a compound of formula 48 and the attendant definitions wherein n is 1 and R is methyl.

In a further embodiment, a sirtuin-activating compound is a compound of formula 48 and the attendant definitions wherein n is 1, R is methyl, and R₁ is C(O)OCH₃.

In a further embodiment, a sirtuin-activating compound is a compound of formula 48 and the attendant definitions wherein n is 1, R is methyl, R₁ is C(O)OCH₃, and R₂ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, and R₃ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, and R₄ is C(O)OCH₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, and R₅ is methyl.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, and R₆ is methyl.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, and R₇ is C(O)CF₃.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, and L₁ is S.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, and L₂ is S.

In a further embodiment, a sirtuin-activating compound is a compound of formula 48 and the attendant definitions wherein n is 1, R is methyl, R₁ is C(O)OCH₃, R₂ is C(O)OCH₃, R₃ is C(O)OCH₃, R₄ is C(O)OCH₃, R₅ is methyl, R₆ is methyl, R₇ is C(O)CF₃, L₁ is S, L₂ is S, and L₃ is S.

In another embodiment, a sirtuin-activating compound is a compound of formula 49: 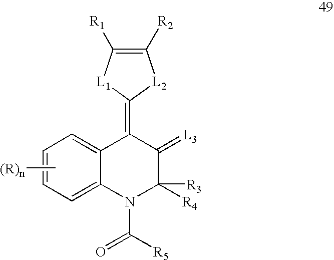
wherein, independently for each occurrence:

R, R₁, R₂, R₃, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₆, or S;

R₆ is H or a substituted or unsubstituted alkyl, aryl, aralkyl, heterocyclyl, heterocyclylalkyl, heteroaryl, or heteroaralkyl; and

n is an integer from 0 to 4 inclusive.

In a further embodiment, a sirtuin-activating compound is a compound of formula 49 and the attendant definitions wherein n is 1.

In a further embodiment, a sirtuin-activating compound is a compound of formula 49 and the attendant definitions wherein R is methyl.

In a further embodiment, a sirtuin-activating compound is a compound of formula 49 and the attendant definitions wherein R₁ is C(O)OCH₃.

In a further embodiment, a sirtuin-activating compound is a compound of formula 49 and the attendant definitions wherein R₂ is C(O)OCH₃.

In a further embodiment, a sirtuin-activating compound is a compound of formula 49 and the attendant definitions wherein R₃ is methyl.

In a further embodiment, a sirtuin-activating compound is a compound of formula 49 and the attendant definitions wherein R₄ is methyl.

In a further embodiment, a sirtuin-activating compound is a compound of formula 49 and the attendant definitions wherein R₅ is CH₂CH(CH₃)₂.

In a further embodiment, a sirtuin-activating compound is a compound of formula 49 and the attendant definitions wherein L₁ is S.

In a further embodiment, a sirtuin-activating compound is a compound of formula 49 and the attendant definitions wherein L₂ is S.

In a further embodiment, a sirtuin-activating compound is a compound of formula 49 and the attendant definitions wherein L₃ is S.

In a further embodiment, a sirtuin-activating compound is a compound of formula 49 and the attendant definitions wherein n is 1 and R is methyl.

In a further embodiment, a sirtuin-activating compound is a compound of formula 49 and the attendant definitions wherein n is 1, R is methyl, and R₁ is C(O)OCH₃.

In a further embodiment, a sirtuin-activating compound is a compound of formula 49 and the attendant definitions wherein n is 1, R is methyl, R₁ is C(O)OCH₃, and R₂ is C(O)OCH₃.

In a further embodiment, a sirtuin-activating compound is a compound of formula 49 and the attendant definitions wherein n is 1, R is methyl, R₁ is C(O)OCH₃, R₂ is C(O)OCH₃, and R₃ is methyl.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, and R₄ is methyl.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, and R₅ is CH₂CH(CH₃)₂.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, and L₁ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, and L₂ is S.

In a further embodiment, a sirtuin-activating compound is a compound of formula 49 and the attendant definitions wherein n is 1, R is methyl, R₁ is C(O)OCH₃, R₂ is C(O)OCH₃, R₃ is methyl, R₄ is methyl, R₅ is CH₂CH(CH₃)₂, L₁ is S, L₂ is S, and L₃ is S.

In another embodiment, a sirtuin-activating compound is a compound of formula 50: 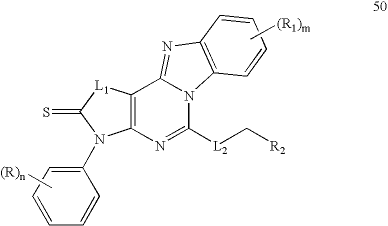
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁ and L₂ are O, NR₃, or S;

R₃ is H or a substituted or unsubstituted alkyl, aryl, aralkyl, heterocyclyl, heterocyclylalkyl, heteroaryl, or heteroaralkyl;

n is an integer from 0 to 5 inclusive; and

m is an integer from 0 to 4 inclusive.

In a further embodiment, a sirtuin-activating compound is a compound of formula 50 and the attendant definitions wherein n is 1.

In a further embodiment, a sirtuin-activating compound is a compound of formula 50 and the attendant definitions wherein R is CO₂Et.

In a further embodiment, a sirtuin-activating compound is a compound of formula 50 and the attendant definitions wherein m is 0.

In a further embodiment, a sirtuin-activating compound is a compound of formula 50 and the attendant definitions wherein R₂ is cyano.

In a further embodiment, a sirtuin-activating compound is a compound of formula 50 and the attendant definitions wherein L₁ is S.

In a further embodiment, a sirtuin-activating compound is a compound of formula 50 and the attendant definitions wherein L₂ is S.

In a further embodiment, a sirtuin-activating compound is a compound of formula 50 and the attendant definitions wherein n is 1 and R is CO₂Et.

In a further embodiment, a sirtuin-activating compound is a compound of formula 50 and the attendant definitions wherein n is 1, R is CO₂Et, and m is 0.

In a further embodiment, a sirtuin-activating compound is a compound of formula 50 and the attendant definitions wherein n is 1, R is CO₂Et, m is 0, and R₂ is cyano.

In a further embodiment, a sirtuin-activating compound is a compound of formula 50 and the attendant definitions wherein n is 1, R is CO₂Et, m is 0, R₂ is cyano, and L₁ is S.

In a further embodiment, a sirtuin-activating compound is a compound of formula 50 and the attendant definitions wherein n is 1, R is CO₂Et, m is 0, R₂ is cyano, L₁ is S, and L₂ is S.

In another embodiment, a sirtuin-activating compound is a compound of formula 51: 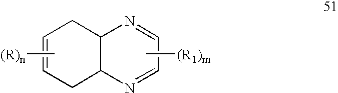
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 2 inclusive.

In a further embodiment, a sirtuin-activating compound is a compound of formula 51 and the attendant definitions wherein n is 2.

In a further embodiment, a sirtuin-activating compound is a compound of formula 51 and the attendant definitions wherein R is Cl or trifluoromethyl.

In a further embodiment, a sirtuin-activating compound is a compound of formula 51 and the attendant definitions wherein m is 2.

In a further embodiment, a sirtuin-activating compound is a compound of formula 51 and the attendant definitions wherein R₁ is phenyl.

In a further embodiment, a sirtuin-activating compound is a compound of formula 51 and the attendant definitions wherein n is 2 and R is Cl or trifluoromethyl.

In a further embodiment, a sirtuin-activating compound is a compound of formula 51 and the attendant definitions wherein n is 2, R is Cl or trifluoromethyl, and m is 2.

In a further embodiment, a sirtuin-activating compound is a compound of formula 51 and the attendant definitions wherein n is 2, R is Cl or trifluoromethyl, m is 2, and R₁ is phenyl.

In a further embodiment, a sirtuin-activating compound is a compound of formula 51 and the attendant definitions wherein n is 1.

In a further embodiment, a sirtuin-activating compound is a compound of formula 51 and the attendant definitions wherein R is F.

In a further embodiment, a sirtuin-activating compound is a compound of formula 51 and the attendant definitions wherein R₁ is 4-methylphenyl.

In a further embodiment, a sirtuin-activating compound is a compound of formula 51 and the attendant definitions wherein n is I and R is F.

In a further embodiment, a sirtuin-activating compound is a compound of formula 51 and the attendant definitions wherein n is 1, R is F, and m is 2.

In a further embodiment, a sirtuin-activating compound is a compound of formula 51 and the attendant definitions wherein n is 1, R is F, m is 2, and R₁ is 4-methylphenyl.

In another embodiment, a sirtuin-activating compound is a compound of formula 52: 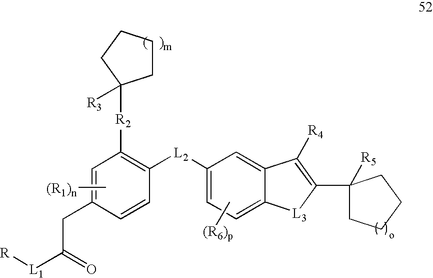
wherein, independently for each occurrence:

R is H or a substituted or unsubstituted alkyl, aryl, aralkyl, heterocyclyl, heterocyclylalkyl, heteroaryl, or heteroaralkyl;

R₁ and R₆ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₂ is alkylene, alkenylene, or alkynylene;

R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR, or S;

n and p are integers from 0 to 3 inclusive; and

m and o are integers from 0 to 2 inclusive.

In a further embodiment, a sirtuin-activating compound is a compound of formula 52 and the attendant definitions wherein R is CH₂CH₂OH.

In a further embodiment, a sirtuin-activating compound is a compound of formula 52 and the attendant definitions wherein n is 1.

In a further embodiment, a sirtuin-activating compound is a compound of formula 52 and the attendant definitions wherein R₁ is I.

In a further embodiment, a sirtuin-activating compound is a compound of formula 52 and the attendant definitions wherein R₂ is alkynylene.

In a further embodiment, a sirtuin-activating compound is a compound of formula 52 and the attendant definitions wherein m is 1.

In a further embodiment, a sirtuin-activating compound is a compound of formula 52 and the attendant definitions wherein R₃ is OH.

In a further embodiment, a sirtuin-activating compound is a compound of formula 52 and the attendant definitions wherein R₄ is C(O)OEt.

In a further embodiment, a sirtuin-activating compound is a compound of formula 52 and the attendant definitions wherein o is 1.

In a further embodiment, a sirtuin-activating compound is a compound of formula 52 and the attendant definitions wherein R₅ is OH.

In a further embodiment, a sirtuin-activating compound is a compound of formula 52 and the attendant definitions wherein p is 0.

In a further embodiment, a sirtuin-activating compound is a compound of formula 52 and the attendant definitions wherein L₁ is NH.

In a further embodiment, a sirtuin-activating compound is a compound of formula 52 and the attendant definitions wherein L₂ is O.

In a further embodiment, a sirtuin-activating compound is a compound of formula 52 and the attendant definitions wherein L₃ is O.

In a further embodiment, a sirtuin-activating compound is a compound of formula 52 and the attendant definitions wherein R is CH₂CH₂OH and n is 1.

In a further embodiment, a sirtuin-activating compound is a compound of formula 52 and the attendant definitions wherein R is CH₂CH₂OH, n is 1, and R₁ is I.

In a further embodiment, a sirtuin-activating compound is a compound of formula 52 and the attendant definitions wherein R is CH₂CH₂OH, n is 1, R₁ is I, and R₂ is alkynylene.

In a further embodiment, a sirtuin-activating compound is a compound of formula 52 and the attendant definitions wherein R is CH₂CH₂OH, n is 1, R₁ is I, R₂ is alkynylene, and m is 1.

In a further embodiment, a sirtuin-activating compound is a compound of formula 52 and the attendant definitions wherein R is CH₂CH₂OH, n is 1, R₁ is I, R₂ is alkynylene, m is 1, and R₃ is OH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, and R₄ is C(O)OEt.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, and o is 1.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, and R₅ is OH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, and p is 0.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, and L₁ is NH.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, and L₂ is O.

In a further embodiment, a sirtuin-activating compound is a compound of formula 52 and the attendant definitions wherein R is CH₂CH₂OH, n is 1, R₁ is I, R₂ is alkynylene, m is 1, R₃ is OH, R₄ is C(O)OEt, o is 1, R₅ is OH, p is 0, L₁ is NH, L₂ is O, and L₃ is O.

In another embodiment, a sirtuin-activating compound is a compound of formula 53: 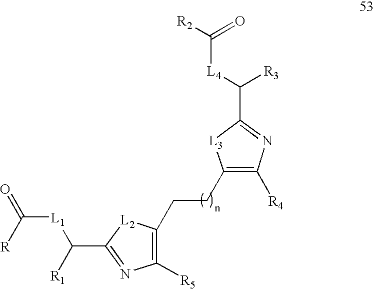
wherein, independently for each occurrence:

R, R₁, R₂, R₃, R₄, and R₅ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, L₃, and L₄ are O, NR₆, or S;

R₆ is and H, or a substituted or unsubstituted alkyl, aryl, aralkyl, heterocyclyl, heterocyclylalkyl, heteroaryl, or heteroaralkyl; and

n is an integer from 0 to 5 inclusive.

In a further embodiment, a sirtuin-activating compound is a compound of formula 53 and the attendant definitions wherein R is O-t-butyl.

In a further embodiment, a sirtuin-activating compound is a compound of formula 53 and the attendant definitions wherein R₁ is t-butyl.

In a further embodiment, a sirtuin-activating compound is a compound of formula 53 and the attendant definitions wherein R₂ is O-t-butyl.

In a further embodiment, a sirtuin-activating compound is a compound of formula 53 and the attendant definitions wherein R₃ is t-butyl.

In a further embodiment, a sirtuin-activating compound is a compound of formula 53 and the attendant definitions wherein R₄ is C(O)OMe.

In a further embodiment, a sirtuin-activating compound is a compound of formula 53 and the attendant definitions wherein R₅ is C(O)OMe.

In a further embodiment, a sirtuin-activating compound is a compound of formula 53 and the attendant definitions wherein L₁ is NH.

In a further embodiment, a sirtuin-activating compound is a compound of formula 53 and the attendant definitions wherein L₂ is O.

In a further embodiment, a sirtuin-activating compound is a compound of formula 53 and the attendant definitions wherein L₃ is O.

In a further embodiment, a sirtuin-activating compound is a compound of formula 53 and the attendant definitions wherein L₄ is NH.

In a further embodiment, a sirtuin-activating compound is a compound of formula 53 and the attendant definitions wherein n is 1.

In a further embodiment, a sirtuin-activating compound is a compound of formula 53 and the attendant definitions wherein R is O-t-butyl and R₁ is t-butyl.

In a further embodiment, a sirtuin-activating compound is a compound of formula 53 and the attendant definitions wherein R is O-t-butyl, R₁ is t-butyl, and R₂ is O-t-butyl.

In a further embodiment, a sirtuin-activating compound is a compound of formula 53 and the attendant definitions wherein R is O-t-butyl, R₁ is t-butyl, R₂ is O-t-butyl, and R₃ is t-butyl.

In a further embodiment, a sirtuin-activating compound is a compound of formula 53 and the attendant definitions wherein R is O-t-butyl, R₁ is t-butyl, R₂ is O-t-butyl, R₃ is t-butyl, and R₄ is C(O)OMe.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, and R₅ is C(O)OMe.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, and L₁ is NH.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, and L₂ is O.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, and L₃ is O.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, and L₄ is NH.

In a further embodiment, a sirtuin-activating compound is a compound of formula 53 and the attendant definitions wherein R is O-t-butyl, R₁ is t-butyl, R₂ is O-t-butyl, R₃ is t-butyl, R₄ is C(O)OMe, R₅ is C(O)OMe, L₁ is NH, L₂ is O, L₃ is O, L₄ is NH, and n is 1.

In another embodiment, a sirtuin-activating compound is a compound of formula 54: 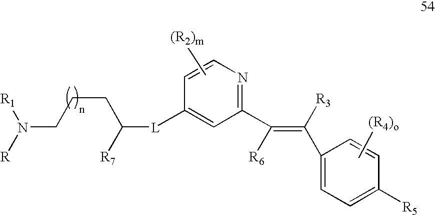
wherein, independently for each occurrence:

R and R₁ are H or a substituted or unsubstituted alkyl, aryl, aralkyl, heterocyclyl, heterocyclylalkyl, heteroaryl, or heteroaralkyl;

R₂, R₄, and R₅ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₃, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR, or S;

n and o are integers from 0 to 4 inclusive; and

m is an integer from 0 to 3 inclusive.

In a further embodiment, a sirtuin-activating compound is a compound of formula 54 and the attendant definitions wherein R is ethyl.

In a further embodiment, a sirtuin-activating compound is a compound of formula 54 and the attendant definitions wherein R₁ is ethyl.

In a further embodiment, a sirtuin-activating compound is a compound of formula 54 and the attendant definitions wherein m is 0.

In a further embodiment, a sirtuin-activating compound is a compound of formula 54 and the attendant definitions wherein R₃ is H.

In a further embodiment, a sirtuin-activating compound is a compound of formula 54 and the attendant definitions wherein o is 0.

In a further embodiment, a sirtuin-activating compound is a compound of formula 54 and the attendant definitions wherein R₅ is Cl.

In a further embodiment, a sirtuin-activating compound is a compound of formula 54 and the attendant definitions wherein R₆ is H.

In a further embodiment, a sirtuin-activating compound is a compound of formula 54 and the attendant definitions wherein R₇ is methyl.

In a further embodiment, a sirtuin-activating compound is a compound of formula 54 and the attendant definitions wherein L is NH.

In a further embodiment, a sirtuin-activating compound is a compound of formula 54 and the attendant definitions wherein n is 1.

In a further embodiment, a sirtuin-activating compound is a compound of formula 54 and the attendant definitions wherein R is ethyl and R₁ is ethyl.

In a further embodiment, a sirtuin-activating compound is a compound of formula 54 and the attendant definitions wherein R is ethyl, R₁ is ethyl, and m is 0.

In a further embodiment, a sirtuin-activating compound is a compound of formula 54 and the attendant definitions wherein R is ethyl, R₁ is ethyl, m is 0, and R₃ is H.

In a further embodiment, a sirtuin-activating compound is a compound of formula 54 and the attendant definitions wherein R is ethyl, R₁ is ethyl, m is 0, R₃ is H, and o is 0.

In a further embodiment, a sirtuin-activating compound is a compound of formula 54 and the attendant definitions wherein R is ethyl, R₁ is ethyl, m is 0, R₃ is H, o is 0, and R₅ is Cl.

In a further embodiment, a sirtuin-activating compound is a compound of formula 54 and the attendant definitions wherein R is ethyl, R₁ is ethyl, m is 0, R₃ is H, o is 0, R₅ is Cl, and R₆ is H.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, and R₇ is methyl.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, R₇ is methyl, and L is NH.

In a further embodiment, a sirtuin-activating compound is a compound of formula 54 and the attendant definitions wherein R is ethyl, R₁ is ethyl, m is 0, R₃ is H, o is 0, R₅ is Cl, R₆ is H, R₇ is methyl, L is NH, and n is 1.

In another embodiment, a sirtuin-activating compound is a compound of formula 55: 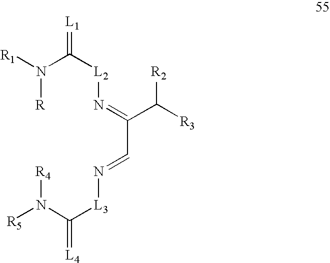
wherein, independently for each occurrence:

R, R₁, R₄, and R₅ are H or a substituted or unsubstituted alkyl, aryl, aralkyl, heterocyclyl, heterocyclylalkyl, heteroaryl, or heteroaralkyl;

R₂ and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁, L₂, L₃, and L₄ are O, NR, or S.

In a further embodiment, a sirtuin-activating compound is a compound of formula 55 and the attendant definitions wherein R is H.

In a further embodiment, a sirtuin-activating compound is a compound of formula 55 and the attendant definitions wherein R₁ is H.

In a further embodiment, a sirtuin-activating compound is a compound of formula 55 and the attendant definitions wherein R₂ is OEt.

In a further embodiment, a sirtuin-activating compound is a compound of formula 55 and the attendant definitions wherein R₃ is methyl.

In a further embodiment, a sirtuin-activating compound is a compound of formula 55 and the attendant definitions wherein R₄ is H.

In a further embodiment, a sirtuin-activating compound is a compound of formula 55 and the attendant definitions wherein R₅ is H.

In a further embodiment, a sirtuin-activating compound is a compound of formula 55 and the attendant definitions wherein L₁ is S.

In a further embodiment, a sirtuin-activating compound is a compound of formula 55 and the attendant definitions wherein L₂ is NH.

In a further embodiment, a sirtuin-activating compound is a compound of formula 55 and the attendant definitions wherein L₃ is NH.

In a further embodiment, a sirtuin-activating compound is a compound of formnula 55 and the attendant definitions wherein L₄ is S.

In a further embodiment, a sirtuin-activating compound is a compound of formula 55 and the attendant definitions wherein R is H and R₁ is H.

In a further embodiment, a sirtuin-activating compound is a compound of formula 55 and the attendant definitions wherein R is H, R₁ is H, and R₂ is OEt.

In a further embodiment, a sirtuin-activating compound is a compound of formula 55 and the attendant definitions wherein R is H, R₁ is H, R₂ is OEt, and R₃ is methyl.

In a further embodiment, a sirtuin-activating compound is a compound of formula 55 and the attendant definitions wherein R is H, R₁ is H, R₂ is OEt, R₃ is methyl, and R₄ is H.

In a further embodiment, a sirtuin-activating compound is a compound of formula 55 and the attendant definitions wherein R is H, R₁ is H, R₂ is OEt, R₃ is methyl, R₄ is H, and R₅ is H.

In a further embodiment, a sirtuin-activating compound is a compound of formula 55 and the attendant definitions wherein R is H, R₁ is H, R₂ is OEt, R₃ is methyl, P4 is H, R₅ is H, and L₁ is S.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, and L₂ is NH.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, L₂ is NH, and L₃ is NH.

In a further embodiment, a sirtuin-activating compound is a compound of formula 55 and the attendant definitions wherein R is H, R₁ is H, R₂ is OEt, R₃ is methyl, R₄ is H, R₅ is H, L₁ is S, L₂ is NH, L₃ is NH, and L₄ is S.

In another embodiment, a sirtuin-activating compound is a compound of formula 56: 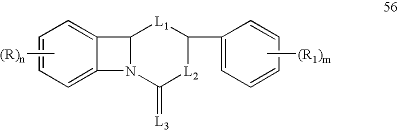
wherein, independently for each occurrence:

R and R₁ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₂, or S;

R₂ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 0 to 5 inclusive.

In a further embodiment, a sirtuin-activating compound is a compound of formula 56 and the attendant definitions wherein n is 0.

In a further embodiment, a sirtuin-activating compound is a compound of formula 56 and the attendant definitions wherein m is 0.

In a further embodiment, a sirtuin-activating compound is a compound of formula 56 and the attendant definitions wherein L₁ is NH.

In a further embodiment, a sirtuin-activating compound is a compound of formula 56 and the attendant definitions wherein L₂ is S.

In a further embodiment, a sirtuin-activating compound is a compound of formula 56 and the attendant definitions wherein L₃ is S.

In a further embodiment, a sirtuin-activating compound is a compound of formula 56 and the attendant definitions wherein m is 0 and n is 0.

In a further embodiment, a sirtuin-activating compound is a compound of formula 56 and the attendant definitions wherein m is 0, n is 0, and L₁ is NH.

In a further embodiment, a sirtuin-activating compound is a compound of formula 56 and the attendant definitions wherein m is 0, n is 0, L₁ is NH, and L₂ is S.

In a further embodiment, a sirtuin-activating compound is a compound of formula 56 and the attendant definitions wherein m is 0, n is 0, L₁ is NH, L₂ is S, and L₃ is S.

In another embodiment, a sirtuin-activating compound is a compound of formula 57: 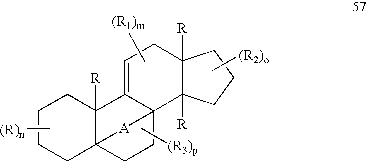
wherein, independently for each occurrence:

R, R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

A is alkylene, alkenylene, or alkynylene;

n is an integer from 0 to 8 inclusive;

m is an integer from 0 to 3 inclusive;

o is an integer from 0 to 6 inclusive; and

p is an integer from 0 to 4 inclusive.

In a further embodiment, a sirtuin-activating compound is a compound of formula 57 and the attendant definitions wherein n is 2.

In a further embodiment, a sirtuin-activating compound is a compound of formula 57 and the attendant definitions wherein R is OH or methyl.

In a further embodiment, a sirtuin-activating compound is a compound of formula 57 and the attendant definitions wherein m is 1.

In a further embodiment, a sirtuin-activating compound is a compound of formula 57 and the attendant definitions wherein R₁ is methyl.

In a further embodiment, a sirtuin-activating compound is a compound of formula 57 and the attendant definitions wherein o is 1.

In a further embodiment, a sirtuin-activating compound is a compound of formula 57 and the attendant definitions wherein R₂ is C(O)CH₃.

In a further embodiment, a sirtuin-activating compound is a compound of formula 57 and the attendant definitions wherein p is 2.

In a further embodiment, a sirtuin-activating compound is a compound of formula 57 and the attendant definitions wherein R₃ is CO₂H.

In a further embodiment, a sirtuin-activating compound is a compound of formula 57 and the attendant definitions wherein A is alkenylene.

In a further embodiment, a sirtuin-activating compound is a compound of formula 57 and the attendant definitions wherein n is 2 and R is OH or methyl.

In a further embodiment, a sirtuin-activating compound is a compound of formula 57 and the attendant definitions wherein n is 2, R is OH or methyl, and m is 1.

In a further embodiment, a sirtuin-activating compound is a compound of formula 57 and the attendant definitions wherein n is 2, R is OH or methyl, m is 1, and R₁ is methyl.

In a further embodiment, a sirtuin-activating compound is a compound of formula 57 and the attendant definitions wherein n is 2, R is OH or methyl, m is 1, R₁ is methyl, and o is 1.

In a further embodiment, a sirtuin-activating compound is a compound of formula 57 and the attendant definitions wherein n is 2, R is OH or methyl, m is 1, R₁ is methyl, o is 1, and R₂ is C(O)CH₃.

In a further embodiment, a sirtuin-activating compound is a compound of formula 57 and the attendant definitions wherein n is 2, R is OH or methyl, m is 1, R₁ is methyl, o is 1, R₂ is C(O)CH₃, and p is 2.

In a further embodiment, a sirtuin-activating compound is a compound of formula 57 and the attendant definitions wherein n is 2, R is OH or methyl, m is 1, R₁ is methyl, o is 1, R₂ is C(O)CH₃, p is 2, and R₃ is CO₂H.

In a further embodiment, a sirtuin-activating compound is a compound of formula 57 and the attendant definitions wherein n is 2, R is OH or methyl, m is 1, R₁ is methyl, o is 1, R₂ is C(O)CH₃, p is 2, R₃ is CO₂H, and A is alkenylene.

In another embodiment, a sirtuin-activating compound is a compound of formula 58: 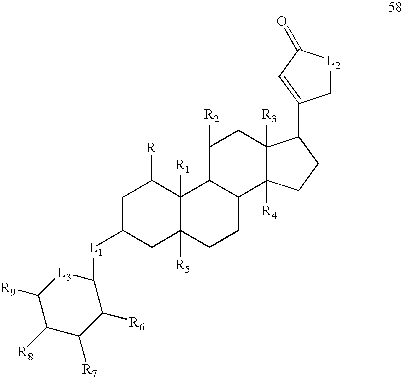
wherein, independently for each occurrence:

R, R₁, R₂, R₃, R₄, R₅, R₆, R₇, R₈, and R9 are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are O, NR₁₀, or S; and

R₁₀ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 58 and the attendant definitions wherein R is OH.

In a further embodiment, a sirtuin-activating compound is a compound of formula 58 and the attendant definitions wherein R₁ is CH₂OH.

In a further embodiment, a sirtuin-activating compound is a compound of formula 58 and the attendant definitions wherein R₂ is OH.

In a further embodiment, a sirtuin-activating compound is a compound of formula 58 and the attendant definitions wherein R₃ is methyl.

In a further embodiment, a sirtuin-activating compound is a compound of formula 58 and the attendant definitions wherein R₄ is OH.

In a further embodiment, a sirtuin-activating compound is a compound of formula 58 and the attendant definitions wherein R₅ is OH.

In a further embodiment, a sirtuin-activating compound is a compound of formula 58 and the attendant definitions wherein R₆ is OH.

In a further embodiment, a sirtuin-activating compound is a compound of formula 58 and the attendant definitions wherein R₇ is OH.

In a further embodiment, a sirtuin-activating compound is a compound of formula 58 and the attendant definitions wherein R₈ is OH.

In a further embodiment, a sirtuin-activating compound is a compound of formula 58 and the attendant definitions wherein R₉ is methyl.

In a further embodiment, a sirtuin-activating compound is a compound of formula 58 and the attendant definitions wherein L₁ is O.

In a further embodiment, a sirtuin-activating compound is a compound of formula 58 and the attendant definitions wherein L₂ is O.

In a further embodiment, a sirtuin-activating compound is a compound of formula 58 and the attendant definitions wherein L₃ is O.

In a further embodiment, a sirtuin-activating compound is a compound of formula 58 and the attendant definitions wherein R is OH and R₁ is CH₂OH.

In a further embodiment, a sirtuin-activating compound is a compound of formula 58 and the attendant definitions wherein R is OH, R₁ is CH₂OH, and R₂ is OH.

In a further embodiment, a sirtuin-activating compound is a compound of formula 58 and the attendant definitions wherein R is OH, R₁ is CH₂OH, R₂ is OH, and R₃ is methyl.

In a further embodiment, a sirtuin-activating compound is a compound of formula 58 and the attendant definitions wherein R is OH, R₁ is CH₂OH, R₂ is OH, R₃ is methyl, and R₄ is OH.

In a further embodiment, a sirtuin-activating compound is a compound of formula 58 and the attendant definitions wherein R is OH, R₁ is CH₂OH, R₂ is OH, R₃ is methyl, R₄ is OH, and R₅ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, and R₆ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, and R₇ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, and R₈ is OH.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, and R₉ is methyl.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, and L₁ is O.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, and L₂ is O.

In a further embodiment, a sirtuin-activating compound is a compound of formula 58 and the attendant definitions wherein R is OH, R₁ is CH₂OH, R₂ is OH, R₃ is methyl, R₄ is OH, R₅ is OH, R₆ is OH, R₇ is OH, R₈ is OH, R₉ is methyl, L₁ is O, L₂ is O, and L₃ is O.

In another embodiment, a sirtuin-activating compound is a compound of formula 59: 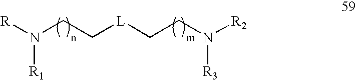
wherein, independently for each occurrence:

R, R₁, R₂, and R₃ are H or a substituted or unsubstituted alkyl, aryl, aralkyl, heterocyclyl, heterocyclylalkyl, heteroaryl, or heteroaralkyl;

L is O, NR, S, or Se; and

n and m are integers from 0 to 5 inclusive.

In a further embodiment, a sirtuin-activating compound is a compound of formula 59 and the attendant definitions wherein R is H.

In a further embodiment, a sirtuin-activating compound is a compound of formula 59 and the attendant definitions wherein R₁ is H.

In a further embodiment, a sirtuin-activating compound is a compound of formula 59 and the attendant definitions wherein R₂ is H.

In a further embodiment, a sirtuin-activating compound is a compound of formula 59 and the attendant definitions wherein R₃ is H.

In a further embodiment, a sirtuin-activating compound is a compound of formula 59 and the attendant definitions wherein L is Se.

In a further embodiment, a sirtuin-activating compound is a compound of formula 59 and the attendant definitions wherein n is 1.

In a further embodiment, a sirtuin-activating compound is a compound of formula 59 and the attendant definitions wherein m is 1.

In a further embodiment, a sirtuin-activating compound is a compound of formula 59 and the attendant definitions wherein R is H and R₁ is H.

In a further embodiment, a sirtuin-activating compound is a compound of formula 59 and the attendant definitions wherein R is H, R₁ is H, and R₂ is H.

In a further embodiment, a sirtuin-activating compound is a compound of formula 59 and the attendant definitions wherein R is H, R₁ is H, R₂ is H, and R₃ is H.

In a further embodiment, a sirtuin-activating compound is a compound of formula 59 and the attendant definitions wherein R is H, R₁ is H, R₂ is H, R₃ is H, and L is Se.

In a further embodiment, a sirtuin-activating compound is a compound of formula 59 and the attendant definitions wherein R is H, R₁ is H, R₂ is H, R₃ is H, L is Se, and n is 1.

In a further embodiment, a sirtuin-activating compound is a compound of formula 59 and the attendant definitions wherein R is H, R₁ is H, R₂ is H, R₃ is H, L is Se, n is 1, and m is 1.

In another embodiment, a sirtuin-activating compound is a compound of formula 60: 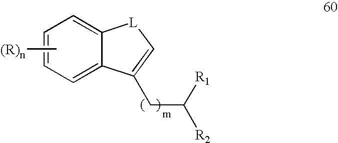
wherein, independently for each occurrence:

R is hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

R₁ and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₃, S, or SO₂;

R₃ is H or a substituted or unsubstituted alkyl, aryl, aralkyl, heterocyclyl, heterocyclylalkyl, heteroaryl, or heteroaralkyl;

n is an integer from 0 to 4 inclusive; and

m is an integer from 1 to 5 inclusive.

In a further embodiment, a sirtuin-activating compound is a compound of formula 60 and the attendant definitions wherein n is 1.

In a further embodiment, a sirtuin-activating compound is a compound of formula 60 and the attendant definitions wherein R is Cl.

In a further embodiment, a sirtuin-activating compound is a compound of formula 60 and the attendant definitions wherein R₁ is NH₂.

In a further embodiment, a sirtuin-activating compound is a compound of formula 60 and the attendant definitions wherein R₂ is CO₂H.

In a further embodiment, a sirtuin-activating compound is a compound of formula 60 and the attendant definitions wherein L is SO₂.

In a further embodiment, a sirtuin-activating compound is a compound of formula 60 and the attendant definitions wherein m is 1.

In a further embodiment, a sirtuin-activating compound is a compound of formula 60 and the attendant definitions wherein n is 1 and R is Cl.

In a further embodiment, a sirtuin-activating compound is a compound of formnula 60 and the attendant definitions wherein n is 1, R is Cl, and R₁ is NH₂.

In a further embodiment, a sirtuin-activating compound is a compound of formula 60 and the attendant definitions wherein n is 1, R is Cl, R₁ is NH₂, and R₂ is CO₂H.

In a further embodiment, a sirtuin-activating compound is a compound of formula 60 and the attendant definitions wherein n is 1, R is Cl, R₁ is NH₂, R₂ is CO₂H, and L is SO₂.

In a further embodiment, a sirtuin-activating compound is a compound of formula 60 and the attendant definitions wherein n is 1, R is Cl, R₁ is NH₂, R₂ is CO₂H, L is SO₂, and m is 1.

In another embodiment, a sirtuin-activating compound is a compound of formula 61: 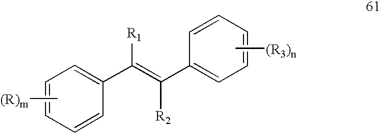
wherein, independently for each occurrence:

R, R₁, R₂, and R₃ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

n and m are integers from 0 to 5 inclusive.

In a further embodiment, a sirtuin-activating compound is a compound of formula 61 and the attendant definitions wherein n is 2.

In a further embodiment, a sirtuin-activating compound is a compound of formula 61 and the attendant definitions wherein R is 3-hydroxy and 5-hydroxy.

In a further embodiment, a sirtuin-activating compound is a compound of formula 61 and the attendant definitions wherein R₁ is H.

In a further embodiment, a sirtuin-activating compound is a compound of formula 61 and the attendant definitions wherein R₂ is H.

In a further embodiment, a sirtuin-activating compound is a compound of formula 61 and the attendant definitions wherein m is 0.

In a further embodiment, a sirtuin-activating compound is a compound of formula 61 and the attendant definitions wherein m is 1.

In a further embodiment, a sirtuin-activating compound is a compound of formula 61 and the attendant definitions wherein R₃ is 4-hydroxy.

In a further embodiment, a sirtuin-activating compound is a compound of formula 61 and the attendant definitions wherein R₃ is 4-methoxy.

In a further embodiment, a sirtuin-activating compound is a compound of formula 61 and the attendant definitions wherein n is 2 and R is 3-hydroxy and 5-hydroxy.

In a further embodiment, a sirtuin-activating compound is a compound of formula 61 and the attendant definitions wherein n is 2, R is 3-hydroxy and 5-hydroxy, and R₁ is H.

In a further embodiment, a sirtuin-activating compound is a compound of formula 61 and the attendant definitions wherein n is 2, R is 3-hydroxy and 5-hydroxy, R₁ is H, and R₂ is H.

In a further embodiment, a sirtuin-activating compound is a compound of formula 61 and the attendant definitions wherein n is 2, R is 3-hydroxy and 5-hydroxy, R₁ is H, R₂ is H, and m is 0.

In a further embodiment, a sirtuin-activating compound is a compound of formula 61 and the attendant definitions wherein n is 2, R is 3-hydroxy and 5-hydroxy, R₁ is H, R₂ is H, and m is 1.

In a further embodiment, a sirtuin-activating compound is a compound of formula 61 and the attendant definitions wherein n is 2, R is 3-hydroxy and 5-hydroxy, R₁ is H, R₂ is H, m is 1, and R₃ is 4-hydroxy.

In a further embodiment, a sirtuin-activating compound is a compound of formula 61 and the attendant definitions wherein n is 2, R is 3-hydroxy and 5-hydroxy, R₁ is H, R₂ is H, m is 1, and R₃ is 4-methoxy.

In another embodiment, a sirtuin-activating compound is a compound of formula 62: 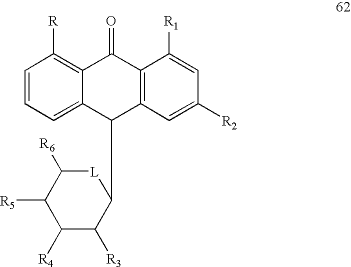
wherein, independently for each occurrence:

R, R₁, R₂, R₃, R₄, R₅, and R₆ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L is O, NR₇, or S; and

R₇ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 62 and the attendant definitions wherein R is OH.

In a further embodiment, a sirtuin-activating compound is a compound of formula 62 and the attendant definitions wherein R₁ is OH.

In a further embodiment, a sirtuin-activating compound is a compound of formula 62 and the attendant definitions wherein R₂ is CH₂OH.

In a further embodiment, a sirtuin-activating compound is a compound of formula 62 and the attendant definitions wherein R₃ is OH.

In a further embodiment, a sirtuin-activating compound is a compound of formula 62 and the attendant definitions wherein R₄ is OH.

In a further embodiment, a sirtuin-activating compound is a compound of formula 62 and the attendant definitions wherein R₅ is OH.

In a further embodiment, a sirtuin-activating compound is a compound of formula 62 and the attendant definitions wherein R₆ is CH₂OH.

In a further embodiment, a sirtuin-activating compound is a compound of formula 62 and the attendant definitions wherein L is O.

In a further embodiment, a sirtuin-activating compound is a compound of formula 62 and the attendant definitions wherein R is OH and R₁ is OH.

In a further embodiment, a sirtuin-activating compound is a compound of formula 62 and the attendant definitions wherein R is OH, R₁ is OH, and R₂ is CH₂OH.

In a further embodiment, a sirtuin-activating compound is a compound of formula 62 and the attendant definitions wherein R is OH, R₁ is OH, R₂ is CH₂OH, and R₃ is OH.

In a further embodiment, a sirtuin-activating compound is a compound of formula 62 and the attendant definitions wherein R is OH, R₁ is OH, R₂ is CH₂OH, R₃ is OH, and R₄ is OH.

In a further embodiment, a sirtuin-activating compound is a compound of formula 62 and the attendant definitions wherein R is OH, R₁ is OH, R₂ is CH₂OH, R₃ is OH, R₄ is OH, and R₅ is OH.

In a further embodiment, a sirtuin-activating compound is a compound of formula 62 and the attendant definitions wherein R is OH, R₁ is OH, R₂ is CH₂OH, R₃ is OH, R₄ is OH, R₅ is OH, and R₆ is CH₂OH.

In a further embodiment, a sirtuin-activating compound is a compound of formula 62 and the attendant definitions wherein R is OH, R₁ is OH, R₂ is CH₂OH, R₃ is OH, R₄ is OH, R₅ is OH, R₆ is CH₂OH, and L is O.

In another embodiment, a sirtuin-activating compound is a compound of formula 63: 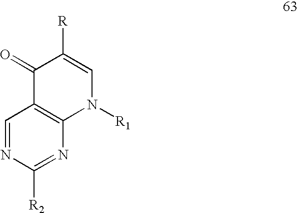
wherein, independently for each occurrence:

R, R₁, and R₂ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 63 and the attendant definitions wherein R is CO₂H.

In a further embodiment, a sirtuin-activating compound is a compound of formula 63 and the attendant definitions wherein R₁ is ethyl.

In a further embodiment, a sirtuin-activating compound is a compound of formula 63 and the attendant definitions wherein R₂ is N-1-pyrrolidine.

In a further embodiment, a sirtuin-activating compound is a compound of formula 63 and the attendant definitions wherein R is CO₂H and R₁ is ethyl.

In a further embodiment, a sirtuin-activating compound is a compound of formula 63 and the attendant definitions wherein R is CO₂H and R₂ is N-1-pyrrolidine.

In a further embodiment, a sirtuin-activating compound is a compound of formula 63 and the attendant definitions wherein R₁ is ethyl and R₂ is N-1-pyrrolidine.

In a further embodiment, a sirtuin-activating compound is a compound of formula 63 and the attendant definitions wherein R is CO₂H, R₁ is ethyl, and R₂ is N-1-pyrrolidine.

In another embodiment, a sirtuin-activating compound is a compound of formula 64: 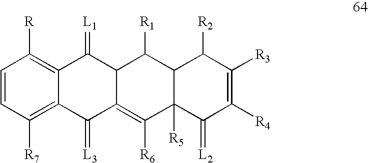
wherein, independently for each occurrence:

R, R₁, R₂, R₃, R₄, R₅, R₆, and R₇ are H, hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl;

L₁, L₂, and L₃ are CH₂, O, NR₈, or S; and

R₈ is H or a substituted or unsubstituted alkyl, aryl, aralkyl, heterocyclyl, heterocyclylalkyl, heteroaryl, or heteroaralkyl.

In a further embodiment, a sirtuin-activating compound is a compound of formula 64 and the attendant definitions wherein R is Cl.

In a further embodiment, a sirtuin-activating compound is a compound of formula 64 and the attendant definitions wherein R is H.

In a further embodiment, a sirtuin-activating compound is a compound of formula 64 and the attendant definitions wherein R₁ is OH.

In a further embodiment, a sirtuin-activating compound is a compound of formula 64 and the attendant definitions wherein R₂ is N(Me)₂.

In a further embodiment, a sirtuin-activating compound is a compound of formula 64 and the attendant definitions wherein R₃ is OH.

In a further embodiment, a sirtuin-activating compound is a compound of formula 64 and the attendant definitions wherein R₄ is C(O)NH₂.

In a further embodiment, a sirtuin-activating compound is a compound of formula 64 and the attendant definitions wherein R₅ is OH.

In a further embodiment, a sirtuin-activating compound is a compound of formula 64 and the attendant definitions wherein R₆ is OH.

In a further embodiment a sirtuin-activating compound is a compound of formnula 64 and the attendant definitions wherein R₇ is OH.

In a further embodiment, a sirtuin-activating compound is a compound of formula 64 and the attendant definitions wherein L₁ is CH₂.

In a further embodiment, a sirtuin-activating compound is a compound of formula 64 and the attendant definitions wherein L₂ is O.

In a further embodiment, a sirtuin-activating compound is a compound of formula 64 and the attendant definitions wherein L₃ is O.

In a further embodiment, a sirtuin-activating compound is a compound of formula 64 and the attendant definitions wherein R is Cl and R₁ is OH.

In a further embodiment, a sirtuin-activating compound is a compound of formula 64 and the attendant definitions wherein R is Cl, R₁ is OH, and R₂ is N(Me)₂.

In a further embodiment, a sirtuin-activating compound is a compound of formula 64 and the attendant definitions wherein R is Cl, R₁ is OH, R₂ is N(Me)₂, and R₃ is OH.

In a further embodiment, a sirtuin-activating compound is a compound of formula 64 and the attendant definitions wherein R is Cl, R₁ is OH, R₂ is N(Me)₂, R₃ is OH, and R₄ is C(O)NH₂.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, and R₅ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, and R₆ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, and R₇ is OH.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin-activating compound is a compound of formula 64 and the attendant definitions wherein R is Cl, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In a further embodiment, a sirtuin-activating compound is a compound of formula 64 and the attendant definitions wherein R is H and R₁ is OH.

In a further embodiment, a sirtuin-activating compound is a compound of formula 64 and the attendant definitions wherein R is H, R₁ is OH, and R₂ is N(Me)₂.

In a further embodiment, a sirtuin-activating compound is a compound of formula 64 and the attendant definitions wherein R is H, R₁ is OH, R₂ is N(Me)₂, and R₃ is OH.

In a further embodiment, a sirtuin-activating compound is a compound of formula 64 and the attendant definitions wherein R is H, R₁ is OH, R₂ is N(Me)₂, R₃ is OH, and R₄ is C(O)NH₂.

In a further embodiment, a sirtuin-activating compound is a compound of formula 64 and the attendant definitions wherein R is H, R₁ is OH, R₂ is N(Me)₂, R₃ is OH, R₄ is C(O)NH₂, and R₅ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, and R₆ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, and R₇ is OH.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, and L₁ is CH₂.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, and L₂ is O.

In a further embodiment, a sirtuin-activating compound is a compound of formula 64 and the attendant definitions wherein R is H, R₁ is OH, R₂ is N(Me)₂, R₃ is OH, R₄ is C(O)NH₂, R₅ is OH, R₆ is OH, R₇ is OH, L₁ is CH₂, L₂ is O, and L₃ is O.

In another embodiment, a sirtuin-activating compound is a compound of formula 65: 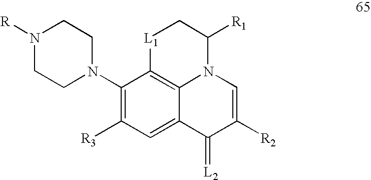
wherein, independently for each occurrence:

R is H or a substituted or unsubstituted alkyl, aryl, aralkyl, heterocyclyl, heterocyclylalkyl, heteroaryl, or heteroaralkyl;

R₁, R₂, and R₃ are hydroxy, amino, cyano, halide, alkoxy, ether, ester, amido, ketone, carboxylic acid, nitro, or a substituted or unsubstituted alkyl, aryl, aralkyl, heterocyclyl, heterocyclylalkyl, heteroaryl, or heteroaralkyl; and

L₁ and L₂ are O, NR, or S.

In a further embodiment, a sirtuin-activating compound is a compound of formula 65 and the attendant definitions wherein R is methyl.

In a further embodiment, a sirtuin-activating compound is a compound of formula 65 and the attendant definitions wherein R₁ is methyl.

In a further embodiment, a sirtuin-activating compound is a compound of formula 65 and the attendant definitions wherein R₂ is CO₂H.

In a further embodiment, a sirtuin-activating compound is a compound of formula 65 and the attendant definitions wherein R₃ is F.

In a further embodiment, a sirtuin-activating compound is a compound of formula 65 and the attendant definitions wherein L₁ is O.

In a further embodiment, a sirtuin-activating compound is a compound of formula 65 and the attendant definitions wherein L₂ is O.

In a further embodiment, a sirtuin-activating compound is a compound of formula 65 and the attendant definitions wherein R is methyl and R₁ is methyl.

In a further embodiment, a sirtuin-activating compound is a compound of formula 65 and the attendant definitions wherein R is methyl, R₁ is methyl, and R₂ is CO₂H.

In a further embodiment, a sirtuin-activating compound is a compound of formula 65 and the attendant definitions wherein R is methyl, R₁ is methyl, R₂ is CO₂H, and R₃ is F.

In a further embodiment, a sirtuin-activating compound is a compound of formula 65 and the attendant definitions wherein R is methyl, R₁ is methyl, R₂ is CO₂H, R₃ is F, and L₁ is O.

In a further embodiment, a sirtuin-activating compound is a compound of formula 65 and the attendant definitions wherein R is methyl, R₁ is methyl, R₂ is CO₂H, R₃ is F, L₁ is O, and L₂ is O.

Exemplary activating compounds are those listed in the appended Tables having a ratio to control rate of more than one. A preferred compound of formula 8 is Dipyridamole; a preferred compound of formula 12 is Hinokitiol; a preferred compound of formula 13 is L-(+)-Ergothioneine; a preferred compound of formula 19 is Caffeic Acid Phenol Ester; a preferred compound of formula 20 is MCI-186 and a preferred compound of formula 21 is HBED (Supplementary Table 6). Activating compounds may also be oxidized forms of the compounds of Table 21.

Also included are pharmaceutically acceptable addition salts and complexes of the compounds of formulas 1-25, 30, 32-65, and 69-88. In cases wherein the compounds may have one or more chiral centers, unless specified, the compounds contemplated herein may be a single stereoisomer or racemic mixtures of stereoisomers.

In one embodiment, a sirtuin-activating compound is a stilbene, chalcone, or flavone compound represented by formula 7:

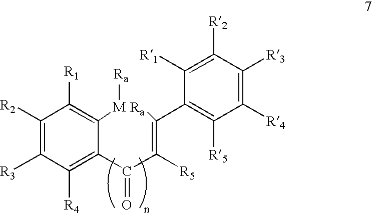

wherein, independently for each occurrence,

M is absent or O;

R₁, R₂, R₃, R₄, R₅, R′₁, R′₂, R′₃, R′₄, and R′₅ represent H, alkyl, aryl, heteroaryl, aralkyl, alkaryl, heteroaralkyl, halide, NO₂, SR, OR, N(R)₂, or carboxyl;

Rₐ represents H or the two instances of Rₐ form a bond;

R represents H, alkyl, or aryl; and

n is 0 or 1.

In a further embodiment, a sirtuin-activating compound is a compound represented by formula 7 and the attendant definitions, wherein n is 0. In a further embodiment, a sirtuin-activating compound is a compound represented by formula 7 and the attendant definitions, wherein n is 1. In a further embodiment, a sirtuin-activating compound is a compound represented by formula 7 and the attendant definitions, wherein M is absent. In a further embodiment, a sirtuin-activating compound is a compound represented by formula 7 and the attendant definitions, wherein M is O. In a further embodiment, a sirtuin-activating compound is a compound represented by formula 7 and the attendant definitions, wherein Rₐ is H. In a further embodiment, a sirtuin-activating compound is a compound represented by formula 7 and the attendant definitions, wherein M is O and the two Rₐ form a bond. In a further embodiment, a sirtuin-activating compound is a compound represented by formula 7 and the attendant definitions, wherein R₅ is H. In a further embodiment, a sirtuin-activating compound is a compound represented by formula 7 and the attendant definitions, wherein R₅ is OH. In a further embodiment, a sirtuin-activating compound is a compound represented by formula 7 and the attendant definitions, wherein R₁, R₃, and R′₃ are OH. In a further embodiment, a sirtuin-activating compound is a compound represented by formula 7 and the attendant definitions, wherein R₂, R₄, R′₂, and R′₃ are OH. In a further embodiment, a sirtuin-activating compound is a compound represented by formula 7 and the attendant definitions, wherein R₂, R′₂, and R′₃ are OH.

In a further embodiment, a sirtuin-activating compound is a compound represented by formula 7 and the attendant definitions, wherein n is 0; M is absent; Rₐ is H; R₅ is H; R₁, R₃, and R′₃ are OH; and R₂, R₄, R′₁, R′₂, R′₄, and R′₅ are H. In a further embodiment, a sirtuin-activating compound is a compound represented by formula 7 and the attendant definitions, wherein n is 1; M is absent; Rₐ is H; R₅ is H; R₂, R₄, R′₂, and R′₃ are OH; and R₁, R₃, R′₁, R′₄, and R′₅ are H. In a further embodiment, a sirtuin-activating compound is a compound represented by formula 7 and the attendant definitions, wherein n is 1; M is O; the two Rₐ form a bond; R₅ is OH; R₂, R′₂, and R′₃ are OH; and R₁, R₃, R₄, R′₁, R′₄, and R′₅ are H.

In another embodiment, exemplary sirtuin-activating compounds are isonicotinamide analogs, such as, for example, the isonicotinamide analogs described in U.S. Pat. Nos. 5,985,848; 6,066,722; 6,228,847; 6,492,347; 6,803,455; and U.S. Patent Publication Nos. 2001/0019823; 2002/0061898; 2002/0132783; 2003/0149261; 2003/0229033; 2003/0096830; 2004/0053944; 2004/0110772; and 2004/0181063, the disclosures of which are hereby incorporated by reference in their entirety. In an exemplary embodiment, sirtuin-activating compounds may be an isonicotinamide analog having any of formulas 69-72 below. In one embodiment, a sirtuin-activating compound is an isonicotinamide analog compound of formula 69:

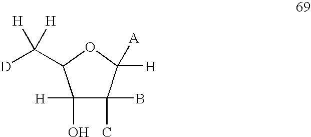

Wherein A is a nitrogen-, oxygen-, or sulfur-linked aryl, alkyl, cyclic, or heterocyclic group. The A moieties thus described, optionally have leaving group characteristics. In embodiments encompassed herein, A is further substituted with an electron contributing moiety. B and C are both hydrogen, or one of B or C is a halogen, amino, or thiol group and the other of B or C is hydrogen; and D is a primary alcohol, a hydrogen, or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge.

In one example, A is a substituted N-linked aryl or heterocyclic group, an O-linked aryl or heterocyclic group having the formula —O—Y, or an S-linked aryl or heterocyclic group having the formula —O—Y; both B and C are hydrogen, or one of B or C is a halogen, amino, or thiol group and the other of B or C is hydrogen; and D is a primary alcohol or hydrogen. Nonlimiting preferred examples of A are set forth below, where each R is H or an electron-contributing moiety and Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl.

Examples of A include i-xiv below: 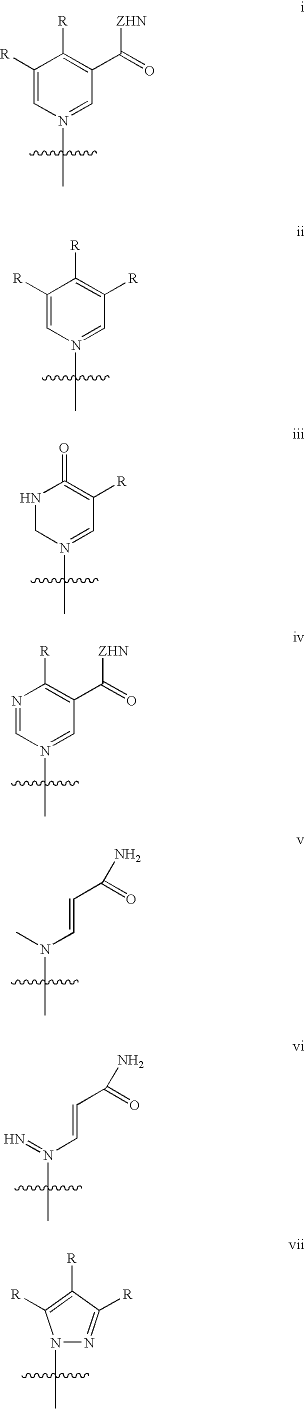 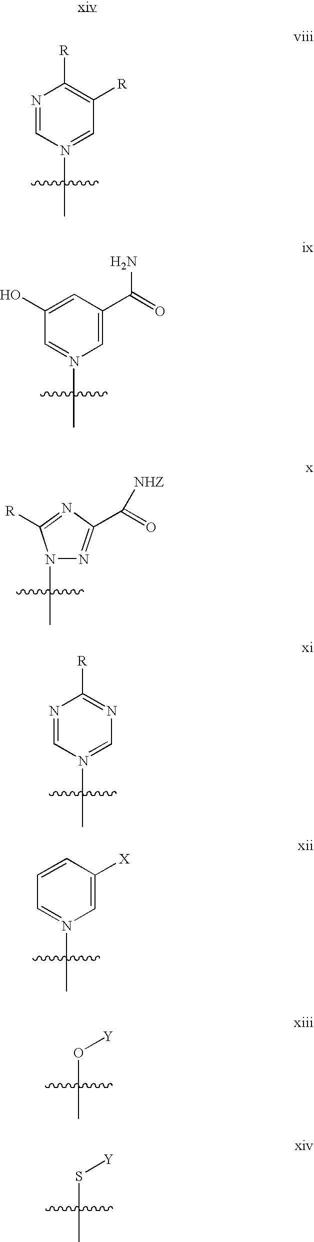
where Y=a group consistent with leaving group function.

Examples of Y include, but are not limited to, xv-xxvii below:

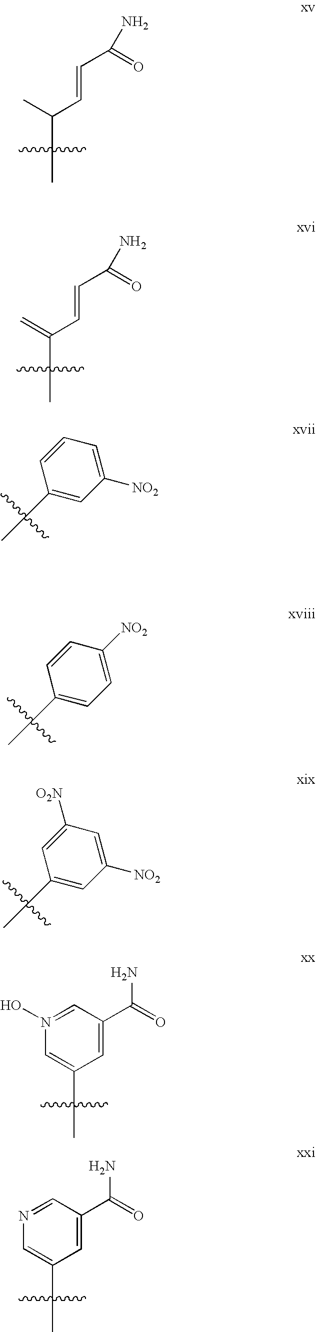

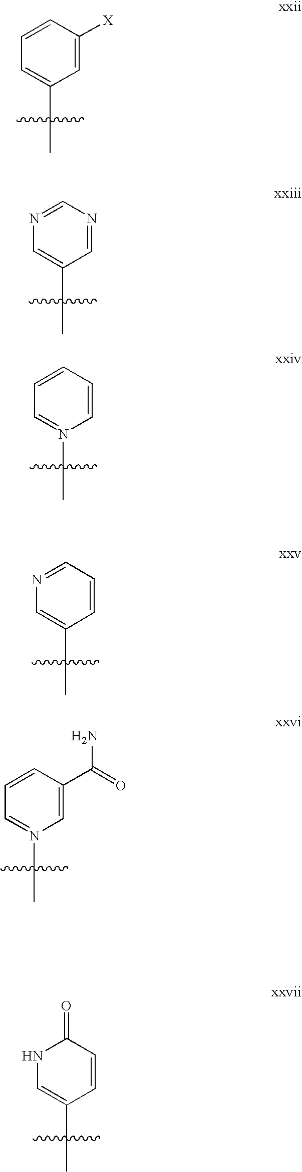

Wherein, for i-xxvii, X is halogen, thiol, or substituted thiol, amino or substituted amino, oxygen or substituted oxygen, or aryl or alkyl groups or heterocycles.

In certain embodiments, A is a substituted nicotinamide group (i above, where Z is H), a substituted pyrazolo group (vii above), or a substituted 3-carboxamid-imidazolo group (x above, where Z is H). Additionally, both B and C may be hydrogen, or one of B or C is a halogen, amino, or thiol group and the other of B or C is hydrogen; and D is a primary alcohol or hydrogen.

In other embodiments, one of B or C may be halogen, amino, or thiol group when the other of B or C is a hydrogen. Furthermore, D may be a hydrogen or an oxygen, nitrogen, carbon, or sulfur linked to phosphate, a phosphoryl group, a pyrophosphoryl group, or adenosine monophosphate through a phosphodiester or carbon-, nitrogen-, or sulfur-substituted phosphodiester bridge, or to adenosine diphosphate through a phosphodiester or carbon-, nitrogen-, or sulfur-substituted pyrophosphodiester bridge. Analogues of adenosine monophosphate or adenosine diphosphate also can replace the adenosine monophosphate or adenosine diphosphate groups.

In some embodiments, A has two or more electron contributing moieties.

In other embodiments, a sirtuin-activating compound is an isonicotinamide analog compound of formulas 70, 71, or 72 below. 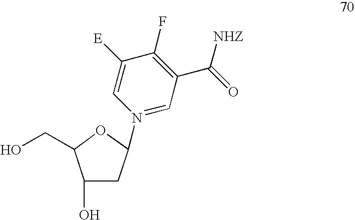
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; E and F are independently H, CH₃, OCH₃, CH₂CH₃, NH₂, OH, NHCOH, NHCOCH₃, N(CH₃)₂, C(CH₃)₂, an aryl or a C3-C10 alkyl, preferably provided that, when one of of E or F is H, the other of E or F is not H; 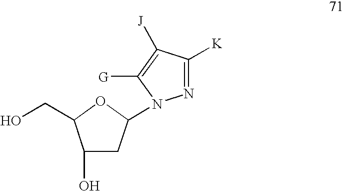
wherein G, J or K is CONHZ, Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl, and the other two of G, J and K is independently CH₃, OCH₃, CH₂CH₃, NH₂, OH, NHCOH, NHCOCH₃; 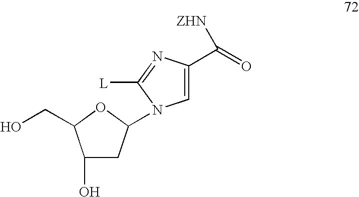
wherein Z is an alkyl, aryl, hydroxyl, OZ′ where Z′ is an alkyl or aryl, amino, NHZ′ where Z′ is an alkyl or aryl, or NHZ′Z″ where Z′ and Z″ are independently an alkyl or aryl; and L is CH₃, OCH₃, CH₂CH₃, NH₂, OH, NHCOH, NHCOCH₃.

In an exemplary embodiment, the compound is formula 70 above, wherein E and F are independently H, CH₃, OCH₃, or OH, preferably provided that, when one of E or F is H, the other of E or F is not H.

In another exemplary embodiment, the compound is β-1′-5-methyl-nicotinamide-2′-deoxyribose, β-D-1′-5-methyl-nico-tinamide-2′-deoxyribofuranoside, β-1′-4,5-dimethyl-nicotinamide-2′-de-oxyribose or β-D-1′-4,5-dimethyl-nicotinamide-2′-deoxyribofuranoside.

In yet another embodiment, the compound is β-1′-5-methyl-nicotinamide-2′-deoxyribose.

Without being bound to any particular mechanism, it is believed that the electron-contributing moiety on A stabilizes the compounds of the invention such that they are less susceptible to hydrolysis from the rest of the compound. This improved chemical stability improves the value of the compound, since it is available for action for longer periods of time in biological systems due to resistance to hydrolytic breakdown. The skilled artisan could envision many electron-contributing moieties that would be expected to serve this stabilizing function. Non-limiting examples of suitable electron contributing moieties are methyl, ethyl, O-methyl, amino, NMe2, hydroxyl, CMe₃, aryl and alkyl groups. Preferably, the electron-contributing moiety is a methyl, ethyl, O-methyl, amino group. In the most preferred embodiments, the electron-contributing moiety is a methyl group.

The compounds of formulas 69-72 are useful both in free form and in the form of salts. The term “pharmaceutically acceptable salts” is intended to apply to non-toxic salts derived from inorganic or organic acids and includes, for example, salts derived from the following acids: hydrochloric, sulfuric, phosphoric, acetic, lactic, fumaric, succinic, tartaric, gluconic, citric, methanesulfonic, and p-toluenesulfonic acids.

Also provided are compounds of formulas 69-72 that are the tautomers, pharmaceutically-acceptable salts, esters, and pro-drugs of the inhibitor compounds disclosed herein.

The biological availability of the compounds of formulas 69-72 can be enhanced by conversion into a pro-drug form. Such a pro-drug can have improved lipophilicity relative to the unconverted compound, and this can result in enhanced membrane permeability. One particularly useful form of pro-drug is an ester derivative. Its utility relies upon the action of one or more of the ubiquitous intracellular lipases to catalyse the hydrolysis of ester groups, to release the active compound at or near its site of action. In one form of pro-drug, one or more hydroxy groups in the compound can be O-acylated, to make an acylate derivative.

Pro-drug forms of a 5-phosphate ester derivative of compounds of formulas 69-72 can also be made. These may be particularly useful, since the anionic nature of the 5-phosphate may limit its ability to cross cellular membranes. Conveniently, such a 5-phosphate derivative can be converted to an uncharged bis(acyloxymethyl)ester derivative. The utility of such a pro-drug relies upon the action of one or more of the ubiquitous intracellular lipases to catalyse the hydrolysis of ester groups, releasing a molecule of formaldehyde and a compound of the present invention at or near its site of action. Specific examples of the utility of, and general methods for making, such acyloxymethyl ester pro-drug forms of phosphorylated carbohydrate derivatives have been described (Kang et al., 1998; Jiang et al., 1998; Li et al., 1997; Kruppa et al., 1997).

In another embodiment, exemplary sirtuin-activating compounds are O-acetyl-ADP-ribose analogs, including 2′-O-acetyl-ADP-ribose and 3′-O-acetyl-ADP-ribose, and analogs thereof. Exemplary O-acetyl-ADP-ribose analogs are described, for example, in U.S. Patent Publication Nos. 2004/0053944; 2002/0061898; and 2003/0149261, the disclosures of which are hereby incorporated by reference in their entirety. In an exemplary embodiment, sirtuin-activating compounds may be an O-acetyl-ADP-ribose analog having any of formulas 73-76 below. In one embodiment, a sirtuin-activating compound is an O-acetyl-ADP-ribose analog compound of formula 73: 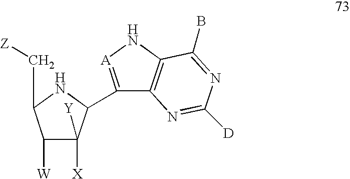
wherein:

A is selected from N, CH and CR, where R is selected from halogen, optionally substituted alkyl, aralkyl and aryl, OH, NH₂, NHR¹, NR¹R² and SR³, where R¹, R² and R³ are each optionally substituted alkyl, aralkyl or aryl groups;

B is selected from OH, NH₂, NHR⁴, H and halogen, where R⁴ is an optionally substituted alkyl, aralkyl or aryl group;

D is selected from OH, NH₂, NHR⁵, H, halogen and SCH₃, where R⁵ is an optionally substituted alkyl, aralkyl or aryl group;

X and Y are independently selected from H, OH and halogen, with the proviso that when one of X and Y is hydroxy or halogen, the other is hydrogen;

Z is OH, or, when X is hydroxy, Z is selected from hydrogen, halogen, hydroxy, SQ and OQ, where Q is an optionally substituted alkyl, aralkyl or aryl group; and

W is OH or H, with the proviso that when W is OH, then A is CR where R is as defined above;

or a tautomer thereof, or a pharmaceutically acceptable salt thereof; or an ester thereof; or a prodrug thereof.

In certain embodiments, when B is NHR⁴ and/or D is NHR⁵, then R⁴ and/or R⁵ are C1-C4 alkyl.

In other embodiments, when one or more halogens are present they are chosen from chlorine and fluorine.

In another embodiment, when Z is SQ or OQ, Q is C1-C5 alkyl or phenyl.

In an exemplary embodiment, D is H, or when D is other than H, B is OH.

In another embodiment, B is OH, D is H, OH or NH₂, X is OH or H, Y is H, most preferably with Z as OH, H, or methylthio, especially OH.

In certain embodiments W is OH, Y is H, X is OH, and A is CR where R is methyl or halogen, preferably fluorine.

In other embodiments, W is H, Y is H, X is OH and A is CH.

In other embodiments, a sirtuin-activating compound is an O-acetyl-ADP-ribose analog compound of formula 74:

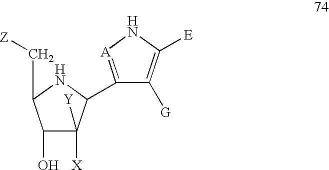

wherein A, X, Y, Z and R are defined for compounds of formula (73) where first shown above; E is chosen from CO₂H or a corresponding salt form, CO₂R, CN, CONH₂, CONHR or CONR₂; and G is chosen from NH₂, NHCOR, NHCONHR or NHCSNHR; or a tautomer thereof, or a pharmaceutically acceptable salt thereof, or an ester thereof, or a prodrug thereof.

In certain embodiments, E is CONH₂ and G is NH₂.

In other embodiments, E is CONH₂, G is NH₂, X is OH or H, is H, most preferable with Z as OH, H or methylthio, especially OH.

Exemplary sirtuin-activating compounds include the following:
(1S)-1,4-dideoxy-1-C-(4-hydroxypyrrolo[3,2-d]pyrimidin-7-yl)-1,4-imino-D-ribitol
(1R)-1-C-(2-amino-4-hydroxypyrrolo[3,2-d]pyrimidin-7-yl)-1,4-dideoxy-1,4-imino-D-ribitol
(1R)-1-C-(4-hydroxypyrrolo[3,2-d]pyrimidin-7-yl)-1,4-imino-1,2,4-trideoxy-D-erythro-pentitol
(1S)-1-C-(4-hydroxypyrrolo[3,2-d]pyrimidin-7-yl)-1,4-imino-1,4,5-trideoxy-D-ribitol
(1S)-1,4-dideoxy-1-C-(4-hydroxypyrrolo[3,2-d]pyrimidin-7-yl)-1,4-imino-5-methylthio-D-ribitol
(1R)-1,4-dideoxy-1-C-(2,4-dihydroxypyrrolo[3,2-d]pyrimidin-7-yl)-1,4-imino-D-ribitol
(1R)-1-C-(2,4-dihydroxypyrrolo[3,2-d]pyrimidin-7-yl)-1,4-imino-1,2,4-trideoxy-D-erthro-pentitol
(1S)-1-C-(2,4-dihydroxypyrrolo[3,2-d]pyrimidin-7-yl)-1,4-imino-1,4,5-trideoxy-D-ribitol
(1S)-1,4-dideoxy-1-C-(2,4-dihydroxypyrrolo[3,2-d]pyrimidin-7-yl)-1,4-imino-5-ethylthio-D-ribitol
(1R)-1-C-(2-amino-4-hydroxypyrrolo[3,2-d]pyrimidin-7-yl)-1,4-imino-1,2,4-trideoxy-D-erythro-pentitol
(1S)-1-C-(2-amino-4-hydroxypyrrolo[3,2-d]pyrimidin-7-yl)-1,4-imino-1,4,5-trideoxy-D-ribitol
(1S)-1-C-(2-amino-4-hydroxypyrrolo[3,2-d]pyrimidin-7-yl)-1,4-dideoxy-1,4-imino-5-methylthio-D-ribitol
(1S)-1,4-dideoxy-1-C-(7-hydroxypyrazolo[4,3-d]pyrimidin-3-yl)-1,4-imino-D-ribitol
(1R)-1-C-(7-hydroxypyrazolo[4,3-d]pyrimidin-3-yl)-1,4-imino-1,2,4-trideoxy-D-erythro-pentitol
(1S)-1-C-(7-hydroxypyrazolo[4,3-d]pyrimidin-3-yl)-1,4-imino-1,4,5-trideoxy-D-ribitol
(1S)-1,4-dideoxy-1-C-(7-hydroxypyrazolo[4,3-d]pyrimidin-3-yl)-1,4-imino-5-ethylthio-D-ribitol
(1S)-1,4-dideoxy-1-C-(5,7-dihydroxypyrazolo[4,3-d]pyrimidin-3-yl)-1,4-imino-D-ribitol
(1R)-1-C-(5,7-dihydroxypyrazolo[4,3-d]pyrimidin-3-yl)-1,4-imino-1,2,4-trideoxy-D-erythro-pentitol
(1S)-1-C-(5,7-dihydroxypyrazolo[4,3-d]pyrimidin-3-yl)-1,4-imino-1,4,5-trideoxy-D-ribitol
(1S)-1,4-dideoxy-1-C-(5,7-dihydroxypyrazolo[4,3-d]pyrimidin-3-yl)-1,4-imino-5-methylthio-D-ribitol
(1S)-1-C-(5-amino-7-hydroxypyrazolo[4,3-d]pyrimidin-3-yl)-1,4-dideoxy-1,4-imino-D-ribitol
(1R)-1-C-(S-amino-7-hydroxypyrazolo[4,3-d]pyrimidin-3-yl)-1,4-imino-1,2,4-trideoxy-D-erythro-pentitol
(1S)-1-C-(5-amino-7-hydroxypyrazolo[4,3-d]pyrimidin-3-yl)-1,4-imino-1,4,5-trideoxy-D-ribitol
(1S)-1-C-(5-amino-7-hydroxypyrazolo[4,3-d]pyrimidin-3-yl)-1,4-dideoxy-1,4-imino-5-methylthio-D-ribitol
(1S)-1-C-(3-amino-2-carboxamido-4-pyrroly)-1,4-dideoxy-1,4-imino-D-ribitol.
(1S)-1,4-dideoxy-1-C-(4-hydroxypyrrolo[3,2-d]pyrimidin-7-yl)-1,4-imino-D-ribitol 5-phosphate
(1S)-1-C-(2-amino-4-hydroxypyrrolo[3,2-d]pyrimidin-7-yl)-1,4-imino-D-ribitol 5-phosphate
(1S)-1-C-(3-amino-2-carboxamido-4-pyrrolyl)-1,4-dideoxy-1,4-imino-D-ribitol

In yet other embodiments, sirtuin-activating compounds are O-acetyl-ADP-ribose analog compounds of formula 75 and 76, their tautomers and pharmaceutically acceptable salts.

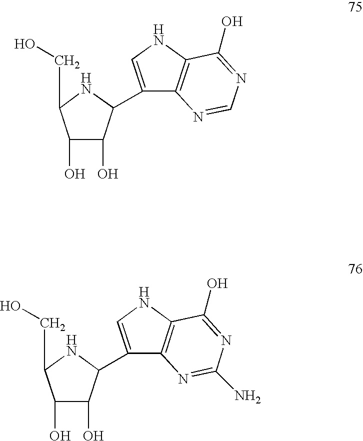

The biological availability of a compound of formula (73) or formula (74) can be enhanced by conversion into a pro-drug form. Such a pro-drug can have improved lipophilicity relative to the compound of formula (73) or formula (74), and this can result in enhanced membrane permeability. One particularly useful form of a pro-drug is an ester derivative. Its utility relies upon the action of one or more of the ubiquitous intracellular lipases to catalyse the hydrolysis of these ester group(s), to release the compound of formula (73) and formula (74) at or near its site of action.

In one form of a prodrug, one or more of the hydroxy groups in a compound of formula (73) or formula (74) can be O-acylated, to make, for example a 5-O-butyrate or a 2,3-di-O-butyrate derivative.

Prodrug forms of 5-phosphate ester derivative of a compounds of formula (73) or formula (74) can also be made and may be particularly useful, since the anionic nature of the 5-phosphate may limit its ability to cross cellular membranes. Conveniently, such a 5-phosphate derivative can be converted to an uncharged bis(acyloxymethyl)ester derivative. The utility of such a pro-drug relies upon the action of one or more of the ubiquitous intracellular lipases to catalyse the hydrolysis of these ester group(s), releasing a molecule of formaldehyde and the compound of formula (73) or formula (74) at or near its site of action.

In an exemplary embodiment, analogs of 2′-AADPR or 3′-AADPR that are designed to have increased stability from esterase action through the use of well-known substitutes for ester oxygen atoms that are subject to esterase attack. The esterase-labile oxygen atoms in 2′-AADPR and 3′-AADPR would be understood to be the ester oxygen linking the acetate group with the ribose, and the ester oxygen between the two phosphorus atoms. As is known in the art, substitution of either or both of these ester oxygen atoms with a CF₂, a NH, or a S would be expected to provide a 2′-AADPR or 3′-AADPR analog that is substantially more stable due to increased resistance to esterase action.

Thus, in some embodiments, the invention is directed to analogs 2′-O-acetyl-ADP-ribose or 3′-O-acetyl-ADP-ribose exhibiting increased stability in cells. The preferred analogs comprise a CF₂, a NH, or a S instead of the acetyl ester oxygen or the oxygen between two phosphorus atoms. The most preferred substitute is CF₂. Replacement of the acetyl ester oxygen is particularly preferred. In other preferred embodiments, both the ester oxygen and the oxygen between the two phosphorus atoms are independently substituted with a CF₂, a NH, or a S.

Also included are pharmaceutically acceptable addition salts and complexes of the sirtuin-activity compounds described herein. In cases wherein the compounds may have one or more chiral centers, unless specified, the compounds contemplated herein may be a single stereoisomer or racemic mixtures of stereoisomers.

In one embodiment, sirtuin modulators for use in the invention are represented by Formula 77 or 78:

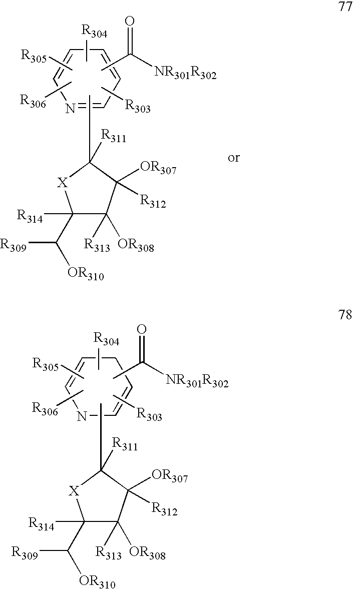

or a pharmaceutically acceptable salt thereof, where:

R₃₀₁ and R₃₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₃₀₁ and R₃₀₂ taken together form a substituted or unsubstituted non-aromatic heterocyclic group;

R₃₀₃, R₃₀₄, R₃₀₅ and R₃₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₃₀₇, R₃₀₈ and R₃₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₃₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₃₁₁, R₃₁₂, R₃₁₃ and R₃₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

A group of suitable compounds encompassed by Formulas 77 and 78 is represented by Structural Formulas 79 and 80:

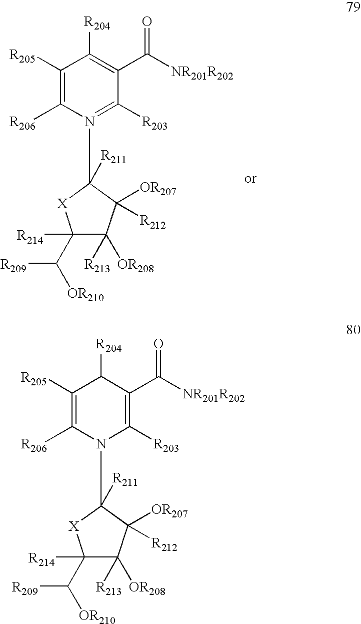

or a pharmaceutically acceptable salt thereof, where:

R₂₀, and R₂₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₂₀₁ and R₂₀₂ taken together form a substituted or unsubstituted non-aromatic heterocyclic group;

R₂₀₃, R₂₀₄, R₂₀₅ and R₂₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₂₀₇, R₂₀₈ and R₂₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₂₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₂₁₁, R₂₁₂, R₂₁₃ and R₂₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2.

In a particular group of compounds represented by Formula 79 or 80, at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR. Typically, at least one of R₂₀₇, R₂₀₈ and R₂₁₀ is —C(O)R or —C(O)OR. More typically, at least one of R₂₀₇, R₂₀₈ and R₂₁₀ is —C(O)R. In such compounds, R is preferably a substituted or unsubstituted alkyl, particularly an unsubstituted alkyl group such as methyl or ethyl.

In another particular group of compounds represented by Formula 79 or 80, R₂₀₄ is a halogen (e.g., fluorine, bromine, chlorine) or hydrogen (including a deuterium and/or tritium isotope). Suitable compounds include those where at least one of R₂₀₇, R₂₀₈ and R₂₁₀ is a substituted or unsubstituted alkyl group, a substituted or unsubstituted aryl group, —C(O)R, —C(O)OR, —C(O)NHR, —C(S)R, —C(S)OR or —C(O)SR and R₂₀₄ is a halogen or hydrogen.

Typically, for compounds represented by Formulas 79 and 80, R₂₀₃-R₂₀₆ are —H. In addition, R₂₀₉ and R₂₁₁-R₂₁₄ are typically —H. Particular compounds represented by Formulas 79 and 80 are selected such that R₂₀₃-R₂₀₆, R₂₀₉ and R₂₁₁-R₂₁₄ are all —H. For these compounds, R₂₀₄, R₂₀₇, R₂₀₈ and R₂₁₀ have the values described above.

R₂₀₁ and R₂₀₂ are typically —H or a substituted or unsubstituted alkyl group, more typically —H. In compounds having these values of R₂₀₁ and R₂₀₂, R₂₀₃-R₂₀₆, R₂₀₉ and R₂₁₁-R₂₁₄ typically have the values described above.

In certain methods of the invention, at least one of R₂₀₁-R₂₁₄ is not —H when X is O.

In certain methods of the invention, R₂₀₆ is not —H or —NH₂ when R₂₀₁-R₂₀₅ and R₂₀₇-R₂₁₄ are each —H.

In one embodiment, a sirtuin modulator is represented by Formula 81 or 82:

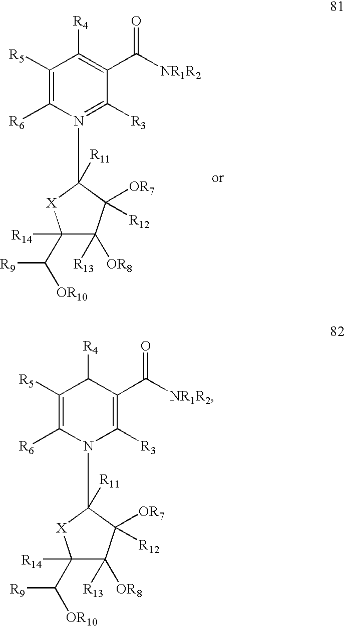

or a pharmaceutically acceptable salt thereof, wherein:

R₁ and R₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁ and R₂ taken together form a substituted or unsubstituted non-aromatic heterocyclic group, provided that when one of R₁ and R₂ is —H, the other is not an alkyl group substituted by —C(O)OCH₂CH₃;

R₃, R₄ and R₅ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₆ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRC(O)OR′, —NO₂ and —NRC(O)R′;

R₇, R₈ and R₁₀ are independently selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR;

R₉ selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁, R₁₂, R₁₃ and R₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S, preferably O; and

n is 1 or 2,

provided that R₁-R₁₄ are not each —H and that R₁-R₉ and R₁-R₁₄ are not each —H when R₁₀ is —C(O)C₆H₅.

In certain embodiments, R₁ is —H.

In certain embodiments, R₇, R₈ and R₁₀ are independently —H, —C(O)R or —C(O)OR, typically —H or —C(O)R such as —H or —C(O)CH₃. In particular embodiments, R₁ is —H and R₇, R₈ and R₁₀ are independently —H, —C(O)R or —C(O)OR.

In certain embodiments, R₉ is —H. In particular embodiments, R₉ is —H when R₁ is —H and/or R₇, R₈ and R₁₀ are independently —H, —C(O)R or —C(O)OR.

In certain embodiments, R₂ is —H. In particular embodiments, R₂ is —H when R₉ is —H, R₁ is —H and/or R₇, R₈ and R₁₀ are independently —H, —C(O)R or —C(O)OR. Typically, R₂ is —H when R₉ is —H, R₁ is —H and R₇, R₈ and R₁₀ are independently —H, —C(O)R or —C(O)OR.

In certain embodiments, R₄ is —H or a halogen, such as deuterium or fluorine.

In one embodiment, a sirtuin modulator is represented by Formula 83 or 84:

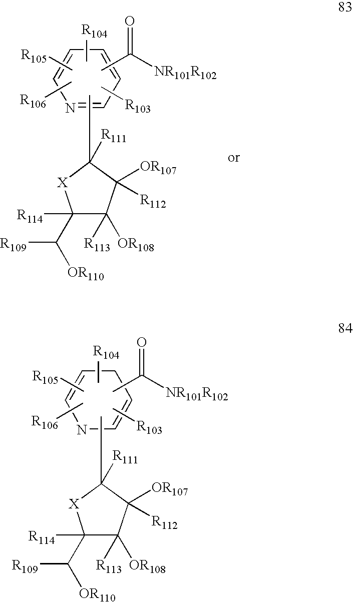

or a pharmaceutically acceptable salt thereof, wherein:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₀₁ and R₁₀₂ taken together form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

In another embodiment, a sirtuin modulator is represented by Formula 85 or 86:

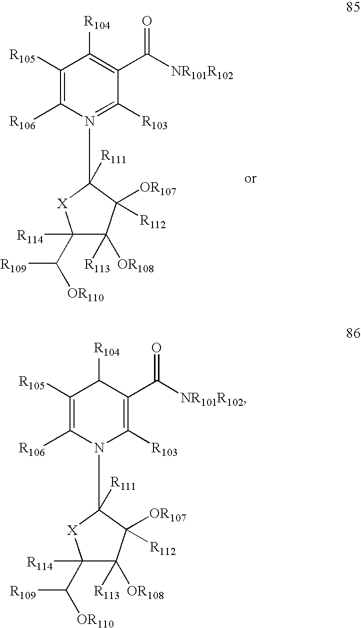

or a pharmaceutically acceptable salt thereof, where:

R₁₀₁ and R₁₀₂ are independently —H, a substituted or unsubstituted alkyl group, a substituted or unsubstituted alkenyl group, a substituted or unsubstituted alkynyl group, a substituted or unsubstituted non-aromatic heterocyclic group or a substituted or unsubstituted aryl group, or R₁₀₁ and R₁₀₂ taken together form a substituted or unsubstituted non-aromatic heterocyclic group;

R₁₀₃, R₁₀₄, R₁₀₅ and R₁₀₆ are independently selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′, —NO₂ and —NRC(O)R′;

R₁₀₇ and R₁₀₈ are selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, wherein at least one of R₁₀₇ and R₁₀₈ is a substituted or unsubstituted alkyl group, a substituted or unsubstituted aryl group, —C(O)R, —C(O)OR, —C(O)NHR, —C(S)R, —C(S)OR or —C(O)SR;

R₁₀₉ is selected from the group consisting of —H, a substituted or unsubstituted alkyl group, a substituted or unsubstituted aryl group, a substituted or unsubstituted non-aromatic heterocyclic group, halogen, —OR, —CN, —CO₂R, —OCOR, —OCO₂R, —C(O)NRR′, —OC(O)NRR′, —C(O)R, —COR, —SR, —OSO₃H, —S(O)ₙR, —S(O)ₙOR, —S(O)ₙNRR′, —NRR′, —NRC(O)OR′ and —NRC(O)R′;

R₁₁₀ is selected from the group consisting of —H, a substituted or unsubstituted alkyl group, a substituted or unsubstituted aryl group, —C(O)R, —C(O)OR, —C(O)NHR, —C(S)R, —C(S)OR and —C(O)SR, provided that R₁₁₀ is not —C(O)C₆H₅;

R₁₁₁, R₁₁₂, R₁₁₃ and R₁₁₄ are independently selected from the group consisting of —H, a substituted or unsubstituted alkyl group, a substituted or unsubstituted aryl group, a substituted or unsubstituted non-aromatic heterocyclic group, halogen, —CN, —CO₂R, —OCOR, —OCO₂R, —C(O)NRR′, —OC(O)NRR′, —C(O)R, —COR, —OSO₃H, —S(O)ₙR, —S(O)ₙOR, —S(O)ₙNRR′, —NRR′, —NRC(O)OR′, —NO₂ and —NRC(O)R′;

R and R′ are independently —H, a substituted or unsubstituted alkyl group, a substituted or unsubstituted aryl group or a substituted or unsubstituted non-aromatic heterocyclic group;

X is O or S; and

n is 1 or 2.

For compounds represented by Formulas 83-86, typically at least one of R₁₀₇ and R₁₀₈ is —C(O)R, such as —C(O)CH₃. In particular embodiments, R₁₀₇, R₁₀₈ and R₁₁₀ are independently —H or —C(O)R (e.g., —C(O)CH₃).

In certain embodiments, such as when R₁₀₇, R₁₀₈ and R₁₁₀ have the values described above, R₁₀₁ and R₁₀₂ are each —H.

In certain embodiments, R₁₀₉ is —H.

In certain embodiments, R₁₀₃-R₁₀₆ are each —H.

In certain embodiments, R₁₁₁-R₁₁₄ are each —H.

In particular embodiments, R₁₀₇, R₁₀₈ and R₁₁₀ have the values described above and R₁₀₁-R₁₀₆, R₁₀₉ and R₁₁₁-R₁₁₄ are each —H.

In certain embodiments, R₁₀₄ is —H or a halogen, typically deuterium or fluorine. The remaining values are as described above.

For sirtuin modulators represented by Formula 87 or 88: 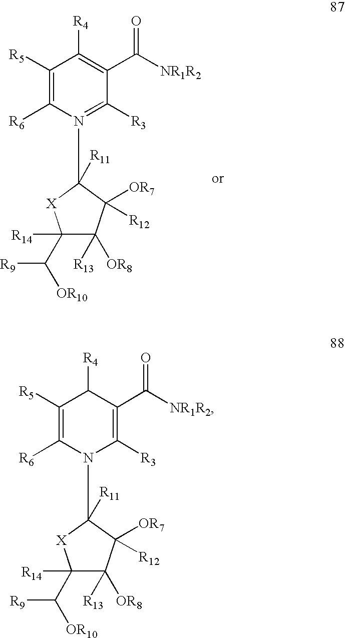
R₄ in certain embodiments is —H (e.g., deuterium, tritium) or a halogen (e.g., fluorine, bromine, chlorine).

In embodiments of the invention where R₁-R₆ can each be —H, they typically are each —H. In embodiments of the invention where one of R₁-R₆ is not —H, typically the remaining values are each —H and the non —H value is a substituted or unsubstituted alkyl group or a halogen (R₁ and R₂ are typically a substituted or unsubstituted alkyl group).

In certain embodiments, R₁₁-R₁₄ are each —H. When R₁₁-R₁₄ are each —H, R₁-R₆ typically have the values described above.

In certain embodiments, R₉ is —H. When R₉ is —H, typically R₁₁-R₁₄ are each —H and R₁-R₆ have the values described above.

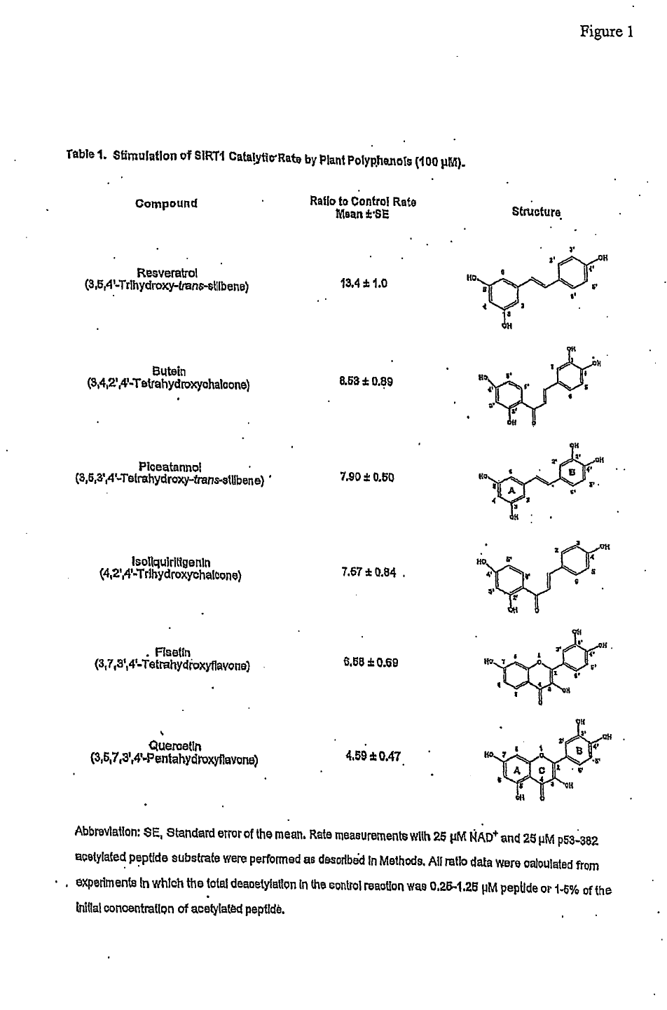

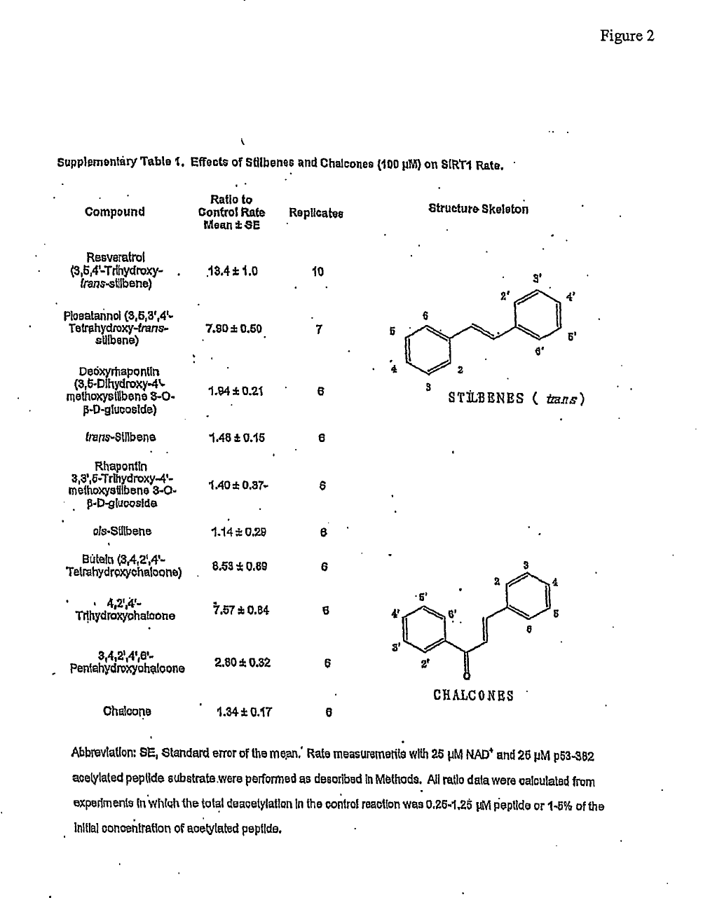

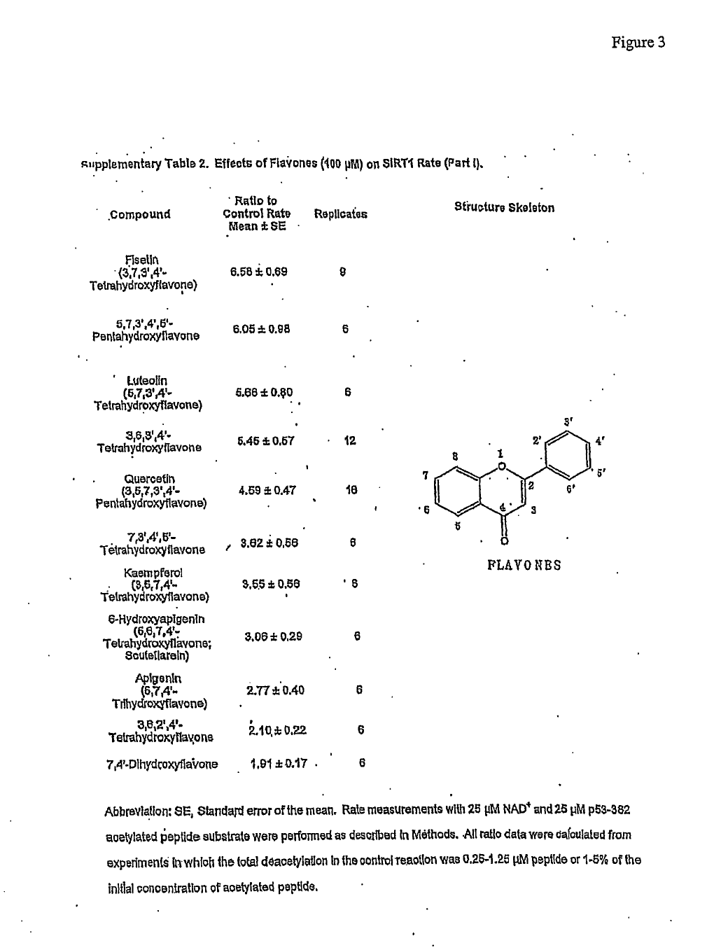

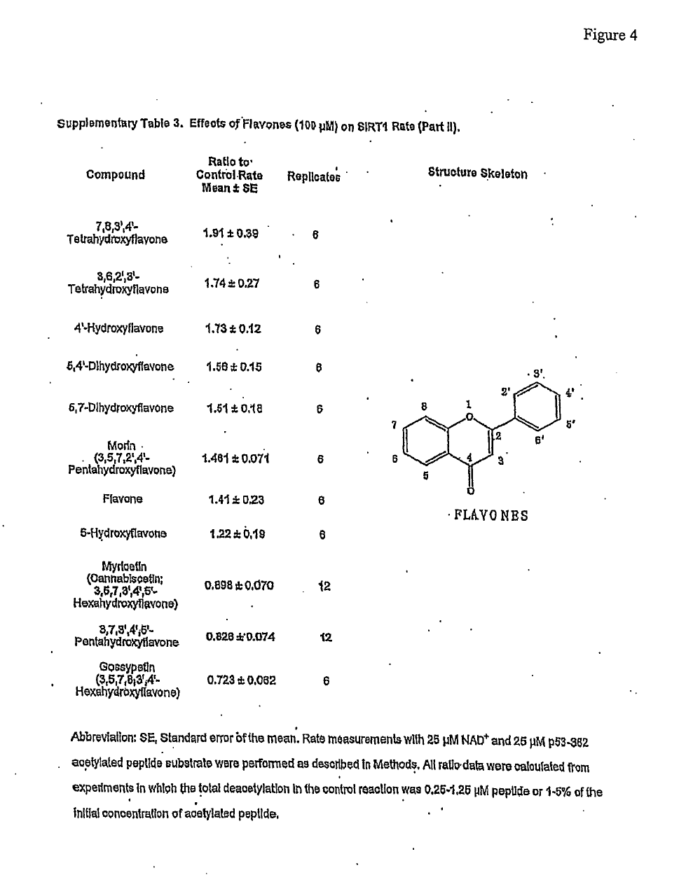

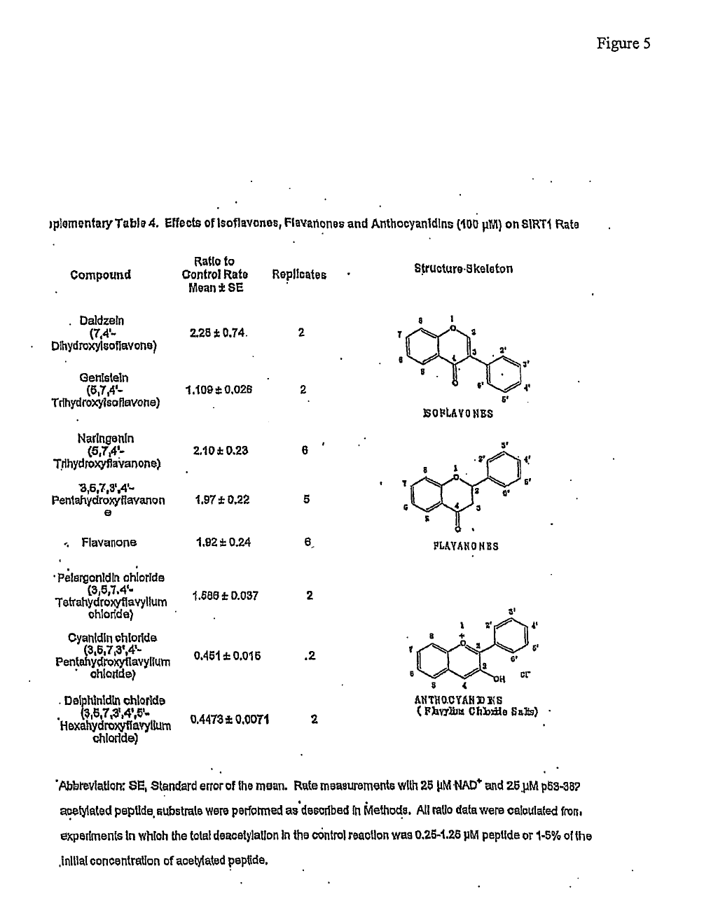

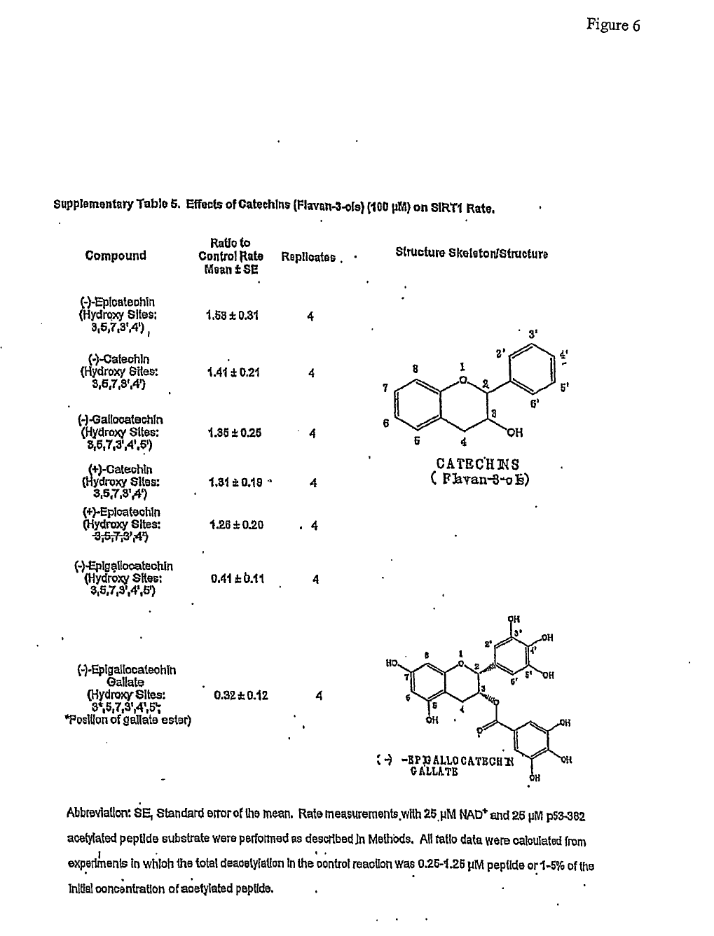

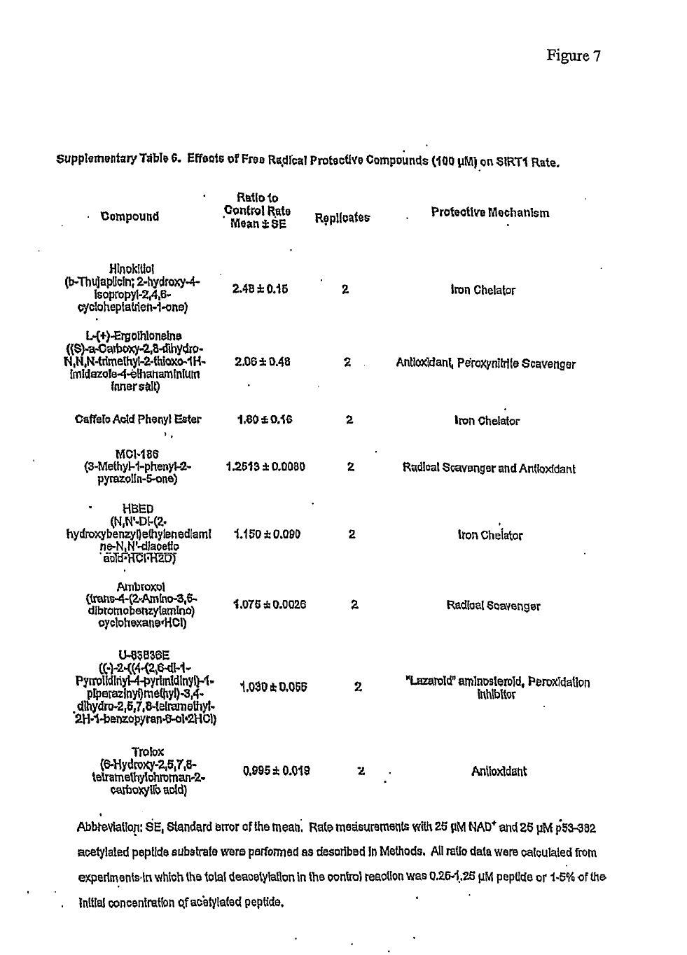

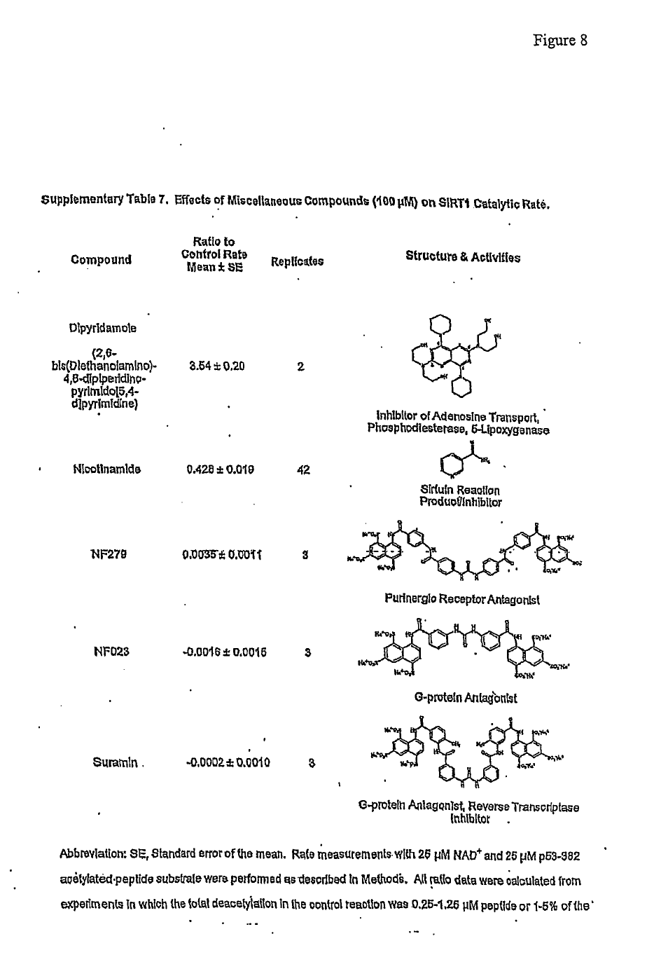

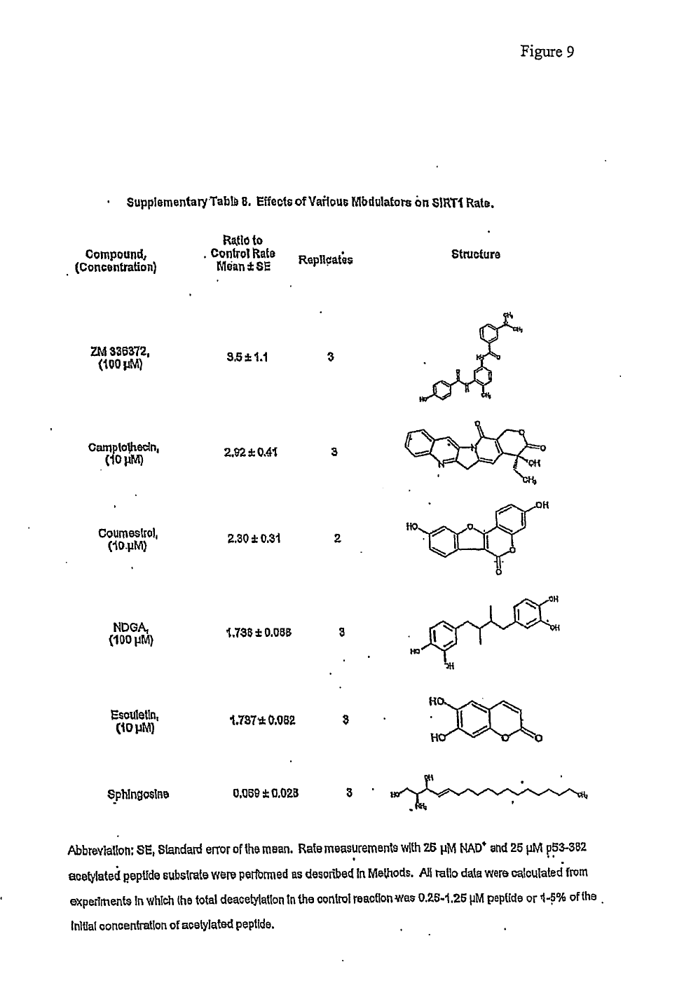

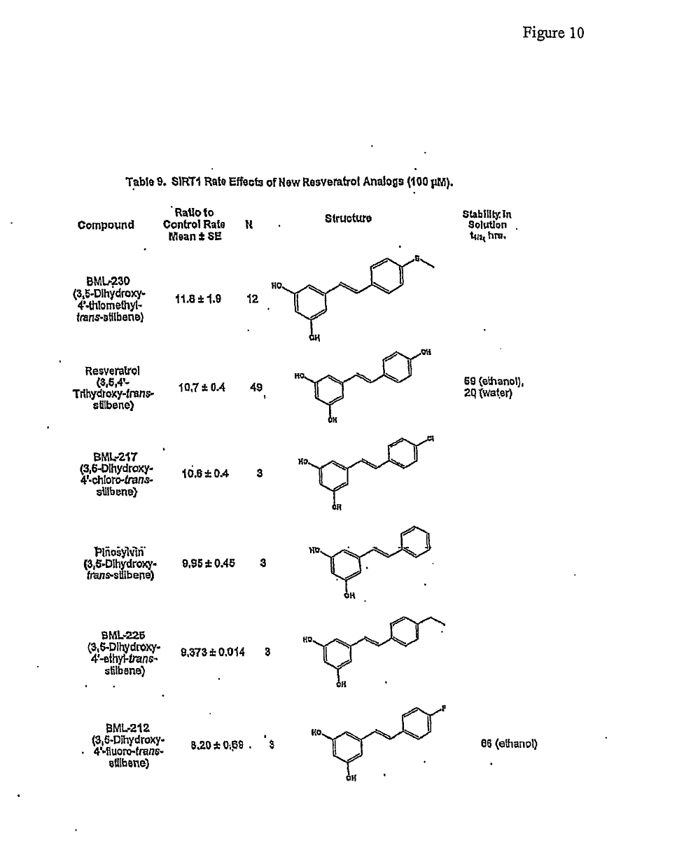

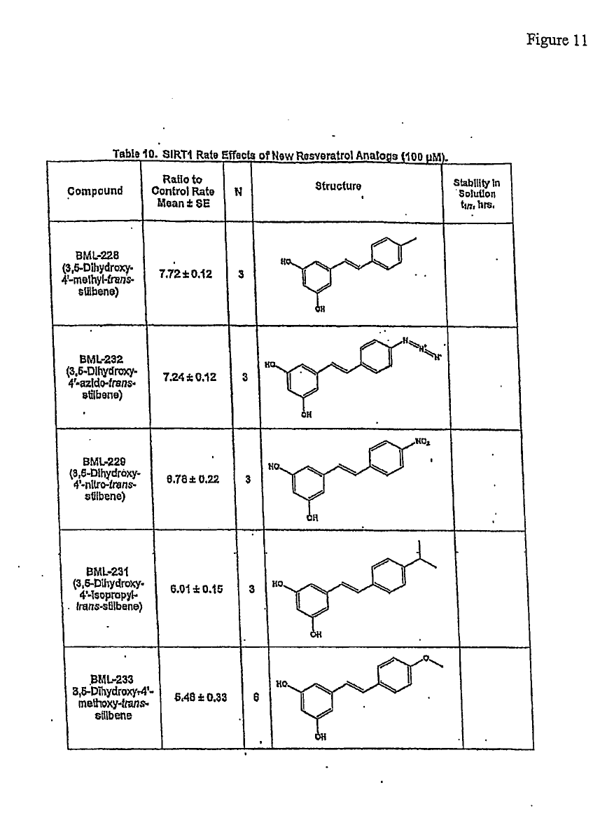

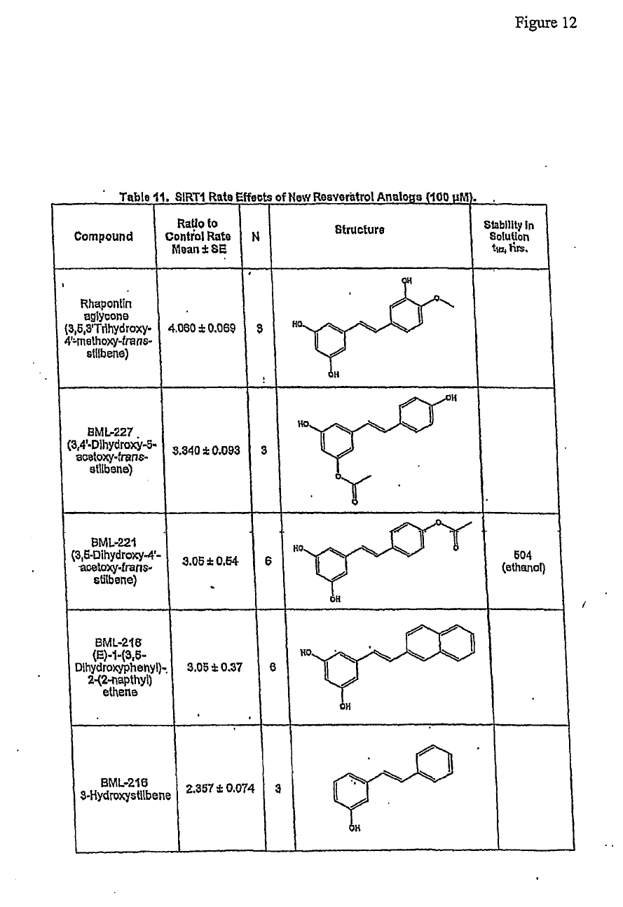

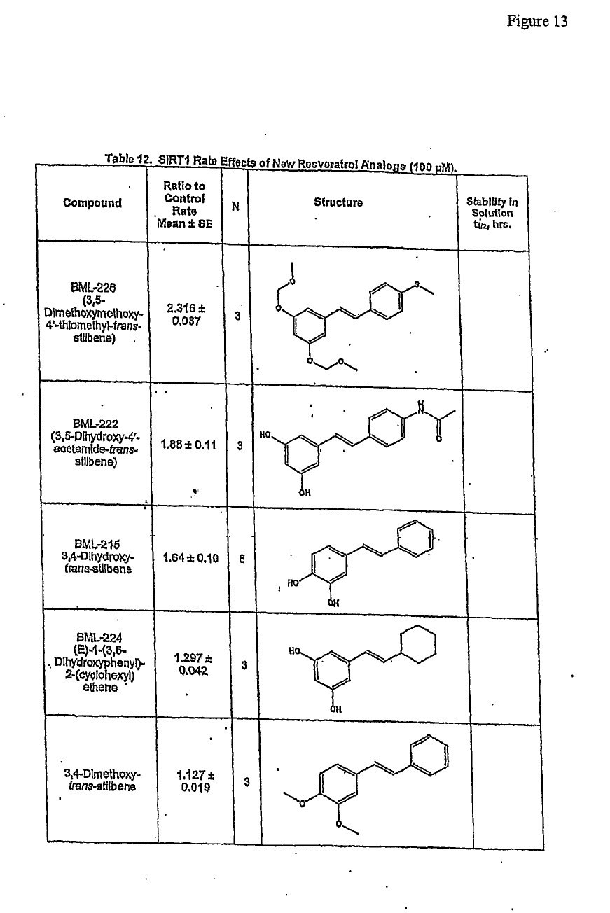

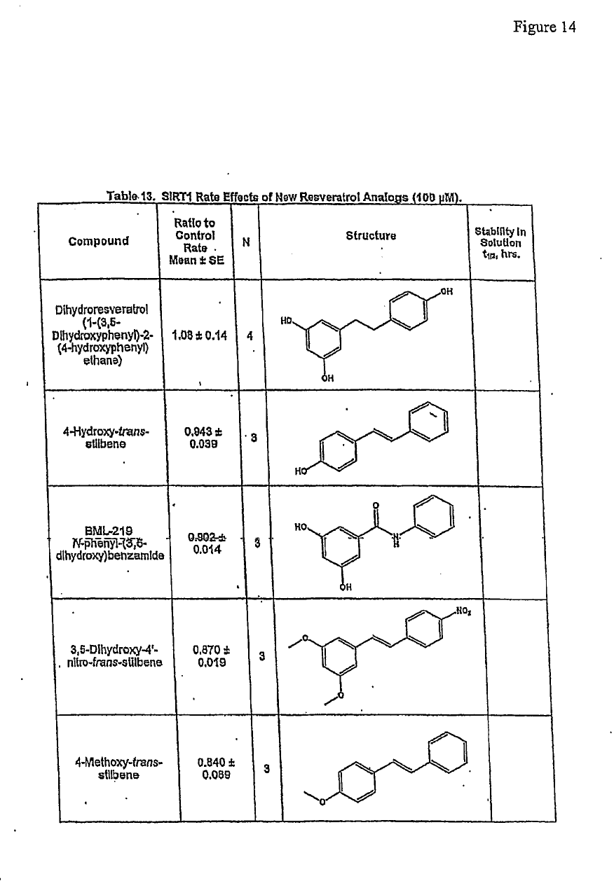

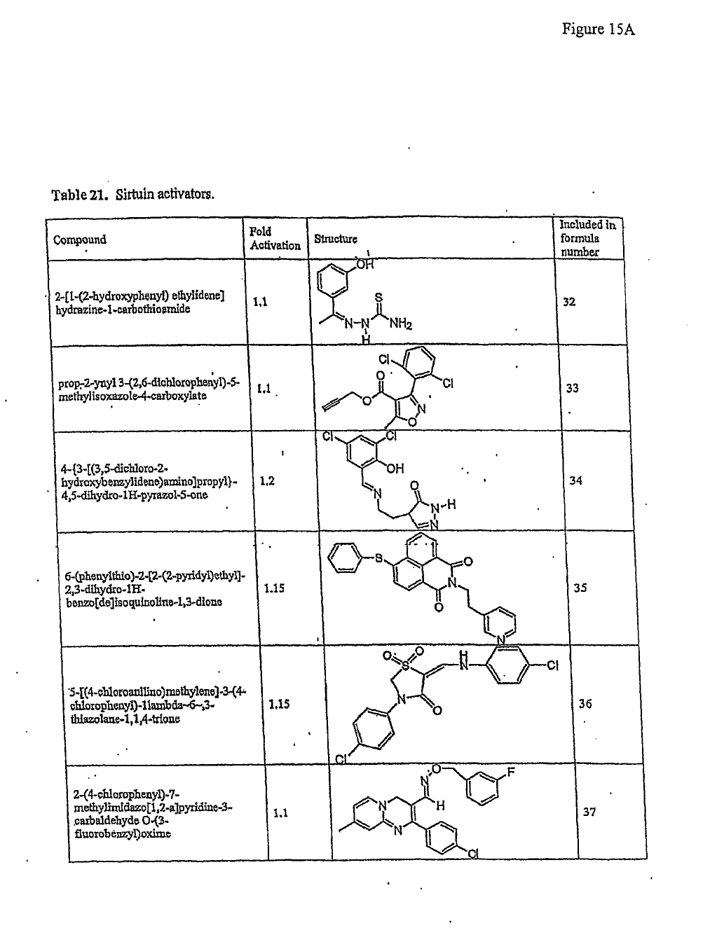

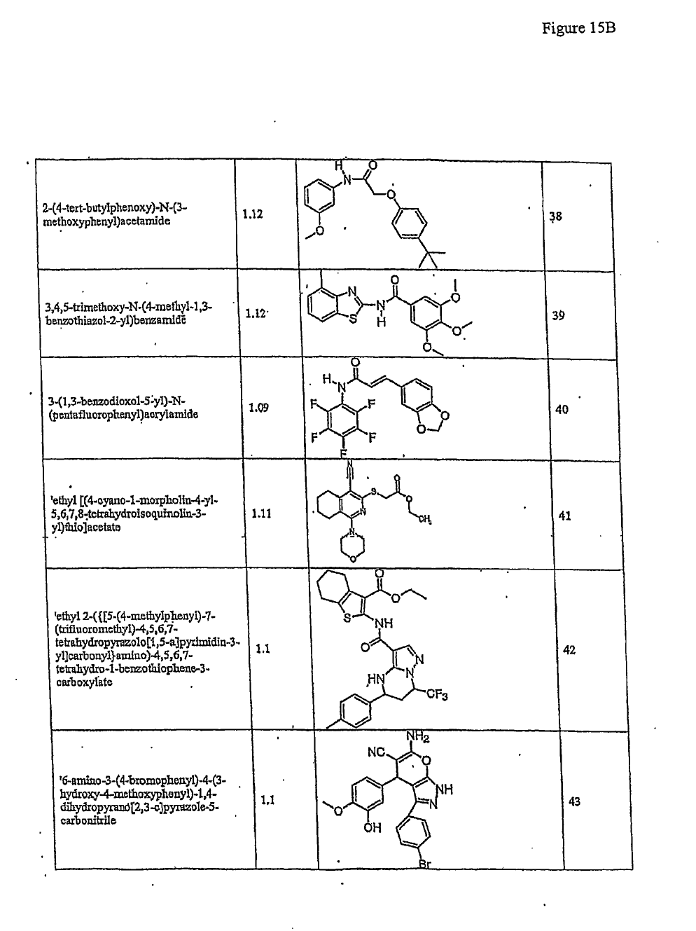

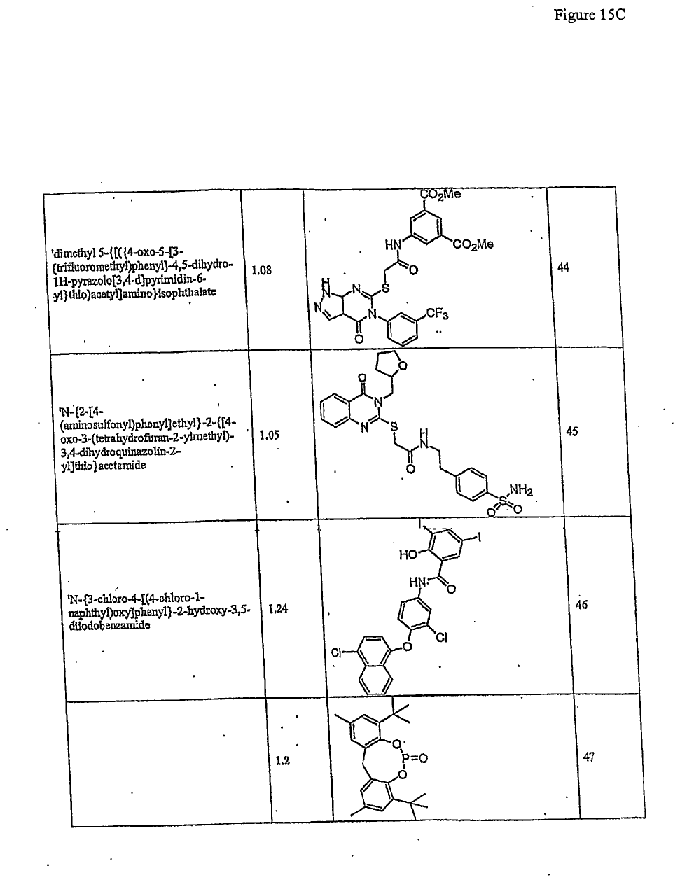

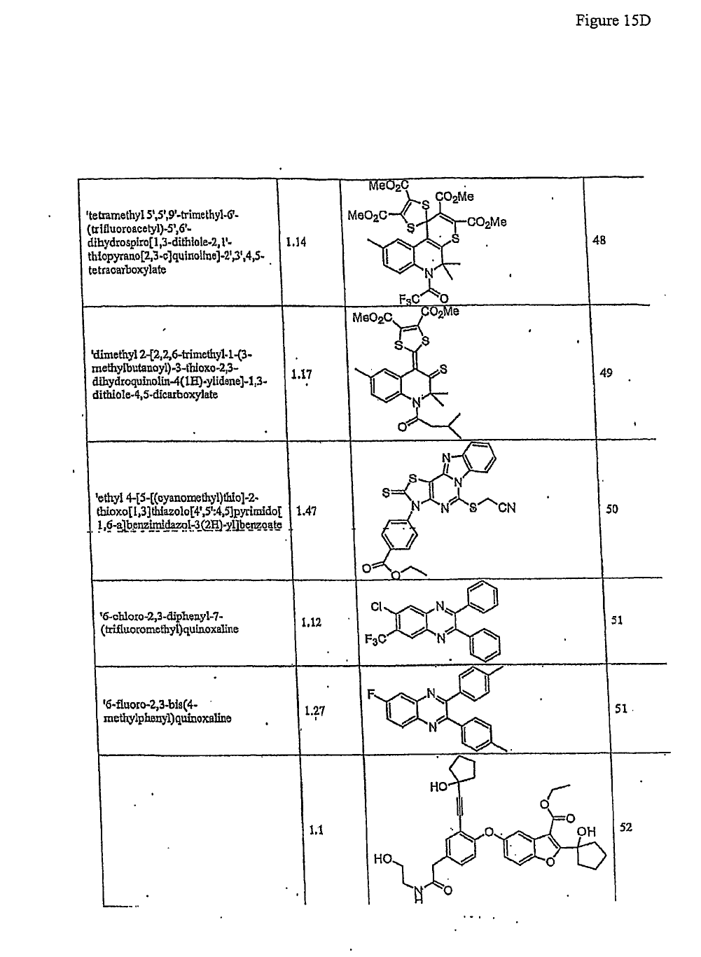

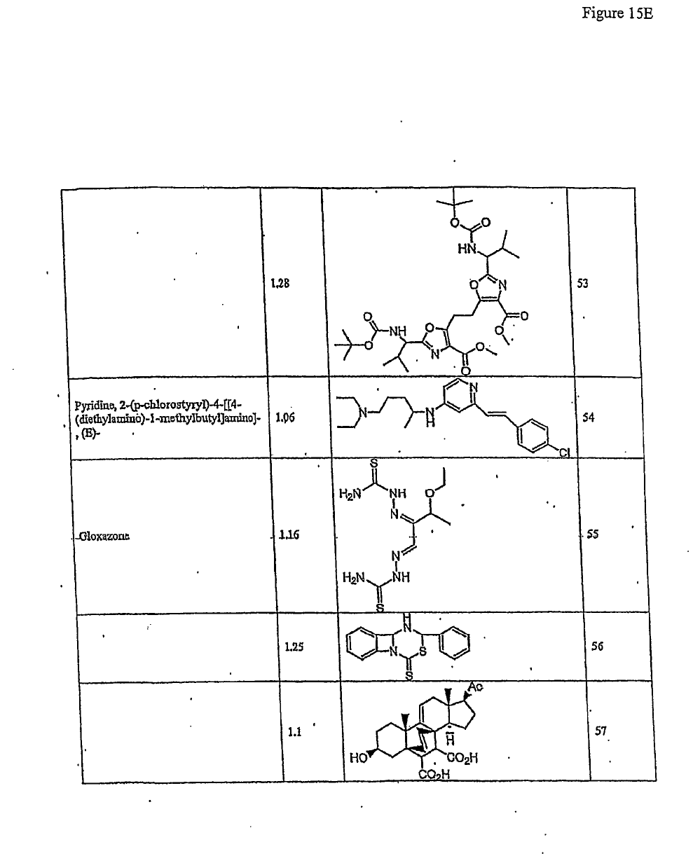

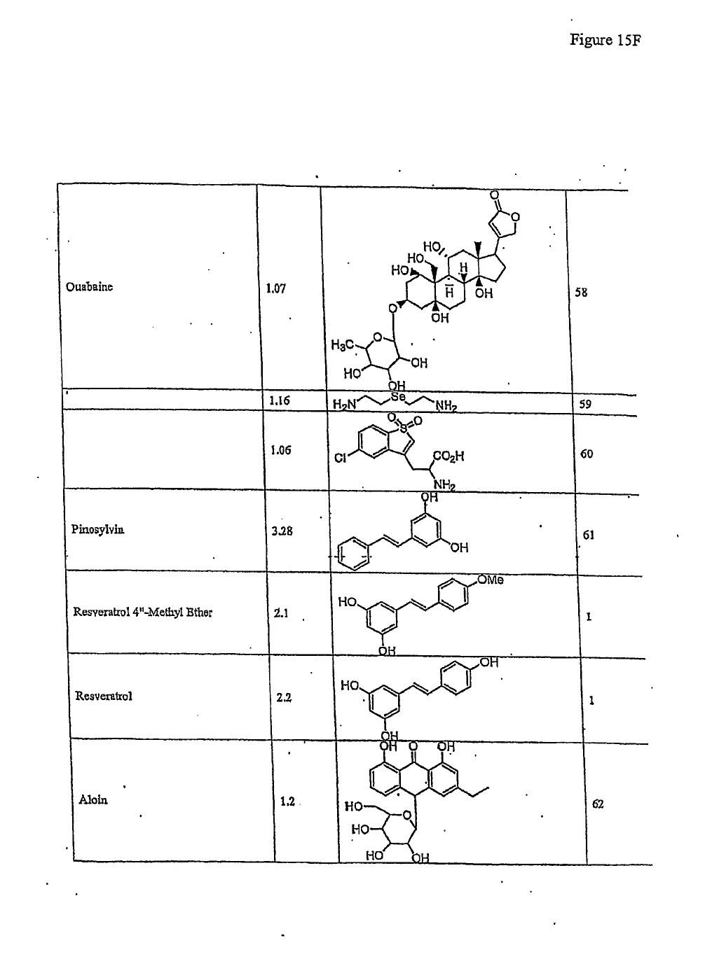

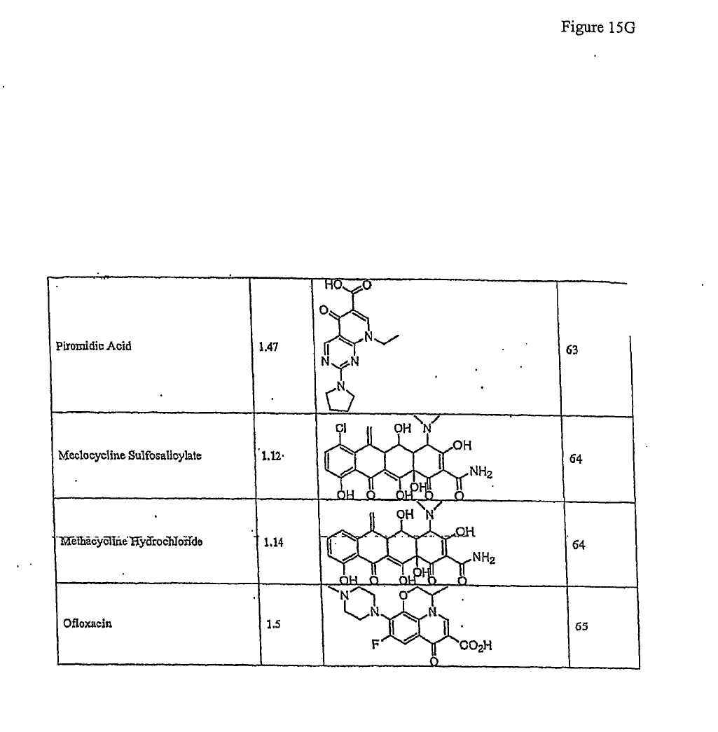

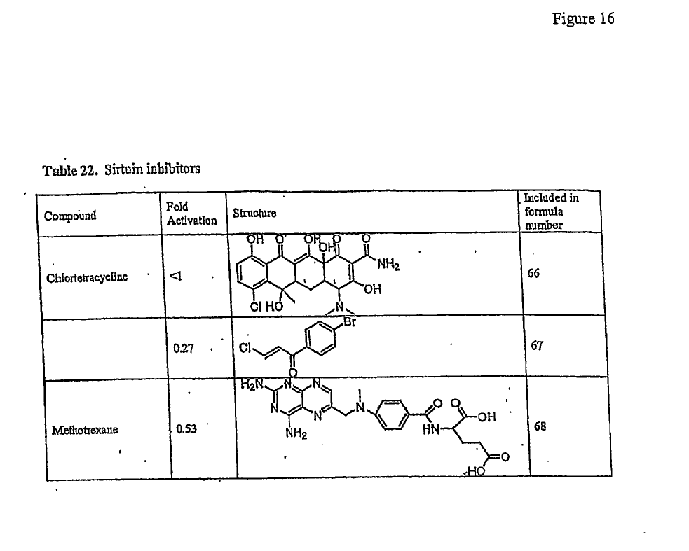

Specific examples of sirtuin modulators (e.g., sirtuin activators and sirtuin inhibitors) are described in U.S. Patent Publication Nos. 2005/0136537 and 2005/0096256 and include, for example, the compounds shown in FIGS. 1-16.

Also included are pharmaceutically acceptable addition salts and complexes of the sirtuin modulators described herein. In cases wherein the compounds may have one or more chiral centers, unless specified, the compounds contemplated herein may be a single stereoisomer or racemic mixtures of stereoisomers.

The compounds and salts thereof described herein also include their corresponding hydrates (e.g., hemihydrate, monohydrate, dihydrate, trihydrate, tetrahydrate) and solvates. Suitable solvents for preparation of solvates and hydrates can generally be selected by a skilled artisan.

The compounds and salts thereof can be present in amorphous or crystalline (including co-crystalline and polymorph) forms.

Sirtuin modulating compounds also include the related secondary metabolites, such as phosphate, sulfate, acyl (e.g., acetyl, fatty acid acyl) and sugar (e.g., glucurondate, glucose) derivatives (e.g., of hydroxyl groups), particularly the sulfate, acyl and sugar derivatives. In other words, substituent groups —OH also include —OSO₃⁻M⁺, where M⁺ is a suitable cation (preferably H⁺, NH₄⁺ or an alkali metal ion such as Na⁺ or K⁺) and sugars such as 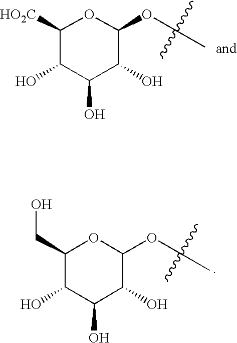
These groups are generally cleavable to —OH by hydrolysis or by metabolic (e.g., enzymatic) cleavage.

In cases in which the sirtuin-activating compounds have unsaturated carbon-carbon double bonds, both the cis (Z) and trans (E) isomers are contemplated herein. In cases wherein the compounds may exist in tautomeric forms, such as keto-enol tautomers, such as 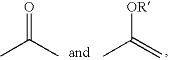
each tautomeric form is contemplated as being included within the methods presented herein, whether existing in equilibrium or locked in one form by appropriate substitution with R′. The meaning of any substituent at any one occurrence is independent of its meaning, or any other substituent's meaning, at any other occurrence.

Also included in the methods presented herein are prodrugs of the sirtuin-activating compounds described herein. Prodrugs are considered to be any covalently bonded carriers that release the active parent drug in vivo.

Analogs and derivatives of the sirtuin-activating compounds described herein can also be used for activating a member of the sirtuin protein family. For example, derivatives or analogs may make the compounds more stable or improve their ability to traverse cell membranes or being phagocytosed or pinocytosed. Exemplary derivatives include glycosylated derivatives, as described, e.g., in U.S. Pat. No. 6,361,815 for resveratrol. Other derivatives of resveratrol include cis- and trans-resveratrol and conjugates thereof with a saccharide, such as to form a glucoside (see, e.g., U.S. Pat. No. 6,414,037). Glucoside polydatin, referred to as piceid or resveratrol 3-O-beta-D-glucopyranoside, can also be used. Saccharides to which compounds may be conjugated include glucose, galactose, maltose, lactose and sucrose. Glycosylated stilbenes are further described in Regev-Shoshani et al. Biochemical J. (published on Apr. 16, 2003 as BJ20030141). Other derivatives of compounds described herein are esters, amides and prodrugs. Esters of resveratrol are described, e.g., in U.S. Pat. No. 6,572,882. Resveratrol and derivatives thereof can be prepared as described in the art, e.g., in U.S. Pat. Nos. 6,414,037; 6,361,815; 6,270,780; 6,572,882; and Brandolini et al. (2002) J. Agric. Food. Chem. 50:7407. Derivatives of hydroxyflavones are described, e.g., in U.S. Pat. No. 4,591,600. Resveratrol and other activating compounds can also be obtained commercially, e.g., from Sigma.

In certain embodiments, if a sirtuin-activating compound occurs naturally, it may be at least partially isolated from its natural environment prior to use. For example, a plant polyphenol may be isolated from a plant and partially or significantly purified prior to use in the methods described herein. An activating compound may also be prepared synthetically, in which case it would be free of other compounds with which it is naturally associated. In an illustrative embodiment, an activating composition comprises, or an activating compound is associated with, less than about 50%, 10%, 1%, 0.1%, 10⁻²% or 10⁻³% of a compound with which it is naturally associated.

In certain embodiments, a certain biological function (e.g., modulating metabolic activity) is modulated by a sirtuin-activating compound with the proviso that the term sirtuin-activating compound does not include one or more specific compounds. For example, in certain embodiments, a sirtuin-activating compound may be any compound that is capable of increasing the level of expression and/or activity of a sirtuin protein with the proviso that the compound is not resveratrol, flavone, any other compound specifically cited herein. In an exemplary embodiment, a sirtuin-activating compound may be a compound of any one of formulas 1-25, 30, 32-65, and 69-88 with the proviso that the compound is not resveratrol, flavone, or any other compound specifically cited herein. In an exemplary embodiment, a sirtuin-activating compound does not include any of the compounds cited in U.S. Pat. Nos. 6,410,596 or 6,552,085, the disclosures of which are hereby incorporated by reference in their entirety.

In certain embodiments, the subject sirtuin activators, such as SIRT1 activators, do not have any substantial ability to inhibit PI3-kinase, inhibit aldoreductase and/or inhibit tyrosine protein kinases at concentrations (e.g., in vivo) effective for activating the deacetylase activity of the sirtuin, e.g., SIRT1. For instance, in preferred embodiments the sirtuin activator is chosen to have an EC₅₀ for activating sirtuin deacetylase activity that is at least 5 fold less than the EC₅₀ for inhibition of one or more of aldoreductase and/or tyrosine protein kinases, and even more preferably at least 10 fold, 100 fold or even 1000 fold less. Methods for assaying PI3-Kinase activity, aldose reductase activity, and tyrosine kinase activity are well known in the art and kits to perform such assays may be purchased commercially. See e.g., U.S. Patent Publication No. 2003/0158212 for PI3-kinase assays; U.S. Patent Publication No. 2002/20143017 for aldose reductase assays; tyrosine kinase assay kits may be purchased commercially, for example, from Promega (Madison, Wis.; world wide web at promega.com), Invitrogen (Carlsbad, Calif.; world wide web at invitrogen.com) or Molecular Devices (Sunnyvale, Calif.; world wide web at moleculardevices.com).

In certain embodiments, the subject sirtuin activators do not have any substantial ability to transactivate EGFR tyrosine kinase activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for transactivating EGFR tyrosine kinase activity, and even more preferably at least 10 fold, 100 fold or even 1000 fold less. Methods for assaying transactivation of EGFR tyrosine kinase activity are well known in the art, see e.g., Pai et al. Nat. Med. 8: 289-93 (2002) and Vacca et al. Cancer Research 60: 5310-5317 (2000).

In certain embodiments, the subject sirtuin activators do not have any substantial ability to cause coronary dilation at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for coronary dilation, and even more preferably at least 10 fold, 100 fold or even 1000 fold less. Methods for assaying vasodilation are well known in the art, see e.g., U.S. Patent Publication No. 2004/0236153.

In certain embodiments, the subject sirtuin activators do not have any substantial spasmolytic activity at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for spasmolytic effects (such as on gastrointestinal muscle), and even more preferably at least 10 fold, 100 fold or even 1000 fold less. Methods for assaying spasmolytic activity are well known in the art, see e.g., U.S. Patent Publication No. 2004/0248987.

In certain embodiments, the subject sirtuin activators do not have any substantial ability to inhibit hepatic cytochrome P450 1B1 (CYP) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of P450 1B1, and even more preferably at least 10 fold, 100 fold or even 1000 fold less. Methods for assaying cytochrome P450 activity are well known in the art and kits to perform such assays may be purchased commercially. See e.g., U.S. Pat. Nos. 6,420,131 and 6,335,428 and Promega (Madison, Wis.; world wide web at promega.com).

In certain embodiments, the subject sirtuin activators do not have any substantial ability to inhibit nuclear factor-kappaB (NF-κB) at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of NF-κB, and even more preferably at least 10 fold, 100 fold or even 1000 fold less. Methods for assaying NF-κB activity are well known in the art and kits to perform such assays may be purchased commercially (e.g., from Oxford Biomedical Research (Ann Arbor, Mich.; world wide web at oxfordbiomed.com)).

In certain embodiments, the subject sirtuin activators do not have any substantial ability to inhibit a histone deacetylase (HDACs) class I, a HDAC class II, or HDACs I and II, at concentrations (e.g., in vivo) effective for activating the deacetylase activity of the sirtuin. For instance, in preferred embodiments the sirtuin activator is chosen to have an EC₅₀ for activating sirtuin deacetylase activity that is at least 5 fold less than the EC₅₀ for inhibition of an HDAC I and/or HDAC II, and even more preferably at least 10 fold, 100 fold or even 1000 fold less. Methods for assaying HDAC I and/or HDAC II activity are well known in the art and kits to perform such assays may be purchased commercially. See e.g., BioVision, Inc. (Mountain View, Calif.; world wide web at biovision.com) and Thomas Scientific (Swedesboro, N.J.; world wide web at tomassci.com).

In certain embodiments, the subject SIRT1 activators do not have any substantial ability to activate SIRT1 orthologs in lower eukaryotes, particularly yeast or human pathogens, at concentrations (e.g., in vivo) effective for activating the deacetylase activity of human SIRT1. For instance, in preferred embodiments the SIRT1 activator is chosen to have an EC₅₀ for activating human SIRT1 deacetylase activity that is at least 5 fold less than the EC₅₀ for activating yeast Sir2 (such as Candida, S. cerevisiae, etc), and even more preferably at least 10 fold, 100 fold or even 1000 fold less.

In certain embodiments, the sirtuin activating compounds may have the ability to activate one or more sirtuin protein homologs, such as, for example, one or more of human SIRT1, SIRT2, SIRT3, SIRT4, SIRT5, SIRT6, or SIRT7. In other embodiments, a SIRT1 activator does not have any substantial ability to activate other sirtuin protein homologs, such as, for example, one or more of human SIRT2, SIRT3, SIRT4, SIRT5, SIRT6, or SIRT7, at concentrations (e.g., in vivo) effective for activating the deacetylase activity of human SIRT1. For instance, the SIRT1 activator may be chosen to have an EC₅₀ for activating human SIRT1 deacetylase activity that is at least 5 fold less than the EC₅₀ for activating one or more of human SIRT2, SIRT3, SIRT4, SIRT5, SIRT6, or SIRT7, and even more preferably at least 10 fold, 100 fold or even 1000 fold less.

In certain embodiments, SIRT3 and SIRT4 modulators may be used to modulate fat mobilization. For example, SIRT3 and/or SIRT4 activators may be used to induce fat mobilization and may be used to treat, e.g., obesity and insulin resistance disorders.

In other embodiments, the subject sirtuin activators do not have any substantial ability to inhibit protein kinases; to phosphorylate mitogen activated protein (MAP) kinases; to inhibit the catalytic or transcriptional activity of cyclo-oxygenases, such as COX-2; to inhibit nitric oxide synthase (iNOS); or to inhibit platelet adhesion to type I collagen at concentrations (e.g., in vivo) effective for activating the deacetylase activity of the sirtuin. For instance, in preferred embodiments, the sirtuin activator is chosen to have an EC₅₀ for activating sirtuin deacetylase activity that is at least 5 fold less than the EC₅₀ for performing any of these activities, and even more preferably at least 10 fold, 100 fold or even 1000 fold less. Methods for assaying protein kinase activity, cyclo-oxygenase activity, nitric oxide synthase activity, and platelet adhesion activity are well known in the art and kits to perform such assays may be purchased commercially. See e.g., Promega (Madison, Wis.; world wide web at promega.com), Invitrogen (Carlsbad, Calif.; world wide web at invitrogen.com); Molecular Devices (Sunnyvale, Calif.; world wide web at moleculardevices.com) or Assay Designs (Ann Arbor, Mich.; world wide web at assaydesigns.com) for protein kinase assay kits; Amersham Biosciences (Piscataway, N.J.; world wide web at amershambiosciences.com) for cyclo-oxygenase assay kits; Amersham Biosciences (Piscataway, N.J.; world wide web at amershambiosciences.com) and R&D Systems (Minneapolis, Minn.; world wide web at mdsystems.com) for nitric oxide synthase assay kits; and U.S. Pat. Nos. 5,321,010; 6,849,290; and 6,774,107 for platelet adhesion assays.

In certain embodiments, a compound described herein, e.g., a sirtuin activator or inhibitor, does not have significant or detectable anti-oxidant activities, as determined by any of the standard assays known in the art. For example, a compound does not significantly scavenge free-radicals, such as O₂ radicals. A compound may have less than about 2, 3, 5, 10, 30 or 100 fold anti-oxidant activity relative to another compound, e.g., resveratrol.

In certain embodiments, a sirtuin activating compound may have a binding affinity for a sirtuin of about 10⁻⁹ M, 10⁻¹⁰ M, 10⁻¹¹ M, 10⁻¹² M or less. A sirtuin activating compound may reduce the Kₘ of a sirtuin for its substrate or NAD⁺ by a factor of at least about 2, 3, 4, 5, 10, 20, 30, 50 or 100. A sirtuin activating compound may increase the Vₘₐₓ of a sirtuin by a factor of at least about 2, 3, 4, 5, 10, 20, 30, 50 or 100. Exemplary compounds that may increase the Vmax of a sirtuin include, for example, analogs of isonicotinamide, such as, for example, compounds of formulas 69-72, and/or analogs of O-acetyl-ADP-ribose, such as, for example, compounds of formulas 73-76. A compound may have an EC₅₀ for activating the deacetylase activity of a sirtuin of less than about 1 nM, less than about 10 nM, less than about 100 nM, less than about 1 μM, less than about 10 μM, less than about 100 μM, or from about 1-10 nM, from about 10-100 nM, from about 0.1-1 μM, from about 1-10 μM or from about 10-100 μM. A compound may activate the deacetylase activity of a sirtuin by a factor of at least about 5, 10, 20, 30, 50, or 100, as measured in an acellular assay or in a cell based assay as described in the Examples. A compound may cause at least a 10%, 30%, 50%, 80%, 2 fold, 5 fold, 10 fold, 50 fold or 100 fold greater induction of the deacetylase activity of SIRT1 relative to the same concentration of resveratrol or other compound described herein. A compound may also have an EC₅₀ for activating SIRT5 that is at least about 10 fold, 20 fold, 30 fold, 50 fold greater than that for activating SIRT1.

In an exemplary embodiment, the methods and compositions described herein may include a combination therapy comprising (i) at least one sirtuin-activating compound that reduce the Kₘ of a sirtuin for its substrate or NAD⁺ by a factor of at least about 2, 3, 4, 5, 10, 20, 30, 50 or 100, and (ii) at least one sirtuin-activating compound that increases the Vₘₐₓ of a sirtuin by a factor of at least about 2, 3, 4, 5, 10, 20, 30, 50 or 100. In one embodiment, a combination therapy may comprise at least two of the following: (i) at least one sirtuin-activating compound of formula 1-25, 30, and 32-65, (ii) at least one sirtuin-activating compound of formula 69-76, and (iii) at least one sirtuin-activating compound of formula 77-88.

A compound may traverse the cytoplasmic membrane of a cell. For example, a compound may have a cell-permeability of at least about 20%, 50%, 75%, 80%, 90% or 95%.

Compounds described herein may also have one or more of the following characteristics: the compound may be essentially non-toxic to a cell or subject; the compound may be an organic molecule or a small molecule of 2000 amu or less, 1000 amu or less; a compound may have a half-life under normal atmospheric conditions of at least about 30 days, 60 days, 120 days, 6 months or 1 year; the compound may have a half-life in solution of at least about 30 days, 60 days, 120 days, 6 months or 1 year; a compound may be more stable in solution than resveratrol by at least a factor of about 50%, 2 fold, 5 fold, 10 fold, 30 fold, 50 fold or 100 fold; a compound may promote deacetylation of the DNA repair factor Ku70; a compound may promote deacetylation of RelA/p65; a compound may increase general turnover rates and enhance the sensitivity of cells to TNF-induced apoptosis.

II. Exemplary Therapeutic Applications of the Sirtuin-activating Compounds

In certain embodiments, the invention provides methods for treating and/or preventing a wide variety of diseases and disorders by administering to a subject a high dose of a sirtuin activator. In an exemplary embodiments, a quantity of a sirtuin activator having a sirtuin activating effect equal to or greater than the sirtuin activating effect of 18 mg/kg resveratrol may be administered to a subject. A high dose may be administered to a subject once, or multiple times (e.g., daily) until a desired therapeutic effect is achieved. For example, a high dose may be administered daily for 1 day, 1 week, 2 weeks, 1 month, 2 months, 3 months, 6 months, 1 year, or more depending on the disease or disorder being treated. A high dose of a sirtuin activator may be administered daily in a single dosage or may be divided into multiple dosages, e.g., that are taken twice or three times per day. In an exemplary embodiment, a high dose of a sirtuin activator may be administered in a sustained release formulation. Exemplary diseases or disorders that may be treated using a high dose of a sirtuin activator include, for example, diseases or disorders related to aging or stress, diabetes, obesity, neurodegenerative diseases, diseases or disorders associated with mitochondrial dysfunction, cardiovascular disease, blood clotting disorders, inflammation, cancer, and/or flushing, etc. The methods comprise administering to a subject in need thereof a high dose of a sirtuin activating compound.

In certain embodiments, a high dose of a sirtuin activating compound may be taken alone or in combination with other compounds. In one embodiment, a mixture of a high dose of two or more sirtuin activating compounds may be administered to a subject in need thereof. In another embodiment, a high dose of a sirtuin activating compound may be administered with one or more of the following compounds: resveratrol, butein, fisetin, piceatannol, or quercetin. In an exemplary embodiment, a high dose of a sirtuin activating compound may be administered in combination with nicotinic acid. In yet another embodiment, a high dose of one or more sirtuin activating compound may be administered with one or more therapeutic agents for the treatment or prevention of various diseases, including, for example, cancer, diabetes, neurodegenerative diseases, diseases or disorders associated with mitochondrial dysfunction, cardiovascular disease, blood clotting, inflammation, flushing, obesity, ageing, stress, etc. In various embodiments, combination therapies comprising a high dose of a sirtuin activating compound may refer to (1) pharmaceutical compositions that comprise a high dose of one or more sirtuin activating compounds in combination with one or more therapeutic agents; and (2) co-administration of a high dose of one or more sirtuin activating compounds with one or more therapeutic agents wherein the sirtuin activating compound and therapeutic agent have not been formulated in the same compositions. When using separate formulations, the high dose of the sirtuin activating compound may be administered at the same, intermittent, staggered, prior to, subsequent to, or combinations thereof, with the administration of another therapeutic agent.

Metabolic Disorders/Diabetes/Weight Control

Described herein are methods for treating or preventing obesity or generally weight gain, in a subject, such as to reduce the weight of the subject or reduce weight gain. A method may comprise administering to a subject, such as a subject in need thereof, a high dose of an agent that increases the activity or protein level of a sirtuin, such as SIRT1 or Sir2, e.g., a sirtuin activator. A subject in need of such a treatment may be a subject who is obese, or likely to become obese, or who has, or is, likely to gain excess weight, as predicted, e.g., from family history. Exemplary agents are those described herein. A combination of agents may also be administered (e.g., a combination of a high dose of a sirtuin activator with an anti-obesity agent). A method may further comprise monitoring the weight of the subject and/or the level of activation of sirtuins, for example, in adipose tissue.

Also described herein are methods for treating or preventing a metabolic disorder, such as insulin-resistance or other precursor symptom of type II diabetes or complications thereof. Methods may increase insulin sensitivity or decrease insulin levels in a subject. A method may comprise administering to a subject, such as a subject in need thereof, a high dose of an agent that increases the activity or protein level of a sirtuin, such as SIRT1 or Sir2. A subject in need of such a treatment may be a subject who has insulin resistance or other precusor symptom of type II diabetes, who has type II diabetes, or who is likely to develop any of these conditions. For example, the subject may be a subject having insulin resistance, e.g., having high circulating levels of insulin and/or associated conditions, such as hyperlipidemia, dyslipogenesis, hypercholesterolemia, impaired glucose tolerance, high blood glucose sugar level, other manifestations of syndrome X, hypertension, atherosclerosis and lipodystrophy. Exemplary agents are those described herein.

In certain embodiments, a high dose of a sirtuin activating compound may be used to decrease the amount of fat absorption in the gastrointestinal tract of an individual thereby promoting weight loss and/or preventing gain. In certain embodiments, a sirtuin modulating compound may be administered in combination with another agent that inhibits fat absorption, such as, Orlistat (also known as tetrahydrolipstatin and sold under the brand name XENICAL™). Orlistat is a potent inhibitor of gastrointestinal lipases, i.e. lipases that are responsible for breaking down ingested fat (gastric lipase, carboxylester lipase, pancreatic lipase). As a consequence of lipase inhibition, the undigested fats cannot be absorbed and are excreted in the feces. In certain embodiments, sirtuin modulating compound may permit a lower dose of a fat absorption inhibitor to be administered and still achieve therapeutically desirable results. Such combination therapy may permit avoidance of undesirable side affects associated with the fat absorption inhibitor.

A combination of agents may also be administered (e.g., a combination of a high dose of a sirtuin activating compound with an anti-diabetic agent). A method may further comprise monitoring in the subject the state of any of these conditions and/or the level of activation of sirtuins, for example, in adipose tissue.

The sirtuin-activating compounds described herein may be taken alone or in combination with other compounds. The other compounds may be other sirtuin and/or AMPK activators. For example, Longevinex™, which is a red wine extract, and contains, in addition to resveratrol, other sirtuin activators, such as quercetin, is a particularly potent agent for mobilizing fat. Longevinex™ can be obtained on the world wide web at longevinex.com.

In an exemplary embodiment, a high dose of a sirtuin-activating compound may be administered as a combination therapy with a lipid lowering, an anti-obesity and/or an anti-diabetic agent. Examples of lipid lowering, anti-obesity or anti-diabetic agents suitable for administration in combination with a high dose of a sirtuin activator include chromium, fat binding polymers, carbohydrate binding polymers, lipase inhibitors, thermogenic agents, catecholamine reuptake inhibitors, and thyroid hormone. For example, for reducing weight, preventing weight gain, or treatment or prevention of obesity, a high dose of one or more sirtuin-activating compounds may be used in combination with one or more anti-obesity agents such as the following: phenylpropanolamine, ephedrine, pseudoephedrine, phentermine, a cholecystokinin-A agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist, a melanin concentrating hormone antagonist, the OB protein (leptin), a leptin analog, a leptin receptor agonist, a cannabinoid receptor modulator (such as ramonibant), a galanin antagonist or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin, anticonvulsants and ciliary neurotrophic factors such as Axokine.

In other embodiments, a high dose of one or more sirtuin activating compounds may be used in combination with one or more anti-diabetic agents such as the following: an aldose reductase inhibitor, a glycogen phosphorylase inhibitor, a sorbitol dehydrogenase inhibitor, a protein tyrosine phosphatase 1B inhibitor, a dipeptidyl protease inhibitor, insulin (including orally bioavailable insulin preparations), an insulin mimetic, metformin, acarbose, a peroxisome proliferator-activated receptor-γ (PPAR-γ) ligand such as troglitazone, rosaglitazone, pioglitazone or GW-1929, a sulfonylurea, glipazide, glyburide, or chlorpropamide wherein the amounts of the first and second compounds result in a therapeutic effect. Other anti-diabetic agents include a glucosidase inhibitor, a glucagon-like peptide-1 (GLP-1), insulin, a PPAR α/γ dual agonist, a meglitimide and an αP2 inhibitor. In an exemplary embodiment, an anti-diabetic agent may be a dipeptidyl peptidase IV (DP-IV or DPP-IV) inhibitor, such as, for example LAF237 from Novartis (NVP DPP728; 1-[[[2-[(5-cyanopyridin-2-yl)amino]ethyl]amino]acetyl]-2-cyano-(S)-pyrrolidine) or MK-04301 from Merck (see e.g., Hughes et al., Biochemistry 38: 11597-603 (1999)).

In other embodiments, a high dose of one or more sirtuin activating compounds may be used in combination with one or more lipid lowering agents such as the following: statins such as simvastatin (Zocor), pravastatin (Pravachol), lovostatin (Mevacor), fleuvastatin (Lescol), cerivastatin (Baycol), rosuvastatin (Crestcor) and atorvastatin (Lipitor) as well as niacin.

In certain embodiments, administration of a high dose of a sirtuin activator in combination with a lipid lowering, anti-obesity and/or anti-diabetic agent may reduce, alleviate or eliminate undesirable side effects associated with the anti-obesity and/or anti-diabetic agents. For example, administration of a lipid lowering, anti-obesity and/or anti-diabetic agent in combination with a high dose of a sirtuin activator may permit a therapeutically beneficial result upon administration of a lower dose of the lipid lowering, anti-obesity and/or anti-diabetic agent than would be necessary in the absence of the combination with the sirtuin activator. For example, a high dose of a sirtuin activator may be administered in combination with a low dose (e.g., an amount that reduces or prevents undesirable side effects such as increased heart rate and/or blood pressure) of a lipid lowering, anti-obesity and/or anti-diabetic drug. In certain embodiments, a high dose of a sirtuin activator may be administered in combination with thyroid hormone. In other embodiments, a high dose of a sirtuin activator may be administered in combination with an absorption blocker, such as an α-glucosidase inhibitor. In yet other embodiments, a high dose of a sirtuin activator may be administered in combination with metformin. In certain embodiments, a high dose of a sirtuin activator may be administered in combination with a catecholamine reuptake inhibitor, such as sibutramine. Additional examples of lipid lowering, anti-obesity or anti-diabetic agents are described herein. In other embodiments, the dose of the lipid lowering, anti-obesity and/or anti-diabetic agent may not be reduced as compared to a normal dose and administration of the sirtuin activator reduces, alleviates or abolishes a side effect of the lipid lowering, anti-obesity and/or anti-diabetic agent.

In certain embodiments, the use of sirtuin activating compounds can be used to reduce the amount of a lipid lowering, anti-obesity or anti-diabetic agent that is taken. This may be desirable in instances where a lipid lowering, anti-obesity and/or anti-diabetic agent dosing regimen produces unwanted side effects in the patient in need thereof. For example, administration of thyroid hormone to a subject typically causes an increase in heart rate and/or blood pressure. In certain embodiments, the invention provides methods to adjust (e.g., reduce) the amount of thyroid hormone administered to the patient to an amount that does not have an undesirable effect on heart rate and/or blood pressure by administering the thyroid hormone in combination with a high dose of a sirtuin activator. In other embodiments, the invention provides methods to adjust (e.g., reduce) the amount of metformin taken by a subject in need thereof by administering metformin in combination with a high dose of a sirtuin activator. In yet other embodiments, a high dose of a sirtuin activator is used to reduce the amount of sibutramine that is taken by a subject in need thereof.

The methods described herein may comprise administering daily, or every other day, or once a week, a high dose of a sirtuin activating compound, e.g., in the form of a pill, to a subject. In embodiments where the high dose of a sirtuin activating compound is administered daily to the subject, the sirtuin activating compound may be administered once a day. In other embodiments, it is administered twice or three times a day.

In some embodiments, the high dose of a sirtuin activating compound is administered in a sustained release formulation, e.g., by embedding or encapsulating the sirtuin activator into nanoparticles for delivery over a period of at least 12 hours, to a subject. In embodiments where the sirtuin activator is administered to a subject in a sustained release formulation, a high dose of the sirtuin activator may be administered for sustained delivery over a period of for example, at least about 12, 15, 18, 24, or 36 hours, or longer. In other embodiments, it is administered for a sustained delivery over a period of one or more days. In yet other embodiments, it is administered for a sustained delivery over a period of one or more weeks.

In certain embodiments, the sirtuin activating compound is administered in a nutraceutical formulation. A “nutraceutical” is any functional food (including beverages) that provides an additional benefit other than its nutritional benefit. In a preferred embodiment, a nutraceutical is provided and contains from about 0.1% to about 99%, or from about 0.1% to about 10% of a sirtuin activator by weight. In preferred embodiments, a high dose as described herein of a sirtuin activator is administered in a single serving of a food or beverage. In a preferred formulation, a single dosage form is provided (e.g., an 8 fluid ounce serving of a beverage such as water, flavored water, or fruit juice) that contains a quantity of total sirtuin activator that has a sirtuin activating effect equal to or greater than the sirtuin activating effect of 25 mg resveratol. In other embodiments, a single dosage form is provided that contains a quantity of total sirtuin activator that has a sirtuin activating effect equal to or greater than the sirtuin activating effect of about 10, 15, 20, 25, 50, 60, 75, 80, 100, 150, 200, or more, mg resveratrol per 8 fluid ounces. In other preferred embodiments, a single dosage form is provided (e.g., a serving of food such as a nutrition bar) that contains a total quantity of sirtuin activator that has a sirtuin activating effect equal to or greater than the sirtuin activating effect of 100 mg resveratol. In some embodiments, the food supplies 100 to 500 kcal per serving. In other embodiments, a single dosage form is provided that contains a total quantity of sirtuin activator that has a sirtuin activating effect equal to or greater than the sirtuin activating effect of 25, 50, 60, 75, 80, 100, 150, 200, 250, or more, mg resveratrol per 100 to 500 kcal. The phrase “total quantity of sirtuin activator” refers to the total amount of sirtuin activator(s) present in the single dosage form.

In various embodiments, a nutraceutical comprising a sirtuin activator may be any variety of food or drink. For example, nutraceuticals may include drinks such as nutritional drinks, diet drinks (e.g., Slimfast™, Boost™ and the like) as well as sports, herbal and other fortified beverages. Additionally, nutraceuticals may include foods intended for human or animal consumption such as baked goods, for example, bread, wafers, cookies, crackers, pretzels, pizza, and rolls, ready-to-eat breakfast cereals, hot cereals, pasta products, snacks such as fruit snacks, salty snacks, grain snacks, nutrition bars, and microwave popcorn, dairy products such as yogurt, cheese, and ice cream, sweet goods such as hard candy, soft candy, and chocolate, beverages, animal feed, pet foods such as dog food and cat food, aqua-culture foods such as fish food and shrimp feed, and special purpose foods such as baby food, infant formulas, hospital food, medical food, sports food, performance food or nutritional bars, or fortified foods, food preblends or mixes for home or food service use, such as preblends for soups or gravy, dessert mixes, dinner mixes, baking mixes such as bread mixes, and cake mixes, and baking flour. In certain embodiments, the food or beverage does not include one or more of grapes, mulberries, blueberries, raspberries, peanuts, milk, yeast, or extracts thereof. The present invention provides nutraceutical compositions that may be used to promote weight loss in a subject in need thereof. For example, in certain aspects, the present invention provides nutraceutical compositions that are useful for treating or preventing obesity and/or diabetes.

In addition to the sirtuin activator, the nutraceutical also may contain a variety of other beneficial components including but not limited to essential fatty acids, vitamins and minerals. Additional disclosure describing the contents and production of nutritional supplements may be found in e.g., U.S. Pat. No. 5,902,797; U.S. Pat. No. 5,834,048; U.S. Pat. No. 5,817,350; U.S. Pat. No. 5,792,461; U.S. Pat. No. 5,707,657 and U.S. Pat. No. 5,656,312 (each incorporated herein by reference).

When ingested in a solid form, a nutraceutical composition of the invention may additionally contain a solid carrier such as a gelatin or an adjuvant. When administered in liquid form, a liquid carrier such as water, petroleum, oils of animal or plant origin such as peanut oil, mineral oil, soybean oil, or sesame oil, or synthetic oils may be added. The nutraceutical composition of the present invention may also contain stabilizers, preservatives, buffers, antioxidants, or other additives known to those of skill in the art.

In other embodiments, a food or beverage comprises a supplement of one or more sirtuin activating compounds. In certain embodiments, the supplement comprises a quantity of a sirtuin activating compound that has a sirtuin activating effect equal to or greater than the sirtuin activating effect of 11 mg/g resveratol. In other embodiments, the supplement comprises a quantity of a sirtuin activating compound that has a sirtuin activating effect equal to or greater than the sirtuin activating effect of 5, 6, 7, 8, 9, 10, 11, 12, 13, 14, 15, 20, 25, 50, or more, mg/g resveratrol.

Other methods include administering to a subject a combination of a high dose of a sirtuin activator and an agent that increases the activity or protein level of an AMPK, e.g., other than an agent that activates a sirtuin. Activators of AMPK include AICAR or Metformin. Alternatively, the protein level of AMPK may be increased by introducing into the cell a nucleic acid encoding AMPK. The nucleotide sequence of the catalytic domain (α1) of human AMPK has the nucleotide sequence set forth in GenBank Accession No. NM_{—}206907 and encodes a protein having the amino acid sequence set forth in GenBank Accession No. NP_{—}996790. The nucleotide sequence of the non-catalytic domain (β1) of human AMPK has the nucleotide sequence set forth in GenBank Accession No. NM_{—}006253 and encodes a protein having the amino acid sequence set forth in GenBank Accession No. NP_{—}006244. The nucleotide sequence of the non-catalytic domain (γ1) of human AMPK has the nucleotide sequence set forth in GenBank Accession No. NM_{—}212461 and encodes a protein having the amino acid sequence sets forth in GenBank Accession No. NP_{—}997626. To increase the protein level of human AMPK in a cell, it may be necessary to introduce nucleic acids encoding each of the subunits of the protein. Nucleic acid sequences encoding the different subunits may be contained on the same or separate nucleic acid molecules.

Other diseases that may be treated by administering a high dose of a sirtuin activator include certain renal diseases including glomerulonephritis, glomerulosclerosis, nephrotic syndrome, hypertensive nephrosclerosis.

In other embodiments, a high dose of a sirtuin activator may be used to treat a disease or condition that will benefit from weight loss such as, for example: high blood pressure, hypertension, high blood cholesterol, dyslipidemia, type 2 diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence); uric acid nephrolithiasis; psychological disorders (such as depression, eating disorders, distorted body image, and low self esteem). Stunkard A J, Wadden T A. (Editors) Obesity: theory and therapy, Second Edition. New York: Raven Press, 1993. Finally, patients with AIDS can develop lipodystrophy or insulin resistance in response to combination therapies for AIDS. Accordingly, any of these conditions can be treated or prevented by the methods described herein for reducing or preventing weight gain.

Other diseases and conditions that can be treated by the methods described herein include chlomicronemia syndrome, polycistic ovarian syndrome, hypothermia, fat pad syndrome in the knee, alcoholic fatty liver, and non-alcoholic fatty liver.

In another embodiment, a high dose of a sirtuin-activating compound may be administered to reduce drug-induced weight gain. For example, a high dose of a sirtuin-activating compound may be administered as a combination therapy with medications that may stimulate appetite or cause weight gain, in particular, weight gain due to factors other than water retention. Examples of medications that may cause weight gain, include for example, diabetes treatments, including, for example, sulfonylureas (such as glipizide and glyburide), thiazolidinediones (such as pioglitazone and rosiglitazone), meglitinides, nateglinide, repaglinide, sulphonylurea medicines, and insulin; anti-depressants, including, for example, tricyclic antidepressants (such as amitriptyline and imipramine), irreversible monoamine oxidase inhibitors (MAOIs), selective serotonin reuptake inhibitors (SSRIs), bupropion, paroxetine, and mirtazapine; steroids, such as, for example, prednisone; hormone therapy; lithium carbonate; valproic acid; carbamazepine; chlorpromazine; thiothixene; beta blockers (such as propranolo); alpha blockers (such as clonidine, prazosin and terazosin); and contraceptives including oral contraceptives (birth control pills) or other contraceptives containing estrogen and/or progesterone (Depo-Provera, Norplant, Ortho), testosterone or Megestrol. In another exemplary embodiment, a high dose of a sirtuin-activating compound may be administered as part of a smoking cessation program to prevent weight gain or reduce weight already gained.

The methods described herein can also be used in veterinary applications, such as to treat metabolic disorders in pets (e.g., obesity or diabetes in dogs, cats, etc.) or farm animals (e.g., fat cow syndrome in cows).

In certain embodiments, a high dose of a sirtuin activator may be used to reduce weight, prevent weight gain, or reduce the rate of weight gain in a subject that consumes a high fat diet. For example, the invention provides methods and compositions to promote weight loss in a subject that consumes a high fat diet where lipids represent at least 30% of the average daily calorie consumption of the subject. In other embodiments, lipids represent at least 35%, at least 40%, at least 45%, at least 50%, at least 55%, at least 60% of the average daily calorie comsumption of the subject. In certain embodiments, a high fat diet includes at least about 10%, 20%, 30%, 40%, 50%, 60%, or more, of the daily calorie consumption from carbohydrates.

The methods and compositions of the present invention may also be used to reduce weight gain in a subject that is refractory to diet and exercise. In exemplary embodiments, a high dose of a sirtuin activating compound may promote weight loss in a subject that does not reduce calorie consumption, increase activity, or a combination thereof, to an extent sufficient to cause weight loss in the absence of a sirtuin activating compound.

In certain embodiments, a high dose of a sirtuin-activating compound may be directed specifically to a certain tissue (e.g., liver) rather than the whole body. Tissue specific treatments may be used to treat, e.g., obesity and insulin resistance disorder.

In certain embodiments the methods are useful for preventing fat accumulation in cells with lipogenic capacity, e.g. liver, pancreas and muscle cells.

In certain embodiments, the invention provides methods for increasing the life span or preventing cell death of pancreatic β-cells. The methods involve contacting pancreatic β-cells with a sirtuin activating compound. In other embodiments, the methods involve administering to a subject in need thereof (e.g., a subject having type 1 diabetes, type 2 diabetes, impaired glucose tolerance, etc.) a therapeutically effective amount of a sirtuin activating compound. Susceptibility to type 2 diabetes requires both genetic and acquired factors. Its continuing pathogenesis involves an interplay of progressive cellular insulin resistance and pancreatic β-cell failure. Free radical generation and induced nitric oxide synthase (iNOS) production secondary to the hyperglycemia of type 2 diabetes can lead to pancreatic β-cell destruction, and the production of diagnostic enzymatic indicators characteristic of type 1 diabetes. In this scenario, β-cells are not only “exhausted” by the progression of pathology from insulin resistance to type 2 diabetes but may also undergo destruction induced by chronic hyperglycemia. Pancreatic β-cell apoptosis is responsible for irreversible progression toward insulin dependence in type 2 diabetes. The compounds described herein can be used to inhibit or prevent progression to type 2 diabetes in a subject in need thereof. For example, in certain embodiments, the compounds of the subject invention inhibit or prevent pancreatic β-cell death. As described below, prevention of pancreatic β-cell death or dysfunction may be through increased mitochondrial activity or number.

In certain embodiments, the invention provides methods for treating a metabolic disorder comprising administration of a sirtuin activating compound in combination with a sirtuin inhibitor. In an exemplary embodiment, the method involves administering a sirtuin activating compound to the fat cells of a patient in combination with administering a sirtuin inhibitor to the liver of a subject in need thereof.

Mitochoyidrial-Associated Diseases and Disorders

In certain embodiments, the invention provides methods for treating diseases or disorders that would benefit from increased mitochondrial activity. The methods involve administering to a subject in need thereof a therapeutically effective amount of a sirtuin activating compound. Increased mitochondrial activity refers to increasing activity of the mitochondria while maintaining the overall numbers of mitochondria (e.g., mitochondrial mass), increasing the numbers of mitochondria thereby increasing mitochondrial activity (e.g., by stimulating mitochondrial biogenesis), or combinations thereof. In an exemplary embodiment, the methods involve administering a high dose of a sirtuin activating compound. In certain embodiments, diseases and disorders that would benefit from increased mitochondrial activity include diseases or disorders associated with mitochondrial dysfunction.

In certain embodiments, methods for treating diseases or disorders that would benefit from increased mitochondrial activity may comprise identifying a subject suffering from a mitochondrial dysfunction. Methods for diagnosing a mitochondrial dysfunction may involve molecular genetic, pathologic and/or biochemical analysis are summarized in Cohen and Gold, Cleveland Clinic Journal of Medicine, 68: 625-642 (2001). One method for diagnosing a mitochondrial dysfunction is the Thor-Byrne-ier scale (see e.g., Cohen and Gold, supra; Collin S. et al., Eur Neurol. 36: 260-267 (1996)).

Mitochondria are critical for the survival and proper function of almost all types of eukaryotic cells. Mitochondria in virtually any cell type can have congenital or acquired defects that affect their function. Thus, the clinically significant signs and symptoms of mitochondrial defects affecting respiratory chain function are heterogeneous and variable depending on the distribution of defective mitochondria among cells and the severity of their deficits, and upon physiological demands upon the affected cells. Nondividing tissues with high energy requirements, e.g. nervous tissue, skeletal muscle and cardiac muscle are particularly susceptible to mitochondrial respiratory chain dysfunction, but any organ system can be affected.

Diseases and disorders associated with mitochondrial dysfunction include diseases and disorders in which deficits in mitochondrial respiratory chain activity contribute to the development of pathophysiology of such diseases or disorders in a mammal. This includes 1) congenital genetic deficiencies in activity of one or more components of the mitochondrial respiratory chain; and 2) acquired deficiencies in the activity of one or more components of the mitochondrial respiratory chain, wherein such deficiencies are caused by a) oxidative damage during aging; b) elevated intracellular calcium; c) exposure of affected cells to nitric oxide; d) hypoxia or ischemia; e) microtubule-associated deficits in axonal transport of mitochondria, or f) expression of mitochondrial uncoupling proteins.

Diseases or disorders that would benefit from increased mitochondrial activity generally include for example, diseases in which free radical mediated oxidative injury leads to tissue degeneration, diseases in which cells inappropriately undergo apoptosis, and diseases in which cells fail to undergo apoptosis. Exemplary diseases or disorders that would benefit from increased mitochondrial activity include, for example, AD (Alzheimer's Disease), ADPD (Alzheimer's Disease and Parkinsons's Disease), AMDF (Ataxia, Myoclonus and Deafness), auto-immune disease, cancer, CIPO (Chronic Intestinal Pseudoobstruction with myopathy and Ophthalmoplegia), congenital muscular dystrophy, CPEO (Chronic Progressive External Ophthalmoplegia), DEAF (Maternally inherited DEAFness or aminoglycoside-induced DEAFness), DEMCHO (Dementia and Chorea), diabetes mellitus (Type I or Type II), DIDMOAD (Diabetes Insipidus, Diabetes Mellitus, Optic Atrophy, Deafness), DMDF (Diabetes Mellitus and Deafness), dystonia, Exercise Intolerance, ESOC (Epilepsy, Strokes, Optic atrophy, and Cognitive decline), FBSN (Familial Bilateral Striatal Necrosis), FICP (Fatal Infantile Cardiomyopathy Plus, a MELAS-associated cardiomyopathy), GER (Gastrointestinal Reflux), HD (Huntington's Disease), KSS (Kearns Sayre Syndrome), “later-onset” myopathy, LDYT (Leber's hereditary optic neuropathy and DYsTonia), Leigh's Syndrome, LHON (Leber Hereditary Optic Neuropathy), LIMM (Lethal Infantile Mitochondrial Myopathy), MDM (Myopathy and Diabetes Mellitus), MELAS (Mitochondrial Encephalomyopathy, Lactic Acidosis, and Stroke-like episodes), MEPR (Myoclonic Epilepsy and Psychomotor Regression), MERME (MERRF/MELAS overlap disease), MERRF (Myoclonic Epilepsy and Ragged Red Muscle Fibers), MHCM (Maternally Inherited Hypertrophic CardioMyopathy), MICM (Maternally Inherited Cardiomyopathy), MILS (Maternally Inherited Leigh Syndrome), Mitochondrial Encephalocardiomyopathy, Mitochondrial Encephalomyopathy, MM (Mitochondrial Myopathy), MMC (Matemal Myopathy and Cardiomyopathy), MNGIE (Myopathy and external ophthalmoplegia, Neuropathy, Gastro-Intestinal, Encephalopathy), Multisystem Mitochondrial Disorder (myopathy, encephalopathy, blindness, hearing loss, peripheral neuropathy), NARP (Neurogenic muscle weakness, Ataxia, and Retinitis Pigmentosa; alternate phenotype at this locus is reported as Leigh Disease), PD (Parkinson's Disease), Pearson's Syndrome, PEM (Progressive Encephalopathy), PEO (Progressive External Ophthalmoplegia), PME (Progressive Myoclonus Epilepsy), PMPS (Pearson Marrow-Pancreas Syndrome), psoriasis, RTT (Rett Syndrome), schizophrenia, SIDS (Sudden Infant Death Syndrome), SNHL (Sensorineural Hearing Loss), Varied Familial Presentation (clinical manifestations range from spastic paraparesis to multisystem progressive disorder & fatal cardiomyopathy to truncal ataxia, dysarthria, severe hearing loss, mental regression, ptosis, ophthalmoparesis, distal cyclones, and diabetes mellitus), or Wolfram syndrome.

Other diseases and disorders that would benefit from increased mitochondrial activity include, for example, Friedreich's ataxia and other ataxias, amyotrophic lateral sclerosis (ALS) and other motor neuron diseases, macular degeneration, epilepsy, Alpers syndrome, Multiple mitochondrial DNA deletion syndrome, MtDNA depletion syndrome, Complex I deficiency, Complex II (SDH) deficiency, Complex III deficiency, Cytochrome c oxidase (COX, Complex IV) deficiency, Complex V deficiency, Adenine Nucleotide Translocator (ANT) deficiency, Pyruvate dehydrogenase (PDH) deficiency, Ethylmalonic aciduria with lactic acidemia, 3-Methyl glutaconic aciduria with lactic acidemia, Refractory epilepsy with declines during infection, Asperger syndrome with declines during infection, Autism with declines during infection, Attention deficit hyperactivity disorder (ADHD), Cerebral palsy with declines during infection, Dyslexia with declines during infection, materially inherited thrombocytopenia and leukemia syndrome, MARIAHS syndrome (Mitrochondrial ataxia, recurrent infections, aphasia, hypouricemia/hypomyelination, seizures, and dicarboxylic aciduria), ND6 dystonia, Cyclic vomiting syndrome with declines during infection, 3-Hydroxy isobutryic aciduria with lactic acidemia, Diabetes mellitus with lactic acidemia, Uridine responsive neurologic syndrome (URNS), Dilated cardiomyopathy, Splenic Lymphoma, and Renal Tubular Acidosis/Diabetes/Ataxis syndrome.

In other embodiments, the invention provides methods for treating a subject suffering from mitochondrial disorders arising from, but not limited to, Post-traumatic head injury and cerebral edema, Stroke (invention methods useful for preventing or preventing reperfusion injury), Lewy body dementia, Hepatorenal syndrome, Acute liver failure, NASH (non-alcoholic steatohepatitis), Anti-metastasis/prodifferentiation therapy of cancer, Idiopathic congestive heart failure, Atrial fibrilation (non-valvular), Wolff-Parkinson-White Syndrome, Idiopathic heart block, Prevention of reperfusion injury in acute myocardial infarctions, Familial migraines, Irritable bowel syndrome, Secondary prevention of non-Q wave myocardial infarctions, Premenstrual syndrome, Prevention of renal failure in hepatorenal syndrome, Anti-phospholipid antibody syndrome, Eclampsia/pre-eclampsia, Oopause infertility, Ischemic heart disease/Angina, and Shy-Drager and unclassified dysautonomia syndromes.

In still another embodiment, there are provided methods for the treatment of mitochondrial disorders associated with pharmacological drug-related side effects. Types of pharmaceutical agents that are associated with mitochondrial disorders include reverse transcriptase inhibitors, protease inhibitors, inhibitors of DHOD, and the like. Examples of reverse transcriptase inhibitors include, for example, Azidothymidine (AZT), Stavudine (D4T), Zalcitabine (ddC), Didanosine (DDI), Fluoroiodoarauracil (FIAU), Lamivudine (3TC), Abacavir and the like. Examples of protease inhibitors include, for example, Ritonavir, Indinavir, Saquinavir, Nelfinavir and the like. Examples of inhibitors of dihydroorotate dehydrogenase (DHOD) include, for example, Leflunomide, Brequinar, and the like.

Reverse transcriptase inhibitors not only inhibit reverse transcriptase but also polymerase gamma which is required for mitochondrial function. Inhibition of polymerase gamma activity (e.g., with a reverse transcriptase inhibitor) therefore leads to mitochondrial dysfunction and/or a reduced mitochondrial mass which manifests itself in patients as hyperlactatemia. This type of condition may benefit from an increase in the number of mitochondria and/or an improvement in mitochondrial function, e.g., by administration of a sirtuin activating compound.

Common symptoms of mitochondrial diseases include cardiomyopathy, muscle weakness and atrophy, developmental delays (involving motor, language, cognitive or executive function), ataxia, epilepsy, renal tubular acidosis, peripheral neuropathy, optic neuropathy, autonomic neuropathy, neurogenic bowel dysfunction, sensorineural deafness, neurogenic bladder dysfunction, dilating cardiomyopathy, migraine, hepatic failure, lactic acidemia, and diabetes mellitus.

In certain embodiments, the invention provides methods for treating a disease or disorder that would benefit from increased mitochondrial activity that involves administering to a subject in need thereof one or more sirtuin activating compounds in combination with another therapeutic agent such as, for example, an agent useful for treating mitochondrial dysfunction (such as antioxidants, vitamins, or respiratory chain cofactors), an agent useful for reducing a symptom associated with a disease or disorder involving mitochondrial dysfunction (such as, an anti-seizure agent, an agent useful for alleviating neuropathic pain, an agent for treating cardiac dysfunction), a cardiovascular agent (as described further below), a chemotherapeutic agent (as described further below), or an anti-neurodegeneration agent (as described further below). In an exemplary embodiment, the invention provides methods for treating a disease or disorder that would benefit from increased mitochondrial activity that involves administering to a subject in need thereof one or more sirtuin activating compounds in combination with one or more of the following: coenzyme Q₁₀, L-carnitine, thiamine, riboflavin, niacinamide, folate, vitamin E, selenium, lipoic acid, or prednisone. Compositions comprising such combinations are also provided herein.

In exemplary embodiments, the invention provides methods for treating diseases or disorders that would benefit from increased mitochondrial acitivty by administering to a subject a therapeutically effective amount of a sirtuin activating compound. Exemplary diseases or disorders include, for example, neuromuscular disorders (e.g., Friedreich's Ataxia, muscular dystrophy, multiple sclerosis, etc.), disorders of neuronal instability (e.g., seizure disorders, migrane, etc.), developmental delay, neurodegenerative disorders (e.g., Alzheimer's Disease, Parkinson's Disease, amyotrophic lateral sclerosis, etc.), ischemia, renal tubular acidosis, age-related neurodegeneration and cognitive decline, chemotherapy fatigue, age-related or chemotherapy-induced menopause or irregularities of menstrual cycling or ovulation, mitochondrial myopathies, mitochondrial damage (e.g., calcium accumulation, excitotoxicity, nitric oxide exposure, hypoxia, etc.), and mitochondrial deregulation.

A gene defect underlying Friedreich's Ataxia (FA), the most common hereditary ataxia, was recently identified and is designated “frataxin”. In FA, after a period of normal—development, deficits in coordination develop which progress to paralysis and death, typically between the ages of 30 and 40. The tissues affected most severely are the spinal cord, peripheral nerves, myocardium, and pancreas. Patients typically lose motor control and are confined to wheel chairs, and are commonly afflicted with heart failure and diabetes. The genetic basis for FA involves GAA trinucleotide repeats in an intron region of the gene encoding frataxin. The presence of these repeats results in reduced transcription and expression of the gene. Frataxin is involved in regulation of mitochondrial iron content. When cellular frataxin content is subnormal, excess iron accumulates in mitochondria, promoting oxidative damage and consequent mitochondrial degeneration and dysfunction. When intermediate numbers of GAA repeats are present in the frataxin gene intron, the severe clinical phenotype of ataxia may not develop. However, these intermediate-length trinucleotide extensions are found in 25 to 30% of patients with non-insulin dependent diabetes mellitus, compared to about 5% of the nondiabetic population. In certain embodiments, sirtuin activating compounds may be used for treating patients with disorders related to deficiencies or defects in frataxin, including Friedreich's Ataxia, myocardial dysfunction, diabetes mellitus and complications of diabetes like peripheral neuropathy.

Muscular dystrophy refers to a family of diseases involving deterioration of neuromuscular structure and function, often resulting in atrophy of skeletal muscle and myocardial dysfunction. In the case of Duchenne muscular dystrophy, mutations or deficits in a specific protein, dystrophin, are implicated in its etiology. Mice with their dystrophin genes inactivated display some characteristics of muscular dystrophy, and have an approximately 50% deficit in mitochondrial respiratory chain activity. A final common pathway for neuromuscular degeneration in most cases is calcium-mediated impairment of mitochondrial function. In certain embodiments, sirtuin activating compounds may be used for reducing the rate of decline in muscular functional capacities and for improving muscular functional status in patients with muscular dystrophy.

Multiple sclerosis (MS) is a neuromuscular disease characterized by focal inflammatory and autoimmune degeneration of cerebral white matter. Periodic exacerbations or attacks are significantly correlated with upper respiratory tract and other infections, both bacterial and viral, indicating that mitochondrial dysfunction plays a role in MS. Depression of neuronal mitochondrial respiratory chain activity caused by Nitric Oxide (produced by astrocytes and other cells involved in inflammation) is implicated as a molecular mechanism contributing to MS. In certain embodiments, sirtuin activating compounds may be used for treatment of patients with multiple sclerosis, both prophylactically and during episodes of disease exacerbation.

Epilepsy is often present in patients with mitochondrial cytopathies, involving a range of seizure severity and frequency, e.g. absence, tonic, atonic, myoclonic, and status epilepticus, occurring in isolated episodes or many times daily. In certain embodiments, sirtuin activating compounds may be used for treating patients with seizures secondary to mitochondrial dysfunction, including reducing frequency and severity of seizure activity.

Metabolic studies on patients with recurrent migraine headaches indicate that deficits in mitochondrial activity are commonly associated with this disorder, manifesting as impaired-oxidative phosphorylation and excess lactate production. Such deficits are not necessarily due to genetic defects in mitochondrial DNA. Migraineurs are hypersensitive to nitric oxide, an endogenous inhibitor of Cytochrome c Oxidase. In addition, patients with mitochondrial cytopathies, e.g. MELAS, often have recurrent migraines. In certain embodiments, sirtuin activating compounds may be used for treating patients with recurrent migraine headaches, including headaches refractory to ergot compounds or serotonin receptor antagonists.

Delays in neurological or neuropsychological development are often found in children with mitochondrial diseases. Development and remodeling of neural connections requires intensive biosynthetic activity, particularly involving synthesis of neuronal membranes and myelin, both of which require pyrimidine nucleotides as cofactors. Uridine nucleotides are involved inactivation and transfer of sugars to glycolipids and glycoproteins. Cytidine nucleotides are derived from uridine nucleotides, and are crucial for synthesis of major membrane phospholipid constituents like phosphatidylcholine, which receives its choline moiety from cytidine diphosphocholine. In the case of mitochondrial dysfunction (due to either mitochondrial DNA defects or any of the acquired or conditional deficits like exicitoxic or nitric oxide-mediated mitochondrial dysfunction) or other conditions resulting in impaired pyrimidine synthesis, cell proliferation and axonal extension is impaired at crucial stages in development of neuronal interconnections and circuits, resulting in delayed or arrested development of neuropsychological functions like language, motor, social, executive function, and cognitive skills. In autism for example, magnetic resonance spectroscopy measurements of cerebral phosphate compounds indicates that there is global undersynthesis of membranes and membrane precursors indicated by reduced levels of uridine diphospho-sugars, and cytidine nucleotide derivatives involved in membrane synthesis. Disorders characterized by developmental delay include Rett's Syndrome, pervasive developmental delay (or PDD-NOS “pervasive developmental delay not otherwise specified” to distinguish it from specific subcategories like autism), autism, Asperger's Syndrome, and Attention Deficit/Hyperactivity Disorder (ADHD), which is becoming recognized as a delay or lag in development of neural circuitry underlying executive functions. In certain embodiments, sirtuin activating compounds may be useful for treating treating patients with neurodevelopmental delays (e.g., involving motor, language, executive function, and cognitive skills), or other delays or arrests of neurological and neuropsychological development in the nervous system and somatic development in non-neural tissues like muscle and endocrine glands.

The two most significant severe neurodegenerative diseases associated with aging, Alzheimer's Disease (AD) and Parkinson's Disease (PD), both involve mitochondrial dysfunction in their pathogenesis. Complex I deficiencies in particular are frequently found not only in the nigrostriatal neurons that degenerate in Parkinson's disease, but also in peripheral tissues and cells like muscle and platelets of Parkinson's Disease patients. In Alzheimer's Disease, mitochondrial respiratory chain activity is often depressed, especially Complex IV (Cytochrome c Oxidase). Moreover, mitochondrial respiratory function altogether is depressed as a consequence of aging, further amplifying the deleterious sequelae of additional molecular lesions affecting respiratory chain function. Other factors in addition to primary mitochondrial dysfunction underlie neurodegeneration in AD, PD, and related disorders. Excitotoxic stimulation and nitric oxide are implicated in both diseases, factors which both exacerbate mitochondrial respiratory chain deficits and whose deleterious actions are exaggerated on a background of respiratory chain dysfunction. Huntington's Disease also involves mitochondrial dysfunction in affected brain regions, with cooperative interactions of excitotoxic stimulation and mitochondrial dysfunction contributing to neuronal degeneration. In certain embodiments, sirtuin activating compounds may be useful for treating and attenuating progression of age-related neurodegenerative disease including AD and PD.

One of the major genetic defects in patients with Amyotrophic Lateral Sclerosis (ALS or Lou Gehrig's Disease) is mutation or deficiency in Copper-Zinc Superoxide Dismutase (SOD 1), an antioxidant enzyme. Mitochondria both produce and are primary targets for reactive oxygen species. Inefficient transfer of electrons to oxygen in mitochondria is the most significant physiological source of free radicals in mammalian systems. Deficiencies in antioxidants or antioxidant enzymes can result in or exacerbate mitochondrial degeneration. Mice transgenic for mutated SOD1 develop symptoms and pathology similar to those in human ALS. The development of the disease in these animals has been shown to involve oxidative destruction of mitochondria followed by functional decline of motor neurons and onset of clinical symptoms. Skeletal muscle from ALS patients has low mitochondrial Complex I activity. In certain embodiments, sirtuin activating compounds may be useful for treating ALS, for reversing or slowing the progression of clinical symptoms.

Oxygen deficiency results in both direct inhibition of mitochondrial respiratory chain activity by depriving cells of a terminal electron acceptor for Cytochrome c reoxidation at Complex IV, and indirectly, especially in the nervous system, via secondary post-anoxic excitotoxicity and nitric oxide formation. In conditions like cerebral anoxia, angina or sickle cell anemia crises, tissues are relatively hypoxic. In such cases, compounds that increase mitochondrial activity provide protection of affected tissues from deleterious effects of hypoxia, attenuate secondary delayed cell death, and accelerate recovery from hypoxic tissue stress and injury. In certain embodiments, sirtuin activating compounds may be useful for preventing delayed cell death (apoptosis in regions like the hippocampus or cortex occurring about 2 to 5 days after an episode of cerebral ischemia) after ischemic or hypoxic insult to the brain.

Acidosis due to renal dysfunction is often observed in patients with mitochondrial disease, whether the underlying respiratory chain dysfunction is congenital or induced by ischemia or cytotoxic agents like cisplatin. Renal tubular acidosis often requires administration of exogenous sodium bicarbonate to maintain blood and tissue pH. In certain embodiments, sirtuin activating compounds may be useful for treating renal tubular acidosis and other forms of renal dysfunction caused by mitochondrial respiratory chain deficits.

During normal aging, there is a progressive decline in mitochondrial respiratory chain function. Beginning about age 40, there is an exponential rise in accumulation of mitochondrial DNA defects in humans, and a concurrent decline in nuclear-regulated elements of mitochondrial respiratory activity. Many mitochondrial DNA lesions have a selection advantage during mitochondrial turnover, especially in postmitotic cells. The proposed mechanism is that mitochondria with a defective respiratory chain produce less oxidative damage to themselves than do mitochondria with intact functional respiratory chains (mitochondrial respiration is the primary source of free radicals in the body). Therefore, normally-functioning mitochondria accumulate oxidative damage to membrane lipids more rapidly than do defective mitochondria, and are therefore “tagged” for degradation by lysosomes. Since mitochondria within cells have a half life of about 10 days, a selection advantage can result in rapid replacement of functional mitochondria with those with diminished respiratory activity, especially in slowly dividing cells. The net result is that once a mutation in a gene for a mitochondrial protein that reduces oxidative damage to mitochondria occurs, such defective mitochondria will rapidly populate the cell, diminishing or eliminating its respiratory capabilities. The accumulation of such cells results in aging or degenerative disease at the organismal level. This is consistent with the progressive mosaic appearance of cells with defective electron transport activity in muscle, with cells almost devoid of Cytochrome c Oxidase (COX) activity interspersed randomly amidst cells with normal activity, and a higher incidence of COX-negative cells in biopsies from older subjects. The organism, during aging, or in a variety of mitochondrial diseases, is thus faced with a situation in which irreplaceable postmitotic cells (e.g. neurons, skeletal and cardiac muscle) must be preserved and their function maintained to a significant degree, in the face of an inexorable progressive decline in mitochondrial respiratory chain function. Neurons with dysfunctional mitochondria become progressively more sensitive to insults like excitotoxic injury. Mitochondrial failure contributes to most degenerative diseases (especially neurodegeneration) that accompany aging. Congenital mitochondrial diseases often involve early-onset neurodegeneration similar in fundamental mechanism to disorders that occur during aging of people born with normal mitochondria. In certain embodiments, sirtuin activating compounds may be useful for treating or attenuating cognitive decline and other degenerative consequences of aging.

Mitochondrial DNA damage is more extensive and persists longer than nuclear DNA damage in cells subjected to oxidative stress or cancer chemotherapy agents like cisplatin due to both greater vulnerability and less efficient repair of mitochondrial DNA. Although mitochondrial DNA may be more sensitive to damage than nuclear DNA, it is relatively resistant, in some situations, to mutagenesis by chemical carcinogens. This is because mitochondria respond to some types of mitochondrial DNA damage by destroying their defective genomes rather than attempting to repair them. This results in global mitochondrial dysfunction for a period after cytotoxic chemotherapy. Clinical use of chemotherapy agents like cisplatin, mitomycin, and cytoxan is often accompanied by debilitating “chemotherapy fatigue”, prolonged periods of weakness and exercise intolerance which may persist even after recovery from hematologic and gastrointestinal toxicities of such agents. In certain embodiments, sirtuin activating compounds may be useful for treatment and prevention of side effects of cancer chemotherapy related to mitochondrial dysfunction.

A crucial function of the ovary is to maintain integrity of the mitochondrial genome in oocytes, since mitochondria passed onto a fetus are all derived from those present in oocytes at the time of conception. Deletions in mitochondrial DNA become detectable around the age of menopause, and are also associated with abnormal menstrual cycles. Since cells cannot directly detect and respond to defects in mitochondrial DNA, but can only detect secondary effects that affect the cytoplasm, like impaired respiration, redox status, or deficits in pyrimidine synthesis, such products of mitochondrial function participate as a signal for oocyte selection and follicular atresia, ultimately triggering menopause when maintenance of mitochondrial genomic fidelity and functional activity can no longer be guaranteed. This is analogous to apoptosis in cells with DNA damage, which undergo an active process of cellular suicide when genomic fidelity can no longer be achieved by repair processes. Women with mitochondrial cytopathies affecting the gonads often undergo premature menopause or display primary cycling abnormalities. Cytotoxic cancer chemotherapy often induces premature menopause, with a consequent increased risk of osteoporosis. Chemotherapy-induced amenorrhea is generally due to primary ovarian failure. The incidence of chemotherapy-induced amenorrhea increases as a function of age in premenopausal women receiving chemotherapy, pointing toward mitochondrial involvement. Inhibitors of mitochondrial respiration or protein synthesis inhibit hormone-induced ovulation, and furthermore inhibit production of ovarian steroid hormones in response to pituitary gonadotropins. Women with Downs syndrome typically undergo menopause prematurely, and also are subject to early onset of Alzheimer-like dementia. Low activity of cytochrome oxidase is consistently found in tissues of Downs patients and in late-onset Alzheimer's Disease. Appropriate support of mitochondrial function or compensation for mitochondrial dysfunction therefore is useful for protecting against age-related or chemotherapy-induced menopause or irregularities of menstrual cycling or ovulation. In certain embodiments, sirtuin activating compounds may be useful for treating and preventing amenorrhea, irregular ovulation, menopause, or secondary consequences of menopause.

In certain embodiments, sirtuin modulating compounds may be useful for treatment mitochondrial myopathies. Mitochondrial myopathies range from mild, slowly progressive weakness of the extraocular muscles to severe, fatal infantile myopathies and multisystem encephalomyopathies. Some syndromes have been defined, with some overlap between them. Established syndromes affecting muscle include progressive external ophthalmoplegia, the Kearns-Sayre syndrome (with ophthalmoplegia, pigmentary retinopathy, cardiac conduction defects, cerebellar ataxia, and sensorineural deafness), the MELAS syndrome (mitochondrial encephalomyopathy, lactic acidosis, and stroke-like episodes), the MERFF syndrome (myoclonic epilepsy and ragged red fibers), limb-girdle distribution weakness, and infantile myopathy (benign or severe and fatal). Muscle biopsy specimens stained with modified Gomori's trichrome stain show ragged red fibers due to excessive accumulation of mitochondria. Biochemical defects in substrate transport and utilization, the Krebs cycle, oxidative phosphorylation, or the respiratory chain are detectable. Numerous mitochondrial DNA point mutations and deletions have been described, transmitted in a maternal, nonmendelian inheritance pattern. Mutations in nuclear-encoded mitochondrial enzymes occur.

In certain embodiments, sirtuin activating compounds may be useful for treating patients suffering from toxic damage to mitochondria, such as, toxic damage due to calcium accumulation, excitotoxicity, nitric oxide exposure, drug induced toxic damage, or hypoxia.

A fundamental mechanism of cell injury, especially in excitable tissues, involves excessive calcium entry into cells, as a result of either leakage through the plasma membrane or defects in intracellular calcium handling mechanisms. Mitochondria are major sites of calcium sequestration, and preferentially utilize energy from the respiratory chain for taking up calcium rather than for ATP synthesis, which results in a downward spiral of mitochondrial failure, since calcium uptake into mitochondria results in diminished capabilities for energy transduction.

Excessive stimulation of neurons with excitatory amino acids is a common mechanism of cell death or injury in the central nervous system. Activation of glutamate receptors, especially of the subtype designated NMDA receptors, results in mitochondrial dysfunction, in part through elevation of intracellular calcium during excitotoxic stimulation. Conversely, deficits in mitochondrial respiration and oxidative phosphorylation sensitizes cells to excitotoxic stimuli, resulting in cell death or injury during exposure to levels of excitotoxic neurotransmitters or toxins that would be innocuous to normal cells.

Nitric oxide (about 1 micromolar) inhibits cytochrome oxidase (Complex IV) and thereby inhibits mitochondrial respiration; moreover, prolonged exposure to nitric oxide (NO) irreversibly reduces Complex I activity. Physiological or pathophysiological concentrations of NO thereby inhibit pyrimidine biosynthesis. Nitric oxide is implicated in a variety of neurodegenerative disorders including inflammatory and autoimmune diseases of the central nervous system, and is involved in mediation of excitotoxic and post-hypoxic damage to neurons.

Oxygen is the terminal electron acceptor in the respiratory chain. Oxygen deficiency impairs electron transport chain activity, resulting in diminished pyrimidine synthesis as well as diminished ATP synthesis via oxidative phosphorylation. Human cells proliferate and retain viability under virtually anaerobic conditions if provided with uridine and pyruvate (or a similarly effective agent for oxidizing NADH to optimize glycolytic ATP production).

In certain embodiments, sirtuin activating compounds may be useful for treating diseases or disorders associated with mitochondrial deregulation.

Transcription of mitochondrial DNA encoding respiratory chain components requires nuclear factors. In neuronal axons, mitochondria must shuttle back and forth to the nucleus in order to maintain respiratory chain activity. If axonal transport is impaired by hypoxia or by drugs like taxol which affect microtubule stability, mitochondria distant from the nucleus undergo loss of cytochrome oxidase activity. Accordingly, treatment with a sirtuin activating compound may be useful for promoting nuclear-mitochondrial interactions.

Mitochondria are the primary source of free radicals and reactive oxygen species, due to spillover from the mitochondrial respiratory chain, especially when defects in one or more respiratory chain components impairs orderly transfer of electrons from metabolic intermediates to molecular oxygen. To reduce oxidative damage, cells can compensate by expressing mitochondrial uncoupling proteins (UCP), of which several have been identified. UCP-2 is transcribed in response to oxidative damage, inflammatory cytokines, or excess lipid loads, e.g. fatty liver and steatohepatitis. UCPs reduce spillover of reactive oxygen species from mitochondria by discharging proton gradients across the mitochondrial inner membrane, in effect wasting energy produced by metabolism and rendering cells vulnerable to energy stress as a trade-off for reduced oxidative injury.

Muscle Performance

In other embodiments, the invention provides methods for enhancing muscle performance by administering a therapeutically effective amount of a sirtuin activating compound. For example, sirtuin activating compounds may be useful for improving physical endurance (e.g., ability to perform a physical task such as exercise, physical labor, sports activities, etc.), inhibiting or retarding physical fatigues, enhancing blood oxygen levels, enhancing energy in healthy individuals, enhance working capacity and endurance, reducing muscle fatigue, reducing stress, enhancing cardiac and cardiovascular function, improving sexual ability, increasing muscle ATP levels, and/or reducing lactic acid in blood. In certain embodiments, the methods involve administering an amount of a sirtuin activating compound that increase mitochondrial activity, increase mitochondrial biogenesis, increase mitochondrial mass, or a high dose of a sirtuin activating compound.

Sports performance refers to the ability of the athlete's muscles to perform when participating in sports activities. Enhanced sports performance, strength, speed and endurance are measured by an increase in muscular contraction strength, increase in amplitude of muscle contraction, shortening of muscle reaction time between stimulation and contraction. Athlete refers to an individual who participates in sports at any level and who seeks to achieve an improved level of strength, speed and endurance in their performance, such as, for example, body builders, bicyclists, long distance runners, short distance runners, etc. An athlete may be hard training, that is, performs sports activities intensely more than three days a week or for competition. An athlete may also be a fitness enthusiast who seeks to improve general health and well-being, improve energy levels, who works out for about 1-2 hours about 3 times a week. Enhanced sports performance in manifested by the ability to overcome muscle fatigue, ability to maintain activity for longer periods of time, and have a more effective workout.

In the arena of athlete muscle performance, it is desirable to create conditions that permit competition or training at higher levels of resistance for a prolonged period of time. However, acute and intense anaerobic use of skeletal muscles often results in impaired athletic performance, with losses in force and work output, and increased onset of muscle fatigue, soreness, and dysfunction. It is now recognized that even a single exhaustive exercise session, or for that matter any acute trauma to the body such as muscle injury, resistance or exhaustive muscle exercise, or elective surgery, is characterized by perturbed metabolism that affects muscle performance in both short and long term phases. Both muscle metabolic/enzymatic activity and gene expression are affected. For example, disruption of skeletal muscle nitrogen metabolism as well as depletion of sources of metabolic energy occur during extensive muscle activity. Amino acids, including branched-chain amino acids, are released from muscles followed by their deamination to elevate serum ammonia and local oxidation as muscle fuel sources, which augments metabolic acidosis. In addition, there is a decline in catalytic efficiency of muscle contraction events, as well as an alteration of enzymatic activities of nitrogen and energy metabolism. Further, protein catabolism is initiated where rate of protein synthesis is decreased coupled with an increase in the degradation of non-contractible protein. These metabolic processes are also accompanied by free radical generation which further damages muscle cells.

Recovery from fatigue during acute and extended exercise requires reversal of metabolic and non-metabolic fatiguing factors. Known factors that participate in human muscle fatigue, such as lactate, ammonia, hydrogen ion, etc., provide an incomplete and unsatisfactory explanation of the fatigue/recovery process, and it is likely that additional unknown agents participate (Baker et al., J. Appl. Physiol. 74:2294-2300, 1993; Bazzarre et al., J Am. Coll. Nutr. 11:505-511, 1992; Dohm et al., Fed. Proc. 44:348-352, 1985; Edwards In: Biochemistry of Exercise, Proceedings of the Fifth International Symposium on the Biochemistry of Exercise (Kutrgen, Vogel, Poormans, eds.), 1983; MacDougall et al., Acta Physiol. Scand. 146:403-404, 1992; Walser et al., Kidney Int. 32:123-128, 1987). Several studies have also analyzed the effects of nutritional supplements and herbal supplements in enhancing muscle performance.

Aside from muscle performance during endurance exercise, free radicals and oxidative stress parameters are affected in pathophysiological states. A substantial body of data now suggests that oxidative stress contributes to muscle wasting or atrophy in pathophysiological states (reviewed in Clarkson, P. M. Antioxidants and physical performance. Crit. Rev. Food Sci. Nutr. 35: 31-41; 1995; Powers, S. K.; Lennon, S. L. Analysis of cellular responses to free radicals: Focus on exercise and skeletal muscle. Proc. Nutr. Soc. 58: 1025-1033; 1999). For example, with respect to muscular disorders where both muscle endurance and function are compensated, the role of nitric oxide (NO), has been implicated. In muscular dystrophies, especially those due to defects in proteins that make up the dystrophin-glycoprotein complex (DGC), the enzyme that synthesizes NO, nitric oxide synthase (NOS), has been associated. Recent studies of dystrophies related to DGC defects suggest that one mechanism of cellular injury is functional ischemia related to alterations in cellular NOS and disruption of a normal protective action of NO. This protective action is the prevention of local ischemia during contraction-induced increases in sympathetic vasoconstriction. Rando (Microsc Res Tech 55(4):223-35, 2001), has shown that oxidative injury precedes pathologic changes and that muscle cells with defects in the DGC have an increased susceptibility to oxidant challenges. Excessive lipid peroxidation due to free radicals has also been shown to be a factor in myopathic diseases such as McArdle's disease (Russo et al., Med Hypotheses. 39(2):147-51, 1992). Furthermore, mitochondrial dysfunction is a well-known correlate of age-related muscle wasting (sarcopenia) and free radical damage has been suggested, though poorly investigated, as a contributing factor (reviewed in Navarro, A.; Lopez-Cepero, J. M.; Sanchez del Pino, M. L. Front. Biosci. 6: D26-44; 2001). Other indications include acute sarcopenia, for example muscle atrophy and/or cachexia associated with bums, bed rest, limb immobilization, or major thoracic, abdominal, and/or orthopedic surgery. It is contemplated that the methods of the present invention will also be effective in the treatment of muscle related pathological conditions.

In certain embodiments, the invention provides novel dietary compositions comprising sirtuin modulators, a method for their preparation, and a method of using the compositions for improvement of sports performance. Accordingly, provided are therapeutic compositions, foods and beverages that have actions of improving physical endurance and/or inhibiting physical fatigues for those people involved in broadly-defined exercises including sports requiring endurance and labors requiring repeated muscle exertions. Such dietary compositions may additional comprise electrolytes, caffeine, vitamins, carbohydrates, etc.

Aging/Stress

In certain embodiments, the invention provides methods for increasing cellular lifespan or preventing apoptosis comprising administering a high dose of a sirtuin activating compound to a subject that would benefit from increased cell lifespan or decreased apoptosis. For example, skin can be protected from aging (e.g., developing wrinkles, loss of elasticity, etc.) by treating skin or epithelial cells with a high dose of a sirtuin activating compound. In an exemplary embodiment, skin is contacted with a pharmaceutical or cosmetic composition comprising a high dose of a sirtuin activating compound. Exemplary skin afflictions or skin conditions that may be treated in accordance with the methods described herein include disorders or diseases associated with or caused by inflammation, sun damage or natural aging. For example, the compositions find utility in the prevention or treatment of contact dermatitis (including irritant contact dermatitis and allergic contact dermatitis), atopic dermatitis (also known as allergic eczema), actinic keratosis, keratinization disorders (including eczema), epidermolysis bullosa diseases (including penfigus), exfoliative dermatitis, seborrheic dermatitis, erythemas (including erythema multiforme and erythema nodosum), damage caused by the sun or other light sources, discoid lupus erythematosus, dernatomyositis, psoriasis, skin cancer and the effects of natural aging. In another embodiment, a high dose of a sirtuin activating compound may be used for the treatment of wounds and/or bums to promote healing, including, for example, first-, second- or third-degree bums and/or a thermal, chemical or electrical bums. The formulations may be administered topically, to the skin or mucosal tissue, as an ointment, lotion, cream, microemulsion, gel, solution or the like, as further described herein, within the context of a dosing regimen effective to bring about the desired result.

Topical formulations comprising a high dose of one or more sirtuin activating compounds may also be used as preventive, e.g., chemopreventive, compositions. When used in a chemopreventive method, susceptible skin is treated prior to any visible condition in a particular individual.

Sirtuin activating compounds may be delivered locally or systemically to a subject. In one embodiment, a high dose of a sirtuin activating compound is delivered locally to a tissue or organ of a subject by injection, topical formulation, etc.

In another embodiment, a high dose of a sirtuin activating compound may be used for treating or preventing a disease or condition induced or exacerbated by cellular senescence in a subject; methods for decreasing the rate of senescence of a subject, e.g., after onset of senescence; methods for extending the lifespan of a subject; methods for treating or preventing a disease or condition relating to lifespan; methods for treating or preventing a disease or condition relating to the proliferative capacity of cells; and methods for treating or preventing a disease or condition resulting from cell damage or death. In certain embodiments, the method does not act by decreasing the rate of occurrence of diseases that shorten the lifespan of a subject. In certain embodiments, a method does not act by reducing the lethality caused by a disease, such as cancer.

In yet another embodiment, a high dose of a sirtuin activating compound may be administered to a subject in order to generally increase the lifespan of its cells and to protect its cells against stress and/or against apoptosis. It is believed that treating a subject with a compound described herein is similar to subjecting the subject to hormesis, i.e., mild stress that is beneficial to organisms and may extend their lifespan.

A high dose of a sirtuin activating compound may be administered to a subject to prevent aging and aging-related consequences or diseases, such as stroke, heart disease, heart failure, arthritis, high blood pressure, and Alzheimer's disease. Other conditions that can be treated include ocular disorders, e.g., associated with the aging of the eye, such as cataracts, glaucoma, and macular degeneration. A high dose of a sirtuin activating compound can also be administered to subjects for treatment of diseases, e.g., chronic diseases, associated with cell death, in order to protect the cells from cell death. Exemplary diseases include those associated with neural cell death, neuronal dysfunction, or muscular cell death or dysfunction, such as Parkinson's disease, Alzheimer's disease, multiple sclerosis, amniotropic lateral sclerosis, and muscular dystrophy; AIDS; fulminant hepatitis; diseases linked to degeneration of the brain, such as Creutzfeld-Jakob disease, retinitis pigmentosa and cerebellar degeneration; myelodysplasis such as aplastic anemia; ischemic diseases such as myocardial infarction and stroke; hepatic diseases such as alcoholic hepatitis, hepatitis B and hepatitis C; joint-diseases such as osteoarthritis; atherosclerosis; alopecia; damage to the skin due to UV light; lichen planus; atrophy of the skin; cataract; and graft rejections. Cell death can also be caused by surgery, drug therapy, chemical exposure or radiation exposure.

A high dose of a sirtuin activating compound can also be administered to a subject suffering from an acute disease, e.g., damage to an organ or tissue, e.g., a subject suffering from stroke or myocardial infarction or a subject suffering from a spinal cord injury. A high dose of a sirtuin activating compound may also be used to repair an alcoholic's liver.

Cardiovascular Disease

In another embodiment, the invention provides a method for treating and/or preventing a cardiovascular disease by administering to a subject in need thereof a high dose of a sirtuin activating compound.

Cardiovascular diseases that can be treated or prevented using a high dose of a sirtuin activating compound include cardiomyopathy or myocarditis; such as idiopathic cardiomyopathy, metabolic cardiomyopathy, alcoholic cardiomyopathy, drug-induced cardiomyopathy, ischemic cardiomyopathy, and hypertensive cardiomyopathy. Also treatable or preventable using compounds and methods described herein are atheromatous disorders of the major blood vessels (macrovascular disease) such as the aorta, the coronary arteries, the carotid arteries, the cerebrovascular arteries, the renal arteries, the iliac arteries, the femoral arteries, and the popliteal arteries. Other vascular diseases that can be treated or prevented include those related to platelet aggregation, the retinal arterioles, the glomerular arterioles, the vasa nervorum, cardiac arterioles, and associated capillary beds of the eye, the kidney, the heart, and the central and peripheral nervous systems. A high dose of a sirtuin activating compound may also be used for increasing HDL levels in plasma of an individual.

Yet other disorders that may be treated with a high dose of a sirtuin activating compound include restenosis, e.g., following coronary intervention, and disorders relating to an abnormal level of high density and low density cholesterol.

In one embodiment, a high dose of a sirtuin activating compound may be administered as part of a combination therapeutic with another cardiovascular agent including, for example, an anti-arrhythmic agent, an antihypertensive agent, a calcium channel blocker, a cardioplegic solution, a cardiotonic agent, a fibrinolytic agent, a sclerosing solution, a vasoconstrictor agent, a vasodilator agent, a nitric oxide donor, a potassium channel blocker, a sodium channel blocker, statins, or a naturiuretic agent.

In one embodiment, a high dose of a sirtuin activating compound may be administered as part of a combination therapeutic with an anti-arrhythmia agent. Anti-arrhythmia agents are often organized into four main groups according to their mechanism of action: type I, sodium channel blockade; type II, beta-adrenergic blockade; type III, repolarization prolongation; and type IV, calcium channel blockade. Type I anti-arrhythmic agents include lidocaine, moricizine, mexiletine, tocainide, procainamide, encainide, flecanide, tocainide, phenytoin, propafenone, quinidine, disopyramide, and flecainide. Type II anti-arrhythmic agents include propranolol and esmolol. Type III includes agents that act by prolonging the duration of the action potential, such as amiodarone, artilide, bretylium, clofilium, isobutilide, sotalol, azimilide, dofetilide, dronedarone, ersentilide, ibutilide, tedisamil, and trecetilide. Type IV anti-arrhythmic agents include verapamil, diltaizem, digitalis, adenosine, nickel chloride, and magnesium ions.

In another embodiment, a high dose of a sirtuin activating compound may be administered as part of a combination therapeutic with another cardiovascular agent. Examples of cardiovascular agents include vasodilators, for example, hydralazine; angiotensin converting enzyme inhibitors, for example, captopril; anti-anginal agents, for example, isosorbide nitrate, glyceryl trinitrate and pentaerythritol tetranitrate; anti-arrhythmic agents, for example, quinidine, procainaltide and lignocaine; cardioglycosides, for example, digoxin and digitoxin; calcium antagonists, for example, verapamil and nifedipine; diuretics, such as thiazides and related compounds, for example, bendrofluazide, chlorothiazide, chlorothalidone, hydrochlorothiazide and other diuretics, for example, fursemide and triamterene, and sedatives, for example, nitrazepam, flurazepam and diazepam.

Other exemplary cardiovascular agents include, for example, a cyclooxygenase inhibitor such as aspirin or indomethacin, a platelet aggregation inhibitor such as clopidogrel, ticlopidene or aspirin, fibrinogen antagonists or a diuretic such as chlorothiazide, hydrochlorothiazide, flumethiazide, hydroflumethiazide, bendroflumethiazide, methylchlorthiazide, trichloromethiazide, polythiazide or benzthiazide as well as ethacrynic acid tricrynafen, chlorthalidone, furosemide, musolimine, bumetanide, triamterene, amiloride and spironolactone and salts of such compounds, angiotensin converting enzyme inhibitors such as captopril, zofenopril, fosinopril, enalapril, ceranopril, cilazopril, delapril, pentopril, quinapril, ramipril, lisinopril, and salts of such compounds, angiotensin II antagonists such as losartan, irbesartan or valsartan, thrombolytic agents such as tissue plasminogen activator (tPA), recombinant tPA, streptokinase, urokinase, prourokinase, and anisoylated plasminogen streptokinase activator complex (APSAC, Eminase, Beecham Laboratories), or animal salivary gland plasminogen activators, calcium channel blocking agents such as verapamil, nifedipine or diltiazem, thromboxane receptor antagonists such as ifetroban, prostacyclin mimetics, or phosphodiesterase inhibitors. Such combination products if formulated as a fixed dose employ the compounds of this invention within the dose range described above and the other pharmaceutically active agent within its approved dose range.

Yet other exemplary cardiovascular agents include, for example, vasodilators, e.g., bencyclane, cinnarizine, citicoline, cyclandelate, cyclonicate, ebumamonine, phenoxezyl, flunarizine, ibudilast, ifenprodil, lomerizine, naphlole, nikamate, nosergoline, nimodipine, papaverine, pentifylline, nofedoline, vincamin, vinpocetine, vichizyl, pentoxifylline, prostacyclin derivatives (such as prostaglandin E1 and prostaglandin I2), an endothelin receptor blocking drug (such as bosentan), diltiazem, nicorandil, and nitroglycerin. Examples of the cerebral protecting drug include radical scavengers (such as edaravone, vitamin E, and vitamin C), glutamate antagonists, AMPA antagonists, kainate antagonists, NMDA antagonists, GABA agonists, growth factors, opioid antagonists, phosphatidylcholine precursors, serotonin agonists, Na⁺/Ca²⁺ channel inhibitory drugs, and K⁺ channel opening drugs. Examples of the brain metabolic stimulants include amantadine, tiapride, and γ-aminobutyric acid. Examples of the anticoagulant include heparins (such as heparin sodium, heparin potassium, dalteparin sodium, dalteparin calcium, heparin calcium, pamaparin sodium, reviparin sodium, and danaparoid sodium), warfarin, enoxaparin, argatroban, batroxobin, and sodium citrate. Examples of the antiplatelet drug include ticlopidine hydrochloride, dipyridamole, cilostazol, ethyl icosapentate, sarpogrelate hydrochloride, dilazep hydrochloride, trapidil, a nonsteroidal antiinflammatory agent (such as aspirin), beraprostsodium, iloprost, and indobufene. Examples of the thrombolytic drug include urokinase, tissue-type plasminogen activators (such as alteplase, tisokinase, nateplase, pamiteplase, monteplase, and rateplase), and nasaruplase. Examples of the antihypertensive drug include angiotensin converting enzyme inhibitors (such as captopril, alacepril, lisinopril, imidapril, quinapril, temocapril, delapril, benazepril, cilazapril, trandolapril, enalapril, ceronapril, fosinopril, imadapril, mobertpril, perindopril, ramipril, spirapril, and randolapril), angiotensin II antagonists (such as losartan, candesartan, valsartan, eprosartan, and irbesartan), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendilin, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline), β-adrenaline receptor blocking drugs (propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, xybenolol, and esmolol), α-receptor blocking drugs (such as amosulalol, prazosin, terazosin, doxazosin, bunazosin, urapidil, phentolamine, arotinolol, dapiprazole, fenspiride, indoramin, labetalol, naftopidil, nicergoline, tamsulosin, tolazoline, trimazosin, and yohimbine), sympathetic nerve inhibitors (such as clonidine, guanfacine, guanabenz, methyldopa, and reserpine), hydralazine, todralazine, budralazine, and cadralazine. Examples of the antianginal drug include nitrate drugs (such as amyl nitrite, nitroglycerin, and isosorbide), β-adrenaline receptor blocking drugs (such as propranolol, pindolol, indenolol, carteolol, bunitrolol, atenolol, acebutolol, metoprolol, timolol, nipradilol, penbutolol, nadolol, tilisolol, carvedilol, bisoprolol, betaxolol, celiprolol, bopindolol, bevantolol, labetalol, alprenolol, amosulalol, arotinolol, befunolol, bucumolol, bufetolol, buferalol, buprandolol, butylidine, butofilolol, carazolol, cetamolol, cloranolol, dilevalol, epanolol, levobunolol, mepindolol, metipranolol, moprolol, nadoxolol, nevibolol, oxprenolol, practol, pronetalol, sotalol, sufinalol, talindolol, tertalol, toliprolol, andxybenolol), calcium channel blocking drugs (such as aranidipine, efonidipine, nicardipine, bamidipine, benidipine, manidipine, cilnidipine, nisoldipine, nitrendipine, nifedipine, nilvadipine, felodipine, amlodipine, diltiazem, bepridil, clentiazem, phendiline, galopamil, mibefradil, prenylamine, semotiadil, terodiline, verapamil, cilnidipine, elgodipine, isradipine, lacidipine, lercanidipine, nimodipine, cinnarizine, flunarizine, lidoflazine, lomerizine, bencyclane, etafenone, and perhexiline) trimetazidine, dipyridamole, etafenone, dilazep, trapidil, nicorandil, enoxaparin, and aspirin. Examples of the diuretic include thiazide diuretics (such as hydrochlorothiazide, methyclothiazide, trichlormethiazide, benzylhydrochlorothiazide, and penflutizide), loop diuretics (such as furosemide, etacrynic acid, bumetanide, piretanide, azosemide, and torasemide), K⁺ sparing diuretics (spironolactone, triamterene, and potassiumcanrenoate), osmotic diuretics (such as isosorbide, D-mannitol, and glycerin), nonthiazide diuretics (such as meticrane, tripamide, chlorthalidone, and mefruside), and acetazolamide. Examples of the cardiotonic include digitalis formulations (such as digitoxin, digoxin, methyldigoxin, deslanoside, vesnarinone, lanatoside C, and proscillaridin), xanthine formulations (such as aminophylline, choline theophylline, diprophylline, and proxyphylline), catecholamine formulations (such as dopamine, dobutamine, and docarpamine), PDE III inhibitors (such as amrinone, olprinone, and milrinone), denopamine, ubidecarenone, pimobendan, levosimendan, aminoethylsulfonic acid, vesnarinone, carperitide, and colforsin daropate. Examples of the antiarrhythmic drug include ajmaline, pirmenol, procainamide, cibenzoline, disopyramide, quinidine, aprindine, mexiletine, lidocaine, phenyloin, pilsicainide, propafenone, flecainide, atenolol, acebutolol, sotalol, propranolol, metoprolol, pindolol, amiodarone, nifekalant, diltiazem, bepridil, and verapamil. Examples of the antihyperlipidemic drug include atorvastatin, simvastatin, pravastatin sodium, fluvastatin sodium, clinofibrate, clofibrate, simfibrate, fenofibrate, bezafibrate, colestimide, and colestyramine. Examples of the immunosuppressant include azathioprine, mizoribine, cyclosporine, tacrolimus, gusperimus, and methotrexate.

Cell Death/Cancer

A high dose of a sirtuin activating compound may be administered to subjects who have recently received or are likely to receive a dose of radiation or toxin. In one embodiment, the dose of radiation or toxin is received as part of a work-related or medical procedure, e.g., working in a nuclear power plant, flying an airplane, an X-ray, CAT scan, or the administration of a radioactive dye for medical imaging; in such an embodiment, the high dose of the sirtuin activating compound is administered as a prophylactic measure. In another embodiment, the radiation or toxin exposure is received unintentionally, e.g., as a result of an industrial accident, habitation in a location of natural radiation, terrorist act, or act of war involving radioactive or toxic material. In such a case, the high dose of the sirtuin activating compound is preferably administered as soon as possible after the exposure to inhibit apoptosis and the subsequent development of acute radiation syndrome.

Neuronal Diseases/Disorders

In certain aspects, a high dose of a sirtuin activating compound can be used to treat patients suffering from neurodegenerative diseases, and traumatic or mechanical injury to the central nervous system (CNS), spinal cord or peripheral nervous system (PNS). Neurodegenerative disease typically involves reductions in the mass and volume of the human brain, which may be due to the atrophy and/or death of brain cells, which are far more profound than those in a healthy person that are attributable to aging. Neurodegenerative diseases can evolve gradually, after a long period of normal brain function, due to progressive degeneration (e.g., nerve cell dysfunction and death) of specific brain regions. Alternatively, neurodegenerative diseases can have a quick onset, such as those associated with trauma or toxins. The actual onset of brain degeneration may precede clinical expression by many years. Examples of neurodegenerative diseases include, but are not limited to, Alzheimer's disease (AD), Parkinson's disease (PD), Huntington's disease (HD), amyotrophic lateral sclerosis (ALS; Lou Gehrig's disease), diffuse Lewy body disease, chorea-acanthocytosis, primary lateral sclerosis, ocular diseases (ocular neuritis), chemotherapy-induced neuropathies (e.g., from vincristine, paclitaxel, bortezomib), diabetes-induced neuropathies and Friedreich's ataxia. Sirtuin-modulating compounds that increase the level and/or activity of a sirtuin protein can be used to treat these disorders and others as described below.

AD is a chronic, incurable, and unstoppable CNS disorder that occurs gradually, resulting in memory loss, unusual behavior, personality changes, and a decline in thinking abilities. These losses are related to the death of specific types of brain cells and the breakdown of connections and their supporting network (e.g. glial cells) between them. AD has been described as childhood development in reverse. In most people with AD, symptoms appear after the age 60. The earliest symptoms include loss of recent memory, faulty judgment, and changes in personality. Later in the disease, those with AD may forget how to do simple tasks like washing their hands. Eventually people with AD lose all reasoning abilities and become dependent on other people for their everyday care. Finally, the disease becomes so debilitating that patients are bedridden and typically develop coexisting illnesses.

PD is a chronic, incurable, and unstoppable CNS disorder that occurs gradually and results in uncontrolled body movements, rigidity, tremor, and dyskinesia. These motor system problems are related to the death of brain cells in an area of the brain that produces dopamine, a chemical that helps control muscle activity. In most people with PD, symptoms appear after age 50. The initial symptoms of PD are a pronounced tremor affecting the extremities, notably in the hands or lips. Subsequent characteristic symptoms of PD are stiffness or slowness of movement, a shuffling walk, stooped posture, and impaired balance. There are wide ranging secondary symptoms such as memory loss, dementia, depression, emotional changes, swallowing difficulties, abnormal speech, sexual dysfunction, and bladder and bowel problems. These symptoms will begin to interfere with routine activities, such as holding a fork or reading a newspaper. Finally, people with PD become so profoundly disabled that they are bedridden.

ALS (motor neuron disease) is a chronic, incurable, and unstoppable CNS disorder that attacks the motor neurons, components of the CNS that connect the brain to the skeletal muscles. In ALS, the motor neurons deteriorate and eventually die, and though a person's brain normally remains fully functioning and alert, the command to move never reaches the muscles. Most people who get ALS are between 40 and 70 years old. The first motor neurons that weaken are those controlling the arms or legs. Those with ALS may have trouble walking, they may drop things, fall, slur their speech, and laugh or cry uncontrollably. Eventually the muscles in the limbs begin to atrophy from disuse. This muscle weakness will become debilitating and a person will need a wheel chair or become unable to function out of bed.

The causes of these neurological diseases have remained largely unknown. They are conventionally defined as distinct diseases, yet clearly show extraordinary similarities in basic processes and commonly demonstrate overlapping symptoms far greater than would be expected by chance alone. Current disease definitions fail to properly deal with the issue of overlap and a new classification of the neurodegenerative disorders has been called for.

HD is another neurodegenerative disease resulting from genetically programmed degeneration of neurons in certain areas of the brain. This degeneration causes uncontrolled movements, loss of intellectual faculties, and emotional disturbance. HD is a familial disease, passed from parent to child through a dominant mutation in the wild-type gene. Some early symptoms of HD are mood swings, depression, irritability or trouble driving, learning new things, remembering a fact, or making a decision. As the disease progresses, concentration on intellectual tasks becomes increasingly difficult and the patient may have difficulty feeding himself or herself and swallowing.

Tay-Sachs disease and Sandhoff disease are glycolipid storage diseases caused by the lack of lysosomal β-hexosaminidase (Gravel et al., in The Metabolic Basis of Inherited Disease, eds. Scriver et al., McGraw-Hill, New York, pp. 2839-2879, 1995). In both disorders, GM2 ganglioside and related glycolipidssubstrates for β-hexosaminidase accumulate in the nervous system and trigger acute neurodegeneration. In the most severe forms, the onset of symptoms begins in early infancy. A precipitous neurodegenerative course then ensues, with affected infants exhibiting motor dysfunction, seizure, visual loss, and deafness. Death usually occurs by 2-5 years of age. Neuronal loss through an apoptotic mechanism has been demonstrated (Huang et al., Hum. Mol. Genet. 6: 1879-1885, 1997).

It is well-known that apoptosis plays a role in AIDS pathogenesis in the immune system. However, HIV-1 also induces neurological disease. Shi et al. (J. Clin. Invest. 98: 1979-1990, 1996) examined apoptosis induced by HIV-1 infection of the CNS in an in vitro model and in brain tissue from AIDS patients, and found that HIV-1 infection of primary brain cultures induced apoptosis in neurons and astrocytes in vitro. Apoptosis of neurons and astrocytes was also detected in brain tissue from 10/11 AIDS patients, including 5/5 patients with HIV-1 dementia and 4/5 nondemented patients.

There are four main peripheral neuropathies associated with HIV, namely sensory neuropathy, AIDP/CIPD, drug-induced neuropathy and CMV-related.

The most common type of neuropathy associated with AIDS is distal symmetrical polyneuropathy (DSPN). This syndrome is a result of nerve degeneration and is characterized by numbness and a sensation of pins and needles. DSPN causes few serious abnormalities and mostly results in numbness or tingling of the feet and slowed reflexes at the ankles. It generally occurs with more severe immunosuppression and is steadily progressive. Treatment with tricyclic antidepressants relieves symptoms but does not affect the underlying nerve damage.

A less frequent, but more severe type of neuropathy is known as acute or chronic inflammatory demyelinating polyneuropathy (AIDP/CIDP). In AIDP/CIDP there is damage to the fatty membrane covering the nerve impulses. This kind of neuropathy involves inflammation and resembles the muscle deterioration often identified with long-term use of AZT. It can be the first manifestation of HIV infection, where the patient may not complain of pain, but fails to respond to standard reflex tests. This kind of neuropathy may be associated with seroconversion, in which case it can sometimes resolve spontaneously. It can serve as a sign of HIV infection and indicate that it might be time to consider antiviral therapy. AIDP/CIDP may be auto-immune in origin.

Drug-induced, or toxic, neuropathies can be very painful. Antiviral drugs commonly cause peripheral neuropathy, as do other drugs e.g. vincristine, dilantin (an anti-seizure medication), high-dose vitamins, isoniazid, and folic acid antagonists. Peripheral neuropathy is often used in clinical trials for antivirals as a dose-limiting side effect, which means that more drugs should not be administered. Additionally, the use of such drugs can exacerbate otherwise minor neuropathies. Usually, these drug-induced neuropathies are reversible with the discontinuation of the drug.

CMV causes several neurological syndromes in AIDS, including encephalitis, myelitis, and polyradiculopathy.

Neuronal loss is also a salient feature of prion diseases, such as Creutzfeldt-Jakob disease in human, BSE in cattle (mad cow disease), Scrapie Disease in sheep and goats, and feline spongiform encephalopathy (FSE) in cats. A high dose of a sirtuin activating compound may be useful for treating or preventing neuronal loss due to these prior diseases.

In another embodiment, a high dose of a sirtuin activating compound may be used to treat or prevent any disease or disorder involving axonopathy. Distal axonopathy is a type of peripheral neuropathy that results from some metabolic or toxic derangement of peripheral nervous system (PNS) neurons. It is the most common response of nerves to metabolic or toxic disturbances, and as such may be caused by metabolic diseases such as diabetes, renal failure, deficiency syndromes such as malnutrition and alcoholism, or the effects of toxins or drugs. The most common cause of distal axonopathy is diabetes, and the most common distal axonopathy is diabetic neuropathy. The most distal portions of axons are usually the first to degenerate, and axonal atrophy advances slowly towards the nerve's cell body. If the noxious stimulus is removed, regeneration is possible, though prognosis decreases depending on the duration and severity of the stimulus. Those with distal axonopathies usually present with symmetrical glove-stocking sensori-motor disturbances. Deep tendon reflexes and autonomic nervous system (ANS) functions are also lost or diminished in affected areas.

Diabetic neuropathies are neuropathic disorders that are associated with diabetes mellitus. These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy. Clinical manifestations of diabetic neuropathy include, for example, sensorimotor polyneuropathy such as numbness, sensory loss, dysesthesia and nighttime pain; autonomic neuropathy such as delayed gastric emptying or gastroparesis; and cranial neuropathy such as oculomotor (3rd) neuropathies or Mononeuropathies of the thoracic or lumbar spinal nerves.

Peripheral neuropathy is the medical term for damage to nerves of the peripheral nervous system, which may be caused either by diseases of the nerve or from the side-effects of systemic illness. Peripheral neuropathies vary in their presentation and origin, and may affect the nerve or the neuromuscular junction. Major causes of peripheral neuropathy include seizures, nutritional deficiencies, and HIV, though diabetes is the most likely cause. Mechanical pressure from staying in one position for too long, a tumor, intraneural hemorrhage, exposing the body to extreme conditions such as radiation, cold temperatures, or toxic substances can also cause peripheral neuropathy.

In an exemplary embodiment, a high dose of a sirtuin activating compound may be used to treat or prevent multiple sclerosis (MS), including relapsing MS and monosymptomatic MS, and other demyelinating conditions, such as, for example, chromic inflammatory demyelinating polyneuropathy (CIDP), or symptoms associated therewith.

MS is a chronic, often disabling disease of the central nervous system. Various and converging lines of evidence point to the possibility that the disease is caused by a disturbance in the immune function, although the cause of this disturbance has not been established. This disturbance permits cells of the immune system to “attack” myelin, the fat containing insulating sheath that surrounds the nerve axons located in the central nervous system (“CNS”). When myelin is damaged, electrical pulses cannot travel quickly or normally along nerve fiber pathways in the brain and spinal cord. This results in disruption of normal electrical conductivity within the axons, fatigue and disturbances of vision, strength, coordination, balance, sensation, and bladder and bowel function.

As such, MS is now a common and well-known neurological disorder that is characterized by episodic patches of inflammation and demyelination which can occur anywhere in the CNS. However, almost always without any involvement of the peripheral nerves associated therewith. Demyelination produces a situation analogous to that resulting from cracks or tears in an insulator surrounding an electrical cord. That is, when the insulating sheath is disrupted, the circuit is “short circuited” and the electrical apparatus associated therewith will function intermittently or nor at all. Such loss of myelin surrounding nerve fibers results in short circuits in nerves traversing the brain and the spinal cord that thereby result in symptoms of MS. It is further found that such demyelination occurs in patches, as opposed to along the entire CNS. In addition, such demyelination may be intermittent. Therefore, such plaques are disseminated in both time and space.

It is believed that the pathogenesis involves a local disruption of the blood brain barrier which causes a localized immune and inflammatory response, with consequent damage to myelin and hence to neurons.

Clinically, MS exists in both sexes and can occur at any age. However, its most common presentation is in the relatively young adult, often with a single focal lesion such as a damage of the optic nerve, an area of anesthesia (loss of sensation), or paraesthesia (localize loss of feeling), or muscular weakness. In addition, vertigo, double vision, localized pain, incontinence, and pain in the arms and legs may occur upon flexing of the neck, as well as a large variety of less common symptoms.

An initial attack of MS is often transient, and it may be weeks, months, or years before a further attack occurs. Some individuals may enjoy a stable, relatively event free condition for a great number of years, while other less fortunate ones may experience a continual downhill course ending in complete paralysis. There is, most commonly, a series of remission and relapses, in which each relapse leaves a patient somewhat worse than before. Relapses may be triggered by stressful events, viral infections or toxins. Therein, elevated body temperature, i.e., a fever, will make the condition worse, or as a reduction of temperature by, for example, a cold bath, may make the condition better.

In yet another embodiment, a high dose of a sirtuin activating compound may be used to treat trauma to the nerves, including, trauma due to disease, injury (including surgical intervention), or environmental trauma (e.g., neurotoxins, alcoholism, etc.).

A high dose of a sirtuin activating compound may also be useful to prevent, treat, and alleviate symptoms of various PNS disorders, such as the ones described below. The PNS is composed of the nerves that lead to or branch off from the spinal cord and CNS. The peripheral nerves handle a diverse array of functions in the body, including sensory, motor, and autonomic functions. When an individual has a peripheral neuropathy, nerves of the PNS have been damaged. Nerve damage can arise from a number of causes, such as disease, physical injury, poisoning, or malnutrition. These agents may affect either afferent or efferent nerves. Depending on the cause of damage, the nerve cell axon, its protective myelin sheath, or both may be injured or destroyed.

The term “peripheral neuropathy” encompasses a wide range of disorders in which the nerves outside of the brain and spinal cord—peripheral nerves—have been damaged. Peripheral neuropathy may also be referred to as peripheral neuritis, or if many nerves are involved, the terms polyneuropathy or polyneuritis may be used.

Peripheral neuropathy is a widespread disorder, and there are many underlying causes. Some of these causes are common, such as diabetes, and others are extremely rare, such as acrylamide poisoning and certain inherited disorders. The most common worldwide cause of peripheral neuropathy is leprosy. Leprosy is caused by the bacterium Mycobacterium leprae, which attacks the peripheral nerves of affected people.

Leprosy is extremely rare in the United States, where diabetes is the most commonly known cause of peripheral neuropathy. It has been estimated that more than 17 million people in the United States and Europe have diabetes-related polyneuropathy. Many neuropathies are idiopathic; no known cause can be found. The most common of the inherited peripheral neuropathies in the United States is Charcot-Marie-Tooth disease, which affects approximately 125,000 persons.

Another of the better known peripheral neuropathies is Guillain-Barre syndrome, which arises from complications associated with viral illnesses, such as cytomegalovirus, Epstein-Barr virus, and human immunodeficiency virus (HIV), or bacterial infection, including Campylobacter jejuni and Lyme disease. The worldwide incidence rate is approximately 1.7 cases per 100,000 people annually. Other well-known causes of peripheral neuropathies include chronic alcoholism, infection of the varicella-zoster virus, botulism, and poliomyelitis. Peripheral neuropathy may develop as a primary symptom, or it may be due to another disease. For example, peripheral neuropathy is only one symptom of diseases such as amyloid neuropathy, certain cancers, or inherited neurologic disorders. Such diseases may affect the PNS and the CNS, as well as other body tissues.

Other PNS diseases treatable with sirtuin-modulating compounds that increase the level and/or activity of a sirtuin protein include: Brachial Plexus Neuropathies (diseases of the cervical and first thoracic roots, nerve trunks, cords, and peripheral nerve components of the brachial plexus. Clinical manifestations include regional pain, paresthesia; muscle weakness, and decreased sensation in the upper extremity. These disorders may be associated with trauma, including birth injuries; thoracic outlet syndrome; neoplasms, neuritis, radiotherapy; and other conditions. See Adams et al., Principles of Neurology, 6th ed, pp 351-2); Diabetic Neuropathies (peripheral, autonomic, and cranial nerve disorders that are associated with diabetes mellitus). These conditions usually result from diabetic microvascular injury involving small blood vessels that supply nerves (vasa nervorum). Relatively common conditions which may be associated with diabetic neuropathy include third nerve palsy; mononeuropathy; mononeuritis multiplex; diabetic amyotrophy; a painful polyneuropathy; autonomic neuropathy; and thoracoabdominal neuropathy (see Adams et al., Principles of Neurology, 6th ed, p 1325); mononeuropathies (disease or trauma involving a single peripheral nerve in isolation, or out of proportion to evidence of diffuse peripheral nerve dysfunction). Mononeuritis multiplex refers to a condition characterized by multiple isolated nerve injuries. Mononeuropathies may result from a wide variety of causes, including ischemia; traumatic injury; compression; connective tissue diseases; cumulative trauma disorders; and other conditions; Neuralgia (intense or aching pain that occurs along the course or distribution of a peripheral or cranial nerve); Peripheral Nervous System Neoplasms (neoplasms which arise from peripheral nerve tissue). This includes neurofibromas; Schwannomas; granular cell tumors; and malignant peripheral nerve sheath tumors. See DeVita Jr et al., Cancer: Principles and Practice of Oncology, 5th ed, pp 1750-1); and Nerve Compression Syndromes (mechanical compression of nerves or nerve roots from internal or external causes. These may result in a conduction block to nerve impulses, due to, for example, myelin sheath dysfunction, or axonal loss. The nerve and nerve sheath injuries may be caused by ischemia; inflammation; or a direct mechanical effect; Neuritis (a general term indicating inflammation of a peripheral or cranial nerve). Clinical manifestation may include pain; paresthesias; paresis; or hyperesthesia; Polyneuropathies (diseases of multiple peripheral nerves). The various forms are categorized by the type of nerve affected (e.g., sensory, motor, or autonomic), by the distribution of nerve injury (e.g., distal vs. proximal), by nerve component primarily affected (e.g., demyelinating vs. axonal), by etiology, or by pattern of inheritance.

In another embodiment, a high dose of a sirtuin activating compound may be used to treat or prevent chemotherapeutic induced neuropathy. The high dose of the sirtuin activating compound may be administered prior to administration of the chemotherapeutic agent, concurrently with administration of the chemotherapeutic drug, and/or after initiation of administration of the chemotherapeutic drug. If the high dose of the sirtuin activating compound is administered after the initiation of administration of the chemotherapeutic drug, it is desirable that the high dose of the sirtuin activating compound be administered prior to, or at the first signs, of chemotherapeutic induced neuropathy.

Chemotherapy drugs can damage any part of the nervous system. Encephalopathy and myelopathy are fortunately very rare. Damage to peripheral nerves is much more common and can be a side effect of treatment experienced by people with cancers, such as lymphoma. Most of the neuropathy affects sensory rather than motor nerves. Thus, the common symptoms are tingling, numbness or a loss of balance. The longest nerves in the body seem to be most sensitive hence the fact that most patients will report numbness or pins and needles in their hands and feet.

The chemotherapy drugs which are most commonly associated with neuropathy, are the Vinca alkaloids (anti-cancer drugs originally derived from a member of the periwinkle—the Vinca plant genus) and a platinum-containing drug called Cisplatin. The Vinca alkaloids include the drugs vinblastine, vincristine and vindesine. Many combination chemotherapy treatments for lymphoma for example CHOP and CVP contain vincristine, which is the drug known to cause this problem most frequently. Indeed, it is the risk of neuropathy that limits the dose of vincristine that can be administered.

Studies that have been performed have shown that most patients will lose some reflexes in their legs as a result of treatment with vincristine and many will experience some degree of tingling (paresthesia) in their fingers and toes. The neuropathy does not usually manifest itself right at the start of the treatment but generally comes on over a period of a few weeks. It is not essential to stop the drug at the first onset of symptoms, but if the neuropathy progresses this may be necessary. It is very important that patients should report such symptoms to their doctors, as the nerve damage is largely reversible if the drug is discontinued. Most doctors will often reduce the dose of vincristine or switch to another form of Vinca alkaloid such as vinblastine or vindesine if the symptoms are mild. Occasionally, the nerves supplying the bowel are affected causing abdominal pain and constipation.

In another embodiment, a high dose of a sirtuin activating compound may be used to treat or prevent a polyglutamine disease. Huntington's Disease (HD) and Spinocerebellar ataxia type 1 (SCA1) are just two examples of a class of genetic diseases caused by dynamic mutations involving the expansion of triplet sequence repeats. In reference to this common mechanism, these disorders are called trinucleotide repeat diseases. At least 14 such diseases are known to affect human beings. Nine of them, including SCA1 and Huntington's disease, have CAG as the repeated sequence (see Table A below). Since CAG codes for an amino acid called glutamine, these nine trinucleotide repeat disorders are collectively known as polyglutamine diseases.

Although the genes involved in different polyglutamine diseases have little in common, the disorders they cause follow a strikingly similar course. Each disease is characterized by a progressive degeneration of a distinct group of nerve cells. The major symptoms of these diseases are similar, although not identical, and usually affect people in midlife. Given the similarities in symptoms, the polyglutamine diseases are hypothesized to progress via common cellular mechanisms. In recent years, scientists have made great strides in unraveling what the mechanisms are.

Above a certain threshold, the greater the number of glutamine repeats in a protein, the earlier the onset of disease and the more severe the symptoms. This suggests that abnormally long glutamine tracts render their host protein toxic to nerve cells.

To test this hypothesis, scientists have generated genetically engineered mice expressing proteins with long polyglutamine tracts. Regardless of whether the mice express full-length proteins or only those portions of the proteins containing the polyglutamine tracts, they develop symptoms of polyglutamine diseases. This suggests that a long polyglutamine tract by itself is damaging to cells and does not have to be part of a functional protein to cause its damage.

For example, it is thought that the symptoms of SCA1 are not directly caused by the loss of normal ataxin-1 function but rather by the interaction between ataxin-1 and another protein called LANP. LANP is needed for nerve cells to communicate with one another and thus for their survival. When the mutant ataxin-1 protein accumulates inside nerve cells, it “traps” the LANP protein, interfering with its normal function. After a while, the absence of LANP function appears to cause nerve cells to malfunction.

TABLE A
Summary of Polyglutamine Diseases.

| | | | | | Normal | Disease |
|---|---|---|---|---|---|---|
| | Gene | Chromosomal | Pattern of | | repeat | repeat |
| Disease | name | location | inheritance | Protein | length | length |
| Spinobulbar | AR | Xq13-21 | X-linked | androgen | 9-36 | 38-62 |
| muscular | | | recessive | receptor | | |
| atrophy | | | | (AR) | | |
| (Kennedy | | | | | | |
| disease) | | | | | | |
| Huntington's | HD | 4p16.3 | autosomal | huntingtin | 6-35 | 36-121 |
| disease | | | dominant | | | |
| Dentatorubral- | DRPLA | 12p13.31 | autosomal | atrophin-1 | 6-35 | 49-88 |
| pallidoluysian | | | dominant | | | |
| atrophy (Haw | | | | | | |
| River | | | | | | |
| syndrome) | | | | | | |
| Spinocerebellar | SCA1 | 6p23 | autosomal | ataxin-1 | 6-44 | 39-82 |
| ataxia type 1 | | | dominant | | | |
| Spinocerebellar | SCA2 | 12q24.1 | autosomal | ataxin-2 | 15-31 | 36-63 |
| ataxia type 2 | | | dominant | | | |
| Spinocerebellar | ScA3 | 14q32.1 | autosomal | ataxin-3 | 12-40 | 55-84 |
| ataxia type 3 | | | dominant | | | |
| (Machado- | | | | | | |
| Joseph disease) | | | | | | |
| Spinocerebellar | SCA6 | 19p13 | autosomal | α1_{A}- | 4-18 | 21-33 |
| ataxia type 6 | | | dominant | voltage- | | |
| | | | | dependent | | |
| | | | | calcium | | |
| | | | | channel | | |
| | | | | subunit | | |
| Spinocerebellar | SCA7 | 3p12-13 | autosomal | ataxin-7 | 4-35 | 37-306 |
| ataxia type 7 | | | dominant | | | |
| Spinocerebellar | SCA17 | 6q27 | autosomal | TATA | 25-42 | 45-63 |
| ataxia type 17 | | | dominant | binding | | |
| | | | | protein | | |

Many transcription factors have also been found in neuronal inclusions in different diseases. It is possible that these transcription factors interact with the polyglutamine-containing proteins and then become trapped in the neuronal inclusions. This in turn might keep the transcription factors from turning genes on and off as needed by the cell. Another observation is hypoacetylation of histones in affected cells. This has led to the hypothesis that Class I/II Histone Deacetylase (HDAC I/II) inhibitors, which are known to increase histone acetylation, may be a novel therapy for polyglutamine diseases (U.S. patent application Ser. No. 10/476,627; “Method of treating neurodegenerative, psychiatric, and other disorders with deacetylase inhibitors”).

In yet another embodiment, the invention provides a method for treating or preventing neuropathy related to ischemic injuries or diseases, such as, for example, coronary heart disease (including congestive heart failure and myocardial infarctions), stroke, emphysema, hemorrhagic shock, peripheral vascular disease (upper and lower extremities) and transplant related injuries.

In certain embodiments, the invention provides a method to treat a central nervous system cell to prevent damage in response to a decrease in blood flow to the cell. Typically the severity of damage that may be prevented will depend in large part on the degree of reduction in blood flow to the cell and the duration of the reduction. By way of example, the normal amount of perfusion to brain gray matter in humans is about 60 to 70 mL/100 g of brain tissue/min. Death of central nervous system cells typically occurs when the flow of blood falls below approximately 8-10 mL/100 g of brain tissue/min, while at slightly higher levels (i.e. 20-35 mL/100 g of brain tissue/min) the tissue remains alive but not able to function. In one embodiment, apoptotic or necrotic cell death may be prevented. In still a further embodiment, ischemic-mediated damage, such as cytoxic edema or central nervous system tissue anoxemia, may be prevented. In each embodiment, the central nervous system cell may be a spinal cell or a brain cell.

Another aspect encompasses administrating a high dose of a sirtuin activating compound to a subject to treat a central nervous system ischemic condition. A number of central nervous system ischemic conditions may be treated by the sirtuin activating compounds described herein. In one embodiment, the ischemic condition is a stroke that results in any type of ischemic central nervous system damage, such as apoptotic or necrotic cell death, cytoxic edema or central nervous system tissue anoxia. The stroke may impact any area of the brain or be caused by any etiology commonly known to result in the occurrence of a stroke. In one alternative of this embodiment, the stroke is a brain stem stroke. Generally speaking, brain stem strokes strike the brain stem, which control involuntary life-support functions such as breathing, blood pressure, and heartbeat. In another alternative of this embodiment, the stroke is a cerebellar stroke. Typically, cerebellar strokes impact the cerebellum area of the brain, which controls balance and coordination. In still another embodiment, the stroke is an embolic stroke. In general terms, embolic strokes may impact any region of the brain and typically result from the blockage of an artery by a vaso-occlusion. In yet another alternative, the stroke may be a hemorrhagic stroke. Like ischemic strokes, hemorrhagic stroke may impact any region of the brain, and typically result from a ruptured blood vessel characterized by a hemorrhage (bleeding) within or surrounding the brain. In a further embodiment, the stroke is a thrombotic stroke. Typically, thrombotic strokes result from the blockage of a blood vessel by accumulated deposits.

In another embodiment, the ischemic condition may result from a disorder that occurs in a part of the subject's body outside of the central nervous system, but yet still causes a reduction in blood flow to the central nervous system. These disorders may include, but are not limited to a peripheral vascular disorder, a venous thrombosis, a pulmonary embolus, arrhythmia (e.g. atrial fibrillation), a myocardial infarction, a transient ischemic attack, unstable angina, or sickle cell anemia. Moreover, the central nervous system ischemic condition may occur as result of the subject undergoing a surgical procedure. By way of example, the subject may be undergoing heart surgery, lung surgery, spinal surgery, brain surgery, vascular surgery, abdominal surgery, or organ transplantation surgery. The organ transplantation surgery may include heart, lung, pancreas, kidney or liver transplantation surgery. Moreover, the central nervous system ischemic condition may occur as a result of a trauma or injury to a part of the subject's body outside the central nervous system. By way of example, the trauma or injury may cause a degree of bleeding that significantly reduces the total volume of blood in the subject's body. Because of this reduced total volume, the amount of blood flow to the central nervous system is concomitantly reduced. By way of further example, the trauma or injury may also result in the formation of a vaso-occlusion that restricts blood flow to the central nervous system.

Of course it is contemplated that the high dose of the sirtuin activating compound may be employed to treat the central nervous system ischemic condition irrespective of the cause of the condition. In one embodiment, the ischemic condition results from a vaso-occlusion. The vaso-occlusion may be any type of occlusion, but is typically a cerebral thrombosis or an embolism. In a further embodiment, the ischemic condition may result from a hemorrhage. The hemorrhage may be any type of hemorrhage, but is generally a cerebral hemorrhage or a subararachnoid hemorrhage. In still another embodiment, the ischemic condition may result from the narrowing of a vessel. Generally speaking, the vessel may narrow as a result of a vasoconstriction such as occurs during vasospasms, or due to arteriosclerosis. In yet another embodiment, the ischemic condition results from an injury to the brain or spinal cord.

In yet another aspect, a high dose of a sirtuin activating compound may be administered to reduce infarct size of the ischemic core following a central nervous system ischemic condition. Moreover, a high dose of a sirtuin activating compound may also be beneficially administered to reduce the size of the ischemic penumbra or transitional zone following a central nervous system ischemic condition.

In one embodiment, a combination drug regimen may include drugs or compounds for the treatment or prevention of neurodegenerative disorders or secondary conditions associated with these conditions. Thus, a combination drug regimen may include a high dose of one or more sirtuin activators and one or more anti-neurodegeneration agents. For example, one or more sirtuin-activating compounds can be combined with an effective amount of one or more of: L-DOPA; a dopamine agonist; an adenosine A_{2A} receptor antagonist; a COMT inhibitor; a MAO inhibitor; an N-NOS inhibitor; a sodium channel antagonist; a selective N-methyl D-aspartate (NMDA) receptor antagonist; an AMPA/kainate receptor antagonist; a calcium channel antagonist; a GABA-A receptor agonist; an acetyl-choline esterase inhibitor; a matrix metalloprotease inhibitor; a PARP inhibitor; an inhibitor of p38 MAP kinase or cjun-N-terminal kinases; TPA; NDA antagonists; beta-interferons; growth factors; glutamate inhibitors; and/or as part of a cell therapy.

Exemplary N-NOS inhibitors include 4-(6-amino-pyridin-2-yl)-3-methoxyphenol 6-[4-(2-dimethylamino-ethoxy)-2-methoxy-phenyl]-pyridin-2-yl-amine, 6-[4-(2-dimethylamino-ethoxy)-2,3-dimet-hyl-phenyl]-pyridin-2-yl-amine, 6-[4-(2-pyrrolidinyl-ethoxy)-2,3-dimethyl-p-henyl]-pyridin-2-yl-amine, 6-[4-(4-(n-methyl)piperidinyloxy)-2,3-dimethyl-p-henyl]-pyridin-2-yl-amine, 6-[4-(2-dimethylamino-ethoxy)-3-methoxy-phenyl]-pyridin-2-yl-amine, 6-[4-(2-pyrrolidinyl-ethoxy)-3-methoxy-phenyl]-pyridin-2-yl-amine, 6-{4-[2-(6,7-dimethoxy-3,4-dihydro-1h-isoquinolin-2-yl)-ethoxy]-3-methoxy-phenyl}-pyridin-2-yl-amine, 6-{3-methoxy-4-[2-(4-phenethyl-piper-azin-1-yl)-ethoxy]-phenyl}-pyridin-2-yl-amine, 6-{3-methoxy-4-[2-(4-methyl-piperazin-1-yl)-ethoxy]-phenyl}-pyridin-2-yl-amine, 6-{4-[2-(4-dimethylamin-o-piperidin-1-yl)-ethoxy]-3-methoxy-phenyl}-pyridin-2-yl-amine, 6-[4-(2-dimethylamino-ethoxy)-3-ethoxy-phenyl]-pyridin-2-yl-amine, 6-[4-(2-pyrrolidinyl-ethoxy)-3-ethoxy-phenyl]-pyridin-2-yl-amine, 6-[4-(2-dimethylamino-ethoxy)-2-isopropyl-phenyl]-pyridin-2-yl-amine, 4-(6-amino-pyridin-yl)-3-cyclopropyl-phenol 6-[2-cyclopropyl-4-(2-dimethy-lamino-ethoxy)-phenyl]-pyridin-2-yl-amine, 6-[2-cyclopropyl-4-(2-pyrrolidin-1-yl-ethoxy)-phenyl]-pyridin-2-yl-amine, 3-[3-(6-amino-pyridin-2yl)-4-cycl-opropyl-phenoxy]-pyrrolidine-1-carboxylic acid tert-butyl ester 6-[2-cyclopropyl-4-(1-methyl-pyrrolidin-3-yl-oxy)-phenyl]-pyridin-2-yl-amine, 4-(6-amino-pyridin-2-yl)-3-cyclobutyl-phenol 6-[2-cyclobutyl-4-(2-dime-thylamino-ethoxy)-phenyl]-pyridin-2-yl-amine, 6-[2-cyclobutyl-4-(2-pyrrolid-in-1-yl-ethoxy)-phenyl]-pyridin-2-yl-amine, 6-[2-cyclobutyl-4-(1-methyl-pyr-rolidin-3-yl-oxy)-phenyl]-pyridin-2-yl-amine, 4-(6-amino-pyridin-2-yl)-3-cy-clopentyl-phenol 6-[2-cyclopentyl-4-(2-dimethylamino-ethoxy)-phenyl]-pyrid-in-2-yl-amine, 6-[2-cyclopentyl-4-(2-pyrrolidin-1-yl-ethoxy)-phenyl]-pyridin-2-yl-amine, 3-[4-(6-amino-pyridin-2-yl)-3-methoxy-phenoxy]-pyrrolidine-1-ca-rboxylic acid tert butyl ester 6-[4-(1-methyl-pyrrolidin-3-yl-oxy)-2-metho-xy-phenyl]-pyridin-2-yl-amine, 4-[4-(6-amino-pyridin-2yl)-3-methoxy-phenoxy-]-piperidine-1-carboxylic acid tert butyl ester 6-[2-methoxy-4-(1-methyl-p-iperidin-4-yl-oxy)-phenyl]-pyridin-2-yl-amine, 6-[4-(allyloxy)-2-methoxy-ph-enyl]-pyridin-2-yl-amine, 4-(6-amino-pyridin-2-yl)-3-methoxy-6-allyl-phenol 12 and 4-(6-amino-pyridin-2-yl)-3-methoxy-2-allyl-phenol 13 4-(6-amino-pyridin-2-yl)-3-methoxy-6-propyl-phenol 6-[4-(2-dimethylamino-ethoxy)-2-methoxy-5-propyl-phenyl]-pyridin-yl-amine, 6-[2-isopropyl-4-(pyrrolidin-3-yl-oxy)-phenyl]-pyridin-2-yl-amine, 6-[2-isopropyl-4-(piperidin-3-yl-oxy)-phenyl]-pyridin-2-yl-amine, 6-[2-isopropyl-4-(1-methyl-azetidin-3-yl-oxy)-phenyl]-pyridin-2-yl-amine, 6-[2-isopropyl-4-(1-methyl-piperidin-4-yl-oxy)-phenyl]-pyridin-2-yl-amine, 6-[2-isopropyl-4-(1-methyl-pyrrolidin-3-yl-oxy)-phenyl]-pyridin-2-yl-amin-e 6-[2-isopropyl-4-(1-methyl-pyrrolidin-3-yl-oxy)-phenyl]-pyridin-2-yl-amine, 6-[2-isopropyl-4-(2-methyl-2-aza-bicyclo[2.2.1]hept-5-yl-oxy)-phenyl]-p-yridin-2-yl-amine, 6-[4-(2-dimethylamino-ethoxy)-2-methoxy-phenyl]-pyridin-2-yl-amine, 6-{4-[2-(benzyl-methyl-amino)-ethoxy]-2-methoxy-phenyl}-pyridin-2-yl-amine, 6-[2-methoxy-4-(2-pyrrolidin-1-yl-ethoxy)-phenyl]-pyridin-2-yl-amine, 2-(6-amino-pyridin-2-yl)-5-(2-dimethylamino-ethoxy)-phenol 2-[4-(6-amino-pyridin-2-yl)-3-methoxy-phenoxy]-acetamide 6-[4-(2-amino-ethoxy)-2-methoxy-phenyl]-pyridin-2-yl-amine, 6-{4-[2-(3,4-dihydro-1h-isoquinolin-2-yl)-ethoxy]-2-methoxy-phenyl}-pyrid-in-2-yl-amine, 2-[4-(6-amino-pyridin-2-yl)-3-methoxy-phenoxy]-ethanol 6-{2-methoxy-4-[2-(2,2,6,6-tetramethyl-piperidin-1-yl)-ethoxy]-phenyl}-py-ridin-2-yl-amine, 6-{4-[2-(2,5-dimethyl-pyrrolidin-1-yl)-ethoxy]-2-methoxy-phenyl}-pyridin-2-yl-amine, 6-{4-[2-(2,5-dimethyl-pyrrolidin-1-yl)-ethoxy]-2-methoxy-phenyl}-pyridin-2-yl-amine, 2-[4-(6-amino-pyridin-2-yl)-3-methoxy-phenoxy]-1-(2,2,6,6-tetramethyl-piperidin-1-yl)-ethanone 6-[2-methoxy-4-(1-methyl-pyrrolidin-2-yl-methoxy)-phenyl]-pyridin-2-y-amine, 6-[4-(2-dimethylamino-ethoxy)-2-propoxy-phenyl]-pyridin-2-yl-amine, 6-{4-[2-(benzyl-methyl-amino)-ethoxy]-2-propoxy-phenyl}-pyridin-2-yl-amin-e 6-[4-(2-ethoxy-ethoxy)-2-methoxy-phenyl]-pyridin-2-yl-amine, 6-[4-(2-dimethylamino-ethoxy)-2-isopropoxy-phenyl]-pyridin-2-yl-amine, 6-[4-(2-ethoxy-ethoxy)-2-isopropoxy-phenyl]-pyridin-2-yl-amine, 6-[2-methoxy-4-(3-methyl-butoxy)-phenyl]-pyridin-2-yl-amine, 6-[4-(2-dimethylamino-ethoxy)-2-ethoxy-phenyl]-pyridin-2-yl-amine, 6-{4-[2-(benzyl-methyl-amino)-ethoxy]-2-ethoxy-phenyl}-pyridin-2-yl-amine, 6-[2-ethoxy-4-(3-methyl-butoxy)-phenyl]-pyridin-2-yl-amine, 1-(6-amino-3-aza-bicyclo[3.1.0]hex-3-yl)-2-[4-(6-amino-pyridin-2-yl)-3-et-hoxy-phenoxy]-ethanone 6-[2-ethoxy-4-(2-pyrrolidin-1-yl-ethoxy)-phenyl]-py-ridin-2-yl-amine, 3-{2-[4-(6-amino-pyridin-2-yl)-3-ethoxy-phenoxy]-ethyl}-3-aza-bicyclo[3.1.0]hex-6-yl-amine, 1-(6-amino-3-aza-bicyclo[3.1.0]hex-3-yl)-2-[4-(6-amino-pyridin-2-yl)-3-methoxy-phenoxy]-ethanone 3-{2-[4-(6-amino-pyridin-2-yl)-3-methoxy-phenoxy]-ethyl}-3-aza-bicyclo[3.-1.0]hex-6-yl-amine, 6-[2-isopropoxy-4-(2-pyrrolidin-1-yl-ethoxy)-phenyl]-py-ridin-2-yl-amine, 6-{4-[2-(benzyl-methyl-amino)-ethoxy]-2-isopropoxy-phenyl-}-pyridin-2-yl-amine, 6-[4-(2-dimethylamino-ethoxy)-2-methoxy-5-propyl-phen-yl]-pyridin-2-yl-amine, 6-[5-allyl-4-(2-dimethylamino-ethoxy)-2-methoxy-phe-nyl]-pyridin-2-yl-amine, 6-[5-allyl-2-methoxy-4-(2-pyrrolidin-1-yl-ethoxy)-phenyl]-pyridin-2-yl-amine, 6-[3-allyl-4-(2-dimethylamino-ethoxy)-2-methoxy-phenyl]-pyridin-2-yl-amine, 6-[2-methoxy-4-(pyrrolidin-3-yl-oxy)-phenyl]-p-yridin-2-yl-amine, 6-[2-methoxy-4-(1-methyl-pyrrolidin-3-yl-oxy)-phenyl]-py-ridin-2-yl-amine, 6-[2-ethoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(piperidin-4-yl-oxy)-phenyl]-pyridin-2-yl-amine, 6-[2-methoxy-4-(2,2,6,6-tetramethyl-piperidin-4-yl-oxy)-phenyl]-pyridin-2-yl-amine, 6-[2-isopropoxy-4-(pyrrolidin-3-yl-oxy)-phenyl]-pyridin-2-yl-amine, 3-[4-(6-amino-pyridin-2-yl)-3-methoxy-phenoxy]-azetidine-1-carboxylic acid tert-butyl ester 6-[4-(azetidin-3-yl-oxy)-2-methoxy-phenyl]-pyridin-2-yl-amine, 6-[2-methoxy-4-(1-methyl-azetidin-3-yl-oxy)-phenyl]-pyridin-2-y-1-amine, 6-[2-isopropoxy-4-(pyrrolidin-3-yl-oxy)-phenyl]-pyridin-2-yl-amine, 6-[2-isopropoxy-4-(pyrrolidin-3-yl-oxy)-phenyl]-pyridin-2-yl-amine, 6-[2-methoxy-4-(pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-methoxy-4-(1-methyl-pyrrolidin-3-yl-oxy)-phenyl]-pyridin-2-yl-amine, 6-[2-methoxy-4-(2-methyl-2-aza-bicyclo[2.2.1]hept-5-yl-oxy)-phenyl]-pyrid-in-2-yl-amine, 6-[2-methoxy-4-(1-methyl-piperidin-4-yl-oxy)-phenyl]-pyridin-2-yl-amine, 6-[4-(1-ethyl-piperidin-4-yl-oxy)-2-methoxy-phenyl]-pyridin-2-yl-amine, 6-[5-allyl-2-methoxy-4-(1-methyl-pyrrolidin-3-yl-oxy)-phenyl]-pyr-idin-2-yl-amine, 6-[4-(2-dimethylamino-ethoxy)-2,6-dimethyl-phenyl]-pyridin-2-yl-amine, 6-[2,6-dimethyl-4-(3-piperidin-1-yl-propoxy)-phenyl]-pyridin-2-yl-amine, 6-[2,6-dimethyl-4-(2-pyrrolidin-1-yl-ethoxy)-phenyl]-pyridin-2-y-1-amine, 6-{2,6-dimethyl-4-[3-(4-methyl-piperazin-1-yl)-propoxy]-phenyl}-py-ridin-2-yl-amine, 6-[2,6-dimethyl-4-(2-morpholin-4-yl-ethoxy)-phenyl]-pyrid-in-2-yl-amine, 6-{4-[2-(benzyl-methyl-amino)-ethoxy]-2,6-dimethyl-phenyl}-p-yridin-2-yl-amine, 2-[4-(6-amino-pyridin-2-yl)-3,5-dimethyl-phenoxy]-acetam-ide 6-[4-(2-amino-ethoxy)-2,6-dimethyl-phenyl]-pyridin-2-yl-amine, 6-[2-isopropyl-4-(2-pyrrolidin-1-yl-ethoxy)-phenyl]-pyridin-2-yl-amine, 2-(2,5-dimethyl-pyrrolidin-1-yl)-6-[2-isopropyl-4-(2-pyrrolidin-1-yl-etho-xy)-phenyl]-pyridine 6-{4-[2-(3,5-dimethyl-piperidin-1-yl)-ethoxy]-2-isopr-opyl-phenyl}-pyridin-2-yl-amine, 6-[4-(2-dimethylamino-ethoxy)-2-isopropyl-phenyl]-pyridin-2-yl-amine, 6-[2-tert-butyl-4-(2-dimethylamino-ethoxy)-phen-yl]-pyridin-2-yl-amine, 6-[2-tert-butyl-4-(2-pyrrolidin-1-yl-ethoxy)-phenyl-]-pyridin-2-yl-amine, 6-[4-(2-pyrrolidinyl-ethoxy)-2,5-dimethyl-phenyl]-pyr-idin-2-yl-amine, 6-[4-(2-dimethylamino-ethoxy)-2,5-dimethyl-phenyl]-pyridin-2-yl-amine, 6-[4-(2-(4-phenethylpiperazin-1-yl)-ethoxy)-2,5-dimethyl-pheny-l]-pyridin-2-yl-amine, 6-[2-cyclopropyl-4-(2-dimethylamino-1-methyl-ethoxy)-phenyl]-pyridin-2-yl-amine, 6-[cyclobutyl-4-(2-dimethylamino-1-methyl-etho-xy)-phenyl]-pyridin-2-yl-amine, 6-[4-(allyloxy)-2-cyclobutyl-phenyl]-pyridi-n-2ylamine, 2-allyl-4-(6-amino-pyridin-2-yl)-3-cyclobutyl-phenol and 2-allyl-4-(6-amino-pyridin-2-yl)-5-cyclobutyl-phenol 4-(6-amino-pyridin-2yl)-5-cyclobutyl-2-propyl-phenol 4-(6-amino-pyridin-2yl)-3-cyclobutyl-2-propyl-phenol 6-[2-cyclobutyl-4-(2-dimethylamino-1-methyl-ethoxy)-5-propyl-phenyl]-pyri-din-2-yl-amine, 6-[2-cyclobutyl-4-(2-dimethylamino-1-methyl-ethoxy)-3-propy-1-phenyl]-pyridin-2-yl-amine, 6-[2-cyclobutyl-4-(2-dimethylamino-ethoxy)-5-propyl-phenyl]-pyridin-2-yl-amine, 6-[2-cyclobutyl-4-(2-dimethylamino-ethox-y)-3-propyl-phenyl]-pyridin-2-yl-amine, 6-[2-cyclobutyl-4-(1-methyl-pyrroli-din-3-yl-oxy)-5-propyl-phenyl]-pyridin-2-yl-amine, 6-[cyclobutyl-4-(1-methy-1-pyrrolidin-3-yl-oxy)-3-propyl-phenyl]-pyridin-2-yl-amine, 2-(4-benzyloxy-5-hydroxy-2-methoxy-phenyl)-6-(2,5-dimethyl-pyrrol-1-yl)-p-yridine 6-[4-(2-dimethylamino-ethoxy)-5-ethoxy-2-methoxy-phenyl]-pyridin-2-yl-amine, 6-[5-ethyl-2-methoxy-4-(1-methyl-piperidin-4-yl-oxy)-phenyl]-pyr-idin-2-yl-amine, 6-[5-ethyl-2-methoxy-4-(piperidin-4-yl-oxy)-phenyl]-pyridi-n-2-yl-amine, 6-[2,5-dimethoxy-4-(1-methyl-pyrrolidin-3-yl-oxy)-phenyl]-pyr-idin-2-yl-amine, 6-[4-(2-dimethylamino-ethoxy)-5-ethyl-2-methoxy-phenyl]-py-ridin-2-yl-amine.

Exemplary NMDA receptor antagonist include (+)-(1S,2S)-1-(4-hydroxy-phenyl)-2-(4-hydroxy-4-phenylpiperidino)-1-pro-panol, (1S,2S)-1-(4-hydroxy-3-methoxyphenyl)-2-(4-hydroxy-4-phenylpiperi-dino)-1-propanol, (3R,4S)-3-(4-(4-fluorophenyl)-4-hydroxypiperidin-1-yl-)-chroman-4,7-diol, (1R*,2R*)-1-(4-hydroxy-3-methylphenyl)-2-(4-(4-fluoro-phenyl)-4-hydroxypiperidin-1-yl)-propan-1-ol-mesylate or a pharmaceutically acceptable acid addition salt thereof.

Exemplary dopamine agonist include ropininole; L-dopa decarboxylase inhibitors such as carbidopa or benserazide, bromocriptine, dihydroergocryptine, etisulergine, AF-14, alaptide, pergolide, piribedil; dopamine D1 receptor agonists such as A-68939, A-77636, dihydrexine, and SKF-38393; dopamine D2 receptor agonists such as carbergoline, lisuride, N-0434, naxagolide, PD-118440, pramipexole, quinpirole and ropinirole; dopamine/β-adrenegeric receptor agonists such as DPDMS and dopexamine; dopamine/5-HT uptake inhibitor/5-HT-1A agonists such as roxindole; dopamine/opiate receptor agonists such as NIH-10494; α2-adrenergic antagonist/dopamine agonists such as terguride; α2-adrenergic antagonist/dopamine D2 agonists such as ergolines and talipexole; dopamine uptake inhibitors such as GBR-12909, GBR-13069, GYKI-52895, and NS-2141; monoamine oxidase-B inhibitors such as selegiline, N-(2-butyl)-N-methylpropargylamine, N-methyl-N-(2-pentyl)propargylamine, AGN-1133, ergot derivatives, lazabemide, LU-53439, MD-280040 and mofegiline; and COMT inhibitors such as CGP-28014.

Exemplary acetyl cholinesterase inhibitors include donepizil, 1-(2-methyl-1H-benzimida-zol-5-yl)-3-[1-(phenylmethyl)-4-piperidinyl]-1-propanone; 1-(2-phenyl-1H-benzimidazol-5-yl)-3-[1-(phenylmethyl)-4-piperidinyl]-1-pr-opanone; 1-(1-ethyl-2-methyl-1H-benzimidazol-5-yl)-3-[1-(phenylmethyl)-4-p-iperidinyl]-1-propanone; 1-(2-methyl-6-benzothiazolyl)-3-[1-(phenylmethyl)-4-piperidinyl]-1-propanone; 1-(2-methyl-6-benzothiazolyl)-3-[1-[(2-methyl-4-thiazolyl)methyl]-4-piperidinyl]-1-propanone; 1-(5-methyl-benzo[b]thie-n-2-yl)-3-[1-(phenylmethyl)-4-piperidinyl]-1-propanone; 1-(6-methyl-benzo[b]thien-2-yl)-3-[1-(phenylmethyl)-4-piperidinyl]-1-prop-anone; 1-(3,5-dimethyl-benzo[b]thien-2-yl)-3-[1-(phenylmethyl)-4-piperidin-yl]-1-propanone; 1-(benzo[b]thien-2-yl)-3-[1-(phenylmethyl)-4-piperidinyl]-1-propanone; 1-(benzofuran-2-yl)-3-[1-(phenylmethyl)-4-piperidinyl]-1-pro-panone; 1-(1-phenylsulfonyl-6-methyl-indol-2-yl)-3-[1-(phenylmethyl)-4-pip-eridinyl]-1-propanone; 1-(6-methyl-indol-2-yl)-3-[1-(phenylmethyl)-4-piper-idinyl]-1-propanone; 1-(1-phenylsulfonyl-5-amino-indol-2-yl)-3-[1-(phenylm-ethyl)-4-piperidinyl]-1-propanone; 1-(5-amino-indol-2-yl)-3-[1-(phenylmet-hyl)-4-piperidinyl]-1-propanone; and 1-(5-acetylamino-indol-2-yl)-3-[1-(ph-enylmethyl)-4-piperidinyl]-1-propanone. 1-(6-quinolyl)-3-[1-(phenylmethyl)-4-piperidinyl]-1-propanone; 1-(5-indolyl)-3-[1-(phenylmethyl)-4-piperidiny-1]-1-propanone; 1-(5-benzthienyl)-3-[1-(phenylmethyl)-4-piperidinyl]-1-pro-panone; 1-(6-quinazolyl)-3-[1-(phenylmethyl)-4-piperidinyl]-1-propanone; 1-(6-benzoxazolyl)-3-[1-(phenylmethyl)-4-piperidinyl]-1-propanone; 1-(5-benzofuranyl)-3-[1-(phenylmethyl)-4-piperidinyl]-1-propanone; 1-(5-methyl-benzimidazol-2-yl)-3-[1-(phenylmethyl)-4-piperidinyl]-1-propa-none; 1-(6-methyl-benzimidazol-2-yl)-3-[1-(phenylmethyl)-4-piperidinyl]-1-propanone; 1-(5-chloro-benzo[b]thien-2-yl)-3-[1-(phenylmethyl)-4-piperidin-yl]-1-propanone; 1-(5-azaindol-2-yl)-3-[1-(phenylmethyl)-4-piperidinyl]-1-p-ropanone; 1-(6-azabenzo[b]thien-2-yl)-3-[1-(phenylmethyl)-4-piperidinyl]-1-propanone; 1-(1H-2-oxo-pyrrolo[2′,3′,5,6]benzo[b]thieno-2-yl)-3-[1-(phenylmethyl)-4-piperidinyl]-1-propanone; 1-(6-methyl-benzothiazol-2$-yl)-3-[1-(phenylmethyl)-4-piperidinyl]-1-propanone; 1-(6-methoxy-indol-2-yl)-3-[1-(phenylmethyl)-4-piperidinyl]-1-propanone; 1-(6-methoxy-benzo[b]thien-2-yl)-3-[1-(phenylmethyl)-4-piperidinyl]-1-pro-panone; 1-(6-acetylamino-benzo[b]tbien-2-yl)-3-[1-(phenylmethyl)-4-piperid-inyl]-1-propanone; 1-(5-acetylamino-benzo[b]thien-2-yl)-3-[1-(phenylmethyl-)-4-piperidinyl]-1-propanone; 6-hydroxy-3-[2-[1-(phenylmethyl)-4-piperidin-yl]ethyl]-1,2-benzisoxazole; 5-methyl-3-[2-[1-(phenylmethyl)-4-piperidinyl-]ethyl]-1,2-benzisoxazole; 6-methoxy-3[2-[1(phenylmethyl)-4-piperidinyl]et-hyl]-1,2-benzisoxazole; 6-acetamide-3-[2-[1-(phenylmethyl)-4-piperidinyl]-ethyl]-1,2-benzisoxazole; 6-amino-3-[2-[1-(phenymethyl)-4-piperidinyl]ethy-1]-1,2-benzisoxazole; 6-(4-morpholinyl)-3-[2-[1-(phenylmethyl)-4-piperidin-yl]ethyl]-1,2-benzisoxazole; 5,7-dihydro-3-[2-[1-(phenylmethyl)-4-piperidi-nyl]ethyl]-6H-pyrrolo[4,5-f]-1,2-benzisoxazol-6-one; 3-[2-[1-(phenylmethyl)-4-piperidinyl]ethyl]-1,2-benzisothiazole; 3-[2-[1-(phenylmethyl)-4-piperidinyl]ethenyl]-1,2-benzisoxazole; 6-phenylamino-3-[2-[1-(phenylmethyl)-4-piperidinyl]ethyl]-1,2,-benzisoxaz-ole; 6-(2-thiazoly)-3-[2-[1-(phenylmethyl)-4-piperidinyl]ethyl]-1,2-benzis-oxazole; 6-(2-oxazolyl)-3-[2-[1-(phenylmethyl)-4-piperidinyl]ethyl]-1,2-be-nzisoxazole; 6-pyrrolidinyl-3-[2-[1-(phenylmethyl)-4-piperidinyl]ethyl]-1,-2-benzisoxazole; 5,7-dihydro-5,5-dimethyl-3-[2-[1-(phenylmethyl)-4-piperid-inyl]ethyl]-6H-pyrrolo[4,5-f]-1,2-benzisoxazole-6-one; 6,8-dihydro-3-[2-[1-(phenylmethyl)-4-piperidinyl]ethyl]-7H-pyrrolo[5,4-g]-1,2-benzisoxazole-7-one; 3-[2-[1-(phenylmethyl)-4-piperidinyl]ethyl]-5,6,-8-trihydro-7H-isoxazolo[4,5-g]-quinolin-7-one; 1-benzyl-4-((5,6-dimethoxy-1-indanon)-2-yl)methylpiperidine, 1-benzyl-4-((5,6-dimethoxy-1-indanon)-2-ylidenyl)methylpiperidine, 1-benzyl-4-((5-methoxy-1-indanon)-2-yl)methylp-iperidine, 1-benzyl-4-((5,6-diethoxy-1-indanon)-2-yl)methylpiperidine, 1-benzyl-4-((5,6-methnylenedioxy-1-indanon)-2-yl)methylpiperidine, 1-(m-nitrobenzyl)-4-((5,6-dimethoxy-1-indanon)-2-yl)methylpiperidine, 1-cyclohexymethyl-4-((5,6-dimethoxy-1-indanon)-2-yl)methylpiperidine, 1-(m-florobenzyl)-4-((5,6-dimethoxy-1-indanon)-2-yl)methylpiperidine, 1-benzyl-4-((5,6-dimethoxy-1-indanon)-2-yl)propylpiperidine, and 1-benzyl-4-((5-isopropoxy-6-methoxy-1-indanon)-2-yl)methylpiperidine.

Exemplary calcium channel antagonists include diltiazem, omega-conotoxin GVIA, methoxyverapamil, amlodipine, felodipine, lacidipine, and mibefradil.

Exemplary GABA-A receptor modulators include clomethiazole; IDDB; gaboxadol (4,5,6,7-tetrahydroisoxazolo[5,4-c]pyridin-3-ol); ganaxolone (3a-hydroxy-3β-methyl-5α-pregnan-20-one); fengabine (2-[(butylimino)-(2-chlorophenyl)methyl]-4-chlorophenol); 2-(4-methoxyphenyl)-2,5,6,7,8,9-hexahydro-pyrazolo[4,3-c]cinnolin-3-one; 7-cyclobutyl-6-(2-methyl-2H-1,2,4-triazol-3-ylmethoxy)-3-phenyl-1,2,4-triazolo[4,3-b]pyridazine; (3-fluoro-4-methylphenyl)-N-({-1-[(2-methylphenyl)methyl]-benzimidazol-2-yl}methyl)-N-pentylcarboxamide; and 3-(aminomethyl)-5-methylhexanoic acid.

Exemplary potassium channel openers include diazoxide, flupirtine, pinacidil, levcromakalim, rilmakalim, chromakalim, PCO-400 and SKP-450 (2-[2″(1″,3″-dioxolone)-2-methyl]-4-(2′-oxo-1′-pyrrolidinyl)-6-nitro-2H-1-benzopyra-n).

Exemplary AMPA/kainate receptor antagonists include 6-cyano-7-nitroquinoxalin-2,3-di-one (CNQX); 6-nitro-7-sulphamoylbenzo[f]quinoxaline-2,3-dione (NBQX); 6,7-dinitroquinoxaline-2,3-dione (DNQX); 1-(4-aminophenyl)-4-methyl-7,8-m-ethylenedioxy-5H-2,3-benzodiazepine hydrochloride; and 2,3-dihydroxy-6-nitro-7-sulfamoylbenzo-[f]quinoxaline.

Exemplary sodium channel antagonists include ajmaline, procainamide, flecainide and riluzole.

Exemplary matrix-metalloprotease inhibitors include 4-[4-(4-fluorophenoxy)benzenesulfon-ylamino]tetrahydropyran-4-carboxylic acid hydroxyamide; 5-Methyl-5-(4-(4′-fluorophenoxy)-phenoxy)-pyrimidine-2,4,6-trione; 5-n-Butyl-5-(4-(4′-fluorophenoxy)-phenoxy)-pyrimidine-2,4,6-trione and prinomistat.

Poly(ADP ribose) polymerase (PARP) is an abundant nuclear enzyme which is activated by DNA strand single breaks to synthesize poly (ADP ribose) from NAD. Under normal conditions, PARP is involved in base excision repair caused by oxidative stress via the activation and recruitment of DNA repair enzymes in the nucleus. Thus, PARP plays a role in cell necrosis and DNA repair. PARP also participates in regulating cytokine expression that mediates inflammation. Under conditions where DNA damage is excessive (such as by acute excessive exposure to a pathological insult), PARP is over-activated, resulting in cell-based energetic failure characterized by NAD depletion and leading to ATP consumption, cellular necrosis, tissue injury, and organ damage/failure. PARP is thought to contribute to neurodegeneration by depleting nicotinamide adenine dinucleotide (NAD+) which then reduces adenosine triphosphate (ATP; Cosi and Marien, Ann. N.Y. Acad. Sci., 890:227, 1999) contributing to cell death which can be prevented by PARP inhibitors. Exemplory PARP inhibitors can be found in Southan and Szabo, Current Medicinal Chemistry, 10:321, 2003.

Exemplary inhibitors of p38 MAP kinase and c-jun-N-terminal kinases include pyridyl imidazoles, such as PD 169316, isomeric PD 169316, SB 203580, SB 202190, SB 220026, and RWJ 67657. Others are described in U.S. Pat. No. 6,288,089, and incorporated by reference herein.

In an exemplary embodiment, a combination therapy for treating or preventing MS comprises a high dose of one or more sirtuin activating compounds and one or more of Avonex® (interferon beta-1a), Tysabri® (natalizumab), or Fumaderm® (BG-12/Oral Fumarate).

In another embodiment, a combination therapy for treating or preventing diabetic neuropathy or conditions associated therewith comprises a high dose of one or more sirtuin activating compounds and one or more of tricyclic antidepressants (TCAs) (including, for example, imipramine, amytriptyline, desipramine and nortriptyline), serotonin reuptake inhibitors (SSRIs) (including, for example, fluoxetine, paroxetine, sertralene, and citalopram) and antiepileptic drugs (AEDs) (including, for example, gabapentin, carbamazepine, and topimirate).

In another embodiment, the invention provides a method for treating or preventing a polyglutamine disease using a combination comprising a high dose of one or more sirtuin activating compounds and at least one HDAC I/II inhibitor. Examples of HDAC I/II inhibitors include hydroxamic acids, cyclic peptides, benzamides, short-chain fatty acids, and depudecin.

Examples of hydroxamic acids and hydroxamic acid derivatives, but are not limited to, trichostatin A (TSA), suberoylanilide hydroxamic acid (SAHA), oxamflatin, suberic bishydroxamic acid (SBHA), m-carboxy-cinnamic acid bishydroxamic acid (CBHA), valproic acid and pyroxamide. TSA was isolated as an antiftingi antibiotic (Tsuji et al (1976) J. Antibiot (Tokyo) 29:1-6) and found to be a potent inhibitor of mammalian HDAC (Yoshida et al. (1990) J. Biol. Chem. 265:17174-17179). The finding that TSA-resistant cell lines have an altered HDAC evidences that this enzyme is an important target for TSA. Other hydroxamic acid-based HDAC inhibitors, SAHA, SBHA, and CBHA are synthetic compounds that are able to inhibit HDAC at micromolar concentration or lower in vitro or in vivo. Glick et al. (1999) Cancer Res. 59:4392-4399. These hydroxamic acid-based HDAC inhibitors all possess an essential structural feature: a polar hydroxamic terminal linked through a hydrophobic methylene spacer (e.g. 6 carbon at length) to another polar site which is attached to a terminal hydrophobic moiety (e.g., benzene ring). Compounds developed having such essential features also fall within the scope of the hydroxamic acids that may be used as HDAC inhibitors.

Cyclic peptides used as HDAC inhibitors are mainly cyclic tetrapeptides. Examples of cyclic peptides include, but are not limited to, trapoxin A, apicidin and depsipeptide. Trapoxin A is a cyclic tetrapeptide that contains a 2-amino-8-oxo-9,10-epoxy-decanoyl (AOE) moiety. Kijima et al. (1993) J. Biol. Chem. 268:22429-22435. Apicidin is a fungal metabolite that exhibits potent, broad-spectrum antiprotozoal activitity and inhibits HDAC activity at nanomolar concentrations. Darkin-Rattray et al. (1996) Proc. Natl. Acad. Sci. USA. 93; 13143-13147. Depsipeptide is isolated from Chromobacterium violaceum, and has been shown to inhibit HDAC activity at micromolar concentrations.

Examples of benzamides include but are not limited to MS-27-275. Saito et al. (1990) Proc. Natl. Acad. Sci. USA. 96:4592-4597. Examples of short-chain fatty acids include but are not limited to butyrates (e.g., butyric acid, arginine butyrate and phenylbutyrate (PB)). Newmark et al. (1994) Cancer Lett. 78:1-5; and Carducci et al. (1997) Anticancer Res. 17:3972-3973. In addition, depudecin which has been shown to inhibit HDAC at micromolar concentrations (Kwon et al. (1998) Proc. Natl. Acad. Sci. USA. 95:3356-3361) also falls within the scope of histone deacetylase inhibitor as described herein.

Blood Coagulation Disorders

In other aspects, a high dose of a sirtuin activating compound can be used to treat or prevent blood coagulation disorders (or hemostatic disorders). As used interchangeably herein, the terms “hemostasis”, “blood coagulation,” and “blood clotting” refer to the control of bleeding, including the physiological properties of vasoconstriction and coagulation. Blood coagulation assists in maintaining the integrity of mammalian circulation after injury, inflammation, disease, congenital defect, dysfunction or other disruption. After initiation of clotting, blood coagulation proceeds through the sequential activation of certain plasma proenzymes to their enzyme forms (see, for example, Coleman, R. W. et al. (eds.) Hemostasis and Thrombosis, Second Edition, (1987)). These plasma glycoproteins, including Factor XII, Factor XI, Factor IX, Factor X, Factor VII, and prothrombin, are zymogens of serine proteases. Most of these blood clotting enzymes are effective on a physiological scale only when assembled in complexes on membrane surfaces with protein cofactors such as Factor VIII and Factor V. Other blood factors modulate and localize clot formation, or dissolve blood clots. Activated protein C is a specific enzyme that inactivates procoagulant components. Calcium ions are involved in many of the component reactions. Blood coagulation follows either the intrinsic pathway, where all of the protein components are present in blood, or the extrinsic pathway, where the cell-membrane protein tissue factor plays a critical role. Clot formation occurs when fibrinogen is cleaved by thrombin to form fibrin. Blood clots are composed of activated platelets and fibrin.

Further, the formation of blood clots does not only limit bleeding in case of an injury (hemostasis), but may lead to serious organ damage and death in the context of atherosclerotic diseases by occlusion of an important artery or vein. Thrombosis is thus blood clot formation at the wrong time and place. It involves a cascade of complicated and regulated biochemical reactions between circulating blood proteins (coagulation factors), blood cells (in particular platelets), and elements of an injured vessel wall.

Accordingly, the present invention provides anticoagulation and antithrombotic treatments aiming at inhibiting the formation of blood clots in order to prevent or treat blood coagulation disorders, such as myocardial infarction, stroke, loss of a limb by peripheral artery disease or pulmonary embolism.

As used interchangeably herein, “modulating or modulation of hemostasis” and “regulating or regulation of hemostasis” includes the induction (e.g., stimulation or increase) of hemostasis, as well as the inhibition (e.g., reduction or decrease) of hemostasis.

In one aspect, the invention provides a method for reducing or inhibiting hemostasis in a subject by administering a high dose of a sirtuin activating compound. The compositions and methods disclosed herein are useful for the treatment or prevention of thrombotic disorders. As used herein, the term “thrombotic disorder” includes any disorder or condition characterized by excessive or unwanted coagulation or hemostatic activity, or a hypercoagulable state. Thrombotic disorders include diseases or disorders involving platelet adhesion and thrombus formation, and may manifest as an increased propensity to form thromboses, e.g., an increased number of thromboses, thrombosis at an early age, a familial tendency towards thrombosis, and thrombosis at unusual sites. Examples of thrombotic disorders include, but are not limited to, thromboembolism, deep vein thrombosis, pulmonary embolism, stroke, myocardial infarction, miscarriage, thrombophilia associated with anti-thrombin III deficiency, protein C deficiency, protein S deficiency, resistance to activated protein C, dysfibrinogenemia, fibrinolytic disorders, homocystinuria, pregnancy, inflammatory disorders, myeloproliferative disorders, arteriosclerosis, angina, e.g., unstable angina, disseminated intravascular coagulation, thrombotic thrombocytopenic purpura, cancer metastasis, sickle cell disease, glomerular nephritis, and drug induced thrombocytopenia (including, for example, heparin induced thrombocytopenia). In addition, a high dose of a sirtuin activating compound may be administered to prevent thrombotic events or to prevent re-occlusion during or after therapeutic clot lysis or procedures such as angioplasty or surgery.

In another embodiment, a combination drug regimen may include drugs or compounds for the treatment or prevention of blood coagulation disorders or secondary conditions associated with these conditions. Thus, a combination drug regimen may include a high dose of one or more sirtuin activating compounds and one or more anti-coagulation or anti-thrombosis agents. For example, a high dose of one or more sirtuin activating compounds can be combined with an effective amount of one or more of: aspirin, heparin, and oral Warfarin that inhibits Vit K-dependent factors, low molecular weight heparins that inhibit factors X and II, thrombin inhibitors, inhibitors of platelet GP IIbIIIa receptors, inhibitors of tissue factor (TF), inhibitors of human von Willebrand factor, inhibitors of one or more factors involved in hemostasis (in particular in the coagulation cascade). In addition, a high dose of one or more sirtuin activating compounds can be combined with thrombolytic agents, such as t-PA, streptokinase, reptilase, TNK-t-PA, and staphylokinase.

Inflammatory Diseases

In other aspects, a high dose of one or more sirtuin activating compounds can be used to treat or prevent a disease or disorder associated with inflammation. A high dose of one or more sirtuin activating compounds may be administered prior to the onset of, at, or after the initiation of inflammation. When used prophylactically, the compounds are preferably provided in advance of any inflammatory response or symptom. Administration of the high dose of the sirtuin activating compound may prevent or attenuate inflammatory responses or symptoms.

Exemplary inflammatory conditions include, for example, multiple sclerosis, rheumatoid arthritis, psoriatic arthritis, degenerative joint disease, spondouloarthropathies, gouty arthritis, systemic lupus erythematosus, juvenile arthritis, rheumatoid arthritis, osteoarthritis, osteoporosis, diabetes (e.g., insulin dependent diabetes mellitus or juvenile onset diabetes), menstrual cramps, cystic fibrosis, inflammatory bowel disease, irritable bowel syndrome, Crohn's disease, mucous colitis, ulcerative colitis, gastritis, esophagitis, pancreatitis, peritonitis, Alzheimer's disease, shock, ankylosing spondylitis, gastritis, conjunctivitis, pancreatis (acute or chronic), multiple organ injury syndrome (e.g., secondary to septicemia or trauma), myocardial infarction, atherosclerosis, stroke, reperfusion injury (e.g., due to cardiopulmonary bypass or kidney dialysis), acute glomerulonephritis, vasculitis, thermal injury (i.e., sunburn), necrotizing enterocolitis, granulocyte transfusion associated syndrome, and/or Sjogren's syndrome. Exemplary inflammatory conditions of the skin include, for example, eczema, atopic dermatitis, contact dermatitis, urticaria, schleroderma, psoriasis, and dermatosis with acute inflammatory components.

In another embodiment, a high dose of one or more sirtuin activating compounds may be used to treat or prevent allergies and respiratory conditions, including asthma, bronchitis, pulmonary fibrosis, allergic rhinitis, oxygen toxicity, emphysema, chronic bronchitis, acute respiratory distress syndrome, and any chronic obstructive pulmonary disease (COPD). The high dose of one or more sirtuin activating compounds may be used to treat chronic hepatitis infection, including hepatitis B and hepatitis C.

Additionally, a high dose of one or more sirtuin activating compounds may be used to treat autoimmune diseases and/or inflammation associated with autoimmune diseases such as organ-tissue autoimmune diseases (e.g., Raynaud's syndrome), scleroderma, myasthenia gravis, transplant rejection, endotoxin shock, sepsis, psoriasis, eczema, dermatitis, multiple sclerosis, autoimmune thyroiditis, uveitis, systemic lupus erythematosis, Addison's disease, autoimmune polyglandular disease (also known as autoimmune polyglandular syndrome), and Grave's disease.

In certain embodiments, a high dose of one or more sirtuin activating compounds may be taken alone or in combination with other compounds useful for treating or preventing inflammation. Exemplary anti-inflammatory agents include, for example, steroids (e.g., cortisol, cortisone, fludrocortisone, prednisone, 6α-methylprednisone, triamcinolone, betamethasone or dexamethasone) and nonsteroidal antiinflammatory drugs (NSAIDS) (e.g., aspirin, acetaminophen, tolmetin, ibuprofen, mefenamic acid, piroxicam, nabumetone, rofecoxib, celecoxib, etodolac or nimesulide). In another embodiment, the other therapeutic agent is an antibiotic (e.g., vancomycin, penicillin, amoxicillin, ampicillin, cefotaxime, ceftriaxone, cefixime, rifampinmetronidazole, doxycycline or streptomycin). In another embodiment, the other therapeutic agent is a PDE4 inhibitor (e.g., roflumilast or rolipram). In another embodiment, the other therapeutic agent is an antihistamine (e.g., cyclizine, hydroxyzine, promethazine or diphenhydramine). In another embodiment, the other therapeutic agent is an anti-malarial (e.g., artemisinin, artemether, artsunate, chloroquine phosphate, mefloquine hydrochloride, doxycycline hyclate, proguanil hydrochloride, atovaquone or halofantrine). In one embodiment, the other therapeutic agent is drotrecogin alfa.

Further examples of anti-inflammatory agents include, for example, aceclofenac, acemetacin, e-acetamidocaproic acid, acetaminophen, acetaminosalol, acetanilide, acetylsalicylic acid, S-adenosylmethionine, alclofenac, alclometasone, alfentanil, algestone, allylprodine, alminoprofen, aloxiprin, alphaprodine, aluminum bis(acetylsalicylate), amcinonide, amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antrafenine, apazone, beclomethasone, bendazac, benorylate, benoxaprofen, benzpiperylon, benzydamine, benzylmorphine, bermoprofen, betamethasone, betamethasone-17-valerate, bezitramide, α-bisabolol, bromfenac, p-bromoacetanilide, 5-bromosalicylic acid acetate, bromosaligenin, bucetin, bucloxic acid, bucolome, budesonide, bufexamac, bumadizon, buprenorphine, butacetin, butibufen, butorphanol, carbamazepine, carbiphene, carprofen, carsalam, chlorobutanol, chloroprednisone, chlorthenoxazin, choline salicylate, cinchophen, cinmetacin, ciramadol, clidanac, clobetasol, clocortolone, clometacin, clonitazene, clonixin, clopirac, cloprednol, clove, codeine, codeine methyl bromide, codeine phosphate, codeine sulfate, cortisone, cortivazol, cropropamide, crotethamide, cyclazocine, deflazacort, dehydrotestosterone, desomorphine, desonide, desoximetasone, dexamethasone, dexamethasone-21-isonicotinate, dexoxadrol, dextromoramide, dextropropoxyphene, deoxycorticosterone, dezocine, diampromide, diamorphone, diclofenac, difenamizole, difenpiramide, diflorasone, diflucortolone, diflunisal, difluprednate, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, enoxolone, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, fluazacort, flucloronide, flufenamic acid, flumethasone, flunisolide, flunixin, flunoxaprofen, fluocinolone acetonide, fluocinonide, fluocinolone acetonide, fluocortin butyl, fluocortolone, fluoresone, fluorometholone, fluperolone, flupirtine, fluprednidene, fluprednisolone, fluproquazone, flurandrenolide, flurbiprofen, fluticasone, formocortal, fosfosal, gentisic acid, glafenine, glucametacin, glycol salicylate, guaiazulene, halcinonide, halobetasol, halometasone, haloprednone, heroin, hydrocodone, hydrocortamate, hydrocortisone, hydrocortisone acetate, hydrocortisone succinate, hydrocortisone hemisuccinate, hydrocortisone 21-lysinate, hydrocortisone cypionate, hydromorphone, hydroxypethidine, ibufenac, ibuprofen, ibuproxam, imidazole salicylate, indomethacin, indoprofen, isofezolac, isoflupredone, isoflupredone acetate, isoladol, isomethadone, isonixin, isoxepac, isoxicam, ketobemidone, ketoprofen, ketorolac, p-lactophenetide, lefetamine, levallorphan, levorphanol, levophenacyl-morphan, lofentanil, lonazolac, lornoxicam, loxoprofen, lysine acetylsalicylate, mazipredone, meclofenamic acid, medrysone, mefenamic acid, meloxicam, meperidine, meprednisone, meptazinol, mesalamine, metazocine, methadone, methotrimeprazine, methylprednisolone, methylprednisolone acetate, methylprednisolone sodium succinate, methylprednisolone suleptnate, metiazinic acid, metofoline, metopon, mofebutazone, mofezolac, mometasone, morazone, morphine, morphine hydrochloride, morphine sulfate, morpholine salicylate, myrophine, nabumetone, nalbuphine, nalorphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paramethasone, paranyline, parsalmide, pentazocine, perisoxal, phenacetin, phenadoxone, phenazocine, phenazopyridine hydrochloride, phenocoll, phenoperidine, phenopyrazone, phenomorphan, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, prednicarbate, prednisolone, prednisone, prednival, prednylidene, proglumetacin, proheptazine, promedol, propacetamol, properidine, propiram, propoxyphene, propyphenazone, proquazone, protizinic acid, proxazole, ramifenazone, remifentanil, rimazolium metilsulfate, salacetamide, salicin, salicylamide, salicylamide o-acetic acid, salicylic acid, salicylsulfuric acid, salsalate, salverine, simetride, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tixocortol, tolfenamic acid, tolmetin, tramadol, triamcinolone, triamcinolone acetonide, tropesin, viminol, xenbucin, ximoprofen, zaltoprofen and zomepirac.

In an exemplary embodiment, a high dose of one or more sirtuin activating compounds may be administered with a selective COX-2 inhibitor for treating or preventing inflammation. Exemplary selective COX-2 inhibitors include, for example, deracoxib, parecoxib, celecoxib, valdecoxib, rofecoxib, etoricoxib, lumiracoxib, 2-(3,5-difluorophenyl)-3-[4-(methylsulfonyl)phenyl]-2-cyclopenten-1-one, (S)-6,8-dichloro-2-(triflu-oromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 4-[5-(4-fluorophenyl)-3-(trifluoromethyl)-1H-pyrazol-1-yl]benzenesulfonamide, tert-butyl 1 benzyl-4-[(4-oxopiperidin-1-yl}sulfonyl]piperidine-4-carboxylate, 4-[5-(phenyl)-3-(trifluoromethyl)-1H-pyrazol-1-yl]benzenesulfonamide, salts and prodrugs thereof.

Flushing

In another aspect, a high dose of one or more sirtuin activating compounds may be used for reducing the incidence or severity of flushing and/or hot flashes which are symptoms of a disorder. For instance, the subject method includes the use of a high dose of one or more sirtuin activating compounds, alone or in combination with other agents, for reducing incidence or severity of flushing and/or hot flashes in cancer patients. In other embodiments, the method provides for the use of a high dose of one or more sirtuin activating compounds to reduce the incidence or severity of flushing and/or hot flashes in menopausal and post-menopausal woman.

In another aspect, a high dose of one or more sirtuin activating compounds may be used as a therapy for reducing the incidence or severity of flushing and/or hot flashes which are side-effects of another drug therapy, e.g., drug-induced flushing. In certain embodiments, a method for treating and/or preventing drug-induced flushing comprises administering to a patient in need thereof a formulation comprising at least one flushing inducing compound and a high dose of at least one sirtuin activating compound. In other embodiments, a method for treating drug induced flushing comprises separately administering one or more compounds that induce flushing and a high dose of one or more sirtuin activating compounds, e.g., wherein the sirtuin-modulating compound and flushing inducing agent have not been formulated in the same compositions. When using separate formulations, the sirtuin-modulating compound may be administered (1) at the same as administration of the flushing inducing agent, (2) intermittently with the flushing inducing agent, (3) staggered relative to administration of the flushing inducing agent, (4) prior to administration of the flushing inducing agent, (5) subsequent to administration of the flushing inducing agent, and (6) various combination thereof. Exemplary flushing inducing agents include, for example, niacin, faloxifene, antidepressants, anti-psychotics, chemotherapeutics, calcium channel blockers, and antibiotics.

In one embodiment, a high dose of one or more sirtuin activating compounds may be used to reduce flushing side effects of a vasodilator or an antilipemic agent (including anticholesteremic agents and lipotropic agents). In an exemplary embodiment, a high dose of one or more sirtuin activating compounds may be used to reduce flushing associated with the administration of niacin.

Nicotinic acid, 3-pyridinecarboxylic acid or niacin, is an antilipidemic agent that is marketed under, for example, the trade names Nicolar®, SloNiacin®, Nicobid® and Time Release Niacin®. Nicotinic acid has been used for many years in the treatment of lipidemic disorders such as hyperlipidemia, hypercholesterolemia and atherosclerosis. This compound has long been known to exhibit the beneficial effects of reducing total cholesterol, low density lipoproteins or “LDL cholesterol,” triglycerides and apolipoprotein a (Lp(a)) in the human body, while increasing desirable high density lipoproteins or “HDL cholesterol”.

Typical doses range from about 1 gram to about 3 grams daily. Nicotinic acid is normally administered two to four times per day after meals, depending upon the dosage form selected. Nicotinic acid is currently commercially available in two dosage forms. One dosage form is an immediate or rapid release tablet which should be administered three or four times per day. Immediate release (“IR”) nicotinic acid formulations generally release nearly all of their nicotinic acid within about 30 to 60 minutes following ingestion. The other dosage form is a sustained release form which is suitable for administration two to four times per day. In contrast to IR formulations, sustained release (“SR”) nicotinic acid formulations are designed to release significant quantities of drug for absorption into the blood stream over specific timed intervals in order to maintain therapeutic levels of nicotinic acid over an extended period such as 12 or 24 hours after ingestion.

As used herein, the term “nicotinic acid” is meant to encompass nicotinic acid or a compound other than nicotinic acid itself which the body metabolizes into nicotinic acid, thus producing essentially the same effect as nicotinic acid. Exemplary compounds that produce an effect similar to that of nicotinic acid include, for example, nicotinyl alcohol tartrate, d-glucitol hexanicotinate, aluminum nicotinate, niceritrol and d,l-alpha-tocopheryl nicotinate. Each such compound will be collectively referred to herein as “nicotinic acid.”

In another embodiment, the invention provides a method for treating and/or preventing hyperlipidemia with reduced flushing side effects. The method comprises the steps of administering to a subject in need thereof a therapeutically effective amount of nicotinic acid and a high dose of one or more sirtuin activating compounds. In an exemplary embodiment, the nicotinic acid and/or sirtuin-modulating compound may be administered nocturnally.

In another representative embodiment, the method involves the use of a high dose of one or more sirtuin activating compounds to reduce flushing side effects of raloxifene. Raloxifene acts like estrogen in certain places in the body, but is not a hormone. It helps prevent osteoporosis in women who have reached menopause. Osteoporosis causes bones to gradually grow thin, fragile, and more likely to break. Evista slows down the loss of bone mass that occurs with menopause, lowering the risk of spine fractures due to osteoporosis. A common side effect of raloxifene is hot flashes (sweating and flushing). This can be uncomfortable for women who already have hot flashes due to menopause.

In another representative embodiment, the method involves the use of a high dose of one or more sirtuin activating compounds to reduce flushing side effects of antidepressants or anti-psychotic agent. For instance, a high dose of one or more sirtuin activating compounds can be used in conjunction (administered separately or together) with a serotonin reuptake inhibitor, a 5HT2 receptor antagonist, an anticonvulsant, a norepinephrine reuptake inhibitor, an a-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT1A receptor antagonist, a 5HT1D receptor antagonist, a CRF antagonist, a monoamine oxidase inhibitor, or a sedative-hypnotic drug.

In certain embodiments, a high dose of one or more sirtuin activating compounds may be used as part of a treatment with a serotonin reuptake inhibitor (SRI) to reduce flushing. In certain preferred embodiments, the SR₁ is a selective serotonin reuptake inhibitor (SSRI), such as a fluoxetinoid (fluoxetine, norfluoxetine) or a nefazodonoid (nefazodone, hydroxynefazodone, oxonefazodone). Other exemplary SSRI's include duloxetine, venlafaxine, milnacipran, citalopram, fluvoxamine, paroxetine and sertraline. A high dose of one or more sirtuin activating compounds can also be used as part of a treatment with sedative-hypnotic drug, such as selected from the group consisting of a benzodiazepine (such as alprazolam, chlordiazepoxide, clonazepam, chlorazepate, clobazam, diazepam, halazepam, lorazepam, oxazepam and prazepam), zolpidem, and barbiturates. In still other embodiments, a high dose of one or more sirtuin activating compounds may be used as part of a treatment with a 5-HT1A receptor partial agonist, such as selected from the group consisting of buspirone, flesinoxan, gepirone and ipsapirone. A high dose of one or more sirtuin activating compounds can also used as part of a treatment with a norepinephrine reuptake inhibitor, such as selected from tertiary amine tricyclics and secondary amine tricyclics. Exemplary tertiary amine tricyclic include amitriptyline, clomipramine, doxepin, imipramine and trimipramine. Exemplary secondary amine tricyclic include amoxapine, desipramine, maprotiline, nortriptyline and protriptyline. In certain embodiments, a high dose of one or more sirtuin activating compounds may be used as part of a treatment with a monoamine oxidase inhibitor, such as selected from the group consisting of isocarboxazid, phenelzine, tranylcypromine, selegiline and moclobemide.

In still another representative embodiment, a high dose of one or more sirtuin activating compounds may be used to reduce flushing side effects of chemotherapeutic agents, such as cyclophosphamide, tamoxifen.

In another embodiment, a high dose of one or more sirtuin activating compounds may be used to reduce flushing side effects of calcium channel blockers, such as amlodipine.

In another embodiment, a high dose of one or more sirtuin activating compounds may be used to reduce flushing side effects of antibiotics. For example, a high dose of one or more sirtuin activating compounds can be used in combination with levofloxacin. Levofloxacin is used to treat infections of the sinuses, skin, lungs, ears, airways, bones, and joints caused by susceptible bacteria. Levofloxacin also is frequently used to treat urinary infections, including those resistant to other antibiotics, as well as prostatitis. Levofloxacin is effective in treating infectious diarrheas caused by E. coli, campylobacter jejuni, and shigella bacteria. Levofloxacin also can be used to treat various obstetric infections, including mastitis.

Ocular Disorders

One aspect of the present invention is a method for inhibiting, reducing or otherwise treating vision impairment by administering to a patient a high dose of one or more sirtuin activating compounds.

In certain aspects of the invention, the vision impairment is caused by damage to the optic nerve or central nervous system. In particular embodiments, optic nerve damage is caused by high intraocular pressure, such as that created by glaucoma. In other particular embodiments, optic nerve damage is caused by swelling of the nerve, which is often associated with an infection or an immune (e.g., autoimmune) response such as in optic neuritis.

Glaucoma describes a group of disorders which are associated with a visual field defect, cupping of the optic disc, and optic nerve damage. These are commonly referred to as glaucomatous optic neuropathies. Most glaucomas are usually, but not always, associated with a rise in intraocular pressure. Exemplary forms of glaucoma include Glaucoma and Penetrating Keratoplasty, Acute Angle Closure, Chronic Angle Closure, Chronic Open Angle, Angle Recession, Aphakic and Pseudophakic, Drug-Induced, Hyphema, Intraocular Tumors, Juvenile, Lens-Particle, Low Tension, Malignant, Neovascular, Phacolytic, Phacomorphic, Pigmentary, Plateau Iris, Primary Congenital, Primary Open Angle, Pseudoexfoliation, Secondary Congenital, Adult Suspect, Unilateral, Uveitic, Ocular Hypertension, Ocular Hypotony, Posner-Schlossman Syndrome and Scleral Expansion Procedure in Ocular Hypertension & Primary Open-angle Glaucoma.

Intraocular pressure can also be increased by various surgical procedures, such as phacoemulsification (i.e., cataract surgery) and implanation of structures such as an artificial lens. In addition, spinal surgeries in particular, or any surgery in which the patient is prone for an extended period of time can lead to increased interoccular pressure.

Optic neuritis (ON) is inflammation of the optic nerve and causes acute loss of vision. It is highly associated with multiple sclerosis (MS) as 15-25% of MS patients initially present with ON, and 50-75% of ON patients are diagnosed with MS. ON is also associated with infection (e.g., viral infection, meningitis, syphilis), inflammation (e.g., from a vaccine), infiltration and ischemia.

Another condition leading to optic nerve damage is anterior ischemic optic neuropathy (AION). There are two types of AION. Arteritic AION is due to giant cell arteritis (vasculitis) and leads to acute vision loss. Non-arteritic AION encompasses all cases of ischemic optic neuropathy other than those due to giant cell arteritis. The pathophysiology of AION is unclear although it appears to incorporate both inflammatory and ischemic mechanisms.

Other damage to the optic nerve is typically associated with demyleination, inflammation, ischemia, toxins, or trauma to the optic nerve. Exemplary conditions where the optic nerve is damaged include Demyelinating Optic Neuropathy (Optic Neuritis, Retrobulbar Optic Neuritis), Optic Nerve Sheath Meningioma, Adult Optic Neuritis, Childhood Optic Neuritis, Anterior Ischemic Optic Neuropathy, Posterior Ischemic Optic Neuropathy, Compressive Optic Neuropathy, Papilledema, Pseudopapilledema and Toxic/Nutritional Optic Neuropathy.

Other neurological conditions associated with vision loss, albeit not directly associated with damage to the optic nerve, include Amblyopia, Bells Palsy, Chronic Progressive External Ophthalmoplegia, Multiple Sclerosis, Pseudotumor Cerebri and Trigeminal Neuralgia.

In certain aspects of the invention, the vision impairment is caused by retinal damage. In particular embodiments, retinal damage is caused by disturbances in blood flow to the eye (e.g., arteriosclerosis, vasculitis). In particular embodiments, retinal damage is caused by disrupton of the macula (e.g., exudative or non-exudative macular degeneration).

Exemplary retinal diseases include Exudative Age Related Macular Degeneration, Nonexudative Age Related Macular Degeneration, Retinal Electronic Prosthesis and RPE Transplantation Age Related Macular Degeneration, Acute Multifocal Placoid Pigment Epitheliopathy, Acute Retinal Necrosis, Best Disease, Branch Retinal Artery Occlusion, Branch Retinal Vein Occlusion, Cancer Associated and Related Autoimmune Retinopathies, Central Retinal Artery Occlusion, Central Retinal Vein Occlusion, Central Serous Chorioretinopathy, Eales Disease, Epimacular Membrane, Lattice Degeneration, Macroaneurysm, Diabetic Macular Edema, Irvine-Gass Macular Edema, Macular Hole, Subretinal Neovascular Membranes, Diffuse Unilateral Subacute Neuroretinitis, Nonpseudophakic Cystoid Macular Edema, Presumed Ocular Histoplasmosis Syndrome, Exudative Retinal Detachment, Postoperative Retinal Detachment, Proliferative Retinal Detachment, Rhegmatogenous Retinal Detachment, Tractional Retinal Detachment, Retinitis Pigmentosa, CMV Retinitis, Retinoblastoma, Retinopathy of Prematurity, Birdshot Retinopathy, Background Diabetic Retinopathy, Proliferative Diabetic Retinopathy, Hemoglobinopathies Retinopathy, Purtscher Retinopathy, Valsalva Retinopathy, Juvenile Retinoschisis, Senile Retinoschisis, Terson Syndrome and White Dot Syndromes.

Other exemplary diseases include ocular bacterial infections (e.g. conjunctivitis, keratitis, tuberculosis, syphilis, gonorrhea), viral infections (e.g. Ocular Herpes Simplex Virus, Varicella Zoster Virus, Cytomegalovirus retinitis, Human Immunodeficiency Virus (HIV)) as well as progressive outer retinal necrosis secondary to HIV or other HIV-associated and other immunodeficiency-associated ocular diseases. In addition, ocular diseases include fungal infections (e.g. Candida choroiditis, histoplasmosis), protozoal infections (e.g. toxoplasmosis) and others such as ocular toxocariasis and sarcoidosis.

One aspect of the invention is a method for inhibiting, reducing or treating vision impairment in a subject undergoing treatment with a chemotherapeutic drug (e.g., a neurotoxic drug, a drug that raises intraocular pressure such as a steroid), by administering to the subject in need of such treatment a high dose of one or more sirtuin activating compounds.

Another aspect of the invention is a method for inhibiting, reducing or treating vision impairment in a subject undergoing surgery, including ocular or other surgeries performed in the prone position such as spinal cord surgery, by administering to the subject in need of such treatment a high dose of one or more sirtuin activating compounds. Ocular surgeries include cataract, iridotomy and lens replacements.

Another aspect of the invention is the treatment, including inhibition and prophylactic treatment, of age related ocular diseases include cataracts, dry eye, retinal damage and the like, by administering to the subject in need of such treatment a high dose of one or more sirtuin activating compounds.

The formation of cataracts is associated with several biochemical changes in the lens of the eye, such as decreased levels of antioxidants ascorbic acid and glutathione, increased lipid, amino acid and protein oxidation, increased sodium and calcium, loss of amino acids and decreased lens metabolism. The lens, which lacks blood vessels, is suspended in extracellular fluids in the anterior part of the eye. Nutrients, such as ascorbic acid, glutathione, vitamin E, selenium, bioflavonoids and carotenoids are required to maintain the transparency of the lens. Low levels of selenium results in an increase of free radical-inducing hydrogen peroxide, which is neutralized by the selenium-dependent antioxidant enzyme glutathione peroxidase. Lens-protective glutathione peroxidase is also dependent on the amino acids methionine, cysteine, glycine and glutamic acid.

Cataracts can also develop due to an inability to properly metabolize galactose found in dairy products that contain lactose, a disaccharide composed of the monosaccharide galactose and glucose. Cataracts can be prevented, delayed, slowed and possibly even reversed if detected early and metabolically corrected.

Retinal damage is attributed, inter alia, to free radical initiated reactions in glaucoma, diabetic retinopathy and age-related macular degeneration (AMD). The eye is a part of the central nervous system and has limited regenerative capability. The retina is composed of numerous nerve cells which contain the highest concentration of polyunsaturated fatty acids (PFA) and subject to oxidation. Free radicals are generated by UV light entering the eye and mitochondria in the rods and cones, which generate the energy necessary to transform light into visual impulses. Free radicals cause peroxidation of the PFA by hydroxyl or superoxide radicals which in turn propagate additional free radicals. The free radicals cause temporary or permanent damage to retinal tissue.

Glaucoma is usually viewed as a disorder that causes an elevated intraocular pressure (IOP) that results in permanent damage to the retinal nerve fibers, but a sixth of all glaucoma cases do not develop an elevated IOP. This disorder is now perceived as one of reduced vascular perfusion and an increase in neurotoxic factors. Recent studies have implicated elevated levels of glutamate, nitric oxide and peroxynitirite in the eye as the causes of the death of retinal ganglion cells. Neuroprotective agents may be the future of glaucoma care. For example, nitric oxide synthase inhibitors block the formation of peroxynitrite from nitric oxide and superoxide. In a recent study, animals treated with aminoguanidine, a nitric oxide synthase inhibitor, had a reduction in the loss of retinal ganglion cells. It was concluded that nitric oxide in the eye caused cytotoxicity in many tissues and neurotoxicity in the central nervous system.

Diabetic retinopathy occurs when the underlying blood vessels develop microvascular abnormalities consisting primarily of microaneurysms and intraretinal hemorrhages. Oxidative metabolites are directly involved with the pathogenesis of diabetic retinopathy and free radicals augment the generation of growth factors that lead to enhanced proliferative activity. Nitric oxide produced by endothelial cells of the vessels may also cause smooth muscle cells to relax and result in vasodilation of segments of the vessel. Ischemia and hypoxia of the retina occur after thickening of the arterial basement membrane, endothelial proliferation and loss of pericytes. The inadequate oxygenation causes capillary obliteration or nonperfusion, arteriolar-venular shunts, sluggish blood flow and an impaired ability of RBCs to release oxygen. Lipid peroxidation of the retinal tissues also occurs as a result of free radical damage.

The macula is responsible for our acute central vision and composed of light-sensing cells (cones) while the underlying retinal pigment epithelium (RPE) and choroid nourish and help remove waste materials. The RPE nourishes the cones with the vitamin A substrate for the photosensitive pigments and digests the cones shed outer tips. RPE is exposed to high levels of UV radiation, and secretes factors that inhibit angiogenesis. The choroid contains a dense vascular network that provides nutrients and removes the waste materials.

In AMD, the shed cone tips become indigestible by the RPE, where the cells swell and die after collecting too much undigested material. Collections of undigested waste material, called drusen, form under the RPE. Photoxic damage also causes the accumulation of lipofuscin in RPE cells. The intracellular lipofuscin and accumulation of drusen in Bruch's membrane interferes with the transport of oxygen and nutrients to the retinal tissues, and ultimately leads to RPE and photoreceptor dysfunction. In exudative AMD, blood vessels grow from the choriocapillaris through defects in Bruch's membrane and may grow under the RPE, detaching it from the choroid, and leaking fluid or bleeding.

Macular pigment, one of the protective factors that prevent sunlight from damaging the retina, is formed by the accumulation of nutritionally derived carotenoids, such as lutein, the fatty yellow pigment that serves as a delivery vehicle for other important nutrients and zeaxanthin. Antioxidants such as vitamins C and E, beta-carotene and lutein, as well as zinc, selenium and copper, are all found in the healthy macula. In addition to providing nourishment, these antioxidants protect against free radical damage that initiates macular degeneration.

Another aspect of the invention is the prevention or treatment of damage to the eye caused by stress, chemical insult or radiation, by administering to the subject in need of such treatment a high dose of one or more sirtuin activating compounds. Radiation or electromagnetic damage to the eye can include that caused by CRT's or exposure to sunlight or UV.

In one embodiment, a combination drug regimen may include drugs or compounds for the treatment or prevention of ocular disorders or secondary conditions associated with these conditions. Thus, a combination drug regimen may include a high dose of one or more sirtuin activating compounds and one or more therapeutic agents for the treatment of an ocular disorder. For example, a high dose of one or more sirtuin activating compounds can be combined with an effective amount of one or more of: an agent that reduces intraocular pressure, an agent for treating glaucoma, an agent for treating optic neuritis, an agent for treating CMV Retinopathy, an agent for treating multiple sclerosis, and/or an antibiotic, etc.

In one embodiment, a high dose of one or more sirtuin activating compounds can be administered in conjunction with a therapy for reducing intraocular pressure. One group of therapies involves blocking aqueous production. For example, topical beta-adrenergic antagonists (timolol and betaxolol) decrease aqueous production. Topical timolol causes IOP to fall in 30 minutes with peak effects in 1-2 hours. A reasonable regimen is Timoptic 0.5%, one drop every 30 minutes for 2 doses. The carbonic anhydrase inhibitor, acetazolamide, also decreases aqueous production and should be given in conjunction with topical beta-antagonists. An initial dose of 500 mg is administered followed by 250 mg every 6 hours. This medication may be given orally, intramuscularly, or intravenously. In addition, alpha 2-agonists (e.g., Apraclonidine) act by decreasing aqueous production. Their effects are additive to topically administered beta-blockers. They have been approved for use in controlling an acute rise in pressure following anterior chamber laser procedures, but has been reported effective in treating acute closed-angle glaucoma. A reasonable regimen is 1 drop every 30 minutes for 2 doses.

A second group of therapies for reducing intraocular pressure involve reducing vitreous volume. Hyperosmotic agents can be used to treat an acute attack. These agents draw water out of the globe by making the blood hyperosmolar. Oral glycerol in a dose of 1 mL/kg in a cold 50% solution (mixed with lemon juice to make it more palatable) often is used. Glycerol is converted to glucose in the liver; persons with diabetes may need additional insulin if they become hyperglycemic after receiving glycerol. Oral isosorbide is a metabolically inert alcohol that also can be used as an osmotic agent for patients with acute angle-closure glaucoma. Usual dose is 100 g taken p.o. (220 cc of a 45% solution). This inert alcohol should not be confused with isosorbide dinitrate, a nitrate-based cardiac medication used for angina and for congestive heart failure. Intravenous mannitol in a dose of 1.0-1.5 mg/kg also is effective and is well tolerated in patients with nausea and vomiting. These hyperosmotic agents should be used with caution in any patient with a history of congestive heart failure.

A third group of therapies involve facilitating aqueous outflow from the eye. Miotic agents pull the iris from the iridocorneal angle and may help to relieve the obstruction of the trabecular meshwork by the peripheral iris. Pilocarpine 2% (blue eyes)-4% (brown eyes) can be administered every 15 minutes for the first 1-2 hours. More frequent administration or higher doses may precipitate a systemic cholinergic crisis. NSAIDS are sometimes used to reduce inflammation.

Exemplary therapeutic agents for reducing intraocular pressure include ALPHAGAN® P (Allergan) (brimonidine tartrate ophthalmic solution), AZOPT® (Alcon) (brinzolamide ophthalmic suspension), BETAGAN® (Allergan) (levobunolol hydrochloride ophthalmic solution, USP), BETIMOL® (Vistakon) (timolol ophthalmic solution), BETOPTIC S® (Alcon) (betaxolol HCl), BRIMONIDINE TARTRATE (Bausch & Lomb), CARTEOLOL HYDROCHLORIDE (Bausch & Lomb), COSOPT® (Merck) (dorzolamide hydrochloride-timolol maleate ophthalmic solution), LUMIGAN® (Allergan) (bimatoprost ophthalmic solution), OPTIPRANOLOL® (Bausch & Lomb) (metipranolol ophthalmic solution), TIMOLOL GFS (Falcon) (timolol maleate ophthalmic gel forming solution), TIMOPTIC® (Merck) (timolol maleate ophthalmic solution), TRAVATAN® (Alcon) (travoprost ophthalmic solution), TRUSOPT® (Merck) (dorzolamide hydrochloride ophthalmic solution) and XALATAN® (Pharmacia & Upjohn) (latanoprost ophthalmic solution).

In one embodiment, a high dose of one or more sirtuin activating compounds can be administered in conjunction with a therapy for treating and/or preventing glaucoma. An example of a glaucoma drug is DARANIDE® Tablets (Merck) (Dichlorphenamide).

In one embodiment, a high dose of one or more sirtuin activating compounds can be administered in conjunction with a therapy for treating and/or preventing optic neuritis. Examples of drugs for optic neuritis include DECADRON® Phosphate Injection (Merck) (Dexamethasone Sodium Phosphate), DEPO-MEDROL® (Pharmacia & Upjohn) (methylprednisolone acetate), HYDROCORTONE® Tablets (Merck) (Hydrocortisone), ORAPRED® (Biomarin) (prednisolone sodium phosphate oral solution) and PEDIAPRED® (Celltech) (prednisolone sodium phosphate, USP).

In one embodiment, a high dose of one or more sirtuin activating compounds can be administered in conjunction with a therapy for treating and/or preventing CMV Retinopathy. Treatments for CMV retinopathy include CYTOVENE® (ganciclovir capsules) and VALCYTE® (Roche Laboratories) (valganciclovir hydrochloride tablets).

In one embodiment, a high dose of one or more sirtuin activating compounds can be administered in conjunction with a therapy for treating and/or preventing multiple sclerosis. Examples of such drugs include DANTRIUM® (Procter & Gamble Pharmaceuticals) (dantrolene sodium), NOVANTRONE® (Serono) (mitoxantrone), AVONEX® (Biogen Idec) (Interferon beta-1a), BETASERON® (Berlex) (Interferon beta-1b), COPAXONE® (Teva Neuroscience) (glatiramer acetate injection) and REBIF® (Pfizer) (interferon beta-1a).

In addition, macrolide and/or mycophenolic acid, which has multiple activities, can be co-administered with a high dose of one or more sirtuin activating compounds. Macrolide antibiotics include tacrolimus, cyclosporine, sirolimus, everolimus, ascomycin, erythromycin, azithromycin, clarithromycin, clindamycin, lincomycin, dirithromycin, josamycin, spiramycin, diacetyl-midecamycin, tylosin, roxithromycin, ABT-773, telithromycin, leucomycins, and lincosamide.

III. Exemplary Assays

In certain aspects, the present invention provides screening methods for identifying compounds (agents) for treating or preventing metabolic disorders. Candidate compounds identified by the subject screening methods can be administered to a subject, such as a subject in need thereof. A subject in need of such a treatment may be a subject who suffers from obesity or diabetes, or who has, or is, likely to have these disorders, as predicted, e.g., from family history. Exemplary agents are those described herein.

The effect of a compound on the activity of a sirtuin, such as SIRT1, may be determined as described, e.g., in Howitz et al., supra or as follows. For instance, sirtuin proteins may be contacted with a compound in vitro, e.g., in a solution or in a cell. In one embodiment, a sirtuin protein is contacted with a compound in a solution and an activity of the sirtuin, e.g., its ability to deacetylate a protein, such as a histone, p53, or portions thereof, is determined. Generally, a sirtuin is activated or inhibited by a compound when at least one of its biological activities, e.g., deacetylation activity, is higher or lower, respectively, in the presence of the compound than in its absence. Activation or inhibition may be by a factor of at least about 10%, 30%, 50%, 100% (i.e., a factor of two), 3, 10, 30, or 100.

Whether a sirtuin is activated or inhibited can be determined, e.g., by contacting the sirtuin or a cell or cell extract containing the sirtuin with a deacetylation target, such as a histone, p53 protein, or portions thereof, and determining the level of acetylation of the deacetylation target. A higher level of acetylation of the target incubated with the sirtuin that is being tested relative to the level of acetylation of a control sirtuin indicates that the sirtuin that is being tested is activated. Conversely, a lower level of acetylation of the target incubated with the sirtuin that is being tested relative to the level of acetylation of a control sirtuin indicates that the sirtuin that is being tested is inhibited. The control sirtuin may be a recombinantly produced sirtuin that has not been contacted with a sirtuin-activating or -inhibiting compound.

Assays for determining the likelihood that a subject has or will develop weight gain, obesity, insulin resistance, diabetes or precursor symptoms or conditions resulting therefrom, are also provided. Such assays may comprise determining the level activity or expression (e.g., mRNA, pre-mRNA or protein) of a sirtuin, such as SIRT1, or AMPK in a subject. A low level of sirtuin activity or expression in a subject is likely to indicate that the subject has or is likely to develop weight gain, obesity, insulin resistance, diabetes, precursor symptoms thereof or secondary conditions thereof. Alternatively, a higher level of sirtuin activity or expression in a subject is likely to indicate that the subject has or is likely to develop weight loss and be protected from developing high weight associated diseases, such as insulin resistance and diabetes. Other assays include determining the activity or level of expression of a sirtuin and AMPK.

Also provided herein are methods for identifying compounds that modulate weight gain and/or treat or prevent insulin resistance (or sensitivity) or diabetes. A method may comprise identifying an agent that modulates the activity or protein level of a sirtuin and testing whether the test agent modulates weight gain and/or can be used for treating or preventing insulin resistance or diabetes. The first step of the method may comprise contacting a sirtuin with a test agent and determining the effect of the test agent on the activity of the sirtuin, e.g., SIRT1, as described, e.g., in Howitz et al., supra. The first step of the method may also comprise contacting a cell comprising a sirtuin with a test agent and determining the effect of the test agent on the activity of or expression level of the sirtuin. Expression levels of a sirtuin may be determined by measuring the mRNA, pre-mRNA or protein level of the sirtuin. Other steps may comprise testing the agent in an animal model for obesity, insulin resistance and/or diabetes. Such animal models are well known in the art. Screening methods may further comprise a step to determine the toxicity or adverse effects of the agents.

Other screening assays comprise identifying agents that modulate AMPK activity or protein levels. There is a need for compounds that activate AMPK but do not have the toxicities or adverse effects of known AMPK activators, such as metformin/phenformin.

In other embodiments, the invention provides methods for determining and/or monitoring a subject's intake of a sirtuin modulating compound. Such methods may be useful for monitoring progress during therapeutic administration of a sirtuin modulator. Such assays may also be used to identify individuals that have been dosed with a sirtuin modulator. For example, such assays may be used to identify individuals, such as student athletes, professional athletes, Olympic athletes, etc., who have taken sirtuin modulators to enhance their athletic performance and/or endurance. Such methods may involve measuring the amount of a sirtuin modulator, or metabolite thereof, in the blood and/urine of an individual. Exemplary metabolites of resveratrol include, for example, resveratrol glucuronides and resveratrol sulfates, such as resveratrol monoglucuronide, dihydroresveratrol monosulfate, resveratrol monosulfate, dihydroresveratrol, trans-resveratrol-3-O-glucuronide, cis-resveratrol-3-O-glucuronide, cis-resveratrol-3-O-glucoside, trans-resveratrol-4′-sulfate, trans-resveratrol-3,5-disulfate, trans-resveratrol-3,4′-disulfate, trans-resveratrol-3,4′,5-trisulfate, and trans-resveratrol-3-O-beta-D-glucuronide, as well as resveratrol aglycone and free trans-resveratrol. The methods may involve obtaining a biological sample, such as urine, blood, saliva, tissue, feces, hair, skin, etc., from an individual and analyzing the sample to identify the presence or a sirtuin modulating compound or metabolite thereof, the amount of a sirtuin modulating compound or metabolite thereof, and/or the type of sirtuin modulating compound or metabolite thereof. Identification, quantitation and characterization of sirtuin acmodulating compounds or metabolites thereof from a biological sample may be achieved by a variety of methods known to one of skill in the art, such as, for example, an immunoassay, chromatography, mass spectroscopy (MS), including liquid chromatography (LC)-MS/MS, LC-ESI-MS/MS (electrospray ionization, ESI), high performance liquid chromatography-diode array detection (HPLC-DAD), or on-line ultraviolet-photodiode array detection and mass spectrometric detection (LC-DAD-MS and LC-UV-MS-MS) (see e.g., Wang et al., J. Chromatrogr B analyt Technol Biomed Life Sci 829: 97-106 (2005); Urpi-sarda et al., Anal Chem 77: 3149-55 (2005); Wenzel et al., Mol Nutr Food Res 49: 472-81 (2005); Wenzel et al., Mol Nutr Food Res 49: 482-94 (2005); Wang et al., J Pharm Sci 93: 2448-57 (2004); Meng et al., J Agric Food Chem 52: 935-42 (2004); and Yu et al., Pharm Res 19: 1907-14 (2002)). In certain embodiments, the methods may involve extracting, purifying or partially purifying the sirtuin modulators or metabolites thereof from the biological sample before analysis. In other embodiments, it may be desirable to compare the results to one or more known standards of sirtuin modulating compounds or metabolites thereof.

In yet other embodiments, provided are methods (e.g., assays such as screening assays or high throughput screens) for identifying agents, such as sirtuin modulating compounds, that are useful for modulating mitochondrial mass and/or mitochondrial function in cells of an animal or human subject. In certain embodiments, candidate agents are screened for their ability to increase mitochondrial mass and/or improve mitochondrial function. In an exemplary embodiment, the methods described herein may be used to identify an agent that increases mitochondrial mass and/or improves mitochondrial function in cells, such as, for example, a sirtuin-activating compound.

In one embodiment, a method for identifying an agent that modulates mitochondrial mass and/or function comprises contacting a candidate agent with a sample comprising a cell containing a mitochondrion, and determining a level of at least one indicator of mitochondrial function, wherein the candidate agent that alters the level of the indicator of mitochondrial function relative to the level of said indicator in the absence of the agent is indicative of an agent that alters mitochondrial function.

In another embodiment, a method for identifying an agent that modulates mitochondrial mass and/or function comprises identifying a regulator of mitochondrial biogenesis. The method may comprise contacting a stimulus with a cell comprising a mitochondrion under conditions and for a time sufficient to induce mitochondrial biogenesis; and detecting an altered level of a candidate signaling molecule, wherein an altered level of the candidate signaling molecule in a cell that has been contacted with the stimulus that induces mitochondrial biogenesis relative to the level of the candidate signaling molecule in a cell that has not been contacted with the stimulus indicates that the candidate signaling molecule is a regulator of mitochondrial biogenesis. In a further embodiment the stimulus is selected cold stress, an electrical stimulus or an adrenergic stimulus. In certain other embodiments mitochondrial biogenesis is detected by determining an indicator of mitochondrial function that is oxygen consumption, amount of mitochondrial DNA, mitochondrial mass or an ATP biosynthesis factor. In certain other embodiments the candidate signaling molecule regulates activity of a gene that is a PGC gene or a NRF gene. In certain other embodiments the candidate signaling molecule is regulated by a gene that is a PGC gene or a NRF gene. In certain other embodiments the altered level of the candidate signaling molecule is a level of a nucleic acid, a level of a polypeptide and a level of phosphorylation of a protein.

In certain embodiments, the indicator of mitochondrial function may be a mitochondrial electron transport chain enzyme. The methods may involve measuring electron transport chain enzyme catalytic activity, determining enzyme activity per mitochondrion in the sample, determining enzyme activity per unit of protein in the sample, measuring electron transport chain enzyme quantity, determining enzyme quantity per mitochondrion in the sample, and/or determining enzyme quantity per unit of protein in the sample. In certain embodiments the mitochondrial electron transport chain enzyme comprises at least one subunit of mitochondrial complex 1, mitochondrial complex II, mitochondrial complex III, mitochondrial complex IV, and/or mitochondrial complex V. The mitochondrial complex IV subunit may be COX1, COX2 or COX4 and the mitochondrial complex V subunit may be an ATP synthase subunit 8 or ATP synthase subunit 6.

In other embodiments, the indicator of mitochondrial function may be a mitochondrial matrix component. a mitochondrial membrane component, and/or a mitochondrial inner membrane component. The mitochondrial membrane component may be an adenine nucleotide translocator (ANT), voltage dependent anion channel (VDAC), malate-aspartate shuttle, calcium uniporter, UCP-1, UCP-2, UCP-3 (e.g., Boss et al., 2000 Diabetes 49:143; Klingenberg 1999 J. Bioenergetics Biomembranes 31:419), a hexokinase, a peripheral benzodiazepine receptor, a mitochondrial intermembrane creatine kinase, cyclophilin D, a Bcl-2 gene family encoded polypeptide, tricarboxylate carrier or dicarboxylate carrier.

In certain embodiments the indicator of mitochondrial function is a Krebs cycle enzyme. The methods may involve measuring Krebs cycle enzyme catalytic activity, determining enzyme activity per mitochondrion in the sample, determining enzyme activity per unit of protein in the sample, measuring Krebs cycle enzyme quantity, determining enzyme quantity per mitochondrion in the sample, and/or determining enzyme quantity per unit of protein in the sample. The Krebs cycle enzyme may be citrate synthase, aconitase, isocitrate dehydrogenase, alpha-ketoglutarate dehydrogenase, succinyl-coenzyme A synthetase, succinate dehydrogenase, fumarase or malate dehydrogenase.

In other embodiments, the indicator of mitochondrial function may be mitochondrial mass per cell in the sample. Mitochondrial mass may be determined using a mitochondria selective agent (such as nonylacridine orange) or by morphometric analysis. In certain embodiments, the indicator of mitochondrial function may be the number of mitochondria per cell in the sample which may be determined using a mitochondrion selective reagent, such as a fluorescent reagent.

In other embodiments, the indicator of mitochondrial function may be the amount of mitochondrial DNA (“mtDNA”) per cell in the sample. The amount of mitochondrial DNA per cell may be measured and/or expressed in absolute (e.g., mass of mtDNA per cell) or relative (e.g., proportion of mtDNA relative to nuclear DNA) termns. In certain embodiments, mitochondrial DNA is measured by contacting a biological sample containing mitochondrial DNA with an oligonucleotide primer having a nucleotide sequence that is complementary to a sequence present in the mitochondrial DNA, under conditions and for a time sufficient to allow hybridization of the primer to the mitochondrial DNA; and detecting hybridization of the primer to the mitochondrial DNA, and therefrom quantifying the mitochondrial DNA. In certain embodiments the step of detecting comprises a technique that may be polymerase chain reaction, oligonucleotide primer extension assay, ligase chain reaction, or restriction fragment length polymorphism analysis. In certain embodiments, mitochondrial DNA is measured by contacting a sample containing amplified mitochondrial DNA with an oligonucleotide primer having a nucleotide sequence that is complementary to a sequence present in the amplified mitochondrial DNA, under conditions and for a time sufficient to allow hybridization of the primer to the mitochondrial DNA; and detecting hybridization of the primer to the mitochondrial DNA, and therefrom quantifying the mitochondrial DNA. In certain embodiments the step of detecting comprises a technique that may be polymerase chain reaction, oligonucleotide primer extension assay, ligase chain reaction, or restriction fragment length polymorphism analysis. In certain embodiments the mitochondrial DNA is amplified using a technique that may be polymerase chain reaction, transcriptional amplification systems or self-sustained sequence replication. In certain embodiments, mitochondrial DNA is measured by contacting a biological sample containing mitochondrial DNA with an oligonucleotide primer having a nucleotide sequence that is complementary to a sequence present in the mitochondrial DNA, under conditions and for a time sufficient to allow hybridization of the primer to the mitochondrial DNA; and detecting hybridization and extension of the primer to the mitochondrial DNA to produce a product, and therefrom quantifying the mitochondrial DNA. In certain embodiments the step of comparing comprises measuring mitochondrial DNA by contacting a sample containing amplified mitochondrial DNA with an oligonucleotide primer having a nucleotide sequence that is complementary to a sequence present in the amplified mitochondrial DNA, under conditions and for a time sufficient to allow hybridization of the primer to the mitochondrial DNA; and detecting hybridization and extension of the primer to the mitochondrial DNA to produce a product, and therefrom quantifying the mitochondrial DNA. In certain embodiments the mitochondrial DNA is amplified using a technique that may be the polymerase chain reaction (PCR), including quantitaive and competitive PCR (Ahmed et al., BioTechniques 26:290-300, 1999), transcriptional amplification systems or self-sustained sequence replication. In certain embodiments, the amount of mitochondrial DNA in the sample is determined using an oligonucleotide primer extension assay. In other embodiments, the amount of mitochondrial DNA is determined by subjecting a sample to a cesium chloride gradient to separate it from nuclear DNA (see, e.g., Welter et al., Mol. Biol. Rep. 13:17-120, 1988) in the presence of a detectably labeled compound that binds to double-stranded nucleic acids (e.g., ethidium bromide) and comparing the relative and/or absolute signals corresponding to the mitochondrial and nuclear DNAs.

In other embodiments, the indicator of mitochondrial function is the amount of ATP per cell in the sample. The methods may comprise measuring the amount of ATP per mitochondrion in the sample, measuring the amount of ATP per unit protein in the sample, measuring the amount of ATP per unit mitochondrial mass in the sample, measuring the amount of ATP per unit mitochondial protein in the sample. In certain embodiments, the indicator of mitochondrial function is the rate of ATP synthesis in the sample or an ATP biosynthesis factor. The methods may comprise measuring ATP biosynthesis factor catalytic activity, determining ATP biosynthesis factor activity per mitochondrion in the sample, determining ATP biosynthesis factor activity per unit mitochondrial mass in the sample, determining ATP biosynthesis factor activity per unit of protein in the sample, measuring ATP biosynthesis factor quantity, determining ATP biosynthesis factor quantity per mitochondrion in the sample, and/or determining ATP biosynthesis factor quantity per unit of protein in the sample.

In other embodiments, the indicator of mitochondrial function may be one or more of the following: free radical production, reactive oxygen species, protein nitrosylation, protein carbonyl modification, DNA oxidation, mtDNA oxidation, protein oxidation, protein carbonyl modification, malondialdehyde adducts of proteins, a glycoxidation product, a lipoxidation product, 8′—OH-guanosine adducts, BARS, cellular response to elevated intracellular calcium, and/or cellular response to at least one apoptogen. In certain embodiments the indicator of mitochondrial function is oxygen consumption, which may be determined according to any of a variety of known methodologies (e.g., Wu et al., 1999 Cell 98:115; Li et al. 1999 J. Biol. Chem. 274:17534).

Functional mitochondria contain gene products encoded by mitochondrial genes situated in mitochondrial DNA (mtDNA) and by extramitochondrial genes (e.g., nuclear genes) not situated in the circular mitochondrial genome. The 16.5 kb mtDNA encodes 22 tRNAs, two ribosomal RNAs (rRNA) and 13 enzymes of the electron transport chain (ETC), the elaborate multi-complex mitochondrial assembly where, for example, respiratory oxidative phosphorylation takes place. The overwhelming majority of mitochondrial structural and functional proteins are encoded by extramitochondrial, and in most cases presumably nuclear, genes. Accordingly, mitochondrial and extramitochondrial genes may interact directly, or indirectly via gene products and their downstream intermediates, including metabolites, catabolites, substrates, precursors, cofactors and the like. Alterations in mitochondrial function, for example impaired electron transport activity, defective oxidative phosphorylation or increased free radical production, may therefore arise as the result of defective mtDNA, defective extramitochondrial DNA, defective mitochondrial or extramitochondrial gene products, defective downstream intermediates or a combination of these and other factors.

In certain embodiments, an enzyme is the indicator of mitochondrial function as provided herein. The enzyme may be a mitochondrial enzyme, which may further be an ETC enzyme or a Krebs cycle enzyme. The enzyme may also be an ATP biosynthesis factor, which may include an ETC enzyme and/or a Krebs cycle enzyme, or other enzymes or cellular components related to ATP production as provided herein. A “non-enzyme” refers to an indicator of mitochondrial function that is not an enzyme (i.e., that is not a mitochondrial enzyme or an ATP biosynthesis factor as provided herein). In certain other embodiments, an enzyme is a co-indicator of mitochondrial function. The following enzymes may not be indicators of mitochondrial function according to the present invention, but may be co-indicators of mitochondrial function as provided herein: citrate synthase (EC 4.1.3.7), hexokinase II (EC 2.7.1.1), cytochrome c oxidase (EC 1.9.3.1), phosphofructokinase (EC 2.7.1.11), glyceraldehyde phosphate dehydrogenase (EC 1.2.1.12), glycogen phosphorylase (EC 2.4.1.1) creatine kinase (EC 2.7.3.2), NADH dehydrogenase (EC 1.6.5.3), glycerol 3-phosphate dehydrogenase (EC 1.1.1.8), triose phosphate dehydrogenase (EC 1.2.1.12) and malate dehydrogenase (EC 1.1.1.37).

In other embodiments, the indicator of mitochondrial function is any ATP biosynthesis factor, ATP production, mitochondrial mass or mitochondrial number, free radical production, a cellular response to elevated intracellular calcium and/or a cellular response to an apoptogen. In certain embodiments, mitochondrial DNA content may not be an indicator of mitochondrial function but may be a co-predictor of mitochondrial function or a co-indicator of mitochondrial function, as provided herein.

i. Indicators of Mitochondrialfunction that are Enzymes

In certain embodiments, methods for identifying agents that modulate mitochondrial mass and/or function include the detection and/or absolute or relative measurement of at least one indicator of mitochondrial function in biological test samples, wherein the indicator of mitochondrial function is an enzyme. As provided herein, such an enzyme may be a mitochondrial enzyme or an ATP biosynthesis factor that is an enzyme, for example an ETC enzyme or a Krebs cycle enzyme.

Reference to “enzyme quantity”, “enzyme catalytic activity” or “enzyme expression level” in the context of the methods for identifying agents that modulate mitochondrial mass and/or function, is meant to include a reference to any of a mitochondrial enzyme quantity, activity or expression level or an ATP biosynthesis factor quantity, activity or expression level; either of which may further include, for example, an ETC enzyme quantity, activity or expression level or a Krebs cycle enzyme quantity, activity or expression level. In the most preferred embodiments of the invention, an enzyme is a natural or recombinant protein or polypeptide that has enzyme catalytic activity as provided herein. Such an enzyme may be, by way of non-limiting examples, an enzyme, a holoenzyme, an enzyme complex, an enzyme subunit, an enzyme fragment, derivative or analog or the like, including a truncated, processed or cleaved enzyme.

A mitochondrial enzyme that may be an indicator of mitochondrial function as provided herein refers to a mitochondrial molecular component that has enzyme catalytic activity and/or functions as an enzyme cofactor capable of influencing enzyme catalytic activity. As used herein, mitochondria are comprised of “mitochondrial molecular components”, which may be a protein, polypeptide, peptide, amino acid, or derivative thereof, a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like; or any covalently or non-covalently complexed combination of these components, or any other biological molecule that is a stable or transient constituent of a mitochondrion.

A mitochondrial enzyme that may be an indicator of mitochondrial function or a co-indicator of mitochondrial function as provided herein, or an ATP biosynthesis factor that may be an indicator of mitochondrial function as provided herein, may comprise an ETC enzyme, which refers to any mitochondrial molecular component that is a mitochondrial enzyme component of the mitochondrial electron transport chain (ETC) complex associated with the inner mitochondrial membrane and mitochondrial matrix. An ETC enzyme may include any of the multiple ETC subunit polypeptides encoded by mitochondrial and nuclear genes. The ETC is typically described as comprising complex I (NADH:ubiquinone reductase), complex II (succinate dehydrogenase), complex III (ubiquinone: cytochrome c oxidoreductase), complex IV (cytochrome c oxidase) and complex V (mitochondrial ATP synthetase), where each complex includes multiple polypeptides and cofactors (for review see, e.g., Walker et al., 1995 Meths. Enzymol. 260:14; Emster et al., 1981 J. Cell Biol. 91:227s-255s, and references cited therein).

A mitochondrial enzyme that may be an indicator of mitochondrial function as provided herein, or an ATP biosynthesis factor that may be an indicator of mitochondrial function as provided herein, may also comprise a Krebs cycle enzyme, which includes mitochondrial molecular components that mediate the series of biochemicalvbioenergetic reactions also known as the citric acid cycle or the tricarboxylic acid cycle (see, e.g., Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.). Krebs cycle enzymes include subunits and cofactors of citrate synthase, aconitase, isocitrate dehydrogenase, the a-ketoglutarate dehydrogenase complex, succinyl CoA synthetase, succinate dehydrogenase, fumarase and malate dehydrogenase. Krebs cycle enzymes further include enzymes and cofactors that are functionally linked to the reactions of the Krebs cycle, such as, for example, nicotinamide adenine dinucleotide, coenzyme A, thiamine pyrophosphate, lipoamide, guanosine diphosphate, flavin adenine dinucloetide and nucleoside diphosphokinase.

The methods described herein also pertain in part to the correlation of type 2 diabetes with an indicator of mitochondrial function that may be an ATP biosynthesis factor, an altered amount of ATP or an altered amount of ATP production. For example, decreased mitochondrial ATP biosynthesis may be an indicator of mitochondrial function from which a risk for type 2 diabetes may be identified.

An “ATP biosynthesis factor” refers to any naturally occurring cellular component that contributes to the efficiency of ATP production in mitochondria. Such a cellular component may be a protein, polypeptide, peptide, amino acid, or derivative thereof, a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like. An ATP biosynthesis factor includes at least the components of the ETC and of the Krebs cycle (see, e.g., Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.) and any protein, enzyme or other cellular component that participates in ATP synthesis, regardless of whether such ATP biosynthesis factor is the product of a nuclear gene or of an extranuclear gene (e.g., a mitochondrial gene). Participation in ATP synthesis may include, but need not be limited to, catalysis of any reaction related to ATP synthesis, transmembrane import and/or export of ATP or of an enzyme cofactor, transcription of a gene encoding a mitochondrial enzyme and/or translation of such a gene transcript.

Compositions and methods for determining whether a cellular component is an ATP biosynthesis factor are well known in the art, and include methods for determining ATP production (including determination of the rate of ATP production in a sample) and methods for quantifying ATP itself. The contribution of an ATP biosynthesis factor to ATP production can be determined, for example, using an isolated ATP biosynthesis factor that is added to cells or to a cell-free system. The ATP biosynthesis factor may directly or indirectly mediate a step or steps in a biosynthetic pathway that influences ATP production. For example, an ATP biosynthesis factor may be an enzyme that catalyzes a particular chemical reaction leading to ATP production. As another example, an ATP biosynthesis factor may be a cofactor that enhances the efficiency of such an enzyme. As another example, an ATP biosynthesis factor may be an exogenous genetic element introduced into a cell or a cell-free system that directly or indirectly affects an ATP biosynthetic pathway. Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, ³²P; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of mitochondrial function. In one embodiment, for instance, ATP may be quantified by measuring luminescence of luciferase catalyzed oxidation of D-luciferin, an ATP dependent process.

“Enzyme catalytic activity” refers to any function performed by a particular enzyme or category of enzymes that is directed to one or more particular cellular function(s). For example, “ATP biosynthesis factor catalytic activity” refers to any function performed by an ATP biosynthesis factor as provided herein that contributes to the production of ATP. Typically, enzyme catalytic activity is manifested as facilitation of a chemical reaction by a particular enzyme, for instance an enzyme that is an ATP biosynthesis factor, wherein at least one enzyme substrate or reactant is covalently modified to form a product. For example, enzyme catalytic activity may result in a substrate or reactant being modified by formation or cleavage of a covalent chemical bond, but the invention need not be so limited. Various methods of measuring enzyme catalytic activity are known to those having ordinary skill in the art and depend on the particular activity to be determined.

For many enzymes, including mitochondrial enzymes or enzymes that are ATP biosynthesis factors as provided herein, quantitative criteria for enzyme catalytic activity are well established. These criteria include, for example, activity that may be defined by international units (ITJ), by enzyme turnover number, by catalytic rate constant (K_{cat}), by Michaelis-Menten constant (Kₘ), by specific activity or by any other enzymological method known in the art for measuring a level of at least one enzyme catalytic activity. Specific activity of a mitochondrial enzyme, such as an ATP biosynthesis factor, may be expressed as units of substrate detectably converted to product per unit time and, optionally, further per unit sample mass (e.g., per unit protein or per unit mitochondrial mass).

In certain embodiments, enzyme catalytic activity may be expressed as units of substrate detectably converted by an enzyme to a product per unit time per unit total protein in a sample, as units of substrate detectably converted by an enzyme to product per unit time per unit mitochondrial mass in a sample, or as units of substrate detectably converted by an enzyme to product per unit time per unit mitochondrial protein mass in a sample. Products of enzyme catalytic activity may be detected by suitable methods that will depend on the quantity and physicochemical properties of the particular product. Thus, detection may be, for example by way of illustration and not limitation, by radiometric, colorimetric, spectrophotometric, fluorimetric, immunometric or mass spectrometric procedures, or by other suitable means that will be readily apparent to a person having ordinary skill in the art.

In certain embodiments, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals--Sixth Ed., Molecular Probes, Eugene, Oreg.; Scopes, R. K., Protein Purification: Principles and Practice, 1987, Springer-Verlag, New York; Hermanson, G. T. et al., Immobilized Affinity Ligand Techniques, 1992, Academic Press, Inc., New York; Luo et al., 1998 J. Biotechnol. 65:225 and references cited therein). Spectroscopic methods may be used to detect dyes (including, for example, colorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well known techniques.

As noted above, enzyme catalytic activity of an ATP biosynthesis factor may further include other functional activities that lead to ATP production, beyond those involving covalent alteration of a substrate or reactant. For example by way of illustration and not limitation, an ATP biosynthesis factor that is an enzyme may refer to a transmembrane transporter molecule that, through its enzyme catalytic activity, facilitates the movement of metabolites between cellular compartments. Such metabolites may be ATP or other cellular components involved in ATP synthesis, such as gene products and their downstream intermediates, including metabolites, catabolites, substrates, precursors, cofactors and the like. As another non-limiting example, an ATP biosynthesis factor that is an enzyme may, through its enzyme catalytic activity, transiently bind to a cellular component involved in ATP synthesis in a manner that promotes ATP synthesis. Such a binding event may, for instance, deliver the cellular component to another enzyme involved in ATP synthesis and/or may alter the conformation of the cellular component in a manner that promotes ATP synthesis. Further to this example, such conformational alteration may be part of a signal transduction pathway, an allosteric activation pathway, a transcriptional activation pathway or the like, where an interaction between cellular components leads to ATP production.

Thus, an ATP biosynthesis factor may include, for example, a mitochondrial membrane protein. Suitable mitochondrial membrane proteins include such mitochondrial components as the adenine nucleotide transporter (ANT; e.g., Fiore et al., 1998 Biochimie 80:137; Klingenberg 1985 Ann. New York Acad. Sci. 456:279), the voltage dependent anion channel (VDAC, also referred to as porin; e.g., Manella, 1997 J. Bioenergetics Biomembr. 29:525), the malate-aspartate shuttle, the mitochondrial calcium uniporter (e.g., Litsky et al., 1997 Biochem. 36:7071), uncoupling proteins (UCP-1, -2, -3; see e.g., Jezek et al., 1998 Int. J. Biochem. Cell Biol. 30:1163), a hexokinase, a peripheral benzodiazepine receptor, a mitochondrial intermembrane creatine kinase, cyclophilin D, a Bcl-2 gene family encoded polypeptide, the tricarboxylate carrier (e.g., locobazzi et al., 1996 Biochim. Biophys. Acta 1284:9; Bisaccia et al., 1990 Biochim. Biophys. Acta 1019:250) and the dicarboxylate carrier (e.g., Fiermonte et al., 1998 J. Biol. Chem. 273:24754; Indiveri et al., 1993 Biochim. Biophys. Acta 1143:310; for a general review of mitochondrial membrane transporters, see, e.g., Zonatti et al., 1994 J. Bioenergetics Biomembr. 26:543 and references cited therein).

Enzyme quantity as used herein with reference to the methods for identifying modulators of mitochondrial mass and/or function refers to an amount of an enzyme including mitochondrial enzymes or enzymes that are ATP biosynthesis factors as provided herein, or of another ATP biosynthesis factor, that is present, i.e., the physical presence of an enzyme or ATP biosynthesis factor selected as an indicator of mitochondrial function, irrespective of enzyme catalytic activity. Depending on the physicochemical properties of a particular enzyme or ATP biosynthesis factor, the preferred method for determining the enzyme quantity will vary. In the most highly preferred embodiments of the invention, determination of enzyme quantity will involve quantitative determination of the level of a protein or polypeptide using routine methods in protein chemistry with which those having skill in the art will be readily familiar, for example by way of illustration and not limitation, those described in greater detail below.

Accordingly, determination of enzyme quantity may be by any suitable method known in the art for quantifying a particular cellular component that is an enzyme or an ATP biosynthesis factor as provided herein, and that in preferred embodiments is a protein or polypeptide. Depending on the nature and physicochemical properties of the enzyme or ATP biosynthesis factor, determination of enzyme quantity may be by densitometric, mass spectrometric, spectrophotometric, fluorimetric, immunometric, chromatographic, electrochemical or any other means of quantitatively detecting a particular cellular component. Methods for determining enzyme quantity also include methods described above that are useful for detecting products of enzyme catalytic activity, including those measuring enzyme quantity directly and those measuring a detectable label or reporter moiety. In certain preferred embodiments of the invention, enzyme quantity is determined by immunometric measurement of an isolated enzyme or ATP biosynthesis factor. In certain preferred embodiments of the invention, these and other immunological and immunochemical techniques for quantitative determination of biomolecules such as an enzyme or ATP biosynthesis factor may be employed using a variety of assay formats known to those of ordinary skill in the art, including but not limited to enzyme linked immunosorbent assay (ELISA), radioimmunoassay (RIA), immunofluorimetry, immunoprecipitation, equilibrium dialysis, immunodiffusion and other techniques. (See, e.g., Harlow and Lane, Antibodies: A Laboratory Manual, Cold Spring Harbor Laboratory, 1988; Weir, D. M., Handbook of Experimental Immunology, 1986, Blackwell Scientific, Boston.) For example, the assay may be performed in a Western blot format, wherein a preparation comprising proteins from a biological sample is submitted to gel electrophoresis, transferred to a suitable membrane and allowed to react with an antibody specific for an enzyme or an ATP biosynthesis factor that is a protein or polypeptide. The presence of the antibody on the membrane may then be detected using a suitable detection reagent, as is well known in the art and described above.

In certain embodiments, an indicator (or co-indicator) of mitochondrial function including, for example, an enzyme as provided herein, may be present in an isolated form, e.g., removed from its original environment (e.g., the natural environment if it is naturally occurring). For example, a naturally occurring polypeptide present in a living animal is not isolated, but the same polypeptide, separated from some or all of the co-existing materials in the natural system, is isolated. Such polypeptides could be part of a composition, and still be isolated in that such composition is not part of its natural environment.

Affinity techniques are useful in the context of isolating an enzyme or an ATP biosynthesis factor protein or polypeptide for use according to the methods of the present invention, and may include any method that exploits a specific binding interaction involving an enzyme or an ATP biosynthesis factor to effect a separation. For example, because an enzyme or an ATP biosynthesis factor protein or polypeptide may contain covalently attached oligosaccharide moieties, an affinity technique such as binding of the enzyme (or ATP biosynthesis factor) to a suitable immobilized lectin under conditions that permit carbohydrate binding by the lectin may be a particularly useful affinity technique.

Other useful affinity techniques include immunological techniques for isolating and/or detecting a specific protein or polypeptide antigen (e.g., an enzyme or ATP biosynthesis factor), which techniques rely on specific binding interaction between antibody combining sites for antigen and antigenic determinants present on the factor. Binding of an antibody or other affinity reagent to an antigen is “specific” where the binding interaction involves a Kₐ of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹. Affinities of binding partners or antibodies can be readily determined using conventional techniques, for example those described by Scatchard et al., Ann. New York Acad. Sci. 51:660 (1949).

Immunological techniques include, but need not be limited to, immunoaffinity chromatography, immunoprecipitation, solid phase immunoadsorption or other immunoaffinity methods. For these and other useful affinity techniques, see, for example, Scopes, R. K., Protein Purification: Principles and Practice, 1987, Springer-Verlag, New York; Weir, D. M., Handbook of Experimental Immunology, 1986, Blackwell Scientific, Boston; and Hermanson, G. T. et al., Immobilized Affinity Ligand Techniques, 1992, Academic Press, Inc., California; which are hereby incorporated by reference in their entireties, for details regarding techniques for isolating and characterizing complexes, including affinity techniques.

As noted above, an indicator of mitochondrial function can be a protein or polypeptide, for example an enzyme or an ATP biosynthesis factor. The protein or polypeptide may be an unmodified polypeptide or may be a polypeptide that has been posttranslationally modified, for example by glycosylation, phosphorylation, fatty acylation including glycosylphosphatidylinositol anchor modification or the like, phospholipase cleavage such as phosphatidylinositol-specific phospholipase c mediated hydrolysis or the like, protease cleavage, dephosphorylation or any other type of protein posttranslational modification such as a modification involving formation or cleavage of a covalent chemical bond.

ii. Indicators of Mitochondrial Function that are Mitochondrial Mass, Mitochondrial Volume or Mitochondrial Number

In certain embodiments, methods for identifying agents that modulate mitochondrial mass and/or function include the detection and/or measurement of at least one indicator of mitochondrial function in biological test samples, wherein the indicator of mitochondrial function is absolute or relative mitochondrial mass, mitochondrial volume or mitochondrial number.

Methods for quantifying mitochondrial mass, volume and/or mitochondrial number are known in the art, and may include, for example, quantitative staining of a representative biological sample. Typically, quantitative staining of mitochondrial may be performed using organelle-selective probes or dyes, including but not limited to mitochondrion selective reagents such as fluorescent dyes that bind to mitochondrial molecular components (e.g., nonylacridine orange, MitoTrackers) or potentiometric dyes that accumulate in mitochondria as a function of mitochondrial inner membrane electrochemical potential (see, e.g., Haugland, 1996 Handbook of Fluorescent Probes and Research Chemicals, Sixth Ed., Molecular Probes, Eugene, Oreg.). As another example, mitochondrial mass, volume and/or number may be quantified by morphometric analysis (e.g., Cruz-Orive et al., 1990 Am. J. Physiol. 258: L148; Schwerzmann et al., 1986 J. Cell Biol. 102:97). These or any other means known in the art for quantifying mitochondrial mass, volume and/or mitochondrial number in a sample are within the contemplated scope of the invention. For example, the use of such quantitative determinations for purposes of calculating mitochondrial density is contemplated and is not intended to be limiting. In certain embodiments, mitochondrial protein mass in a sample is determined using well known procedures. For example, a person having ordinary skill in the art can readily prepare an isolated mitochondrial fraction from a biological sample using established cell fractionation techniques, and therefrom determine protein content using any of a number of protein quantification methodologies well known in the art.

iii. Indicators of Mitochondrial Function that Include Mitochondrial DNA Content

In other embodiments, methods for identifying modulators of mitochondrial mass and/or function include the detection and/or measurement of at least one indicator of mitochondrial function in biological test samples, wherein the indicator of mitochondrial function is the absolute or relative amount of mitochondrial DNA. Quantification of mitochondrial DNA (mtDNA) content may be accomplished by any of a variety of established techniques that are useful for this purpose, including but not limited to oligonucleotide probe hybridization or polymerase chain reaction (PCR) using oligonucleotide primers specific for mitochondrial DNA sequences (see, e.g., Miller et al., 1996 J. Neurochem. 67:1897; Fahy et al., 1997 Nucl. Ac. Res. 25:3102; U.S. patent application Ser. No. 09/098,079; Lee et al., 1998 Diabetes Res. Clin. Practice 42:161; Lee et al., 1997 Diabetes 46(suppl. 1):175A). A particularly useful method is the primer extension assay disclosed by Fahy et al. (Nucl. Acids Res. 25:3102, 1997) and by Ghosh et al. (Am. J. Hum. Genet. 58:325, 1996). Suitable hybridization conditions may be found in the cited references or may be varied according to the particular nucleic acid target and oligonucleotide probe selected, using methodologies well known to those having ordinary skill in the art (see, e.g., Ausubel et al., Current Protocols in Molecular Biology, Greene Publishing, 1987; Sambrook et al., Molecular Cloning: A Laboratory Manual, Cold Spring Harbor Press, 1989).

Examples of other useful techniques for determining the amount of specific nucleic acid target sequences (e.g., mtDNA) present in a sample based on specific hybridization of a primer to the target sequence include specific amplification of target nucleic acid sequences and quantification of amplification products, including but not limited to polymerase chain reaction (PCR, Gibbs et al., Nucl. Ac. Res. 17:2437, 1989), transcriptional amplification systems (e.g., Kwoh et al., 1989 Proc. Nat. Acad. Sci. 86:1173); strand displacement amplification (e.g., Walker et al., Nucl. Ac. Res. 20:1691, 1992; Walker et al., Proc. Nat. Acad. Sci. 89:392, 1992) and self-sustained sequence replication (3SR, see, e.g., Ghosh et al, in Molecular Methods for Virus Detection, 1995 Academic Press, New York, pp. 287-314; Guatelli et al., Proc. Nat. Acad. Sci. 87:1874, 1990), the cited references for which are incorporated herein by reference in their entireties. Other useful amplification techniques include, for example, ligase chain reaction (e.g., Barany, Proc. Nat. Acad. Sci. 88:189, 1991), Q-beta replicase assay (Cahill et al., Clin. Chem. 37:1482, 1991; Lizardi et al., Biotechnol. 6:1197, 1988; Fox et al., J. Clin. Lab. Analysis 3:378, 1989) and cycled probe technology (e.g., Cloney et al., Clin. Chem. 40:656, 1994), as well as other suitable methods that will be known to those familiar with the art.

Sequence length or molecular mass of primer extension assay products may be determined using any known method for characterizing the size of nucleic acid sequences with which those skilled in the art are familiar. In one embodiment, primer extension products are characterized by gel electrophoresis. In another embodiment, primer extension products are characterized by mass spectrometry (MS), which may further include matrix assisted laser desorption ionization/time of flight (MALDI-TOF) analysis or other MS techniques known to those skilled in the art. See, for example, U.S. Pat. Nos. 5,622,824, 5,605,798 and 5,547,835. In another embodiment, primer extension products are characterized by liquid or gas chromatography, which may further include high performance liquid chromatography (HPLC), gas chromatography-mass spectrometry (GC-MS) or other well known chromatographic methodologies.

iv. Indicators of Mitochondrial Function that are Cellular Responses to Elevated Intracellular Calcium

Certain aspects of the present invention, as it relates detecting and/or measuring an indicator of mitochondrial function, involve monitoring intracellular calcium homeostasis and/or cellular responses to perturbations of this homeostasis, including physiological and pathophysiological calcium regulation. The range of cellular responses to elevated intracellular calcium is broad, as is the range of methods and reagents for the detection of such responses. Many specific cellular responses are known to those having ordinary skill in the art; these responses will depend on the particular cell types present in a selected biological sample. As non-limiting examples, cellular responses to elevated intracellular calcium include secretion of specific secretory products, exocytosis of particular pre-formed components, increased glycogen metabolism and cell proliferation (see, e.g., Clapham, 1995 Cell 80:259; Cooper, The Cell—A Molecular Approach, 1997 ASM Press, Washington, D.C.; Alberts, B., Bray, D., et al., Molecular Biology of the Cell, 1995 Garland Publishing, New York).

As a brief background, normal alterations of intramitochondrial calcium are associated with normal metabolic regulation (Dykens, 1998 in Mitochondria & Free Radicals in Neurodegenerative Diseases, Beal, Howell and Bodis-Wollner, Eds., Wiley-Liss, New York, pp. 29-55; Radi et al., 1998 in Mitochondria & Free Radicals in Neurodegenerative Diseases, Beal, Howell and Bodis-Wollner, Eds., Wiley-Liss, New York, pp. 57-89; Gunter and Pfeiffer, 1991, Am. J. Physio. 27: C755; Gunter et al., 1994, Am. J. Physiol. 267:313). For example, fluctuating levels of mitochondrial free Calcium may be responsible for regulating oxidative metabolism in response to increased ATP utilization, via allosteric regulation of enzymes (reviewed by Crompton et al., 1993 Basic Res. Cardiol. 88: 513-523; ) and the glycerophosphate shuttle (Gunter et al., 1994 J. Bioenerg. Biomembr. 26: 471).

Normal mitochondrial function includes regulation of cytosolic free calcium levels by sequestration of excess calcium within the mitochondrial matrix. Depending on cell type, cytosolic calcium concentration is typically 50-100 nM. In normally functioning cells, when calcium levels reach 200-300 nM, mitochondria begin to accumulate calcium as a function of the equilibrium between influx via a calcium uniporter in the inner mitochondrial membrane and calcium efflux via both sodium dependent and sodium independent calcium carriers. In certain instances, such perturbation of intracellular calcium homeostasis is a feature of diseases (such as type 2 diabetes) associated with mitochondrial function, regardless of whether the calcium regulatory dysfunction is causative of, or a consequence of, mitochondrial function.

Elevated mitochondrial calcium levels thus may accumulate in response to an initial elevation in cytosolic free calcium, as described above. Such elevated mitochondrial calcium concentrations in combination with reduced ATP or other conditions associated with mitochondrial pathology, can lead to collapse of mitochondrial inner membrane potential (see Gunter et al., 1998 Biochim. Biophys. Acta 1366:5; Rottenberg and Marbach, 1990, Biochim. Biophys. Acta 1016:87). The extramitochondrial (cytosolic) level of calcium in a biological sample that is greater than that present within mitochondria may be used as a risk factor for type 2 diabetes in an individual. In the case of type 2 diabetes, mitochondrial or cytosolic calcium levels may vary from the above ranges and may range from, e.g., about 1 nM to about 500 mM, more typically from about 10 nM to about 100 mM and usually from about 20 nM to about 1 mM, where “about” indicates+/−10%. A variety of calcium indicators are known in the art, including but not limited to, for example, fura-2 (McCormack et al., 1989 Biochim. Biophys. Acta 973:420); mag-fura-2; BTC (U.S. Pat. No. 5,501,980); fluo-3, fluo-4 and fluo-5N (U.S. Pat. No. 5,049,673); rhod-2; benzothiaza-1; and benzothiaza-2 (all of which are available from Molecular Probes, Eugene, Oreg.). These or any other means for monitoring intracellular calcium are contemplated according to the subject invention method for identifying a risk for type 2 diabetes.

For monitoring an indicator of mitochondrial function that is a cellular response to elevated intracellular calcium, compounds that induce increased cytoplasmic and mitochondrial concentrations of calcium, including calcium ionophores, are well known to those of ordinary skill in the art, as are methods for measuring intracellular calcium and intramitochondrial calcium (see, e.g., Gunter and Gunter, 1994 J. Bioenerg. Biomembr. 26: 471; Gunter et al., 1998 Biochim. Biophys. Acta 1366:5; McCormack et al., 1989 Biochim. Biophys. Acta 973:420; Orrenius and Nicotera, 1994 J. Neural. Transm. Suppl. 43:1; Leist and Nicotera, 1998 Rev. Physiol. Biochem. Pharmacol. 132:79; and Haugland, 1996 Handbook of Fluorescent Probes and Research Chemicals, Sixth Ed., Molecular Probes, Eugene, Oreg.). Accordingly, a person skilled in the art may readily select a suitable ionophore (or another compound that results in increased cytoplasmic and/or mitochondrial concentrations of calcium ions) and an appropriate means for detecting intracellular and/or intramitochondrial calcium for use in the present invention, according to the instant disclosure and to well known methods.

Calcium ion influx into mitochondria appears to be largely dependent, and may be completely dependent, upon the negative transmembrane electrochemical potential (DY) established at the inner mitochondrial membrane by electron transfer, and such influx fails to occur in the absence of DY even when an eight-fold Calcium concentration gradient is imposed (Kapus et al., 1991 FEBS Lett. 282:61). Accordingly, mitochondria may release Calcium when the membrane potential is dissipated, as occurs with uncouplers like 2,4-dinitrophenol and carbonyl cyanide p-trifluoro-methoxyphenylhydrazone (FCCP). Thus, according to certain embodiments of the present invention, collapse of DY may be potentiated by influxes of cytosolic free calcium into the mitochondria, as may occur under certain physiological conditions including those encountered by cells of a subject having type 2 DM. Detection of such collapse may be accomplished by a variety of means as provided herein.

Typically, mitochondrial membrane potential may be determined according to methods with which those skilled in the art will be readily familiar, including but not limited to detection and/or measurement of detectable compounds such as fluorescent indicators, optical probes and/or sensitive pH and ion-selective electrodes (See, e.g., Ernster et al., 1981 J. Cell Biol. 91:227s and references cited; see also Haugland, 1996 Handbook of Fluorescent Probes and Research Chemicals, Sixth Ed., Molecular Probes, Eugene, Oreg., pp. 266-274 and 589-594.). For example, by way of illustration and not limitation, the fluorescent probes 2-,4-dimethylaminostyryl-N-methyl pyridinium (DASPMI) and tetramethylrhodamine esters (e.g., tetramethylrhodamine methyl ester, TMRM; tetramethylrhodamine ethyl ester, TMRE) or related compounds (see, e.g., Haugland, 1996, supra) may be quantified following accumulation in mitochondria, a process that is dependent on, and proportional to, mitochondrial membrane potential (see, e.g., Murphy et al., 1998 in Mitochondria & Free Radicals in Neurodegenerative Diseases, Beal, Howell and Bodis-Wollner, Eds., Wiley-Liss, New York, pp. 159-186 and references cited therein; and Molecular Probes On-line Handbook of Fluorescent Probes and Research Chemicals, on the world wide web at probes.com/handbook/toc.html). Other fluorescent detectable compounds that may be used include but are not limited to rhodamine 123, rhodamine B hexyl ester, DiOC₆(3), JC-1 [5,5′,6,6′-Tetrachloro-1,1′,3,3′-Tetraethylbez-imidazolcarbocyanine Iodide] (see Cossarizza, et al., 1993 Biochem. Biophys. Res. Comm. 197:40; Reers et al., 1995 Meth. Enzymol. 260:406), rhod-2 (see U.S. Pat. No. 5,049,673; all of the preceding compounds are available from Molecular Probes, Eugene, Oreg.) and rhodamine 800 (Lambda Physik, GmbH, Gottingen, Germany; see Sakanoue et al., 1997 J. Biochem. 121:29). Methods for monitoring mitochondrial membrane potential are also disclosed in U.S. patent application Ser. No. 09/161,172.

Mitochondrial membrane potential can also be measured by non-fluorescent means, for example by using TTP (tetraphenylphosphonium ion) and a TTP-sensitive electrode (Kamo et al., 1979 J. Membrane Biol. 49:105; Porter and Brand, 1995 Am. J. Physiol. 269: RI213). Those skilled in the art will be able to select appropriate detectable compounds or other appropriate means for measuring DYm. By way of example and not limitation, TMRM is somewhat preferable to TMRE because, following efflux from mitochondria, TMRE yields slightly more residual signal in the endoplasmic reticulicum and cytoplasm than TMRM.

As another non-limiting example, membrane potential may be additionally or alternatively calculated from indirect measurements of mitochondrial permeability to detectable charged solutes, using matrix volume and/or pyridine nucleotide redox determination combined with spectrophotometric or fluorimetric quantification. Measurement of membrane potential dependent substrate exchange-diffusion across the inner mitochondrial membrane may also provide an indirect measurement of membrane potential. (See, e.g., Quinn, 1976, The Molecular Biology of Cell Membranes, University Park Press, Baltimore, Md., pp. 200-217 and references cited therein.)

Exquisite sensitivity to extraordinary mitochondrial accumulations of calcium that result from elevation of intracellular calcium, as described above, may also characterize type 2 diabetes. Such mitochondrial sensitivity may provide an indicator of mitochondrial function according to the present invention. Additionally, a variety of physiologically pertinent agents, including hydroperoxide and free radicals, may synergize with calcium to induce collapse of DY (Novgorodov et al., 1991 Biochem. Biophys. Acta 1058: 242; Takeyama et al., 1993 Biochem. J. 294: 719; Guidox et al., 1993 Arch. Biochem. Biophys. 306:139).

v. Indicators of Mitochondrial Function that Include Responses to Apoptogenic Stimuli

In another embodiment, methods for identifying a modulator of mitochondrial mass and/or function may include the detection and/or measurement of an indicator of mitochondrial function, wherein the mitochondrial function involves programmed cell death or apoptosis. The range of responses to various known apoptogenic stimuli is broad, as is the range of methods and reagents for the detection of such responses.

Mitochondrial dysfunction is thought to be critical in the cascade of events leading to apoptosis in various cell types (Kroemer et al., FASEB J 9:1277-87, 1995). Mitochondrial physiology may be among the earliest events in programmed cell death (Zamzami et al., J. Exp. Med. 182:367-77, 1995; Zamzami et al., J. Exp. Med. 181:1661-72, 1995) and elevated reactive oxygen species (ROS) levels that result from such mitochondrial function may initiate the apoptotic cascade (Ausserer et al., Mol Cell Biol 14:5032-42, 1994). In several cell types, reduction in the mitochondrial membrane potential (DYm) precedes the nuclear DNA degradation that accompanies apoptosis. In cell-free systems, mitochondrial, but not nuclear, enriched fractions are capable of inducing nuclear apoptosis (Newmeyer et al., Cell 70:353-64, 1994). Perturbation of mitochondrial respiratory activity leading to altered cellular metabolic states, such as elevated intracellular ROS, may occur in type 2 diabetes and may further induce pathogenetic events via apoptotic mechanisms.

Oxidatively stressed mitochondria may release a pre-formed soluble factor that can induce chromosomal condensation, an event preceding apoptosis (Marchetti et al., Cancer Res. 56:2033-38, 1996). In addition, members of the Bcl-2 family of anti-apoptosis gene products are located within the outer mitochondrial membrane (Monaghan et al., J. Histochem. Cytochem. 40:1819-25, 1992) and these proteins appear to protect membranes from oxidative stress (Korsmeyer et al., Biochim. Biophys. Act. 1271:63, 1995). Localization of Bcl-2 to this membrane appears to be indispensable for modulation of apoptosis (Nguyen et al., J. Biol. Chem. 269:16521-24, 1994). Thus, changes in mitochondrial physiology may be important mediators of apoptosis.

Impaired mitochondrial function may therefore be reflected in a lower threshold for induction of apoptosis by one or more apoptogens. A variety of apoptogens are known to those familiar with the art (see, e.g., Green et al., 1998 Science 281:1309 and references cited therein) and may include by way of illustration and not limitation: tumor necrosis factor-alpha (TNF-a); Fas ligand; glutamate; N-methyl-D-aspartate (NMDA); interleukin-3 (IL-3); herbimycin A (Mancinitet al., 1997 J. Cell. Biol. 138:449-469); paraquat (Costantini et al., 1995 Toxicology 99:1-2); ethylene glycols; protein kinase inhibitors, e.g., staurosporine, calphostin C, caffeic acid phenethyl ester, chelerythrine chloride, genistein; 1-(5-isoquinolinesulfonyl)-2-methylpiperazine; KN-93; N-[2-((p-bromocinnamyl)amino)ethyl]-5-5-isoquinolinesulfonamide; d-erythrosphingosine derivatives; UV irradiation; ionophores, e.g., ionomycin and valinomycin; MAP kinase inducers, e.g., anisomycin, anandamine; cell cycle blockers, e.g., aphidicolin, colcemid, 5-fluorouracil, homoharringtonine; acetylcholinesterase inhibitors, e.g. berberine; anti-estrogens, e.g., tamoxifen; pro-oxidants, e.g.,: tert-butyl peroxide, hydrogen peroxide; free radicals, e.g., nitric oxide; inorganic metal ions, e.g., cadmium; DNA synthesis inhibitors, e.g., actinomycin D; DNA intercalators, e.g., doxorubicin, bleomycin sulfate, hydroxyurea, methotrexate, mitomycin C, camptothecin, daunorubicin; protein synthesis inhibitors, e.g., cycloheximide, puromycin, rapamycin; agents that affect microtubulin formation or stability, e.g., vinblastine, vincristine, colchicine, 4-hydroxyphenylretinamide, paclitaxel; Bad protein, Bid protein and Bax protein (see, e.g., Jurgenmeier et al., 1998 Proc. Nat. Acad. Sci. USA 95:4997-5002 and references cited therein); calcium and inorganic phosphate (Kroemer et al., 1998 Ann. Rev. Physiol 60:619).

In one embodiment, wherein the indicator of mitochondrial function is a cellular response to an apoptogen, cells in a biological sample that are suspected of undergoing apoptosis may be examined for morphological, permeability or other changes that are indicative of an apoptotic state. For example by way of illustration and not limitation, apoptosis in many cell types may cause altered morphological appearance such as plasma membrane blebbing, cell shape change, loss of substrate adhesion properties or other morphological changes that can be readily detected by a person having ordinary skill in the art, for example by using light microscopy. As another example, cells undergoing apoptosis may exhibit fragmentation and disintegration of chromosomes, which may be apparent by microscopy and/or through the use of DNA-specific or chromatin-specific dyes that are known in the art, including fluorescent dyes. Such cells may also exhibit altered plasma membrane permeability properties as may be readily detected through the use of vital dyes (e.g., propidium iodide, trypan blue) or by the detection of lactate dehydrogenase leakage into the extracellular milieu. These and other means for detecting apoptotic cells by morphologic criteria, altered plasma membrane permeability and related changes will be apparent to those familiar with the art.

In another embodiment, wherein the indicator of mitochondrial function is a cellular response to an apoptogen, cells in a biological sample may be assayed for translocation of cell membrane phosphatidylserine (PS) from the inner to the outer leaflet of the plasma membrane, which may be detected, for example, by measuring outer leaflet binding by the PS-specific protein annexin. (Martin et al., J. Exp. Med. 182:1545, 1995; Fadok et al., J. Immunol. 148:2207, 1992.) In still another embodiment, a cellular/biochemical response to an apoptogen is determined by an assay for induction of specific protease activity in any member of a family of apoptosis-activated proteases known as the caspases (see, e.g., Green et al., 1998 Science 281:1309). Those having ordinary skill in the art will be readily familiar with methods for determining caspase activity, for example by determination of caspase-mediated cleavage of specifically recognized protein substrates. These substrates may include, for example, poly-(ADP-ribose) polymerase (PARP) or other naturally occurring or synthetic peptides and proteins cleaved by caspases that are known in the art (see, e.g., Ellerby et al., 1997 J. Neurosci. 17:6165). Synthetic peptide substrates have been defined (Kluck et al., 1997 Science 275:1132; Nicholson et al., 1995 Nature 376:37). Other non-limiting examples of substrates include nuclear proteins such as U1-70 kDa and DNA-PKcs (Rosen and Casciola-Rosen, 1997 J. Cell. Biochem. 64:50; Cohen, 1997 Biochem. J. 326:1).

As described above, the mitochondrial inner membrane may exhibit highly selective and regulated permeability for many small solutes, but is impermeable to large (less than around 10 kDa) molecules. (See, e.g., Quinn, 1976 The Molecular Biology of Cell Membranes, University Park Press, Baltimore, Md.). In cells undergoing apoptosis, however, collapse of mitochondrial membrane potential may be accompanied by increased permeability permitting macromolecule diffusion across the mitochondrial membrane. Thus, in another embodiment of the subject invention method wherein the indicator of mitochondrial function is a cellular response to an apoptogen, detection of a mitochondrial protein, for example cytochrome c that has escaped from mitochondria in apoptotic cells, may provide evidence of a response to an apoptogen that can be readily determined. (Liu et al., Cell 86:147, 1996) Such detection of cytochrome c may be performed spectrophotometrically, immunochemically or by other well established methods for determining the presence of a specific protein.

For instance, release of cytochrome c from cells challenged with apoptotic stimuli (e.g., ionomycin, a well known calcium ionophore) can be followed by a variety of immunological methods. Matrix-assisted laser desorption ionization time-of-flight (MALDI-TOF) mass spectrometry coupled with affinity capture is particularly suitable for such analysis since apo-cytochrome c and holo-cytochrome c can be distinguished on the basis of their unique molecular weights. For example, the Surface-Enhanced Laser Desorption/Ionization (SELDI) system (Ciphergen, Palo Alto, Calif.) may be utilized to detect cytochrome c release from mitochondria in apoptogen treated cells. In this approach, a cytochrome c specific antibody immobilized on a solid support is used to capture released cytochrome c present in a soluble cell extract. The captured protein is then encased in a matrix of an energy absorption molecule (EAM) and is desorbed from the solid support surface using pulsed laser excitation. The molecular mass of the protein is determined by its time of flight to the detector of the SELDI mass spectrometer.

A person having ordinary skill in the art will readily appreciate that there may be other suitable techniques for quantifying apoptosis, and such techniques for purposes of determining an indicator of mitochondrial function that is a cellular response to an apoptogenic stimulus are within the scope of the methods provided by the present invention.

vi. Free Radical Production as an Indicator of Mitochondrial Function

In certain embodiments methods for identifying modulators of mitochondrial mass and/or function involve detecting free radical production in a biological sample as an indicator of mitochondrial function. Although mitochondria are a primary source of free radicals in biological systems (see, e.g., Murphy et al., 1998 in Mitochondria and Free Radicals in Neurodegenerative Diseases, Beal, Howell and Bodis-Wollner, Eds., Wiley-Liss, New York, pp. 159-186 and references cited therein), the methods described herein should not be so limited and free radical production can be an indicator of mitochondrial function regardless of the particular subcellular source site. For example, numerous intracellular biochemical pathways that lead to the formation of radicals through production of metabolites such as hydrogen peroxide, nitric oxide or superoxide radical via reactions catalyzed by enzymes such as flavin-linked oxidases, superoxide dismutase or nitric oxide synthetase, are known in the art, as are methods for detecting such radicals (see, e.g., Kelver, 1993 Crit. Rev. Toxicol. 23:21; Halliwell B. and J. M. C. Gutteridge, Free Radicals in Biology and Medicine, 1989 Clarendon Press, Oxford, UK; Davies, K. J. A. and F. Ursini, The Oxygen Paradox, Cleup Univ. Press, Padova, IT). Mitochondrial function, such as failure at any step of the ETC, may also lead to the generation of highly reactive free radicals. As noted above, radicals resulting from mitochondrial function include reactive oxygen species (ROS), for example, superoxide, peroxynitrite and hydroxyl radicals, and potentially other reactive species that may be toxic to cells. Accordingly, in certain embodiments, an indicator of mitochondrial function may be a detectable free radical species present in a biological sample. In certain embodiments, the detectable free radical will be a ROS.

Methods for detecting a free radical that may be useful as an indicator of mitochondrial function are known in the art and will depend on the particular radical.

Typically, a level of free radical production in a biological sample may be determined according to methods with which those skilled in the art will be readily familiar, including but not limited to detection and/or measurement of: glycoxidation products including pentosidine, carboxymethylysine and pyrroline; lipoxidation products including glyoxal, malondialdehyde and 4-hydroxynonenal; thiobarbituric acid reactive substances (TBARS; see, e.g., Stemnbrecher et al., 1984 Proc. Nat. Acad. Sci. USA 81:3883; Wolff, 1993 Br. Med. Bull. 49:642) and/or other chemical detection means such as salicylate trapping of hydroxyl radicals (e.g., Ghiselli et al., 1998 Meths. Mol. Biol. 108:89; Halliwell et al., 1997 Free Radic. Res. 27:239) or specific adduct formation (see, e.g., Mecocci et al. 1993 Ann. Neurol. 34:609; Giulivi et al., 1994 Meths. Enzymol. 233:363) including malondialdehyde formation, protein nitrosylation, DNA oxidation including mitochondrial DNA oxidation, 8-OH-guanosine adducts (e.g., Beckman et al., 1999 Mutat. Res. 424:5 1), protein oxidation, protein carbonyl modification (e.g., Baynes et al., 1991 Diabetes 40:405; Baynes et al., 1999 Diabetes 48: 1); electron spin resonance (ESR) probes; cyclic voltametry; fluorescent and/or chemiluminescent indicators (see also e.g., Greenwald, R. A. (ed.), Handbook of Methods for Oxygen Radical Research, 1985 CRC Press, Boca Raton, Fla.; Acworth and Bailey, (eds.), Handbook of Oxidative Metabolism, 1995 ESA, Inc., Chelmsford, Mass.; Yla-Herttuala et al., 1989 J. Clin. Invest. 84:1086; Velazques et al., 1991 Diabetic Medicine 8:752; Belch et al., 1995 Int. Angiol. 14:385; Sato et al., 1979 Biochem. Med. 21:104; Traverso et al., 1998 Diabetologia 41:265; Haugland, 1996 Handbook of Fluorescent Probes and Research Chemicals—Sixth Ed., Molecular Probes, Eugene, Oreg., pp. 483-502, and references cited therein). For example, by way of illustration and not limitation, oxidation of the fluorescent probes dichlorodihydrofluorescein diacetate and its carboxylated derivative carboxydichlorodihydrofluorescein diacetate (see, e.g., Haugland, 1996, supra) may be quantified following accumulation in cells, a process that is dependent on, and proportional to, the presence of reactive oxygen species (see also, e.g., Molecular Probes On-line Handbook of Fluorescent Probes and Research Chemicals, world wide web at probes.com/handbook/toc.html). Other fluorescent detectable compounds that may be used in the invention for detection of free radical production include but are not limited to dihydrorhodamine and dihydrorosamine derivatives, cis-parinaric acid, resorufin derivatives, lucigenin and any other suitable compound that may be known to those familiar with the art.

Thus, as also described above, free radical mediated damage may inactivate one or more of the myriad proteins of the ETC and in doing so, may uncouple the mitochondrial chemiosmotic mechanism responsible for oxidative phosphorylation and ATP production. Indicators of mitochondrial function that are ATP biosynthesis factors, including determination of ATP production, are described in greater detail herein. Free radical mediated damage to mitochondrial functional integrity is also just one example of multiple mechanisms associated with mitochondrial fuction that may result in collapse of the electrochemical potential maintained by the inner mitochondrial membrane.

In other embodiments, provided are methods for treating an individual that may benefit from increased mitochondrial mass and/or function. The methods may involve first identifying a patient suffering from a mitochondrial dysfunction. The methods described above for identifying an agent that modulates mitochondrial mass and/or function may also be used for identifying an individual that would benefit from increased mitochondrial mass and/or activity. For example, the methods described above may be used to measure mitochondrial mass and/or function in a biological sample from one individual as compared to an individual (e.g., an individual having normal mitochondrial mass and/or function), a control population, or standard predetermined values of mitochondrial mass and/or function.

IV. Pharmaceutical Formulations and Administration Modes

Pharmaceutical compositions for use in accordance with the present methods may be formulated in any conventional manner using one or more physiologically acceptable carriers or excipients. Thus, sirtuin-activating or -inhibiting compounds and their physiologically acceptable salts and solvates may be formulated for administration by, for example, injection (e.g. subcutaneous, intramuscular, intraperitoneal), inhalation or insufflation (either through the mouth or the nose) or oral, buccal, parenteral, sublingual or rectal administration. In one embodiment, the compound is administered locally, at the site where of target cells, e.g., adipose cells.

Compounds can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets, lozanges, or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated to give controlled release of the active compound.

Polyphenols such as resveratrol can oxidize and lose sirtuin-stimulatory activity, especially in a liquid or semi-solid form. To prevent oxidation and preserve the sirtuin-stimulatory activity of polyphenol-containing compounds, the compounds may be stored in a nitrogen atmosphere or sealed in a type of capsule and/or foil package that excludes oxygen (e.g., CapSugel™).

For administration by inhalation, the compounds may be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin, for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches.

In certain embodiments, pharmaceutical compositions can be administered with medical devices known in the art. For example, a pharmaceutical composition described herein can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules also are known.

In certain embodiments, the compounds described herein can be formulated for delivery to the central nervous system (CNS) (reviewed in Begley, Pharmacology & Therapeutics 104: 29-45 (2004)). Conventional approaches for drug delivery to the CNS include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the blood-brain barrier (BBB)) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide).

One possibility to achieve sustained release kinetics is embedding or encapsulating the active compound into nanoparticles. Nanoparticles can be administrated as powder, as a powder mixture with added excipients or as suspensions. Colloidal suspensions of nanoparticles can easily be administrated through a cannula with small diameter.

Nanoparticles are particles with a diameter from about 5 nm to up to about 1000 nm. The term “nanoparticles” as it is used hereinafter refers to particles formed by a polymeric matrix in which the active compound is dispersed, also known as “nanospheres”, and also refers to nanoparticles which are composed of a core containing the active compound which is surrounded by a polymeric membrane, also known as “nanocapsules”. In certain embodiments, nanoparticles are preferred having a diameter from about 50 nm to about 500 nm, in particular from about 100 nm to about 200 nm.

Nanoparticles can be prepared by in situ polymerization of dispersed monomers or by using preformed polymers. Since polymers prepared in situ are often not biodegradable and/or contain toxicological serious byproducts, nanoparticles from preformed polymers are preferred. Nanoparticles from preformed polymers can be prepared by different techniques, e.g., by emulsion evaporation, solvent displacement, salting-out, mechanical grinding, microprecipitation, and by emulsification diffusion.

With the methods described above, nanoparticles can be formed with various types of polymers. For use in the method of the present invention, nanoparticles made from biocompatible polymers are preferred. The term “biocompatible” refers to material that after introduction into a biological environment has no serious effects to the biological environment. From biocompatible polymers those polymers are especially preferred which are also biodegradable. The term “biodegradable” refers to material that after introduction into a biological environment is enzymatically or chemically degraded into smaller molecules, which can be eliminated subsequently. Examples are polyesters from hydroxycarboxylic acids such as poly(lactic acid) (PLA), poly(glycolic acid) (PGA), polycaprolactone (PCL), copolymers of lactic acid and glycolic acid (PLGA), copolymers of lactic acid and caprolactone, polyepsilon caprolactone, polyhyroxy butyric acid and poly(ortho)esters, polyurethanes, polyanhydrides, polyacetals, polydihydropyrans, polycyanoacrylates, natural polymers such as alginate and other polysaccharides including dextran and cellulose, collagen and albumin.

Suitable surface modifiers can preferably be selected from known organic and inorganic pharmaceutical excipients. Such excipients include various polymers, low molecular weight oligomers, natural products and surfactants. Preferred surface modifiers include nonionic and ionic surfactants. Representative examples of surface modifiers include gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, e.g., macrogol ethers such as cetomacrogol 1000, polyoxyethylene castor oil derivatives, polyoxyethylene sorbitan fatty acid esters, e.g., the commercially available Tweens™, polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthalate, noncrystalline cellulose, magnesium aluminum silicate, triethanolamine, polyvinyl alcohol, and polyvinylpyrrolidone (PVP). Most of these surface modifiers are known pharmaceutical excipients and are described in detail in the Handbook of Pharmaceutical Excipients, published jointly by the American Pharmaceutical Association and The Pharmaceutical Society of Great Britain, the Pharmaceutical Press, 1986.

Further description on preparing nanoparticles can be found, for example, in U.S. Pat. No. 6,264,922, the contents of which are incorporated herein by reference.

Liposomes are a further drug delivery system which is easily injectable. Accordingly, in the method of invention the active compounds can also be administered in the form of a liposome delivery system. Liposomes are well-known by a person skilled in the art. Liposomes can be formed from a variety of phospholipids, such as cholesterol, stearylamine of phosphatidylcholines. Liposomes being usable for the method of invention encompass all types of liposomes including, but not limited to, small unilamellar vesicles, large unilamellar vesicles and multilamellar vesicles.

Liposomes are used for a variety of therapeutic purposes, and in particular, for carrying therapeutic agents to target cells. Advantageously, liposome-drug formulations offer the potential of improved drug-delivery properties, which include, for example, controlled drug release. An extended circulation time is often needed for liposomes to reach a target region, cell or site. In particular, this is necessary where the target region, cell or site is not located near the site of administration. For example, when liposomes are administered systemically, it is desirable to coat the liposomes with a hydrophilic agent, for example, a coating of hydrophilic polymer chains such as polyethylene glycol (PEG) to extend the blood circulation lifetime of the liposomes. Such surface-modified liposomes are commonly referred to as “long circulating” or “sterically stabilized” liposomes.

One surface modification to a liposome is the attachment of PEG chains, typically having a molecular weight from about 1000 daltons (Da) to about 5000 Da, and to about 5 mole percent (%) of the lipids making up the liposomes (see, for example, Stealth Liposomes, CRC Press, Lasic, D. and Martin, F., eds., Boca Raton, Fla., (1995)), and the cited references therein. The pharmacokinetics exhibited by such liposomes are characterized by a dose-independent reduction in uptake of liposomes by the liver and spleen via the mononuclear phagocyte system (MPS), and significantly prolonged blood circulation time, as compared to non-surface-modified liposomes, which tend to be rapidly removed from the blood and accumulated in the liver and spleen.

In certain embodiments, the complex is shielded to increase the circulatory half-life of the complex or shielded to increase the resistance of nucleic acid to degradation, for example degradation by nucleases.

As used herein, the term “shielding”, and its cognates such as “shielded”, refers to the ability of “shielding moieties” to reduce the non-specific interaction of the complexes described herein with serum complement or with other species present in serum in vitro or in vivo. Shielding moieties may decrease the complex interaction with or binding to these species through one or more mechanisms, including, for example, non-specific steric or non-specific electronic interactions. Examples of such interactions include non-specific electrostatic interactions, charge interactions, Van der Waals interactions, steric-hindrance and the like. For a moiety to act as a shielding moiety, the mechanism or mechanisms by which it may reduce interaction with, association with or binding to the serum complement or other species does not have to be identified. One can determine whether a moiety can act as a shielding moiety by determining whether or to what extent a complex binds serum species.

It should be noted that “shielding moieties” can be multifunctional. For example, a shielding moiety may also function as, for example, a targeting factor. A shielding moiety may also be referred to as multifunctional with respect to the mechanism(s) by which it shields the complex. While not wishing to be limited by proposed mechanism or theory, examples of such a multifunctional shielding moiety are pH sensitive endosomal membrane-disruptive synthetic polymers, such as PPAA or PEAA. Certain poly(alkylacrylic acids) have been shown to disrupt endosomal membranes while leaving the-outer cell surface membrane intact (Stayton et al. (2000) J. Controll. Release 65:203-220; Murthy et al. (1999) J. Controll. Release 61:137-143; WO 99/34831), thereby increasing cellular bioavailability and functioning as a targeting factor. However, PPAA reduces binding of serum complement to complexes in which it is incorporated, thus functioning as a shielding moiety.

Another way to produce a formulation, particularly a solution, of a sirtuin modulator such as resveratrol or a derivative thereof, is through the use of cyclodextrin. By cyclodextrin is meant α-, β-, or γ-cyclodextrin. Cyclodextrins are described in detail in Pitha et al., U.S. Pat. No. 4,727,064, which is incorporated herein by reference. Cyclodextrins are cyclic oligomers of glucose; these compounds form inclusion complexes with any drug whose molecule can fit into the lipophile-seeking cavities of the cyclodextrin molecule.

The cyclodextrin of the compositions according to the invention may be α-, β-, or γ-cyclodextrin. α-cyclodextrin contains six glucopyranose units; β-cyclodextrin contains seven glucopyranose units; and γ-cyclodextrin contains eight glucopyranose units. The molecule is believed to form a truncated cone having a core opening of 4.7-5.3 angstroms, 6.0-6.5 angstroms, and 7.5-8.3 angstroms in α-, β-, or γ-cyclodextrin respectively. The composition according to the invention may comprise a mixture of two or more of the α-, β-, or γ-cyclodextrins. Typically, however, the composition according to the invention will comprise only one of the α-, β-, or γ-cyclodextrins.

Most preferred cyclodextrins in the compositions according to the invention are amorphous cyclodextrin compounds. By amorphous cyclodextrin is meant non-crystalline mixtures of cyclodextrins wherein the mixture is prepared from α-, β-, or γ-cyclodextrin. In general, the amorphous cyclodextrin is prepared by non-selective alkylation of the desired cyclodextrin species. Suitable alkylation agents for this purpose include but are not limited to propylene oxide, glycidol, iodoacetamide, chloroacetate, and 2-diethylaminoethlychloride. Reactions are carried out to yield mixtures containing a plurality of components thereby preventing crystallization of the cyclodextrin. Various alkylated cyclodextrins can be made and of course will vary, depending upon the starting species of cyclodextrin and the alkylating agent used. Among the amorphous cyclodextrins suitable for compositions according to the invention are hydroxypropyl, hydroxyethyl, glucosyl, maltosyl and maltotriosyl derivatives of β-cyclodextrin, carboxyamidomethyl-β-cyclodextrin, carboxymethyl-β-cyclodextrin, hydroxypropyl-β-cyclodextrin and diethylamino-β-cyclodextrin.

One example of resveratrol dissolved in the presence of a cyclodextrin is provided in Marier et al., J. Pharmacol. Exp. Therap. 302:369-373 (2002), the contents of which are incorporated herein by reference, where a 6 mg/mL solution of resveratrol was prepared using 0.9% saline containing 20% hydroxylpropyl-β-cyclodextrin.

As mentioned above, the compositions of matter of the invention comprise an aqueous preparation of preferably substituted amorphous cyclodextrin and one or more sirtuin modulators. The relative amounts of sirtuin modulators and cyclodextrin will vary depending upon the relative amount of each of the sirtuin modulators and the effect of the cyclodextrin on the compound. In general, the ratio of the weight of compound of the sirtuin modulators to the weight of cyclodextrin compound will be in a range between 1:1 and 1:100. A weight to weight ratio in a range of 1:5 to 1:50 and more preferably in a range of 1:10 to 1:20 of the compound selected from sirtuin modulators to cyclodextrin are believed to be the most effective for increased circulating availability of the sirtuin modulator.

Importantly, if the aqueous solution comprising the sirtuin modulators and a cyclodextrin is to be administered parenterally, especially via the intravenous route, a cyclodextrin will be substantially free of pyrogenic contaminants. Various forms of cyclodextrin, such as forms of amorphous cyclodextrin, may be purchased from a number of vendors including Sigma-Aldrich, Inc. (St. Louis, Mo., USA). A method for the production of hydroxypropyl-β-cyclodextrin is disclosed in Pitha et al., U.S. Pat. No. 4,727,064 which is incorporated herein by reference.

Additional description of the use of cyclodextrin for solubilizing compounds can be found in US 2005/0026849, the contents of which are incorporated herein by reference.

Rapidly disintegrating or dissolving dosage forms are useful for the rapid absorption, particularly buccal and sublingual absorption, of pharmaceutically active agents. Fast melt dosage forms are beneficial to patients, such as aged and pediatric patients, who have difficulty in swallowing typical solid dosage forms, such as caplets and tablets. Additionally, fast melt dosage forms circumvent drawbacks associated with, for example, chewable dosage forms, wherein the length of time an active agent remains in a patient's mouth plays an important role in determining the amount of taste masking and the extent to which a patient may object to throat grittiness of the active agent.

To overcome such problems manufacturers have developed a number of fast melt solid dose oral formulations. These are available from manufacturers including Cima Labs, Fuisz Technologies Ltd., Prographarm, R. P. Scherer, Yamanouchi-Shaklee, and McNeil-PPC, Inc. All of these manufacturers market different types of rapidly dissolving solid oral dosage forms. See e.g., patents and publications by Cima Labs such as U.S. Pat. Nos. 5,607,697, 5,503,846, 5,223,264, 5,401,513, 5,219,574, and 5,178,878, WO 98/46215, WO 98/14179; patents to Fuisz Technologies, now part of BioVail, such as U.S. Pat. Nos. 5,871,781, 5,869,098, 5,866,163, 5,851,553, 5,622,719, 5,567,439, and 5,587,172; U.S. Pat. No. 5,464,632 to Prographarm; patents to R. P. Scherer such as U.S. Pat. Nos. 4,642,903, 5,188,825, 5,631,023 and 5,827,541; patents to Yamanouchi-Shaklee such as U.S. Pat. Nos. 5,576,014 and 5,446,464; patents to Janssen such as U.S. Pat. Nos. 5,807,576, 5,635,210, 5,595,761, 5,587,180 and 5,776,491; U.S. Pat. Nos. 5,639,475 and 5,709,886 to Eurand America, Inc.; U.S. Pat. Nos. 5,807,578 and 5,807,577 to L.A.B. Pharmaceutical Research; patents to Schering Corporation such as U.S. Pat. Nos. 5,112,616 and 5,073,374; U.S. Pat. No. 4,616,047 to Laboratoire L. LaFon; U.S. Pat. No. 5,501,861 to Takeda Chemicals Inc., Ltd.; and U.S. Pat. No. 6,316,029 to Elan.

In one example of fast melt tablet preparation, granules for fast melt tablets made by either the spray drying or pre-compacting processes are mixed with excipients and compressed into tablets using conventional tablet making machinery. The granules can be combined with a variety of carriers including low density, high moldability saccharides, low moldability saccharides, polyol combinations, and then directly compressed into a tablet that exhibits an improved dissolution and disintegration profile.

The tablets according to the present invention typically have a hardness of about 2 to about 6 Strong-Cobb units (scu). Tablets within this hardness range disintegrate or dissolve rapidly when chewed. Additionally, the tablets rapidly disentegrate in water. On average, a typical 1.1 to 1.5 gram tablet disintegrates in 1-3 minutes without stirring. This rapid disintegration facilitates delivery of the active material.

The granules used to make the tablets can be, for example, mixtures of low density alkali earth metal salts or carbohydrates. For example, a mixture of alkali earth metal salts includes a combination of calcium carbonate and magnesium hydroxide. Similarly, a fast melt tablet can be prepared according to the methods of the present invention that incorporates the use of A) spray dried extra light calcium carbonate/maltodextrin, B) magnesium hydroxide and C) a eutectic polyol combination including Sorbitol Instant, xylitol and mannitol. These materials have been combined to produce a low density tablet that dissolves very readily and promotes the fast disintegration of the active ingredient. Additionally, the pre-compacted and spray dried granules can be combined in the same tablet.

For fast melt tablet preparation, a sirtuin modulator useful in the present invention can be in a form such as solid, particulate, granular, crystalline, oily or solution. The sirtuin modulator for use in the present invention may be a spray dried product or an adsorbate that has been pre-compacted to a harder granular form that reduces the medicament taste. A pharmaceutical active ingredient for use in the present invention may be spray dried with a carrier that prevents the active ingredient from being easily extracted from the tablet when chewed.

In addition to being directly added to the tablets of the present invention, the medicament drug itself can be processed by the pre-compaction process to achieve an increased density prior to being incorporated into the formulation.

The pre-compaction process used in the present invention can be used to deliver poorly soluble pharmaceutical materials so as to improve the release of such pharmaceutical materials over traditional dosage forms. This could allow for the use of lower dosage levels to deliver equivalent bioavailable levels of drug and thereby lower toxicity levels of both currently marketed drug and new chemical entities. Poorly soluble pharmaceutical materials can be used in the form of nanoparticles, which are nanometer-sized particles.

In addition to the active ingredient and the granules prepared from low density alkali earth metal salts and/or water soluble carbohydrates, the fast melt tablets can be formulated using conventional carriers or excipients and well established pharmaceutical techniques. Conventional carriers or excipients include, but are not limited to, diluents, binders, adhesives (i.e., cellulose derivatives and acrylic derivatives), lubricants (i.e., magnesium or calcium stearate, vegetable oils, polyethylene glycols, talc, sodium lauryl sulphate, polyoxy ethylene monostearate), disintegrants, colorants, flavorings, preservatives, sweeteners and miscellaneous materials such as buffers and adsorbents.

Additional description of the preparation of fast melt tablets can be found, for example, in U.S. Pat. No. 5,939,091, the contents of which are incorporated herein by reference.

Pharmaceutical compositions (including cosmetic preparations) may comprise from about 0.00001 to 100% such as from 0.001 to 10% or from 0.1% to 5% by weight of one or more compounds described herein.

In one embodiment, a compound described herein, is incorporated into a topical formulation containing a topical carrier that is generally suited to topical drug administration and comprising any such material known in the art. The topical carrier may be selected so as to provide the composition in the desired form, e.g., as an ointment, lotion, cream, microemulsion, gel, oil, solution, or the like, and may be comprised of a material of either naturally occurring or synthetic origin. It is preferable that the selected carrier not adversely affect the active agent or other components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like.

Formulations may be colorless, odorless ointments, lotions, creams, microemulsions and gels.

Compounds may be incorporated into ointments, which generally are semisolid preparations which are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington's, cited in the preceding section, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Exemplary water-soluble ointment bases are prepared from polyethylene glycols (PEGs) of varying molecular weight; again, reference may be had to Remington's, supra, for further information.

Compounds may be incorporated into lotions, which generally are preparations to be applied to the skin surface without friction, and are typically liquid or semiliquid preparations in which solid particles, including the active agent, are present in a water or alcohol base. Lotions are usually suspensions of solids, and may comprise a liquid oily emulsion of the oil-in-water type. Lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. An exemplary lotion formulation for use in conjunction with the present method contains propylene glycol mixed with a hydrophilic petrolatum such as that which may be obtained under the trademark Aquaphor®™ from Beiersdorf, Inc. (Norwalk, Conn.).

Compounds may be incorporated into creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington's, supra, is generally a nonionic, anionic, cationic or amphoteric surfactant.

Compounds may be incorporated into microemulsions, which generally are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

Compounds may be incorporated into gel formulations, which generally are semisolid systems consisting of either suspensions made up of small inorganic particles (two-phase systems) or large organic molecules distributed substantially uniformly throughout a carrier liquid (single phase gels). Single phase gels can be made, for example, by combining the active agent, a carrier liquid and a suitable gelling agent such as tragacanth (at 2 to 5%), sodium alginate (at 2-10%), gelatin (at 2-15%), methylcellulose (at 3-5%), sodium carboxymethylcellulose (at 2-5%), carbomer (at 0.3-5%) or polyvinyl alcohol (at 10-20%) together and mixing until a characteristic semisolid product is produced. Other suitable gelling agents include methylhydroxycellulose, polyoxyethylene-polyoxypropylene, hydroxyethylcellulose and gelatin. Although gels commonly employ aqueous carrier liquid, alcohols and oils can be used as the carrier liquid as well.

Various additives, known to those skilled in the art, may be included in formulations, e.g., topical formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation.

A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C₁₀ MSO) and tetradecylmethyl sulfboxide; pyrrolidones such as 2-pyrrolidone, N-methyl-2-pyrrolidone and N-(-hydroxyethyl)pyrrolidone; urea; N,N-diethyl-m-toluamide; C₂-C₆ alkanediols; miscellaneous solvents such as dimethyl formamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark Azone®™ from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol®™) and diethylene glycol monoethyl ether oleate (available commercially as Softcutol®™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol®™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein.

Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate).

In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation.

Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition as herein defined.

In an alternative embodiment, a pharmaceutical formulation is provided for oral or parenteral administration, in which case the formulation may comprise an activating compound-containing microemulsion as described above, and may contain alternative pharmaceutically acceptable carriers, vehicles, additives, etc. particularly suited to oral or parenteral drug administration. Alternatively, an activating compound-containing microemulsion may be administered orally or parenterally substantially as described above, without modification.

Administration of a sirtuin activator or inhibitor may be followed by measuring a factor in the subject, such as measuring the activity of the sirtuin. In an illustrative embodiment, a cell is obtained from a subject following administration of an activating or inhibiting compound to the subject, such as by obtaining a biopsy, and the activity of the sirtuin or sirtuin expression level is determined in the biopsy. Alternatively, biomarkers, such as plasma biomarkers may be followed. The cell may be any cell of the subject, but in cases in which an activating compound is administered locally, the cell is preferably a cell that is located in the vicinity of the site of administration. For example, the cell may be an adipose cell.

V. Exemplary Kits

Also provided herein are kits, e.g., kits for therapeutic purposes, including kits for treating or preventing metabolic disorders, such as obesity or diabetes, or secondary conditions thereof. A kit may comprise one or more high dosage formulations of a sirtuin activator, such as those described herein, and optionally devices for contacting cells with the agents. Devices include syringes, stents and other devices for introducing a compound into a subject or applying it to the skin of a subject.

Further, a kit may also contain components for measuring a factor, e.g., described above, such as the activity of sirtuin proteins, e.g., in tissue samples.

Other kits include kits for diagnosing the likelihood of having or developing a metabolic disorder, such as obesity or diabetes, or secondary conditions thereof. A kit may comprise an agent for measuring the activity and or expression level of a sirtuin.

Kits for screening assays are also provided. Exemplary kits comprise one or more agents for conducting a screening assay, such as a sirtuin or a biologically active portion thereof, or a cell or cell extract comprising such. Any of the kits may also comprise instructions for use.

The present description is further illustrated by the following examples, which should not be construed as limiting in any way. The contents of all cited references (including literature references, issued patents, published patent applications and GenBank Accession numbers as cited throughout this application) are hereby expressly incorporated by reference.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2^{nd} Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### EXEMPLIFICATION

The invention now being generally described, it will be more readily understood by reference to the following examples which are included merely for purposes of illustration of certain aspects and embodiments of the present invention, and are not intended to limit the invention in any way.

### Example 1

### Metabolic Activities of Sirtuin Activators in a Diet Induced Obesity (DIO) Mouse Model

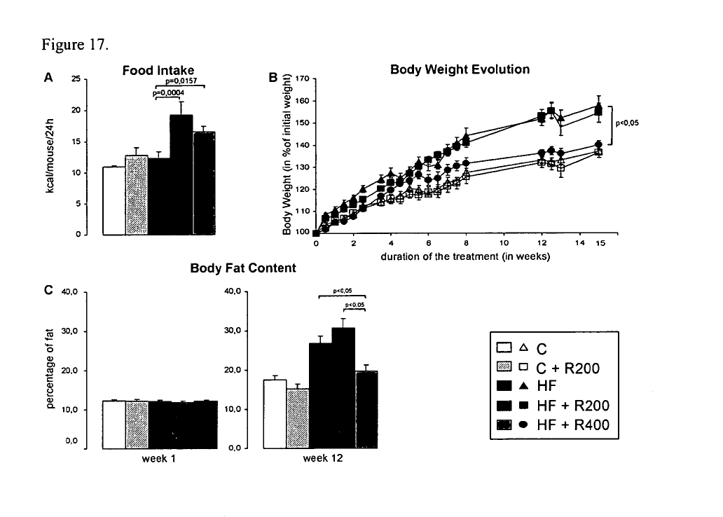

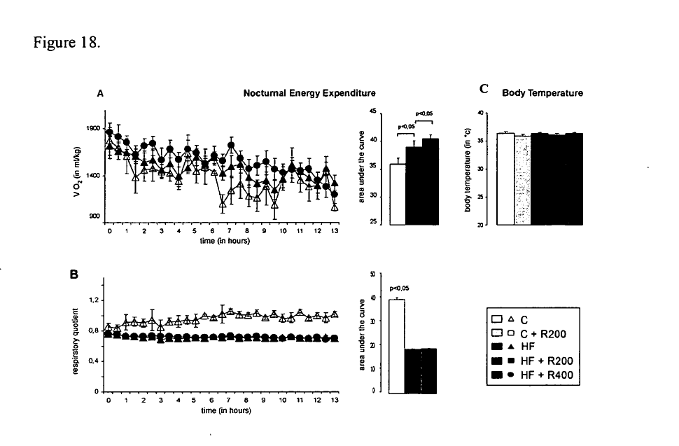

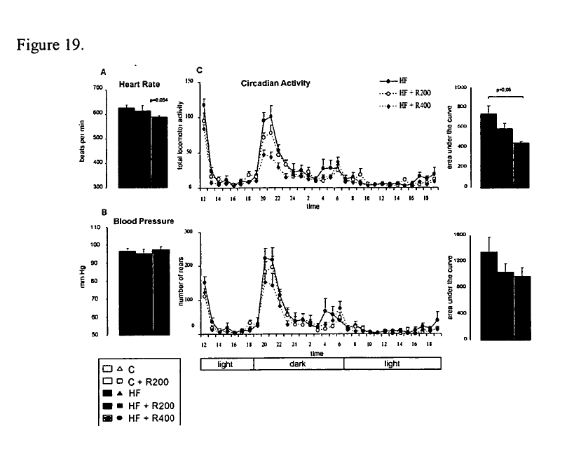

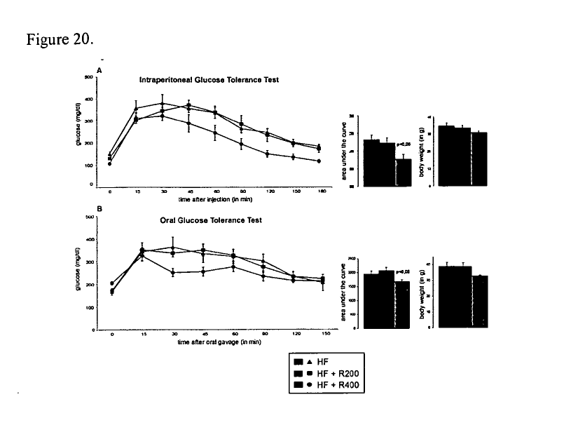

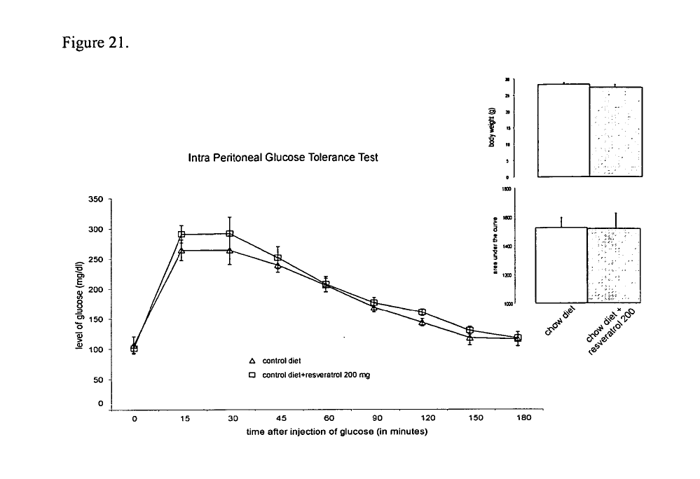

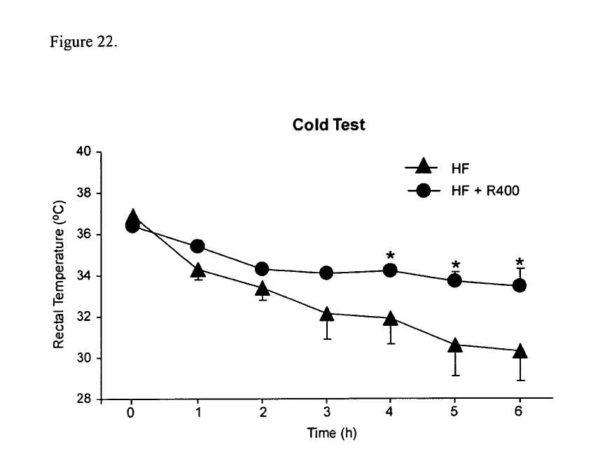

In order to define whether SIRT-1 activators protect against the development of obesity and associated insulin-resistance, resveratrol is chronically administered (via food admix) to male C57BL6J mice that are subjected during 16 weeks to a high fat diet. The mice undergo an extensive phenotypic and molecular analysis to define the regulatory pathways affected by Sirt-1 activation. See, for example, the results presented in FIGS. 17-21.

Resveratrol, as a food additive, has been shown to be well tolerated and does not cause food aversion. In this long-term study, 50 male C57BL6J mice (5 weeks of age) are analyzed during a period of 18 weeks. Five groups of 10 animals are assigned as follows:
1: chow diet
2: chow diet+resveratrol (200 mg/kg/day)
3: high fat diet
4: high fat diet+resveratrol (200 mg/kg/day)
5: high fat diet+resveratrol (400 mg/kg/day)

During the entire study, body weight and food intake

are monitored twice weekly.

During week 1, body composition is analyzed, for all groups, by dual energy X-ray absorptiometry (dexascan).

During week 2, serum levels of glucose, triglycerides, cholesterol, HDL-C, LDL-C and insulin are measured in all groups after a fasting period of 12 h and mice are then placed on the diets as indicated (Day 0).

During week 10, glucose tolerance is determined by subjecting all the animals to an intraperitoneal glucose tolerance test (IPGTT). Animals are fasted for 12 h prior to this test.

Nocturnal energy expenditure of groups 1, 3 and 5 (chow diet, high fat diet and high fat diet 400 mg) is measured by indirect calorimetry.

During week 12, body weight composition is again analysed by dexascan for all groups.

During week 13, circadian activity of groups 3, 4 and 5 (high fat diet fed mice) is studied during a period of 30 h.

During week 14, measurement of blood pressure and heart rate is performed on groups 3, 4 and 5.

During week 15, rectal temperature of all animals is measured at room temperature at 10:00 am.

A circadian activity measurement is performed on groups 1, 2 and 3.

During week 16, glucose tolerance is analysed by performing an oral glucose tolerance test (OGTT) on a subset of animals (n=5) of groups 3, 4 and 5, and an intraperitoneal insulin sensitivity test (IPIST) on another subset of animals (n=5). During these experiments, blood is also collected to analyze insulin levels. Animals are fasted 12 h prior these tests.

Feces are collected in all groups over a 24 h time period and fecal lipids content are measured.

During week 17, serum levels of resveratrol are measured on a subset of mice (n=5) at 7:00 am which corresponds to the beginning of the light cycle and on another subset of mice (n=5) three hours later (10:00 am). Moreover, thyroid hormone T3 levels are measured in the blood collected at 7:00 am and plasma lipoproteins levels are measured in the blood collected at 10:00 am.

During week 18, a cold test is performed on all animals by measuring body temperature of animals exposed to 4° C.

Three days later, animals are sacrified.

At sacrifice, blood is collected and analyzed for: plasma lipids (TC, TG, HDL-C, FFAs); liver functions (ALAT, ASAT, alkaline Pase, γ-GT); and glucose and insulin lipoprotein profiles of selected groups of plasma (size-exclusion chomatography).

Liver, small intestine, adipose tissues (WAT and BAT), pancreas, heart and muscle are collected and weighed. These can be analyzed by standard histology (HE staining, succinate dehydrogenase staining, oil-red-O staining and cell morphology); for tissue lipid content; and by electron microscopy on BAT and muscle to analyze mitochondria. RNA isolation can be conducted for expression studies of selected genes involved in metabolism and energy homeostasis by quantitative RT-PCR. Microarray experiments can also be performed on selected tissues. In addition, protein extraction can be performed for the study of changes in protein level and post-translational modifications such as acetylation of proteins of interest (e.g. PGC-1α).

Methods

Animal housing and handling. Mice are group housed (5 animals/cage) in specific pathogen-free conditions with a 12 h: 12 h (on at 7:00) light-dark cycle, in a temperature (20-22° C.) and humidity controlled vivarium, according to the European Community specifications. Animals are allowed free access to water and food.

Drinking water. Chemical composition of the tap water is regularly analyzed to verify the absence of potential toxic substances at the Institut d'Hydrologie, ULP, Strasbourg. Drinking water is treated with HCl and HClO₄ to maintain pH between 5 and 5.5 and chlorin concentration between 5 and 6 ppm.

Diet. The standard rodent chow diet is obtained from UAR and the high fat diet is obtained from Research Diet. Mice are fed, either with chow diet (16% protein, 3% fat, 5% fiber, 5% ash) or with high fat diet (26.2% protein, 26.3% carbohydrate, 34.9% fat). Resveratrol is mixed with either powdered chow diet or powdered high fat diet and pellets are reconstituted. Control groups receive pellets as provided by the company. Due to the consistency of the high fat diet, it is not necessary to add water to mix it with the resveratrol. In case of the chow, which is harder to reconstitute, a minimal amount of water is added to the powder to reconstitute pellets, which are then air-dried. New batches of food are prepared weekly.

Blood collection. Blood is collected either from the retro-orbital sinus or from the tail vein.

Anesthesia. For the dexa scanning experiment, animals are anesthesized with a mixture of ketamine (200 mg/kg)/Xylasine (10 mg/kg) administred by intra-peritoneal injection.

Biochemistry

Tests are performed with an Olympus AU-400 automated laboratory work station using commercial reagents (Olympus).

Analysis of lipids and lipoproteins. Serum triglycerides, total and HDL cholesterol are determined by enzymatic assays. Serum HDL cholesterol content is determined after precipitation of apo B-containing lipoproteins with phosphotungstic acid/Mg (Roche Diagnostics, Mannheim, Germany). Free fatty acids level is determined with a kit from Wako (Neuss, Germany) as specified by the provider.

Metabolic and endocrine exploration. Blood glucose concentration is measured by a Precision Q.I.D analyzer (Medisense system), using Medisense Precis electrodes (Abbot Laboratories, Medisense products, Bedford, USA). This method has been validated, by comparing Precision Q.I.D analyzer values with classical glucose measurements. The Precision Q.I.D method was chosen since it requires a minimal amount of blood and can hence be employed for multiple measurements such as during an IPGTT. Plasma insulin (Crystal Chem, Chicago, Ill.) is determined by ELISA according to the manufacturer's specifications. Plasma level of T3 is determined by standard radio-immunoassays (RIA) according to the protocol specified by the providers.

Metabolic Testing

Lipoprotein profiles. Lipoprotein profiles are obtained by fast protein liquid chromatography, allowing separation of the three major lipoprotein classes VLDL, LDL, and HDL.

Intraperitoneal glucose tolerance test—Oral glucose tolerance test. IPGTT and OGTT are performed in mice which are fasted overnight (12 h). Mice are either injected intraperitoneally (IPGTT) or orally gavaged (OGTT) with a solution of 20% glucose in sterile saline (0.9% NaCl) at a dose of 2 g glucose/kg body weight. Blood is collected from the tail vein, for glucose and insulin monitoring, prior to and at 15, 30, 45, 75, 90, 120, 150, 180 min after administration of the glucose solution. The incremental area of the glucose curve is calculated as a measure of insulin sensitivity, whereas the corresponding insulin levels indicate insulin secretory reserves.

Intraperitoneal insulin sensitivity test. Fasted animals are submitted to an IP injection of regular porcine insulin (0.5-1.0 IU/kg; Lilly, Indianapolis, Ind.). Blood is collected at 0, 15, 30, 45, 60, and 90 min after injection and glucose analyzed as described above. Insulin sensitivity is measured as the slope of the fall in glucose over time after injection of insulin.

Energy expenditure. Energy expenditure is evaluated through indirect calorimetry by measuring oxygen consumption with the Oxymax apparatus (Columbus Instruments, Columbus, Ohio) during 12 h. This system consists of an open circuit with air coming in and out of plastic cages (one mouse per cage). Animals are allowed free access to food and water. A very precise CO₂ and O₂ sensor measures the difference in O₂ and CO₂ concentrations in both air volumes, which gives the amount of oxygen consumed in a period of time given that the air flow of air coming in the cage is constant. The data coming out of the apparatus are processed in a connected computer, analyzed, and shown in an exportable Excel file. The values are expressed as ml.kg⁻¹.h⁻¹, which is commonly known as the VO₂.

Determination of body fat content by Dexa scanning. The Dexa analyses are performed by the ultra high resolution PIXIMUS Series Densitometer (0.18×0.18 mm pixels, GE Medical Systems, Madison, Wis., USA). Bone mineral density (BMD in g/cm²) and body composition are determined by using the PIXIMUS software (version 1.4x, GE Medical Systems).

Non-invasive Blood Pressure and Heart Rate Measurements

The Visitech BP-2000 Blood Pressure Analysis System is a computer-automated tail cuff system that is used for taking multiple measurements on 4 awake mice simultaneously without operator intervention. The mice are contained in individual dark chambers on a heated platform with their tails threaded through a tail cuff. The system measures blood pressure by determining the cuff pressure at which the blood flow to the tail is eliminated. A photoelectric sensor detects the specimen's pulse. The system generates results that Applicants have shown correspond closely with the mean intra-arterial pressure measured simultaneously in the carotid artery. This allows obtaining reproducible values of systolic blood pressure and heart beat rate. This requires training of the animals for one week in the system.

Circadian Activity

Spontaneous locomotor activity is measured using individual boxes, each composed with a sliding floor, a detachable cage, and equipped with infra-red captors allowing measurement of ambulatory locomotor activity and rears. Boxes are linked to a computer using an electronic interface (Imetronic, Pessac, France). Mice are tested for 32 h in order to measure habituation to the apparatus as well as nocturnal and diurnal activities. The quantity of water consumed is measured during the test period using an automated lickometer.

### Example 2

### Metabolic Activities of Sirtuin Activators in a Zucker Diabetic Rat Model

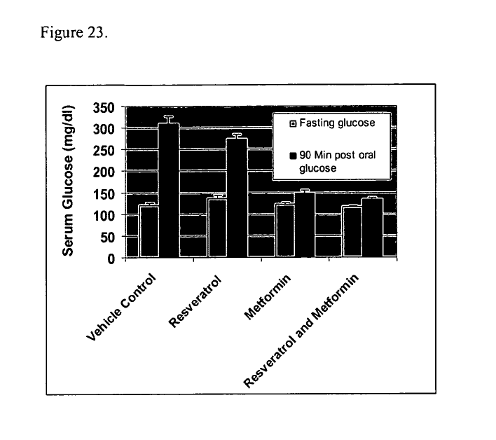

Resveratrol (200 mg/kg), metformin (200 mg/kg), the combination (200 mg/kg each), or vehicle (2% Tween 80, 10 ml/kg) were administered orally twice a day (total dose 400 mg/kg/day) for 42 days to Zucker diabetic fatty rats (ZOF/Gmicrl-fa/fa). Groups of 8 rats were used for each group (6 weeks old, 190±10 grams). Animals were fasted for 24 hours prior to Oral Glucose Tolerance Test on day 43 (2 g/kg glucose load, PO). Blood samples were collected from the retro-orbital sinus 35 minutes before glucose load (Fasting glucose) and 90 minutes post oral glucose load. Serum glucose levels were determined by means of a Hitachi Model 750 Automatic Analyzer. The results of this experiment are shown in FIG. 23. Daily body weights and food intake over the same 43 days demonstrated no statistical difference between the four groups. In addition, no difference in fasting serum glucose levels (day 8, 15, 22, 29, 36 and 43) was seen between the four groups.

### Example 3

### Biochemical and Histological Analysis in a Diet Induced Obesity (DIO) Mouse Model

Diet induced obesity was established in mice as described above in Example 1. Biochemical and histological analyses were conducted on mice fed with a control diet (C), high fat diet (HF), or high fat diet plus 400 mg/kg/day resveratrol (HF+R400) (see Example 1).

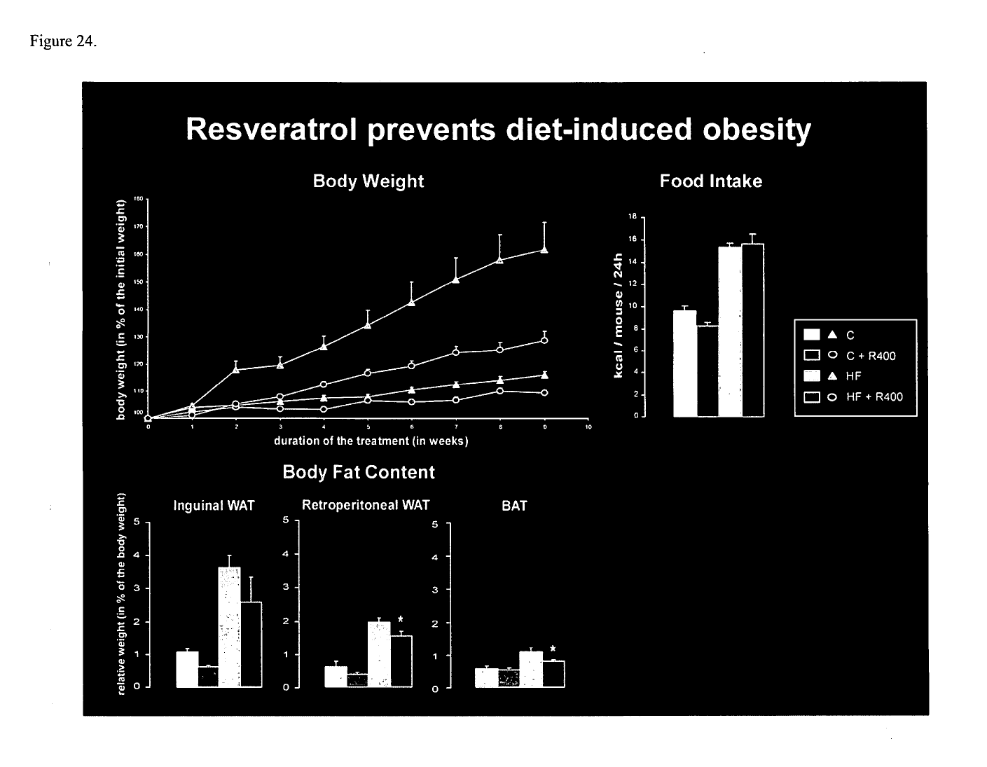

FIG. 24 shows the results of body weight evolution experiments, food intake experiments and body fat content experiment which were conduced as described above in Example 1. Animals were maintained on a control diet (C), control diet plus 400 mg/kg/day resveratrol (C+R400), high fat diet (HF) or high fat diet plus 400 mg/kg/day resveratrol (HF+R₄₀₀) diets for a 9 week period. The top left panel shows a graph of body weight evolution for mice in the four dietary groups over a nine week period. The top right panel shows a graph of food intake of mice in the four dietary groups expressed as kcal per 24 h. The bottom panels show comparisons of body fat content, as analyzed by dexa scanning, at week 9 of treatment for mice in the four dietary groups. Values are represented as the mean±SEM (n=10). BAT is brown adipose tissue (bottom right panel); Inguinal WAT is inguinal white adipose tissue (bottom left panel); and Retroperitoneal WAT is retroperitoneal white adipose tissue (bottom middle panel). Significant differences are indicated (p value).

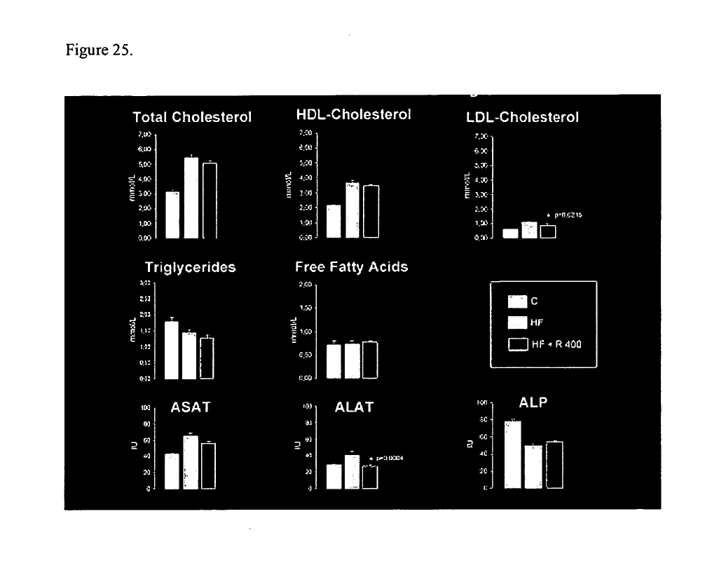

FIG. 25 shows the results of serum biochemical analysis of animals following 16 weeks on a control (C), high fat (HF) or high fat plus 400 mg/kg/day reservatrol (HF+R₄₀₀). The values shown are based on an average of measurements from 10 animals for each dietary group. Levels of total cholesterol, HDL-cholestrol, LDL-cholesterol, triglycerides, free fatty acids, aspartate aminotransferase (ALAT), alanine aminotransferase (ALAT), and alkaline phosphate (ALP) were determined using standard procedures. ASAT, ALP and ALAT were measured by kinetic UV and colour tests using methods based on the recommendations of the ‘International Federation for Clinical Chemistry’ (IFCC) on an Olympus AU-400 automated laboratory work station. AST was quantified using the OSR6109 reagent system which is based on the activity of AST that catalyzes the transamination of aspartate and 2-oxoglutarate to L-glutamate and oxalacetate. The subsequent reduction of oxalacetate to L-malate by malate dehydrogenase results in the conversion of NADH to NAD. The decrease in absorbance due to the consumption of NADH is measured at 340 nM and is proportional to the AST activity in the sample. ALT was quantified using the OSR6107 reagent system which is based on the activity of ALT that transfers the amino group from alanine to 2-oxoglutarate to form pyruvate and glutamate. The pyruvate is then reacted upon by lactate dehydrogenase which results in the conversion of NADH to NAD. As with the AST measurement, consumption of NADH is measured at 340 nM and is proportional to the amount of ALT activity in the sample. ALP is measured by determining the rate of conversion of p-nitro-phenyl phosphate to p-nitrophenol (pNP). The rate of change in absorbance due to the formation of pNP is measured bichromatically at 410/480 nM and is directly proportional to the amount of ALP activity in the sample. Values of all serum biochemical markers fell within the normal range for each dietary group.

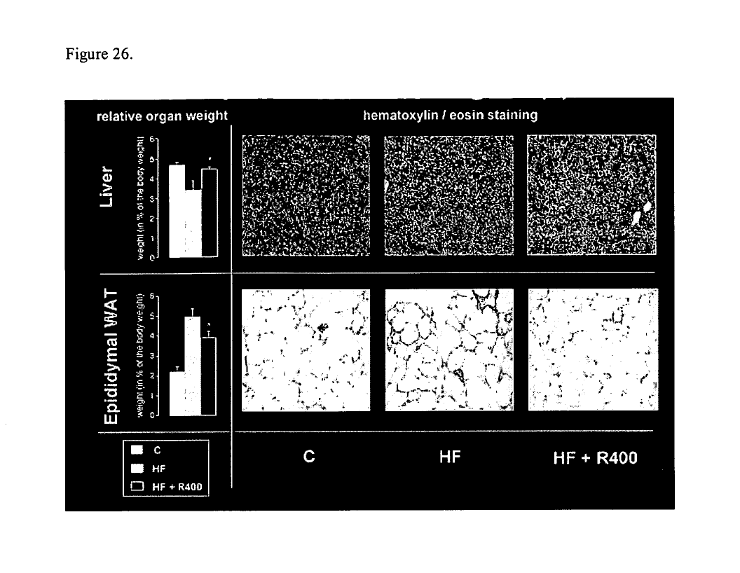

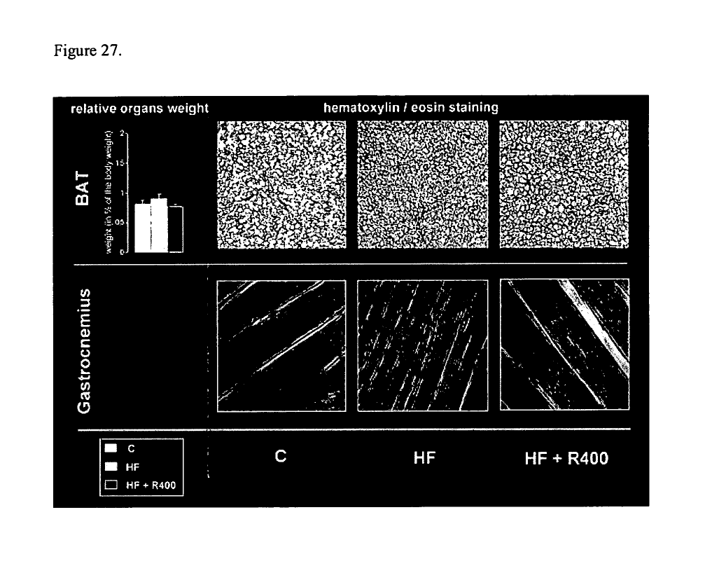

FIGS. 26 and 27 shows hematoxylin and eosin staining of liver, epididymal white adipose tissue (WAT), brown adipose tissue (BAT), and gastrocnemius muscle sections of animals following 16 weeks on control (C), high fat (HF), or high fat plus 400 mg/kg/day resveratrol (HF+R400) diets. After collection, tissues were fixed in 4% paraformaldehyde, processed and embedded in paraffin prior to sectioning (10 microns) and staining. Tissue processing, paraffin embedding, tissue sectioning and hematoxylin and eosin staining of histological sections were carried out using standard procedures and commercially available materials (see e.g., McManus J. F. A. and Mowry, R. W., Staining Methods. Histologic and Histochemcial, Harper and Row, New York 1960; Luna L. G., Hitopathological Methods and Color Atlas of Special Stains and Tissue Artifacts, Johnson Printers, Downers Grove, Ill. 1992; Gabe M., Techniques histologiques. Masson, Paris 1968; and world wide web at statlab.com). As shown in the figures, few histological changes were observed in any of the tissues between the various dietary groups.

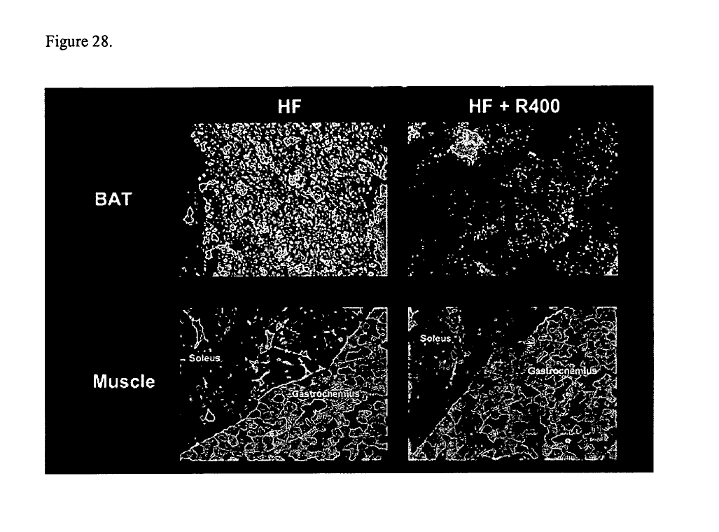

FIG. 28 shows succinate dehydrogenase staining of brown adipose tissue and muscle tissue (soleus and gastrocnemius) from mice following 16 weeks of high fat (HF) or high fat plus 400 mg/kg/day resveratrol (HF+R400) diets. Succinate dehydrogenase is a marker of mitochondrial activity and produces a dark stain in the photos. Tissues were collected and immediately frozen in methylbutane, then kept at −80° C. prior to sectioning and staining. Succinate Dehydrogenase staining was carried out using standard procedures and commercially available reagents (see e.g., Reichmann H and Wildenauer D, Histochemistry, 96: 251-3 (1991)). As shown in FIG. 28, succinate dehydrogenase staining was significantly higher for the mice receiving the HF+R400 diet in the brown adipose tissue and gastrocnemius tissues indicating the mitochondrial activity was higher in these tissues following administration of resveratrol. In contrast, little change was observed in the succinate dehydrogenase staining in the soleus tissue for mice on the HF and HF+R400 diets.

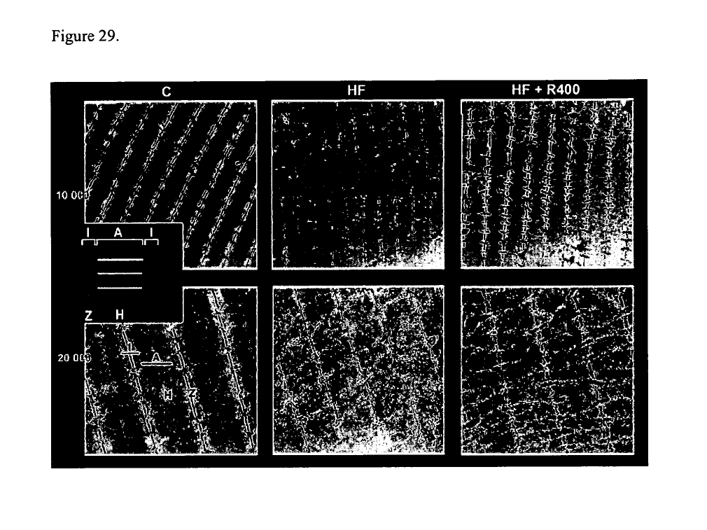

FIG. 29 shows transmission electron microscopy of gastrocnemius muscle (non-oxidative fibers) of mice following 16 weeks on control (C), high fat (HF) or high fat plus 400 mg/kg/day resveratrol (HF+R400) diets at 10,000 and 20,000 fold magnification. Transmission electron microscopy was carried out using standard techniques as described below. The mitochondria can be seen as the darker oblong Z lines crossing the I lines. As shown in the figure, the mice fed with the HF+R400 diet display more mitochondria than the mice fed the control or high fat diets.

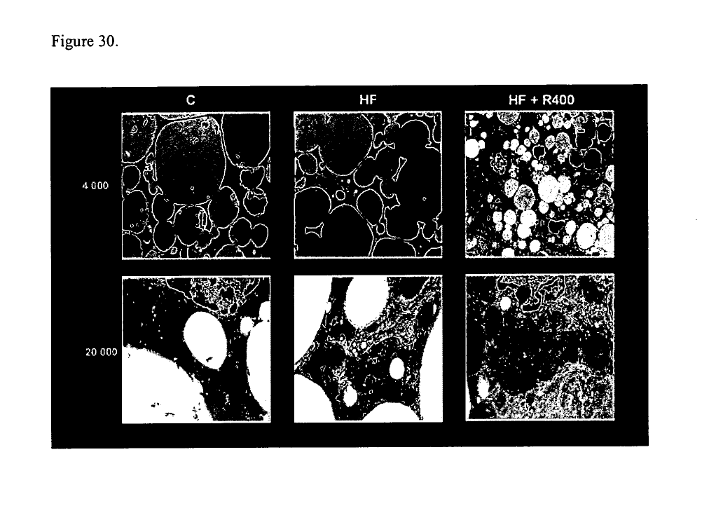

FIG. 30 shows transmission electron microscopy of brown adipose tissue of mice following 16 weeks on control (C), high fat (HF), or high fat plus 400 mg/kd/day resveratrol (HF+R400) at 4,000 and 20,000 fold magnification. Transmission electron microscopy was carried out using standard techniques as described below. Fat droplets may be observed at either magnification as white or light gray droplets and mitochondria can be observed at the higher magnification as roundish striated structures. Animals fed the high fat diet plus resveratrol had smaller fat droplets (top panel) and more mitochondria (bottom panel).

Transmission Electronic Microscopy/Preparation of Samples: The biopsies of gastrocnemius muscle and brown adipose tissue were cut in pieces of 1 mm and fixed immediately after collection in Kamovsky fixative (glutaraldehyde in cacodylate buffer) and kept at 4° C. without time limitation. The second step is the post-fixation with 1% osmium tetraoxide in 0.1M cacodylate buffer for 1 h at 4° C. Tissues were then dehydrated through successive baths of graded alcohol followed by a propylene oxide bath, and then a treatment with a propylene oxide and resine mix before to be embedded in a pure epoxy resine (araldite, Epon 812) which becomes solid after 48 h at 60° C. Semithin sections were cut at 2 μm and stained with toluidine blue, and histologically analysed by light microscopy. Ultrathin sections were cut at 70 nm and contrasted with uranyl acetate and lead citrate, and examined with a Philips 208 electron microscope.

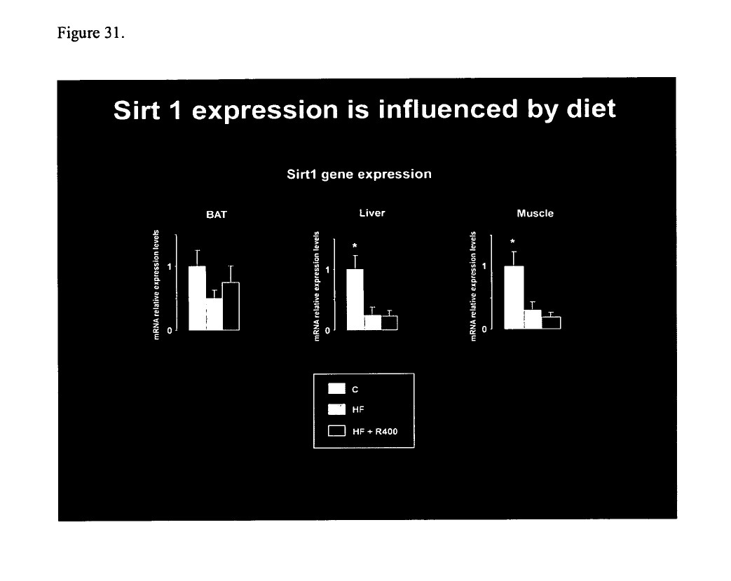

FIG. 31 shows Sirt1 mRNA levels measured in brown adipose tissue, liver and muscle of mice treated with control (C), high fat (HF), or high fat plus 400 mg/kg/day resveratrol (HF+R400) diets. The values shown are based on the average values from 6 animals for each dietary group. Values are expressed relative to housekeeping gene 18s and then expressed relative to chow diet (arbitrarily equal to 1). Relative gene expression was performed by real-time quantitative PCR using Sybrgreen incorporation (Lightcycler®, Roche Applied Science, Indianapolis, Ind.). Levels of protein expression and protein activity may also be determined. Protein expression level is determined by separating liver nuclear extracts by SDS-polyacrylamide gel electrophoresis and then immunoblotting is performed using a primary antibody specific for Sirt1 (rabbit IgG anti-Sir2, Upstate® Biotechnology, Lake Placid, N.Y.). Determination of Sirt1 activity is performed in liver nuclear extracts that are immunoprecipitated with an anti-PGC1α antibody (PGC1 H300: sc-13063, Santa Cruz Biotecnology, Inc., Santa Cruz, Calif.) followed by separation by SDS-PAGE and immunoblotting with an anti-acetylated lysine antibody (Cell Signaling Technology, Inc., Beverly, Mass.).

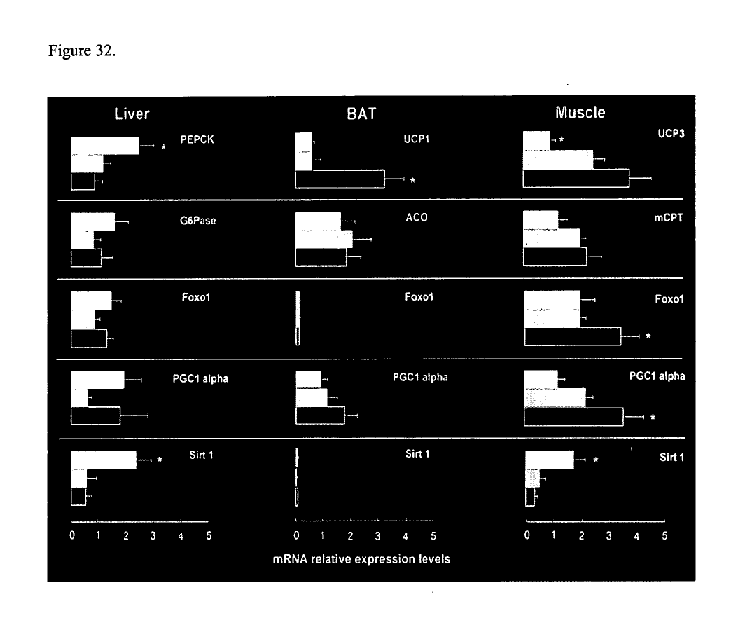

FIG. 32 shows relative gene expression of phosphenolpyruvate carboxykinase (PEPCK), glucose-6-phosphate (G6Pase), Foxol, PGCL-alpha (peroxisome proliferative activated receptor, gamma, coactivator 1, alpha), and Sirt1 from liver, uncoupling protein 1 (UCP1), acyl-CoA oxidase (ACO), Foxol, PGC1-alpha, and Sirt1 from brown adipose tissue (BAT), and uncoupling protein 3 (UCP3), muscle-type carnitine palmitoyltransferase (mCPT), Foxol, PGC1-alpha, and Sirt1 from muscle. Relative gene expression was performed by real-time quantitative PCR using Sybrgreen incorporation (Lightcycler®, Roche Applied Science, Indianapolis, Ind.).

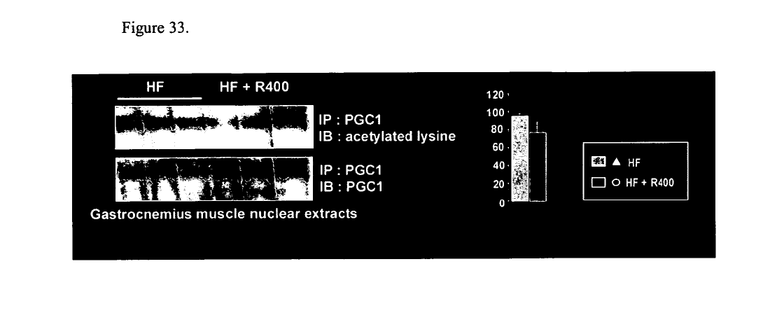

FIG. 33 shows the results of an immunoblot which demonstrates that resveratrol increases PGC1 alpha deacetylation. Nuclear extracts were prepared from gastrocnemius muscle from individual mice fed either a high fat diet (HF) or high fat diet with 400 mg/kg resveratrol (HF+R400) for 15 weeks. Following immunoprecipitation with a PGC 1 alpha antibody (Santa Cruz Biotechnology, cat #SC-13067), secondary western blots were probed with either a acetylated lysine specific monoclonal (Cell Signaling Technology, Cat #9441; top left panel) or a PGC1alpha antibody (Santa Cruz Biotechnology, cat #SC-13067; lower left panel). Exposures were scanned and the acetylation status of PGC1alpha compared to total PGC1alpha of animals on either the HF or HF+R400 is shown in the right hand panel.

### Example 4

### Analysis of Fat Absorption in a Diet Induced Obesity (Dio) Mouse Model

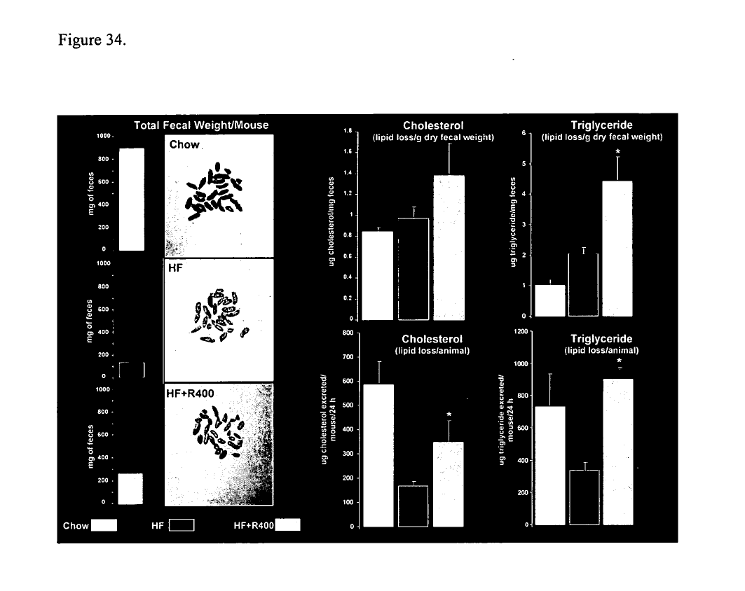

The fecal lipid content of mice fed diets of chow (C), high fat (HF) or high fat plus 400 mg/kg resveratrol (HF+R400) as described above was determined to investigate fat abosorption in the mice on the different dietary protocols. To conduct the analysis of fecal lipid content, mice are placed in metabolic cages consisting of a metal floor grid in place of mouse bedding. Food intake during a 24 h period is monitored and feces are collected in parallel to determine fat balance. Feces are dried in a vacuum oven at 70° C. and then carefully cleaned free of contaminating mouse bedding and/or food bits. Lipids are extracted from 100 mg aliquots using chloroform/methanol (2:1, v/v) for 30 min at 60° C. under constant agitation. Samples are cooled and then filtered through a Whatman No. 1 filter into a glass tube. An additional volume of chloroform/methanol is added and the sample is back-extracted by adding water and mixed well by vortexing. Phase separation is induced by low speed centrifugation and then the lower chloroform phase is transferred to a new tube. The sample is then evaporated to dryness and initially resuspended in chloroform/triton and then finally water. Fat extracts are partitioned according to total cholesterol (Biomerieux, enzymatic colour test CHOD-PAP) and triglyceride (Biomerieux, enzymatic colour test, GPO-PAP) content using enzymatic kit assays and manufacture provided protocols. Data is expressed as the amount of lipid per total amount of fecal weight. The results of the fecal lipid content analysis are presented in FIG. 34.

### Example 5

### Analysis of Endurance and Fat Absorption in a Diet Induced Obesity (Dio) Mouse Model

A second group of animals were subjected to a diet induced obesity study involving 16 weeks of a high fat diet as described above in Example 1. The animals in this study were divided into the following four dietary groups:
1: chow diet
2: chow diet+resveratrol (400 mg/kg/day)
3: high fat diet
4: high fat diet+resveratrol (400 mg/kg/day).

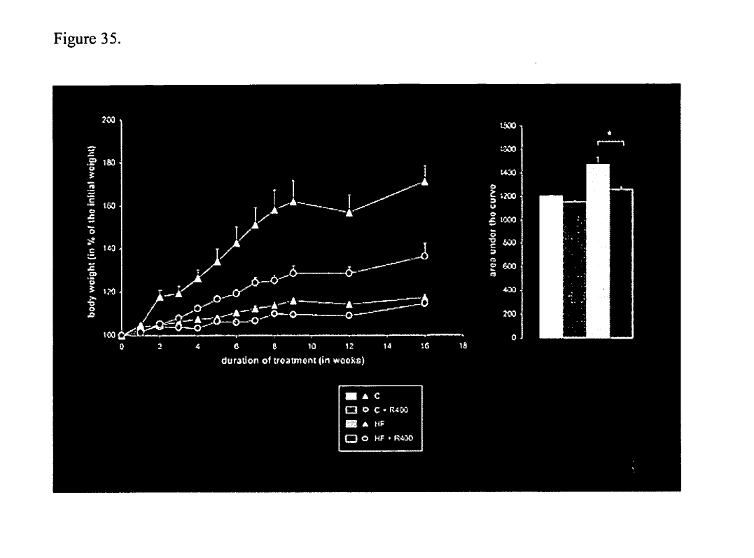

Body weight analyses for mice in all four dietary categories over a 16 week period are presented in FIG. 35.

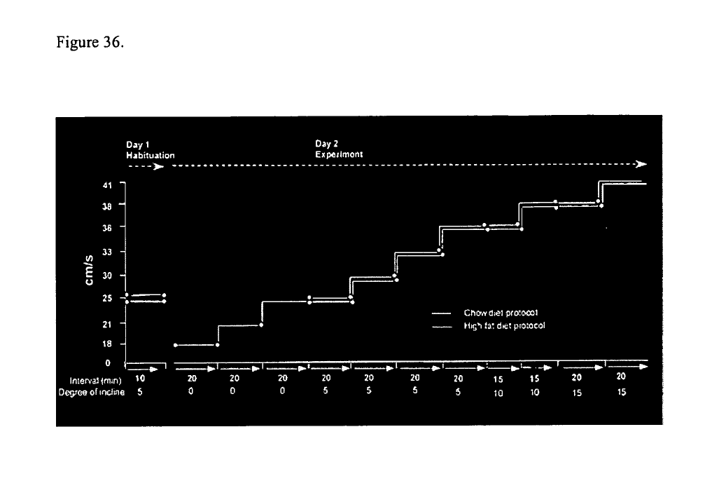

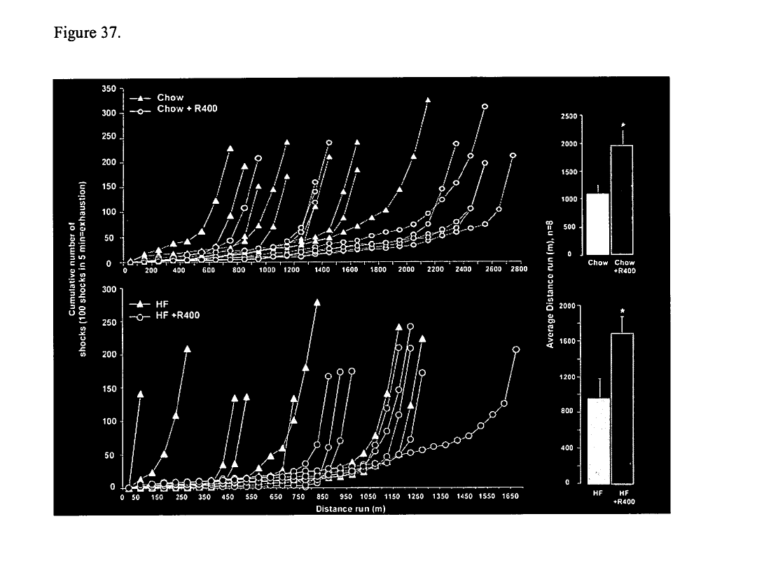

After 14-15 weeks on the indicated diet, the mice were subjected to an endurance study. A standard method for assessing exercise performance uses the treadmill, a system composed of a variable speed belt, enclosed in a plexiglass chamber, with a stimulus device consisting of a metal shock grid attached to the rear of the belt. Initially animals are acclimatized to the treadmill by using a habituation protocol on the day preceding the running test. With this procedure mice are placed in the chamber and run at 27 cm/s for 10 minutes with a 5° incline. For the actual running test, two incremental exercise protocols are used, one for high fat fed animals and one for chow. For chow animals, the experiment starts at 25 cm/s and a 5° incline and then increases in speed and incline are adjusted according to the outline in FIG. 36. For the high fat fed animals which generally weigh more and perform less easily, the beginning speed is 18 cm/s with a 0° incline and then increased according to FIG. 36. The distance run and the number of shocks obtained over 5 minute intervals are recorded. A mouse is considered exhausted and removed from the experiment when it receives approximately 100 shocks (at 2 mA each shock) in a period of five minutes. The duration of running and the total distance covered evaluates the performance of the mice (FIG. 37). All mice were fasted for 2 hours prior to running; the habituation protocol is performed in the afternoon and the running experiment the following morning.

### Example 6

### Effect of Resveratrol on Insulin Resistance

The current gold standard method for measuring insulin resistance is the euglycemic clamp. In this method glucose is “clamped” at a predetermined value (5 mmol/L for euglycaemia) by titrating a variable-rate of glucose (glucose infusion rate: GIR) against a fixed-infusion rate of insulin. Two to three days in advance of the study, a catheter is established in the femoral vein, under anesthesia (ketamine and xylazine), with the catheter fed underneath the mouse's skin and affixed behind their head. After surgery, mice are housed individually and allowed to recover for at least 48 hours, preferably enough time for them to regain their body weight. The clamps are performed in awake, unrestrained, unstressed and light-cycle inverted mice following a 5 hour fast. Mice are acclimatized (1 hour) to the tops of cages while their catheter is attached to a syringe-infusion pump. The catheter from the mouse is bifurcated to allow for simultaneous constant and variable injection of insulin and glucose, respectively. Base-line glucose values are measured by tail vein sampling prior to the injection of insulin. Catheter placement is assessed with a short priming dose (6 μl/min, 1 min) of insulin prior to the constant infusion of insulin at a flow rate of 2 μl/min equivalent to 18 mU of insulin/kg/min. Blood glucose values are monitored every 5 minutes throughout the test and within 15 minutes blood glucose is lowered and glucose infusion (20% solution in saline) can be started. The glucose infusion rate (GIR) is varied until euglycemia (±15%) has been reached and maintained. At this point the animal is “clamped” and the degree of insulin resistance is inversely related to the amount of glucose necessary to maintain the required blood glucose concentrations. The GIR (mg glucose/kg animal*min) is then calculated as an average during the last 60 minutes of the clamp. When the average GIR of one animal is greater than another, it indicates better insulin sensitivity or that the clearance of glucose from the plasma is much faster.

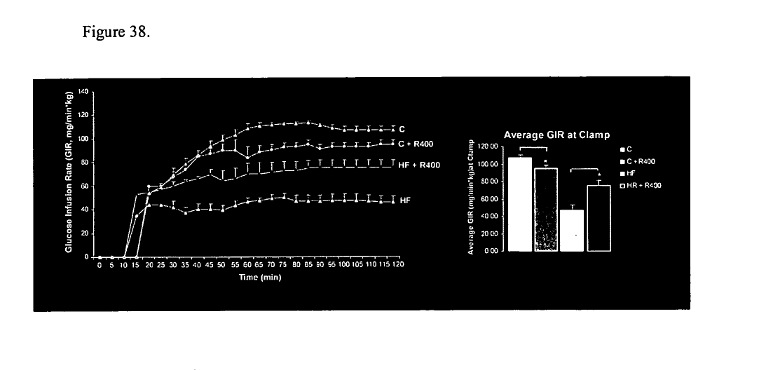

FIG. 38 shows the effect of resveratrol on insulin sensitivity as measured by hyperinsulinemic (18 mU/kg/min) euglycemic (5.5 mmol/l) clamp. The left hand panel shows glucose infusion rates (GIR) for groups of animals following 14 weeks on either a control diet (C), control diet plus 400 mg/kg resveratrol (C+R400), high fat diet (HF) or high fat diet plus 400 mg/kg resvertrol (HF+R400). The right hand panel shows average GIR at steady state clamp.

### Equivalents

The present invention provides among other things sirtuin-activating compounds and methods of use thereof. While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

### Incorporation by Reference

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) (www.tigr.org) and/or the National Center for Biotechnology Information (NCBI) (www.ncbi.nlm.nih.gov).

Also incorporated by reference are the following: PCT Publications WO 2005/002672; 2005/002555; and 2004/016726.

1-123. (canceled)

124. A pharmaceutical dosage form, comprising a quantity of a sirtuin activating compound having a sirtuin activating effect equal to or greater than 200 mg resveratrol.

125. The pharmaceutical dosage form of claim 124, wherein the sirtuin activating compound is resveratrol or nicotinamide riboside.

126. The pharmaceutical dosage form of claim 124, further comprising a lipid-lowering, an anti-obesity or an anti-diabetic agent or a combination thereof.

127. A method of treating obesity, reducing the weight, preventing weight gain, and/or treating an insulin resistance disorder in a subject in need thereof, comprising administering daily to the subject an amount of a sirtuin activating compound that has a sirtuin activating effect equal to or greater than 18 mg/kg resveratrol.

128. A method of treating obesity in a subject in need thereof that consumes a high fat diet, comprising administering to the subject an amount of a sirtuin activating compound.

129. A method of treating obesity in a subject in need thereof, comprising administering to the subject an effective amount of a sirtuin activating compound, wherein said subject does not reduce calorie consumption, increase activity or a combination thereof to an extent sufficient to cause weight loss in the absence of a sirtuin activating compound.

130. A method of protecting pancreatic beta cells in a subject in need thereof, comprising administering to the subject an effective amount of a sirtuin activating compound.

131. The method of any of claims 127-130, wherein the sirtuin activating compound is resveratrol or nicotinamide riboside.

132. The method of any of claims 127-130, further comprising administering to the subject a lipid-lowering, an anti-obesity or an anti-diabetic agent or a combination thereof.

133. A food or beverage fit for consumption by a mammal, wherein said food or beverage comprises a supplement of one or more sirtuin activating compounds and wherein (a) the concentration of the one or more sirtuin activating compounds in said food or beverage has a sirtuin activating effect equal to or greater than the sirtuin activating effect of 11 mg/g resveratrol; (b) said food or beverage does not include grapes, mulberries, blueberries, raspberries, peanut, milk, yeast or an extract thereof; (c) an 8 fluid ounce serving of the beverage comprising a quantity of a sirtuin activating compound having a sirtuin activating effect equal to or greater than the sirtuin activating effect of 25 mg resveratrol; (d) a single serving of the food comprises a quantity of a sirtuin activating compound having a sirtuin activating effect equal to or greater than the sirtuin activating effect of 100 mg resveratrol; or (e) said food or beverage comprises a supplement of one or more stabilized sirtuin activating compounds.

134. The food or beverage of claim 133, wherein the sirtuin activating compound is resveratrol or nicotinamide riboside.

135. The food or beverage of claim 133, further comprising one or more lipid lowering, anti-obesity or anti-diabetic agents or a combination thereof.

136. A method for treating a disease or disorder in a subject that would benefit from increased mitochonadrial activity, comprising administering daily to a subject in need thereof an amount of a sirtuin activating compound that has a sirtuin activating effect equal to or greater tan 18 mg/kg resveratrol.

137. The method of claim 136, wherein the sirtuin modulating compound increases mitochondrial activity without increasing mitochondrial mass or wherein the sirtuin modulating compound increases mitochondrial mass.

138. The method of claim 136, wherein the sirtuin activating compound is resveratrol or nicotinamide riboside.

139. The method of claim 136, further comprising administering to the subject one or more of the following: a vitamin, cofactor or antioxidant.

140. The method of claim 136, further comprising administering to the subject one or more agents that alleviate a symptom of the disease or disorder.

141. A method for treating hypothermia, enhancing motor performance or muscle endurance, decreasing fatigue, or increasing recovery from fatigue, comprising administering to a subject a therapeutically effective amount of at least one sirtuin activating compound.

142. The method of claim 141, wherein the sirtuin activating compound is resveratrol or nicotinamide riboside.

143. A method for treating or preventing muscle tissue damage associated with hypoxia or ischemia or a condition wherein motor performance or muscle endurance is reduced comprising administering daily to a subject in need thereof an amount of a sirtuin activating compound that has a sirtuin activating effect equal to or greater than 18 mg/kg resveratrol.

144. A method for increasing cellular ATP levels in a subject, comprising administering to the subject a therapeutically effective amount of a sirtuin activating compound.

145. A method for prolonging the lifespan of a subject, treating or preventing a neurodegenerative disorder in a subject, treating or preventing a blood coagulation disorder, treating or preventing an ocular disease or disorder, treating or preventing chemotherapeutic induced neuropathy, treating or preventing neuropathy associated with an ischemic event or disease, treating or preventing a polyglutamine disease or treating or preventing a disease or disorder associated with cell death or aging in a subject, comprising administering daily to a subject in need thereof an amount of a sirtuin activating compound that has a sirtuin activating effect equal to or greater than 18 mg/kg resveratrol.

146. The method of claim 145, wherein the sirtuin activating compound is resveratrol or nicotinamide riboside.
